(19) United States
(12) Patent Application Publication
(10) Pub. No.: US 2022/0017516 A1
(43) Pub. Date: Jan. 20, 2022

(54) LOX INHIBITORS

(71) Applicant: THE INSTITUTE OF CANCER RESEARCH: ROYAL CANCER HOSPITAL, London (GB)

(72) Inventors: Mohammed ALJARAH, Macclesfield (GB); Dan NICULESCU-DUVAZ, Macclesfield (GB); Leo LEUNG, Macclesfield (GB); Deborah SMITHEN, Macclesfield (GB); Michael BROWN, Macclesfield (GB); Lawrence Christopher DAVIES, London (GB); Caroline SPRINGER, Macclesfield (GB)

(21) Appl. No.: 17/293,970

(22) PCT Filed: Nov. 15, 2019
(86) PCT No.: PCT/GB2019/053242

§ 371 Date: May 14, 2021

(30) Foreign Application Priority Data
Nov. 16, 2018 (GB) 1818750.0

(51) Int. Cl. C07D 471/08; C07D 487/08; C07D 295/088; C07D 487/04; C07D 239/48; C07D 295/108; C07D 487/10; A61P 35/00

(52) U.S. Cl. CPC C07D 471/08; C07D 487/08; C07D 295/088; A61P 35/00; C07D 239/48; C07D 295/108; C07D 487/10; C07D 487/04

(57) ABSTRACT

The disclosure relates to compounds of Formula I, or pharmaceutically acceptable salts thereof, Formula (I) as defined herein. Compounds according to Formula I are pharmacologically effective as lysyl oxidase (LOX) inhibitors and are believed to be useful in the treatment of, for instance, cancer.

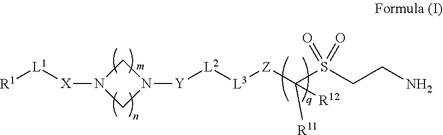

### TECHNICAL FIELD

The present invention relates to compounds useful as lysyl oxidase (LOX) and lysyl oxidase-like (LOXL) family members (LOXL1, LOXL2, LOXL3, LOXL4) inhibitors, to pharmaceutical compositions comprising the compounds, to the compounds for use in the treatment of conditions mediated by LOX and/or LOXL, for example cancer; to a LOX inhibitor for use in the treatment of a cancer associated with EGFR.

### BACKGROUND

LOX (protein-6-lysine-oxidase; EC 1.4.3.13) is an extracellular enzyme that catalyses oxidative deamination of the primary amines of lysine and hydroxylysine in proteins such as collagen and tropoelastin to generate peptidyl-[α]-aminoadipic-[δ]-semialdehyde, an aldehyde that spontaneously condenses to form inter- and intra-chain cross-links (Lucero and Kagan 2006). LOX regulates maturation of proteins in the extracellular matrix (ECM), thereby contributing to ECM tensile strength and function and so playing an important role in connective tissue remodelling. Other proteins have been reported as substrates for oxidation by LOX, such as basic fibroblast growth factor, PDGFR-β and other cationic proteins (Kagan and Li 2003, Li, Nugent et al. 2003, Lucero and Kagan 2006, Lucero, Ravid et al. 2008).

LOX is secreted as a precursor protein that is proteolytically processed by procollagen C-proteinases (bone morphogenetic protein 1—BMP-1) and mammalian tolloid-like protein (mTLL-1)(Uzel, Scott et al. 2001) to generate an 18 kDa pro-peptide and the 32 kDa active LOX enzyme (Lucero and Kagan 2006). The catalytic domain contains copper and a lysine-tyrosylquinone (LTQ) cofactor. LTQ is formed by post-translational oxidation of a catalytic site tyrosine (Tyr349), which then condenses onto a lysine, also within the catalytic site (Lys314), to form a stable covalent modification that is an essential part of the catalytic mechanism (Lucero and Kagan 2006) (Kagan and Li 2003).

LOX is part of a protein family consisting of five paralogues, LOX, LOX-like 1 [LOXL1], LOX-like 2 [LOXL2], LOX-like 3 [LOXL3] and LOX-like 4 [LOXL4]), all containing a conserved catalytic region. LOX enzymes play a crucial role in maintaining ECM stability, by initiating and regulating the crosslinking of collagens and elastin within the extracellular matrix (ECM). The activity of these enzymes is key to maintaining the normal tensile and elastic features of connective tissue of many organ systems within the body. LOX expression decreases during ageing indicating that its activity is especially important during development.

In addition to its role in tissue remodelling, LOX also plays a critical role in primary cancer and metastasis. Studies have shown that LOX plays a fundamental role in the growth of primary tumours in colorectal and lung cancer (Gao, Xiao et al. 2010, Baker, Cox et al. 2011) and glioblastoma (Mammoto, Jiang et al. 2013).

Expression of LOX is elevated in more than 70% of breast cancer patients with Estrogen Receptor negative disease, in 80% of head and neck cancer patients, in 33% of primary colorectal carcinomas (CRC) and 48% of metastatic tissues from patients with CRC (Baker, Cox et al. 2011), and in cirrhotic hepatocellular carcinoma (HCC) patients with a history of alcoholism (Huang, Ho et al. 2013). As discussed in more detail in the description, LOX is also overexpressed in numerous other cancers including lung, prostate and pancreatic cancers.

Elevated LOX expression is also associated with metastasis and decreased patient survival (Baker, Cox et al. 2011, Wilgus, Borczuk et al. 2011)

Other members of the LOX family have been implicated in proliferative diseases such as cancer. LOXL2 is another member of the LOX family that is involved in the cross-linking of extracellular collagens and elastin (Vadasz, Kessler et al. 2005) (Kim, Kim et al. 2010). In addition to conserved C-terminal region, the LOXL2 protein has scavenger receptor cysteine-rich regions that are commonly found in cell surface receptors and adhesion molecules, as well as a cytokine receptor-like domain.

LOXL2 expression has been found upregulated in breast, gastric, colon, esophageal, head and neck, lung and laryngeal carcinomas, as reviewed in Barker et al (Barker, Cox et al. 2012) and in renal cells carcinoma (Hase, Jingushi et al. 2014) (Nishikawa, Chiyomaru et al. 2015).

Studies have suggested that LOX and LOXL2 do not compensate one another, as manipulation of LOX expression did not affect LOXL2 levels in a colorectal cancer model (Baker, Cox et al. 2011). Thus, while LOX and LOXL2 are involved in similar extra-cellular processes, it appears that they have distinct roles.

LOXL1 was found to be overexpressed in metastatic non-small cells lung cancer (NSCLC), and the metastatic phenotype can be reduced by inhibition with LOXL1 siRNA (Lee, Kim et al. 2011).

LOXL3 mRNA was expressed in Hs578T highly invasive breast cancer cells, but not in poorly invasive and non-metastatic breast cancer cells MCF7 and T47D (Kirschmann, Seftor et al. 2002). Overexpression of LOXL3 in MDCK epithelial cells induces an epithelial-mesenchymal transition (EMT) process, which is a key step in the progression of metastasis (Peinado, Del Carmen Iglesias-de la Cruz et al. 2005).

In a study on the mRNA levels of LOXL4 in head and neck squamous cell carcinomas, high expression of LOXL4 gene was detected in 71% of all carcinomas and only in 9% of the healthy mucosa samples, indicating that LOXL4 may serve as a selective molecular marker in primary and metastatic head and neck carcinoma (Scola and Gorogh 2010). Up-regulation of LOXL4 was demonstrated in invasive HNC and revealed a significant correlation between LOXL4 expression and local lymph node metastases and higher tumour stages (Goeroegh, Weise et al. 2007). LOXL4 promotes metastasis in gastric cancer (Li, Zhao et al. 2015). LOXL4 together with LOXL2 has been found to be required for metastatic niche formation in a breast orthotopic mouse model (Wong, Gilkes et al. 2011).

LOX and LOXL are implicated in fibrotic diseases, such as liver fibrosis, lung fibrosis, kidney fibrosis, cardiac fibrosis, myelofibrosis and schleroderma. Both LOX and LOXL are highly expressed in fibrotic areas, in surrounding myofibroblasts and in serum of patients with fibrotic conditions (Kagan 1994) (Kim, Peyrol et al. 1999) (Siegel, Chen et al. 1978) (Jourdan-Le Saux, Gleyzal et al. 1994) (Murawaki, Kusakabe et al. 1991).

LOX is also implicated in cardiovascular disease. As discussed in the detailed description of the invention, LOX inhibition may prove beneficial in the treatment or prevention of cardiovascular conditions, including hypertensive heart disease, heart failure, cardiac hypertrophy and atherosclerosis.

LOX is associated with the amyloid-beta (Δβ) related pathological hallmarks (such as cerebral amyloid angiopathy and senile plaques) of both Alzheimer's disease (AD) and hereditary cerebral hemorrhage with amyloidosis of the Dutch type (HCHWA-D) pathogenesis (Wilhelmus, Bol et al. 2013). LOX activity is increased in the hippocampal samples of Alzheimer's disease and also in non-Alzheimer's dementia (Gilad, Kagan et al. 2005). LOX is increased at the site of brain injury (Gilad, Kagan et al. 2001) and spinal cord injury and its inhibition lead to accelerated functional recovery in an unilateral spinal cord dissection model (Gilad and Gilad 2001).

LOXLs are implicated in pulmonary diseases. LOXL2 and LOXL3 are likely to have a role in Primary Alveolar Proteinosis (PAP) since both are expressed in PAP tissue, but not normal lung tissue (Neufeld and Brekhman 2009).

LOX inhibition may be beneficial in the treatment of various ocular conditions. Inhibition of LOX or LOXL2 prevents neovascularization and fibrosis following laser-induced choroidal neovascularization (CNV). Therefore LOX and LOXL inhibitors can be useful in the treatment of conditions characterized by neovascularization, such as age-related macular degeneration (AMD), diabetic retinopathy and retinopathy of prematurity (Stalmans, Marshall et al. 2010).

LOX is implicated in inflammatory conditions and may be useful in the treatment of conditions including, but not limited to acute respiratory distress syndrome (ARDS) (Mambetsariev, Tian et al. 2014).

LOX is the main isoenzyme expressed in human adipose tissue and that its expression is strongly upregulated in samples from obese patients. β-aminopropionitrile reduces body weight gain and improves the metabolic profile in diet-induced obesity in rats (Miana, Galan et al. 2015) and reduces local adipose tissue inflammation (Halberg, Khan et al. 2009).

LOX is upregulated in endometriosis and may be implicated in the establishment and progression of endometriotic lesions (Ruiz, Dutil et al. 2011) (Dentillo, Meola et al. 2010).

Certain LOX inhibitors are known. These include β-aminopropionitrile (BAPN), haloamines, 1,2-diamines, allyl and propargyl amines, hydrazines, semicarbazide and thiolactones, benzylamines, mercaptopyridine and pyridazinone compounds (Pinnell and Martin 1968) (Tang, Simpson et al. 1984) (Palfreyman, McDonald et al. 1989) (Sayre 2007) (Carrington, Bird et al. 1984) (Levene, Sharman et al. 1992) (Liu, Nellaiappan et al. 1997) (Williamson and Kagan 1987) (Anderson, Bartlett et al. 2007) (Schohe-Loop, Burchardt et al. 2003) (Burchardt 2006, Aslam, Miele et al. 2015). However, in general these compounds are either non-selective, lack potency or are unsuitable for use in patients. It is believed that the only LOX inhibitor which has progressed to clinical trials in humans is BAPN. However, it is believed that this compound has not been used clinically since 1978. More recent LOX and LOXL2 inhibitors have been described: LOX inhibitors containing hydrazine and hydrazide groups (Burke et al, 2017); LOXL2 inhibitors: derivatives of haloallylamine (Chang et al, 2017), pyridines (Rowbottom et al, 2016a; Rowbottom et al, 2016b), pyrimidines (Rowbottom & Hutchinson, 2017a) and chromenones (Rowbottom & Hutchinson, 2017b).

WO 2017/141049 A1 discloses methylamine derivatives as LOX inhibitors.

WO 2004/110996 A1 relates to compounds disclosed to exhibit neurokinin (NK) inhibitory properties and useful for treatment of neurokinin-mediated conditions.

WO 2007/027734 A2 relates to bicyclic and bridged nitrogen heterocycles as which are disclosed to be effective as modulators of one or more chemokine receptors (CCRs) and useful in treating inflammatory and immune disorder conditions and diseases.

WO 2011/050198 A1 and WO 2012/145581 A1 relate to disubstituted octahydropyrrolo[3,4-c]pyrroles disclosed to be orexin receptor modulators and useful for treatment of diseases or conditions mediated by orexin activity, such as insomnia.

WO 2009/137308 A1 relate to disclosed to selective ligands for neuronal nicotinic receptors (NNRs) and useful as for treating a condition or disorder where modulation of α4β2 NNR activity is of therapeutic benefit.

### BRIEF SUMMARY OF THE DISCLOSURE

It is an object of the present invention to provide novel compounds that are useful for the treatment of diseases, disorders and/or conditions which is affected and/or mediated by LOX, such as cancer or fibrosis.

In one aspect, the present invention provides a compound having the structure of Formula (I):

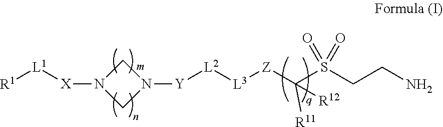

or a pharmaceutically acceptable salt thereof, wherein
m and n are each independently selected from 1, 2, 3 or 4, and where two ring carbon atoms of the cyclic diamine moiety of formula

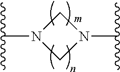

may be
(i) optionally linked by a bond,
(ii) optionally bridged by —(CR⁹R¹⁰)ₒ—, where R⁹ and R¹⁰ are at each occurrence independently selected from H and unsubstituted C₁₋₄ alkyl and o is 1, 2, 3 or 4; or
(iii) optionally linked by a spiro carbon atom; and
each ring carbon atom of said cyclic diamine moiety may be optionally substituted by one or two substituents independently selected from C₁-C₆ alkyl, C₁-C₆ alkyl substituted with —OR^{2a}, or C₁-C₆ alkyl substituted with —NR^{2a}R^{2b};
   L¹ and L² are each independently selected from a bond, —O—, —C(O)—, —C(O)O—, —OC(O)—, —C(O)NR³—, —NR³C(O)—, —NR³—, —SO₂NR³—, —NR³SO₂—, —S—, —SO₂—, —SO₂O—, —OSO₂—, —NR³SO₂NR⁴—, —NR³C(O)NR⁴—, —NR³C(O)O— or —OC(O)NR³—;
   L³ is selected from a bond, C₁-C₄ alkylene, C₂-C₄ alkenylene or C₂-C₄ alkynylene, where
any alkylene, alkenylene or alkynylene in L³ may be optionally substituted by one or two substituents independently selected from halo, cyano, oxo, hydroxy, carboxy, R², —OR², —C(O)R², —C(O)OR², —OC(O)R², —C(O)NR⁶R⁷, —NR⁶C(O)R⁷, —NR⁶R⁷, —SO₂NR⁶R⁷, —NR⁶SO₂R⁷, —SR⁶, —SO₂R⁶, —SO₂OR⁶, —OSO₂R⁶, —NR⁶SO₂NR⁷R⁸, —NR⁶C(O)NR⁷R⁸, —NR⁶C(O)OR⁷ or —OC(O)NR⁶R⁷;
   X, Y and Z are each independently selected from a bond or a 3- to 12-membered ring system, including 0, 1, 2 or 3 heteroatoms selected from N, O or S in the ring system, where
any ring system in X, Y and Z may be optionally substituted by one or more substituents independently selected from halo, cyano, oxo, hydroxy, carboxy, R², —OR², —C(O)R², —C(O)OR², —OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵, —R³C(O)NR⁴R⁵, —NR³C(O)OR⁴ or —OC(O)NR⁴R⁵;
   R¹ is selected from hydrogen, halogen, C₁-C₆ alkyl, C₂-C₆ alkenyl, C₂-C₆ alkynyl or a 3- to 12-membered ring system (e.g. cycloalkyl, heterocyclyl, aryl or heteroaryl), including 0, 1, 2 or 3 heteroatoms selected from N, O or S in the ring system, where
any alkyl, alkenyl or alkynyl in R¹ may be optionally substituted by one, two or three substituents independently selected from halo, cyano, amino, oxo, hydroxy, carboxy, R², —OR², —C(O)R², —C(O)OR², —OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵, —NR³C(O)NR⁴R⁵, —NR³C(O)OR⁴ or —OC(O)NR⁴R⁵; and
any ring system in R¹ may be optionally substituted by one or more substituents independently selected from halo, cyano, oxo, hydroxy, carboxy, R², —OR², —C(O)R², —C(O)OR², —OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵, —NR³C(O)NR⁴R⁵, —NR³C(O)OR⁴ or —OC(O)NR⁴R⁵;
   R² is at each occurrence independently selected from C₁-C₆ alkyl, C₂-C₆ alkenyl, C₂-C₆ alkynyl or C₃-C₆ cycloalkyl, where
any alkyl, alkenyl, alkynyl or cycloalkyl in R² may be optionally substituted by one, two or three substituents independently selected from halo, cyano, oxo, R^{2a}, —OR^{2a}, —C(O)R^{2a}, —C(O)OR^{2a}, —OC(O)R^{2a}, —C(O)NR^{2a}R^{2b}, —NR^{2a}C(O)R^{2b}, —NR^{2a}R^{2b}, —SO₂NR^{2a}R^{2b}, —NR^{2a}SO₂R^{2b}, —SR^{2a}, —SO₂R^{2a}, —SO₂OR^{2a}, —OSO₂R^{2a}, —NR^{2a}SO₂NR^{2b}R^{2c}, —NR^{2a}C(O)NR^{2b}R^{2c}, —NR^{2a}C(O)OR^{2b} or —OC(O)NR^{2a}R^{2b};
   R^{2a}, R^{2b} and R^{2c} are at each occurrence independently selected from hydrogen or unsubstituted C₁-C₄ alkyl;
   R³, R⁴ and R⁵ are at each occurrence independently selected from hydrogen, C₁-C₆ alkyl or C₃-C₆ cycloalkyl, where
any alkyl or cycloalkyl in R³, R⁴ and R⁵ may be optionally substituted by one, two or three substituents independently selected from halo, cyano, oxo, hydroxy, carboxy, R², —OR², —C(O)R² or —C(O)OR², and
when R⁴ is optionally substituted C₁-C₆ alkyl and R⁵ is optionally substituted C₁-C₆ alkyl, then R⁴ and R⁵ together with the nitrogen atom to which they are attached in —C(O)NR⁴R⁵, —NR⁴R⁵, —SO₂NR⁴R⁵, —NR³SO₂NR⁴R⁵, —R³C(O)NR⁴R⁵ or —OC(O)NR⁴R⁵ may form a 3- to 6-membered heterocycloalkyl;
   R⁶, R⁷ and R⁸ are at each occurrence independently selected from hydrogen, C₁-C₆ alkyl or C₃-C₆ cycloalkyl, where
any alkyl or cycloalkyl in R⁶, R⁷ and R⁸ may be optionally substituted by one, two or three substituents independently selected from halo, cyano, oxo, hydroxy, carboxy, R^{2a}, —OR^{2a}, —C(O)R^{2a}, —C(O)OR^{2a}, —C(O)NR^{2a}R^{2b}, —NR^{2a}C(O)R^{2b}, —NR^{2a}R^{2b}, —SO₂NR^{2a}R^{2b}, —NR^{2a}SO₂R^{2b}, —SR^{2a}, —SO₂R^{2a}, —SO₂OR^{2a}, —OSO₂R^{2a}, —NR₂SO₂NR^{2a}R^{2b}, —NR^{2a}C(O)NR^{2b}R^{2c}, —NR^{2a}C(O)OR^{2b} or —OC(O)NR^{2a}R^{2b};
   R¹¹ and R¹² are independently selected from hydrogen and C₁-C₆ alkyl; and
   q is 0, 1 or 2;
   provided at least one of L², Y, L³ and Z is not a bond;
   provided —SO₂—(CH₂)₂—NH₂ in Formula I is linked to the remainder of the compound of Formula I via a carbon atom;
   provided when X is a bond, then L¹ is selected from a bond, —C(O)—, —OC(O)—, —NR³C(O)—, —NR³SO₂—, —SO₂— and —OSO₂—;
   provided when Y is a bond, then L² is selected from a bond, —C(O)—, —C(O)O—, —C(O)NR³—, —SO₂NR³—, —SO₂— and —SO₂O; and
   provided the compound is not

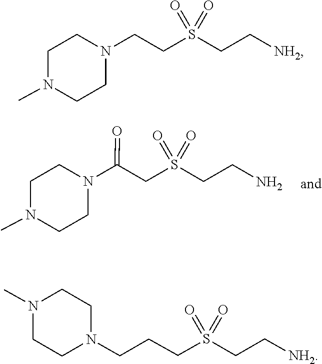 [SMILES: CN1CCN(CCS(=O)(=O)CCN)CC1 ; CN1CCN(C(=O)CS(=O)(=O)CCN)CC1 ; CN1CCN(CCCS(=O)(=O)CCN)CC1]

Also provided is a pharmaceutical composition comprising a compound of the invention, or a pharmaceutically acceptable salt thereof, and a pharmaceutically acceptable excipient.

Also provided is a compound of the invention, or a pharmaceutically acceptable salt thereof, or a pharmaceutical composition of the invention, for use as a medicament. In some embodiments, there is provided a compound of the invention, or a pharmaceutically acceptable salt thereof, for use in the treatment of a disease or a medical condition mediated by LOX.

Also provided is a method of treating a disease or a medical condition mediated by LOX in a subject, the method comprising administering to the subject an effective amount of a compound of the invention or a pharmaceutical composition of the invention.

In some embodiments, a compound of the invention, or a pharmaceutically acceptable salt thereof, or a pharmaceutical composition of the invention, is for use in the treatment of a proliferative disease, particularly cancer.

In some embodiments, a compound of the invention, or a pharmaceutically acceptable salt thereof, or a pharmaceutical composition of the invention, is for use in the treatment or prevention of cancer associated with overexpression of EGFR.

In some embodiments, a compound of the invention, or a pharmaceutically acceptable salt thereof, or a pharmaceutical composition of the invention, is for use in the treatment of fibrotic disease, such as liver fibrosis, lung fibrosis, kidney fibrosis, cardiac fibrosis, myelofibrosis or schleroderma.

### DETAILED DESCRIPTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The term “halo” or “halogen” refers to one of the halogens, group 17 of the periodic table. In particular the term refers to fluorine, chlorine, bromine and iodine. Preferably, the term refers to fluorine or chlorine.

The term Cₘ₋ₙ refers to a group with m to n carbon atoms. Cₘ₋ₙ is herein also referred to as Cₘ-Cₙ.

The term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl and n-hexyl. “C₁₋₄ alkyl” similarly refers to such groups containing up to 4 carbon atoms. Alkylene groups are divalent alkyl groups and may likewise be linear or branched and have two points of attachment to the remainder of the molecule. Furthermore, an alkylene group may, for example, correspond to one of those alkyl groups listed in this paragraph. The alkyl and alkylene groups may be unsubstituted or substituted by one or more substituents. Possible substituents are described below. Substituents for the alkyl group may be halogen, e.g. fluorine, chlorine, bromine and iodine, OH, C₁-C₄ alkoxy. Other substituents for the alkyl group may alternatively be used.

The term “C₁₋₆ haloalkyl”, e.g. “C₁₋₄ haloalkyl”, refers to a hydrocarbon chain substituted with at least one halogen atom independently chosen at each occurrence, for example fluorine, chlorine, bromine and iodine. The halogen atom may be present at any position on the hydrocarbon chain. For example, C₁₋₆ haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl.

The term “C₂₋₆ alkenyl” includes a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂₋₆ alkenyl” may be ethenyl, propenyl, butenyl, butadienyl, pentenyl, pentadienyl, hexenyl and hexadienyl.

The term “C₂₋₆ alkynyl” includes a branched or linear hydrocarbon chain containing at least one triple bond and having 2, 3, 4, 5 or 6 carbon atoms. The triple bond may be at any possible position of the hydrocarbon chain. For example, the “C₂₋₆ alkynyl” may be ethynyl, propynyl, butynyl, pentynyl and hexynyl.

As used herein, the term “cycloalkyl” represents a saturated monocyclic or polycyclic (e.g. bicyclic) aliphatic ring system containing ring carbon atoms. The term cycloalkyl includes both fused and bridged polycyclic systems.

The term “C₃₋₆ cycloalkyl” includes a saturated hydrocarbon ring system containing 3, 4, 5 or 6 carbon atoms. For example, the “C₃-C₆ cycloalkyl” may be cyclopropyl, cyclobutyl, cyclopentyl, cyclohexyl, bicycle[2.1.1]hexane or bicycle[1.1.1]pentane.

As used herein, “cycloalkenyl” refers to a cycloalkyl group having at least one carbon-carbon double bond in at least one ring.

The term “heterocyclyl”, “heterocyclic” or “heterocycle” includes a non-aromatic saturated or partially saturated monocyclic or fused, bridged, or spiro bicyclic heterocyclic ring system(s). Monocyclic heterocyclic rings may contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles may contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles comprising at least one nitrogen in a ring position include, for example, azetidinyl, pyrrolidinyl, piperidinyl, piperazinyl, morpholinyl, thiomorpholinyl, tetrahydrotriazinyl, tetrahydropyrazolyl, tetrahydropyridinyl, homopiperidinyl, homopiperazinyl, 3,8-diaza-bicyclo[3.2.1]octanyl, 8-aza-bicyclo[3.2.1]octanyl, 2,5-Diaza-bicyclo[2.2.1]heptanyl and the like. Typical sulfur containing heterocycles include tetrahydrothienyl, dihydro-1,3-dithiol, tetrahydro-2H-thiopyran, and hexahydrothiepine. Other heterocycles include dihydro oxathiolyl, tetrahydro oxazolyl, tetrahydro-oxadiazolyl, tetrahydrod ioxazolyl, tetrahydrooxathiazolyl, hexahydrotriazinyl, tetrahydro oxazinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O), for example, 2 oxopyrrolidinyl, 2-oxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. For example, the term “piperidino” or “morpholino” refers to a piperidin-1-yl or morpholin-4-yl ring that is linked via the ring nitrogen.

As used herein, the term “heterocycloalkyl” is a subset of heterocyclyls and represents a saturated moncyclic or polycyclic (e.g. bicyclic) aliphatic ring system containing, for instance, from 3 to 12 ring atoms, at least one being a heteroatom selected from nitrogen, oxygen and sulphur in the ring.

The term “bridged ring systems” includes ring systems in which two rings share more than two atoms, see for example Advanced Organic Chemistry, by Jerry March, 4th Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane, and quinuclidine.

The term “spiro bi-cyclic ring systems” includes ring systems in which two ring systems share one common spiro carbon atom, i.e. the heterocyclic ring is linked to a further carbocyclic or heterocyclic ring through a single common spiro carbon atom. Examples of spiro ring systems include 3,8-diaza-bicyclo[3.2.1]octane, 2,5-Diaza-bicyclo[2.2.1]heptane, 6-azaspiro[3.4]octane, 2-oxa-6-azaspiro[3.4]octane, 2-azaspiro[3.3]heptane, 2-oxa-6-azaspiro[3.3]heptane, 6-oxa-2-azaspiro[3.4]octane, 2,7-diaza-spiro[4.4]nonane, 2-azaspiro[3.5]nonane, 2-oxa-7-azaspiro[3.5]nonane and 2-oxa-6-azaspiro[3.5]nonane.

“Heterocyclyl-Cₘ₋ₙ alkyl” includes a heterocyclyl group covalently attached to a Cₘ₋ₙ alkylene group, both of which are defined herein.

The term “aromatic” when applied to a substituent as a whole includes a single ring or polycyclic ring system with 4n+2 electrons in a conjugated π system within the ring or ring system where all atoms contributing to the conjugated π system are in the same plane.

The term “aryl” includes an aromatic hydrocarbon ring system. The ring system has 4n+2 electrons in a conjugated π system within a ring where all atoms contributing to the conjugated π system are in the same plane. For example, the “aryl” may be phenyl and naphthyl. The aryl system itself may be substituted with other groups.

The term “heteroaryl” includes an aromatic mono- or bicyclic ring incorporating one or more (for example 1-4, particularly 1, 2 or 3) heteroatoms selected from nitrogen, oxygen or sulfur. The ring or ring system has 4n+2 electrons in a conjugated π system where all atoms contributing to the conjugated π system are in the same plane.

Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl and imidazo[1,2-b][1,2,4]triazinyl. Examples of heteroaryl groups comprising at least one nitrogen in a ring position include pyrrolyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, indolyl, isoindolyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl and pteridinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

Particular examples of bicyclic heteroaryl groups containing a six membered ring fused to a five membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl, pyrrolopyridine, and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

“Heteroaryl-Cₘ₋ₙ alkyl-” includes a heteroaryl group covalently attached to a Cₘ₋ₙ alkylene group, both of which are defined herein. Examples of heteroaralkyl groups include pyridin-3-ylmethyl and the like.

As used herein, the term “alkoxy” denotes —O-alkyl wherein alkyl is as defined above. C₁-C₆ alkoxy includes an alkyl having from 1 to 6 carbon atoms. Non-limiting examples of C₁-C₆ alkoxy are methoxy, ethoxy, n-propyloxy, iso-propyloxy, 2-methyl-1-propyloxy, 2-methyl-2-propyloxy, 2-methyl-1-butyloxy, 3-methyl-1-butyloxy, 2-methyl-3-butyloxy, 2,2-dimethyl-1-propyloxy, 2-methyl-1-pentyloxy, 3-methyl-1-pentyloxy, 4-methyl-1-pentyloxy, 2-methyl-2-pentyloxy, 3-methyl-2-pentyloxy, 4-methyl-2-pentyloxy, 2,2-dimethyl-1-butyloxy, 3,3-dimethyl-1-butyloxy, 2-ethyl-1-butyloxy, butyloxy, iso-butyloxy, t-butyloxy, pentyloxy, iso-pentyloxy, neo-pentyloxy, hexyloxy, and the like.

As used herein, the term “alkoxy-carbonyl” refers to —C(O)—O-alkyl.

As used herein, the term “alkyl-carbonyl” refers to —C(O)-alkyl.

As used herein, the term “halo” refers to fluoro, chloro, bromo and iodo.

As used herein, the term “nitro” refers to —NO₂.

As used herein, the term “hydroxy” refers to —OH.

As used herein, the term “carboxy” refers to —COOH.

As used herein, the term “nitrile” (sometimes also called “cyano”) refers to —CN.

As used herein, the term “oxo” refers to ═O.

When reference herein is made to “the cyclic diamine moiety” of any formula herein, the term refers to

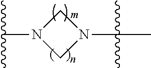

of the formula.

As used herein “spiro carbon atom” refers to a carbon atom which is shared by two rings. Thus, when the cyclic diamine moiety of formula I is connected via a spiro carbon atom, the cyclic diamine moiety forms two rings. For example, when the cyclic diamine moiety is connected by a spiro carbon atom and ₘ and ₙ are both 4, the cyclic diamine moiety may have the following structure:

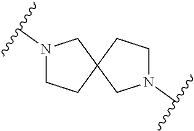 [SMILES: *N1CCC2(CCN(*)C2)C1]

(i.e. the shared spiro carbon counts toward one carbon of m and one carbon of n).

The term “optionally substituted” includes either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

A bond terminating in a “ [symbol] ” represents that the bond is connected to another atom that is not shown in the structure and denotes the point of attachment of a chemical moiety to the remainder of a molecule or chemical formula. A bond terminating inside a cyclic structure and not terminating at an atom of the ring structure represents that the bond may be connected to any of the atoms in the ring structure where allowed by valency.

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without undue effort which substitutions are chemically possible, and which are not.

Ortho, meta and para substitution are well understood terms in the art. For the absence of doubt, “ortho” substitution is a substitution pattern where adjacent carbons possess a substituent, whether a simple group, for example the fluoro group in the example below, or other portions of the molecule, as indicated by the bond ending in “ [symbol] ”.

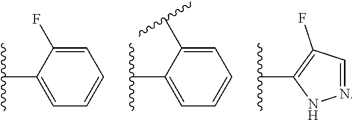

“Meta” substitution is a substitution pattern where two substituents are on carbons one carbon removed from each other, i.e. with a single carbon atom between the substituted carbons. In other words there is a substituent on the second atom away from the atom with another substituent. For example the groups below are meta substituted.

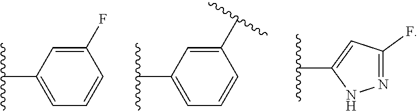

“Para” substitution is a substitution pattern where two substituents are on carbons two carbons removed from each other, i.e. with two carbon atoms between the substituted carbons. In other words there is a substituent on the third atom away from the atom with another substituent. For example the groups below are para substituted.

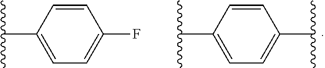

The term “acyl” includes an organic radical derived from, for example, an organic acid by the removal of the hydroxyl group, e.g. a radical having the formula R—C(O)—, where R may be selected from H, C₁₋₆ alkyl, C₃₋₈ cycloalkyl, phenyl, benzyl or phenethyl group, e.g. R is H or C₁₋₃ alkyl. In one embodiment acyl is alkyl-carbonyl. Examples of acyl groups include, but are not limited to, formyl, acetyl, propionyl and butyryl. A particular acyl group is acetyl (also represented as Ac).

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The various functional groups and substituents making up the compounds of the present invention are typically chosen such that the molecular weight of the compound does not exceed 1000. More usually, the molecular weight of the compound will be less than 750, for example less than 700, or less than 650, or less than 600, or less than 550. More preferably, the molecular weight is less than 525 and, for example, is 500 or less.

Suitable or preferred features of any compounds of the present invention may also be suitable features of any other aspect.

The invention contemplates pharmaceutically acceptable salts of the compounds of the invention. These may include the acid addition and base salts of the compounds. These may be acid addition and base salts of the compounds.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of the invention may be prepared by for example, one or more of the following methods:

(i) by reacting the compound of the invention with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

These methods are typically carried out in solution. The resulting salt may precipitate out and be collected by filtration or may be recovered by evaporation of the solvent. The degree of ionisation in the resulting salt may vary from completely ionised to almost non-ionised.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Where a compound of the invention has two or more stereo centres any combination of (R) and (S) stereoisomers is contemplated. The combination of (R) and (S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%

The compounds of this invention may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess LOX inhibitory activity.

Compounds and salts described in this specification may be isotopically-labelled (or “radio-labelled”). Accordingly, one or more atoms are replaced by an atom having an atomic mass or mass number different from the atomic mass or mass number typically found in nature. Examples of radionuclides that may be incorporated include ²H (also written as “D” for deuterium), ³H (also written as “T” for tritium), ¹¹C, ¹³C, ¹⁴C, ¹⁵O, ¹⁷O, ¹⁸O, ¹⁸F and the like. The radionuclide that is used will depend on the specific application of that radio-labelled derivative. For example, for in vitro competition assays, ³H or ¹⁴C are often useful. For radio-imaging applications, ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F.

It is also to be understood that certain compounds of the invention may exist in solvated as well as unsolvated forms such as, for example, hydrated forms. It is to be understood that the invention encompasses all such solvated forms that possess LOX inhibitory activity.

It is also to be understood that certain compounds of the invention may exhibit polymorphism, and that the invention encompasses all such forms that possess LOX inhibitory activity.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

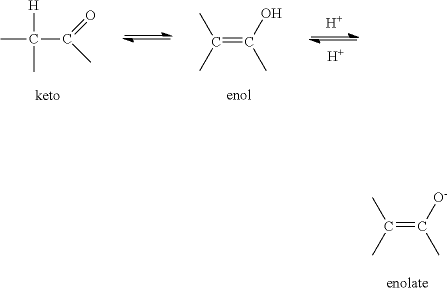

The in vivo effects of a compound of the invention may be exerted in part by one or more metabolites that are formed within the human or animal body after administration of a compound of the invention.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

Further information on the preparation of the compounds of the invention is provided in the Examples section. The general reaction schemes and specific methods described in the Examples form a further aspect of the invention.

The resultant compound of the invention from the processes defined above can be isolated and purified using techniques well known in the art.

Compounds of the invention may exist in a single crystal form or in a mixture of crystal forms or they may be amorphous. Thus, compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

The processes defined herein may further comprise the step of subjecting the compound of the invention to a salt exchange, particularly in situations where the compound of the invention is formed as a mixture of different salt forms. The salt exchange suitably comprises immobilising the compound of the invention on a suitable solid support or resin, and eluting the compounds with an appropriate acid to yield a single salt of the compound of the invention.

In a further aspect of the invention, there is provided a compound of the invention obtainable by any one of the processes defined herein.

Certain of the intermediates described in the reaction schemes above and in the Examples herein are novel. Such novel intermediates, or a salt thereof, particularly a pharmaceutically acceptable salt thereof, form a further aspect of the invention.

### Compounds

In one embodiment, in compounds of formula (I), q is 0 or 1.

In one embodiment, R¹¹ and R¹² are independently selected from hydrogen and C₁₋₃ alkyl. Suitably, R¹¹ and R¹² are independently selected from hydrogen, methyl and ethyl. Suitably, R¹¹ and R¹² are independently selected from hydrogen and methyl.

In another embodiment, in compounds of formula (I), q is 0. Thus, Z is directly linked to the aminoethylsulfonyl group.

In one embodiment, the cyclic diamine moiety is (iii) optionally linked by a spiro carbon atom; wherein each ring carbon atom of said cyclic diamine moiety may be optionally substituted by one or two substituents independently selected from C₁-C₆ alkyl, C₁-C₆ alkyl substituted with —OR^{2a}, or C₁-C₆ alkyl substituted with —NR^{2a}R^{2b}.

In one embodiment, the cyclic diamine moiety is (i) optionally linked by a bond or (ii) optionally bridged by —(CR⁹R¹⁰)ₒ—, where R⁹ and R¹⁰ are at each occurrence independently selected from H and unsubstituted C₁₋₄ alkyl and o is 1, 2, 3 or 4.

In one embodiment, the cyclic diamine moiety is (i) optionally linked by a bond or (ii) optionally bridged by —(CR⁹R¹⁰)ₒ—, where R⁹ and R¹⁰ are at each occurrence independently selected from H and unsubstituted C₁₋₄ alkyl and o is 1, 2, 3 or 4; and q is 0. Thus the compound of formula (I) is a compound according to the following formula Ia:

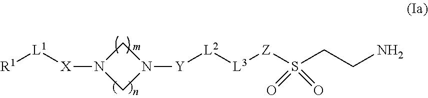

or a pharmaceutically acceptable salt thereof, wherein
m and n are each independently selected from 1, 2, 3 or 4, and where two ring carbon atoms of the cyclic diamine moiety of formula

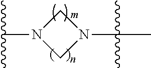

may be optionally linked by a bond or optionally bridged by —(CR⁹R¹⁰)ₒ—, where R⁹ and R¹⁰ are at each occurrence independently selected from H and unsubstituted C₁₋₄ alkyl and o is 1, 2, 3 or 4;
each ring carbon atom of said cyclic diamine moiety may be optionally substituted by one or two substituents independently selected from C₁-C₆ alkyl, C₁-C₆ alkyl substituted with —OR^{2a}, or C₁-C₆ alkyl substituted with —NR^{2a}R^{2b};
   L¹ and L² are each independently selected from a bond, —O—, —C(O)—, —C(O)O—, —OC(O)—, —C(O)NR³—, —NR³C(O)—, —NR³—, —SO₂NR³—, —NR³SO₂—, —S—, —SO₂—, —SO₂O—, —OSO₂—, —NR³SO₂NR⁴—, —NR³C(O)NR⁴—, —NR³C(O)O— or —OC(O)NR³—;
   L³ is selected from a bond, C₁-C₄ alkylene, C₂-C₄ alkenylene or C₂-C₄ alkynylene, where
any alkylene, alkenylene or alkynylene in L³ may be optionally substituted by one or two substituents independently selected from halo, cyano, oxo, hydroxy, carboxy, R², —OR², —C(O)R², —C(O)OR², —OC(O)R², —C(O)NR⁶R⁷, —NR⁶C(O)R⁷, —NR⁶R⁷, —SO₂NR⁶R⁷, —NR⁶SO₂R⁷, —SR⁶, —SO₂R⁶, —SO₂OR⁶, —OSO₂R⁶, —NR⁶SO₂NR⁷R⁸, —NR⁶C(O)NR⁷R⁸, —NR⁶C(O)OR⁷ or —OC(O)NR⁶R⁷;
   X, Y and Z are each independently selected from a bond or a 3- to 12-membered ring system, including 0, 1, 2 or 3 heteroatoms selected from N, O or S in the ring system, where
any ring system in X, Y and Z may be optionally substituted by one, two or three substituents independently selected from halo, cyano, oxo, hydroxy, carboxy, R², —OR², —C(O)R², —C(O)OR², —OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵, —R³C(O)NR⁴R⁵, —NR³C(O)OR⁴ or —OC(O)NR⁴R⁵;
   R¹ is selected from hydrogen, C₁-C₆ alkyl, C₂-C₆ alkenyl, C₂-C₆ alkynyl or a 3- to 12-membered ring system (e.g. cycloalkyl, heterocyclyl, aryl or heteroaryl), including 0, 1, 2 or 3 heteroatoms selected from N, O or S in the ring system, where
any alkyl, alkenyl or alkynyl in R¹ may be optionally substituted by one, two or three substituents independently selected from halo, cyano, amino, oxo, hydroxy, carboxy, R², —OR², —C(O)R², —C(O)OR², —OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵, —NR³C(O)NR⁴R⁵, —NR³C(O)OR⁴ or —OC(O)NR⁴R⁵; and
any ring system in R¹ may be optionally substituted by one, two or three substituents independently selected from halo, cyano, oxo, hydroxy, carboxy, R², —OR², —C(O)R², —C(O)OR², —OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵, —NR³C(O)NR⁴R⁵, —NR³C(O)OR⁴ or —OC(O)NR⁴R⁵;
   R² is at each occurrence independently selected from C₁-C₆ alkyl, C₂-C₆ alkenyl, C₂-C₆ alkynyl or C₃-C₆ cycloalkyl, where
any alkyl, alkenyl, alkynyl or cycloalkyl in R² may be optionally substituted by one, two or three substituents independently selected from halo, cyano, oxo, R^{2a}, —OR^{2a}, —C(O)R^{2a}, —C(O)OR^{2a}, —OC(O)R^{2a}, —C(O)NR^{2a}R^{2b}, —NR^{2a}C(O)R^{2b}, —NR^{2a}R^{2b}, —SO₂NR^{2a}R^{2b}, —NR^{2a}SO₂R^{2b}, —SR^{2a}, —SO₂R^{2a}, —SO₂OR^{2a}, —OSO₂R^{2a}, —NR^{2a}SO₂NR^{2b}R^{2c}, —NR^{2a}C(O)NR^{2b}R^{2c}, —NR^{2a}C(O)OR^{2b} or —OC(O)NR^{2a}R^{2b};
   R^{2a}, R^{2b} and R^{2c} are at each occurrence independently selected from hydrogen or unsubstituted C₁-C₄ alkyl;
   R³, R⁴ and R⁵ are at each occurrence independently selected from hydrogen, C₁-C₆ alkyl or C₃-C₆ cycloalkyl, where
any alkyl or cycloalkyl in R³, R⁴ and R⁵ may be optionally substituted by one, two or three substituents independently selected from halo, cyano, oxo, hydroxy, carboxy, R², —OR², —C(O)R² or —C(O)OR², and
when R⁴ is optionally substituted C₁-C₆ alkyl and R⁵ is optionally substituted C₁-C₆ alkyl, then R⁴ and R⁵ together with the nitrogen atom to which they are attached in —C(O)NR⁴R⁵, —NR⁴R⁵, —SO₂NR⁴R⁵, —NR³SO₂NR⁴R⁵, —R³C(O)NR⁴R⁵ or —OC(O)NR⁴R⁵ may form a 3- to 6-membered heterocycloalkyl;
   R⁶, R⁷ and R⁸ are at each occurrence independently selected from hydrogen, C₁-C₆ alkyl or C₃-C₆ cycloalkyl, where
any alkyl or cycloalkyl in R⁶, R⁷ and R⁸ may be optionally substituted by one, two or three substituents independently selected from halo, cyano, oxo, hydroxy, carboxy, R^{2a}, —OR^{2a}, —C(O)R^{2a}, —C(O)OR^{2a}, —C(O)NR^{2a}R^{2b}, —NR^{2a}C(O)R^{2b}, —NR^{2a}R^{2b}, —SO₂NR^{2a}R^{2b}, —NR^{2a}SO₂R^{2b}, —SR^{2a}, —SO₂R^{2a}, —SO₂OR^{2a}, —OSO₂R^{2a}, —NR₂SO₂NR^{2a}R^{2b}, —NR^{2a}C(O)NR^{2b}R^{2c}, —NR^{2a}C(O)OR^{2b} or —OC(O)NR^{2a}R^{2b};
   provided at least one of L², Y, L³ and Z is not a bond;
   provided —SO₂—(CH₂)₂—NH₂ in Formula Ia is linked to the remainder of the compound of Formula Ia via a carbon atom;
   provided when X is a bond, then L¹ is selected from a bond, —C(O)—, —OC(O)—, —NR³C(O)—, —NR³SO₂—, —SO₂— and —OSO₂—;
   provided when Y is a bond, then L² is selected from a bond, —C(O)—, —C(O)O—, —C(O)NR³—, —SO₂NR³—, —SO₂— and —SO₂O; and
   provided the compound is not

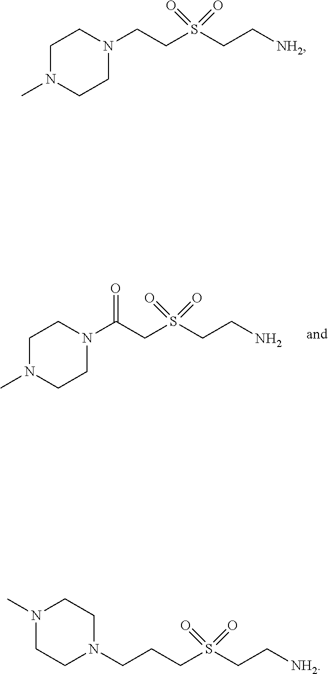 [SMILES: CN1CCN(CCS(=O)(=O)CCN)CC1 ; CN1CCN(C(=O)CS(=O)(=O)CCN)CC1 ; CN1CCN(CCCS(=O)(=O)CCN)CC1]

In compounds of Formula (I) or (Ia), when Y is a bond, then L² is selected from a bond, —C(O)—, —C(O)O—, —C(O)NR³—, —SO₂NR³—, —SO₂— and —SO₂O, particularly L² is then selected from a bond, —C(O)—, —C(O)O—, —C(O)NH—, —SO₂NH—, —SO₂— and —SO₂O.

In compounds of Formula (I) or (Ia), when X is a bond, then L¹ is selected from a bond, —C(O)—, —OC(O)—, —NR³C(O)—, —NR³SO₂—, —SO₂— and —OSO₂—, particularly L¹ is then selected from a bond, —C(O)—, —OC(O)—, —NHC(O)—, —NHSO₂—, —SO₂— and —OSO₂—.

In some embodiments of compounds as disclosed herein, the compound of Formula I is a compound of the structure of Formula (Ib):

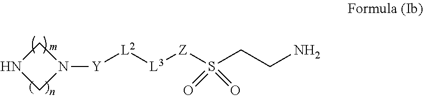

In some embodiments of compounds of Formula (I) or (Ia), when X is a bond and L¹ is a bond then R¹ is not hydrogen.

In embodiments, a compound of Formula (I) or (Ia) is a compound of the structure of Formula (II):

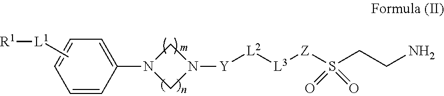

or a pharmaceutically acceptable salt thereof.

In embodiments, a compound of Formula (I) or (Ia) is a compound of the structure of Formula (III):

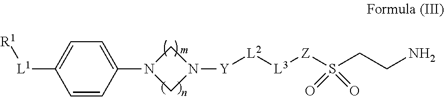

or a pharmaceutically acceptable salt thereof.

In embodiments, a compound of Formula (I) or (Ia) is a compound of the structure of Formula (IV):

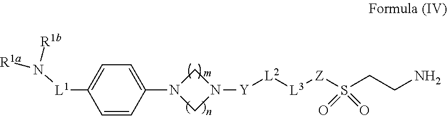

or a pharmaceutically acceptable salt thereof, wherein
   R^{1b} and R^{1c} together form a 3- to 7-membered heterocycloalkyl, optionally including one additional heteroatom selected from O, N or S in the ring,
said heterocyclalkyl formed by R_{1b} and R_{1c} may be optionally substituted by one, two or three substituents independently selected from halo, cyano, oxo, hydroxy, carboxy, R², —OR², —C(O)R², —C(O)OR², —OC(O)R², —C(O)NR⁴R⁵, —NR³C(O)R⁴, —NR³R⁴, —SO₂NR³R⁴, —NR³SO₂R⁴, —SR³, —SO₂R³, —SO₂OR³, —OSO₂R³, —NR₂SO₂NR⁴R⁵, —NR³C(O)NR⁴R⁵, —NR³C(O)OR⁴ or —OC(O)NR⁴R⁵.

In embodiments of compounds of Formula (IV), R^{1b} and R^{1c} together form a 3- to 7-membered heterocycloalkyl, optionally including one additional heteroatom selected from O, N or S in the ring, where said heterocyclalkyl formed by R_{1b} and R_{1c} may be optionally substituted by one or two substituents independently selected from oxo, —SO₂R³, —SO₂OR³ or —OSO₂R³, where R³ is unsubstituted C₁-C₆ alkyl, in particular R³ is unsubstituted C₁-C₄ alkyl.

In embodiments of compounds of Formula (IV), R^{1b} and R^{1c} together form a 3- to 7-membered heterocycloalkyl, optionally including one additional heteroatom selected from O, N or S in the ring, where said heterocyclalkyl formed by R_{1b} and R_{1c} may be optionally substituted by one or two substituents independently selected from oxo or —SO₂R³, where R³ is unsubstituted C₁-C₆ alkyl, in particular R³ is unsubstituted C₁-C₄ alkyl.

In embodiments, a compound of Formula (I) or (Ia) is a compound of the structure of Formula (V):

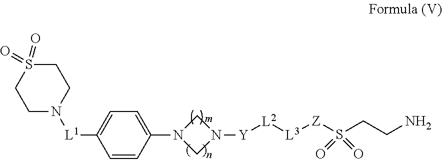

or a pharmaceutically acceptable salt thereof.

In embodiments, a compound of Formula (I) or (Ia) is a compound of the structure of Formula (VI):

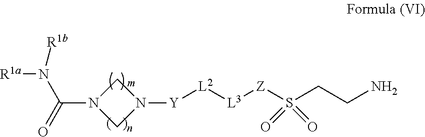

or a pharmaceutically acceptable salt thereof, wherein
R^{1a} and R^{1b} together form a 3- to 7-membered heterocycloalkyl, optionally including one additional heteroatom selected from O, N or S in the ring, where said heterocyclalkyl formed by R^{1a} and R^{1b} may be optionally substituted by one or two substituents independently selected from oxo, —SO₂R³, —SO₂OR³ or —OSO₂R³, where R³ is unsubstituted C₁-C₆ alkyl, in particular R³ is unsubstituted C₁-C₄ alkyl.

In embodiments, a compound of Formula (I) or (Ia) is a compound of the structure of Formula (VII):

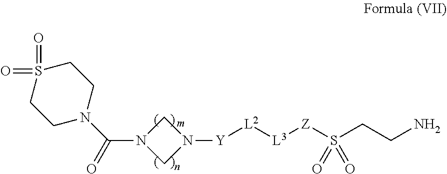

or a pharmaceutically acceptable salt thereof.

In embodiments, a compound of Formula (I) or (Ia) is a compound of the structure of Formula (VIII):

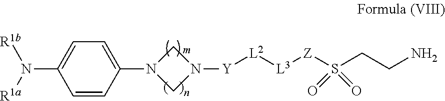

or a pharmaceutically acceptable salt thereof, wherein
R^{1a} and R^{1b} together form a 3- to 7-membered heterocycloalkyl, optionally including one additional heteroatom selected from O, N or S in the ring, where said heterocyclalkyl formed by R^{1a} and R^{1b} may be optionally substituted by one or two substituents independently selected from oxo, —SO₂R³, —SO₂OR³ or —OSO₂R³, where R³ is unsubstituted C₁-C₆ alkyl, in particular R³ is unsubstituted C₁-C₄ alkyl.

In embodiments, a compound of Formula (I) or (Ia) is a compound of the structure of Formula (IX):

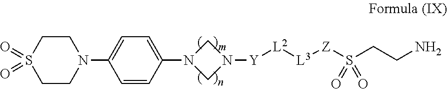

or a pharmaceutically acceptable salt thereof.

In embodiments, a compound of Formula (I) or (Ia) is a compound of the structure of Formula (X):

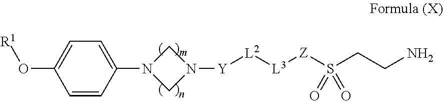

or a pharmaceutically acceptable salt thereof, wherein
   R¹ is C₁-C₆ alkyl optionally substituted by one, two or three substituents independently selected from halo, cyano, amino, oxo, hydroxy or carboxy, in particular R¹ is unsubstituted C₁-C₆ alkyl or C₁-C₆ alkyl substituted by hydroxy, more particularly R¹ is unsubstituted C₁-C₄ alkyl.

In embodiments, a compound of Formula (I) or (Ia) is a compound of the structure of Formula (XI):

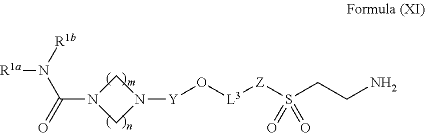

or a pharmaceutically acceptable salt thereof, wherein
R^{1a} and R^{1b} together form a 3- to 7-membered heterocycloalkyl, optionally including one additional heteroatom selected from O, N or S in the ring, where said heterocyclalkyl formed by R^{1a} and R^{1b} may be optionally substituted by one or two substituents independently selected from oxo, —SO₂R³, —SO₂OR³ or —OSO₂R³, where R³ is unsubstituted C₁-C₆ alkyl, in particular R³ is unsubstituted C₁-C₄ alkyl.

In embodiments, a compound of Formula (I) or (Ia) is a compound of the structure of Formula (XII):

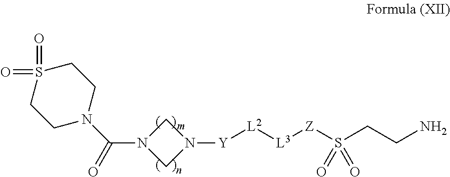

or a pharmaceutically acceptable salt thereof, wherein
   R¹ is C₁-C₆ alkyl optionally substituted by one, two or three substituents independently selected from halo, cyano, amino, oxo, hydroxy or carboxy, in particular R¹ is unsubstituted C₁-C₆ alkyl or C₁-C₆ alkyl substituted by hydroxy, more particularly R¹ is unsubstituted C₁-C₄ alkyl.

In embodiments, a compound of Formula (I) or (Ia) is a compound of the structure of Formula (XIII):

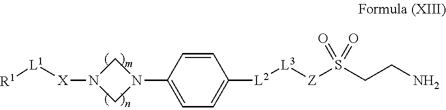

or a pharmaceutically acceptable salt thereof.

In embodiments, a compound of Formula (I) or (Ia) is a compound of the structure of Formula (XIV):

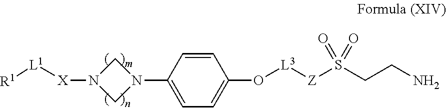

or a pharmaceutically acceptable salt thereof.

In embodiments, a compound of Formula (I) or (Ia) is a compound of the structure of Formula (XV):

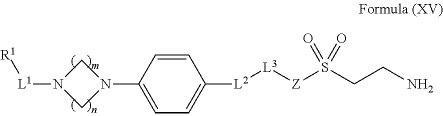

or a pharmaceutically acceptable salt thereof.

In embodiments, a compound of Formula (I) or (Ia) is a compound of the structure of Formula (XVI):

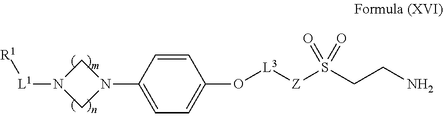

or a pharmaceutically acceptable salt thereof.

In embodiments, a compound of Formula (I) or (Ia) is a compound of the structure of Formula (XVII):

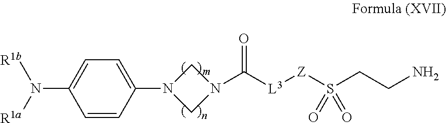

or a pharmaceutically acceptable salt thereof, wherein
R^{1a} and R^{1b} together form a 3- to 7-membered heterocycloalkyl, optionally including one additional heteroatom selected from O, N or S in the ring, where said heterocyclalkyl formed by R^{1a} and R^{1b} may be optionally substituted by one or two substituents independently selected from oxo, —SO₂R³, —SO₂OR³ or —OSO₂R³, where R³ is unsubstituted C₁-C₆ alkyl, in particular R³ is unsubstituted C₁-C₄ alkyl.

In embodiments, a compound of Formula (I) or (Ia) is a compound of the structure of Formula (XVIII):

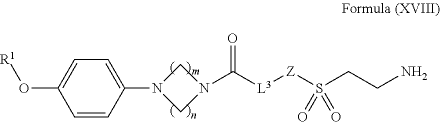

or a pharmaceutically acceptable salt thereof, wherein
   R¹ is C₁-C₆ alkyl optionally substituted by one, two or three substituents independently selected from halo, cyano, amino, oxo, hydroxy or carboxy, in particular R¹ is unsubstituted C₁-C₆ alkyl or C₁-C₆ alkyl substituted by hydroxy, more particularly R¹ is unsubstituted C₁-C₄ alkyl.

In embodiments, a compound of Formula (I) or (Ia) is a compound of the structure of Formula (XIX):

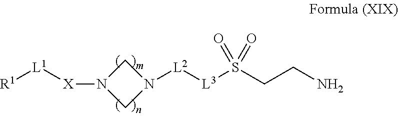

or a pharmaceutically acceptable salt thereof.

In embodiments, a compound of Formula (I) or (Ia) is a compound of the structure of Formula (XX):

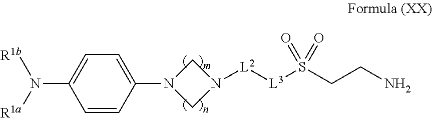

or a pharmaceutically acceptable salt thereof, wherein
   R^{1a} and R^{1b} together form a 3- to 7-membered heterocycloalkyl, optionally including one additional heteroatom selected from O, N or S in the ring, where said heterocycloalkyl formed by R^{1a} and R^{1b} may be optionally substituted by one or two substituents independently selected from oxo, —SO₂R³, —SO₂OR³ or —OSO₂R³, where R³ is unsubstituted C₁-C₆ alkyl, in particular R³ is unsubstituted C₁-C₄ alkyl.

In embodiments, a compound of Formula (I) or (Ia) is a compound of the structure of Formula (XX):

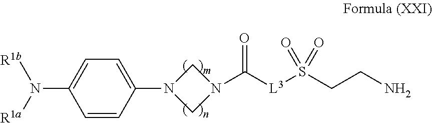

or a pharmaceutically acceptable salt thereof, wherein
   R^{1a} and R^{1b} together form a 3- to 7-membered heterocycloalkyl, optionally including one additional heteroatom selected from O, N or S in the ring, where said heterocyclalkyl formed by R^{1a} and R^{1b} may be optionally substituted by one or two substituents independently selected from oxo, —SO₂R³, —SO₂OR³ or —OSO₂R³, where R³ is unsubstituted C₁-C₆ alkyl, in particular R³ is unsubstituted C₁-C₄ alkyl.

In embodiments, a compound of Formula (I) or (Ia) is a compound of the structure of Formula (XXII):

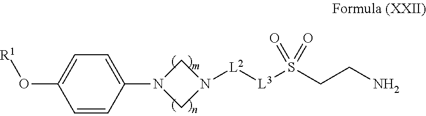

or a pharmaceutically acceptable salt thereof, wherein
   R¹ is C₁-C₅ alkyl optionally substituted by one, two or three substituents independently selected from halo, cyano, amino, oxo, hydroxy or carboxy, in particular R¹ is unsubstituted C₁-C₆ alkyl or C₁-C₆ alkyl substituted by hydroxy, more particularly R¹ is unsubstituted C₁-C₄ alkyl.

In embodiments, a compound of Formula (I) or (Ia) is a compound of the structure of Formula (XXIII):

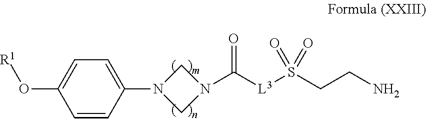

or a pharmaceutically acceptable salt thereof, wherein
   R¹ is C₁-C₆ alkyl optionally substituted by one, two or three substituents independently selected from halo, cyano, amino, oxo, hydroxy or carboxy, in particular R¹ is unsubstituted C₁-C₆ alkyl or C₁-C₆ alkyl substituted by hydroxy, more particularly R¹ is unsubstituted C₁-C₄ alkyl.

In embodiments, a compound of Formula (I) or (Ia) is a compound of the structure of Formula (XXIV):

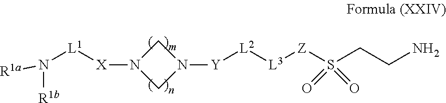

m and n are each independently selected from 2 or 3;

the cyclic diamine moiety of formula

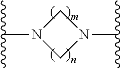

is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2; and the ring carbon atoms of the cyclic diamine moiety is unsubstituted;

L¹ is selected from a bond, —C(O)—, —C(O)O—, —C(O)NR³— (e.g. —C(O)NH—), —SO₂NR³— (e.g. —SO₂NH—), —SO₂— or —SO₂O—, particularly L¹ is selected from a bond or —C(O)—, more particularly L¹ is a bond;

L² is selected from a bond, —O—, —C(O)— or —C(O)NH—;

one of X and Y is unsubstituted phenyl or unsubstituted 5- to 6-membered heteroaryl and one of X and Y is a bond;

provided when Y is a bond, then L² is selected from a bond, —C(O)— or —C(O)NH;

provided when X is a bond, then L¹ is —C(O)—;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene;

Z is selected from a bond, C₃-C₆ cycloalkyl, C₃-C₆ heterocycloalkyl, phenyl or 5- to 6-membered heteroaryl,
any cycloalkyl, heterocycloalkyl, phenyl or heteroaryl in Z may be optionally substituted by one, two or three substituents independently selected from halo, cyano, oxo, hydroxy or carboxy;
any heterocycloalkyl or heteroaryl in Z including 1 or 2 heteroatoms selected from N, O or S in the ring; and

R^{1a} and R^{1b} together form a 3- to 7-membered heterocycloalkyl, optionally including one additional heteroatom selected from O, N or S in the ring, where said heterocyclalkyl formed by R^{1a} and R^{1b} may be optionally substituted by one or two substituents independently selected from oxo, —SO₂R³, —SO₂OR³ or —OSO₂R³, where R³ is unsubstituted C₁-C₄ alkyl.

In specific embodiments of compounds of Formula (XXIV), X is unsubstituted phenyl or unsubstituted 5- to 6-membered heteroaryl, Y is a bond and Z is a bond.

In further specific embodiments of compounds of Formula (XXIV), X is unsubstituted phenyl, Y is a bond and Z is a bond.

In embodiments, a compound of Formula (I) or (Ia) is a compound of the structure of Formula (XXV):

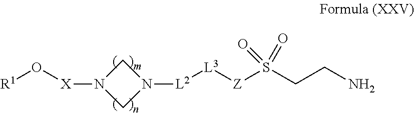

or a pharmaceutically acceptable salt thereof, wherein

m and n are each independently selected from 2 or 3;

the cyclic diamine moiety of formula

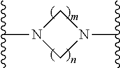

is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2; and the ring carbon atoms of the cyclic diamine moiety is unsubstituted;

L² is selected from a bond, —C(O)— or —C(O)NH—;

X is unsubstituted phenyl or unsubstituted 5- to 6-membered heteroaryl;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene;

Z is selected from a bond, C₃-C₆ cycloalkyl, C₃-C₆ heterocycloalkyl, phenyl or 5- to 6-membered heteroaryl,
any cycloalkyl, heterocycloalkyl, phenyl or heteroaryl in Z may be optionally substituted by one, two or three substituents independently selected from halo, cyano, oxo, hydroxy or carboxy;
any heterocycloalkyl or heteroaryl in Z including 1 or 2 heteroatoms selected from N, O or S in the ring; and

R¹ is C₁-C₆ alkyl optionally substituted by one, two or three substituents independently selected from halo, cyano, amino, oxo, hydroxy or carboxy, in particular R¹ is unsubstituted C₁-C₆ alkyl or C₁-C₆ alkyl substituted by hydroxy, more particularly R¹ is unsubstituted C₁-C₄ alkyl.

In specific embodiments of compounds of Formula (XXV), X is unsubstituted phenyl and Z is a bond or unsubstituted phenyl.

In further specific embodiments of compounds of Formula (XXV), X is unsubstituted phenyl and Z is a bond.

In embodiments, a compound of Formula (I) or (Ia) is a compound of the structure of Formula (XXVI):

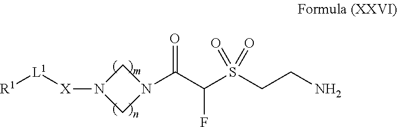

or a pharmaceutically acceptable salt thereof.

In particular embodiments of formula (XXVI), X is phenyl.

Particular embodiments of the invention include, for example, compounds of the invention (such as compounds of any one of Formulas (I) to (XXVI), or a pharmaceutically acceptable salt thereof, wherein, unless otherwise stated, each of R¹, R², R^{2a}, R^{2b}, R^{2c}, R³, R⁴, R⁵, R⁶, R⁷, R⁸, R⁹, R¹⁰, R¹¹, R¹², L¹, L², L³, X, Y, Z, m, n, o, p and the cyclic diamine moiety has any of the meanings defined hereinbefore or in any one or more of paragraphs (1) to (110) hereinafter:

1. Two ring carbon atoms of the cyclic diamine moiety are linked by a bond thereby forming a fused cyclic diamine moiety, and preferably the ring carbon atoms of the cyclic diamine moiety is unsubstituted, for instance

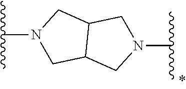 [SMILES: *N1CC2CN(*)CC2C1]

wherein * indicates the point of attachment to —Y-L²-L³-Z—SO₂—CH₂CH₂—NH₂.
2. Two ring carbon atoms of the cyclic diamine moiety are bridged by —(CR⁹R¹⁰)ₒ—, where R⁹ and R¹⁰ are at each occurrence independently selected from H and unsubstituted C₁₋₄ alkyl and o is 1, 2, 3 or 4, thereby forming a non-fused, bridged cyclic diamine moiety, and preferably the ring carbon atoms of the cyclic diamine moiety is unsubstituted, for instance

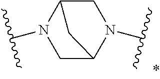 [SMILES: *N1CC2CC1CN2*]

wherein * indicates the point of attachment to —Y-L²-L³-Z—SO₂—CH₂CH₂—NH₂.
3. The cyclic diamine moiety is a non-fused, non-bridged cyclic diamine moiety, and preferably the ring carbon atoms of the cyclic diamine moiety is unsubstituted, for instance

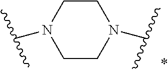 [SMILES: *N1CCN(*)CC1]

wherein * indicates the point of attachment to —Y-L²-L³-Z—SO₂—CH₂CH₂—NH₂.
4. The cyclic diamine moiety is a 6- to 8-membered cyclic diamine moiety, and preferably the ring carbon atoms of the cyclic diamine moiety are unsubstituted.
5. The ring carbon atoms of the cyclic diamine moiety are unsubstituted.
6. The cyclic diamine moiety is bridged by —(CR⁹R¹⁰)ₒ—, where R⁹ and R¹⁰ are at each occurrence independently selected from H and unsubstituted C₁₋₂ alkyl, and preferably the ring carbon atoms of the cyclic diamine moiety is unsubstituted.
7. The cyclic diamine moiety is bridged by —(CR⁹R¹⁰)ₒ—, where R⁹ and R¹⁰ are at each occurrence independently selected from H and unsubstituted C₁₋₂ alkyl, and o is 1, 2 or 3, (particularly o is 1 or 2), and preferably the ring carbon atoms of the cyclic diamine moiety are unsubstituted.
8. The cyclic diamine moiety is bridged by —(CR⁹R¹⁰)ₒ—, where o is 1 or 2, and preferably the ring carbon atoms of the cyclic diamine moiety are unsubstituted.
9. The cyclic diamine moiety is bridged by —(CR⁹R¹⁰)ₒ—, where o is 1 or 2 and R⁹ and R¹⁰ are at each occurrence independently selected from hydrogen and methyl, and preferably the ring carbon atoms of the cyclic diamine moiety are unsubstituted.
10. The cyclic diamine moiety is bridged by —CR⁹R¹⁰—, where R⁹ and R¹⁰ are each independently selected from H and unsubstituted C₁₋₂ alkyl, for instance the cyclic diamine moiety is bridged by —CH₂—, —CH(CH₃)— or —C(CH₃)₂—, and preferably the ring carbon atoms of the cyclic diamine moiety are unsubstituted.
11. The cyclic diamine moiety is bridged by —(CR⁹R¹⁰)₂—, where R⁹ and R¹⁰ are at each occurrence independently selected from H and unsubstituted C₁₋₂ alkyl, for instance the cyclic diamine moiety is bridged by —(CH₂)₂, —CH₂CH(CH₃)—, —CH₂C(CH₃)₂—, —CH₂CH(CH₂CH₃)— or —CH₂C(CH₂CH₃)₂—, and preferably the ring carbon atoms of the cyclic diamine moiety are unsubstituted.
12. The cyclic diamine moiety of formula

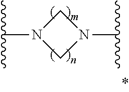

wherein * indicates the point of attachment to —Y-L²-L³-Z—SO₂—CH₂CH₂—NH₂, is selected from:

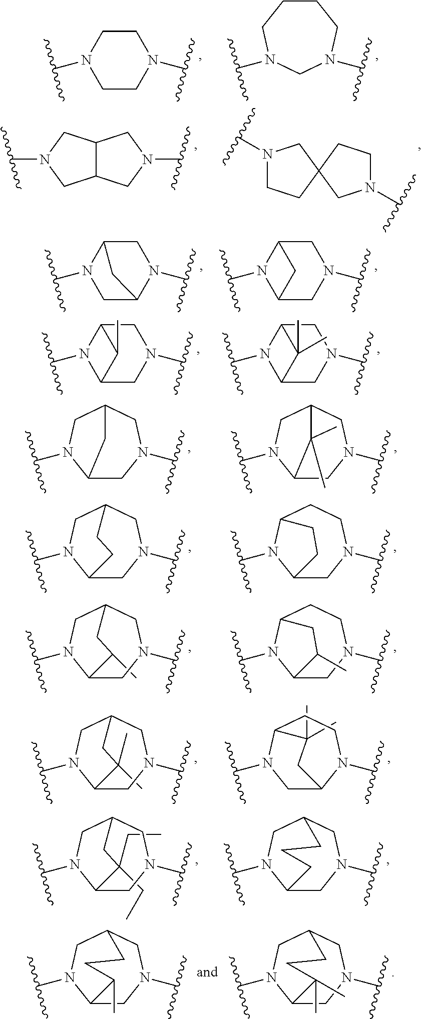

13. The cyclic diamine moiety of formula

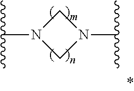

wherein * indicates the point of attachment to —Y-L²-L³-Z—SO₂—CH₂CH₂—NH₂, is selected from:

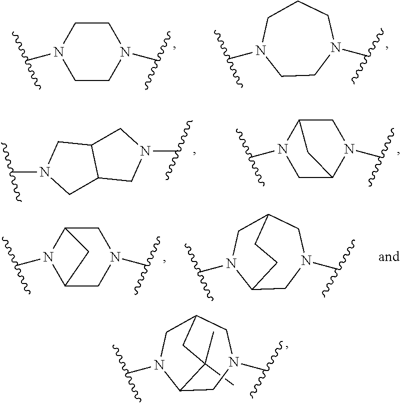

particularly selected from

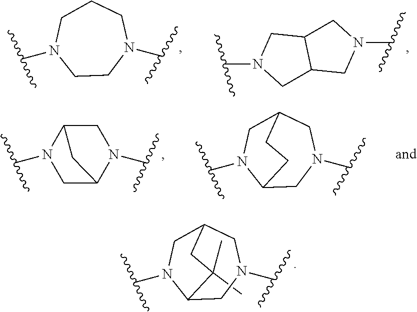

14. The cyclic diamine moiety of formula

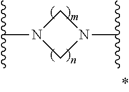

wherein * indicates the point of attachment to —Y-L²-L³-Z—SO₂—CH₂CH₂—NH₂, is selected from:

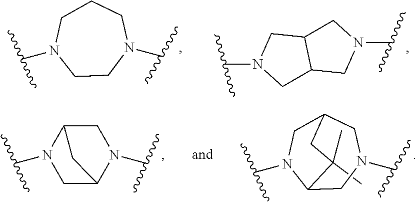

15. The cyclic diamine moiety of formula

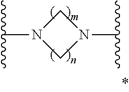

wherein * indicates the point of attachment to —Y-L²-L³-Z—SO₂—CH₂CH₂—NH₂, is

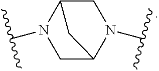 [SMILES: *N1CC2CC1CN2*]

16. The cyclic diamine moiety of formula

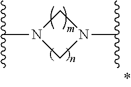

wherein * indicates the point of attachment to —Y-L²-L³-Z—SO₂—CH₂CH₂—NH₂, is

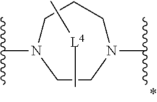

wherein * indicates the point of attachment to —Y-L²-L³-Z—SO₂—CH₂CH₂—NH₂; and wherein L⁴ is —(CR⁹R¹⁰)ₒ—, where R⁹ and R¹⁰ are at each occurrence independently selected from H and C₁₋₄ alkyl; and o is 1, 2, 3 or 4, particularly o is 1 or 2.
17. The cyclic diamine moiety of formula

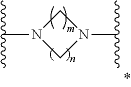

wherein * indicates the point of attachment to —Y-L²-L³-Z—SO₂—CH₂CH₂—NH₂, is selected from:

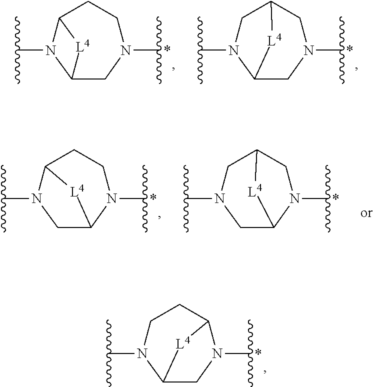

wherein * indicates the point of attachment to —Y-L²-L³-Z—SO₂—CH₂CH₂—NH₂; and wherein L⁴ is —(CR⁹R¹⁰)ₒ—, where R⁹ and R¹⁰ are at each occurrence independently selected from H and unsubstituted C₁₋₄ alkyl; and o is 1, 2, 3 or 4, particularly o is 1 or 2.
18. The cyclic diamine moiety of formula

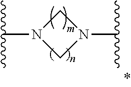

wherein * indicates the point of attachment to —Y-L²-L³-Z—SO₂—CH₂CH₂—NH₂, is selected from:

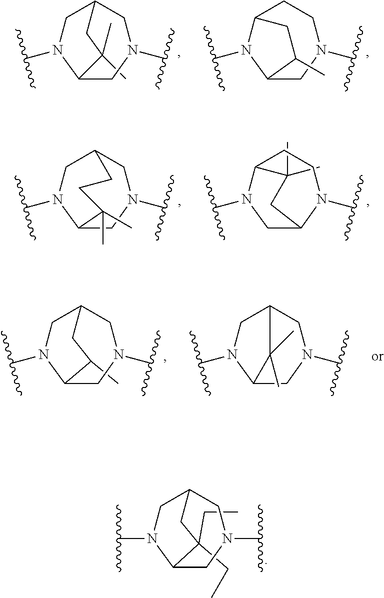

19. The cyclic diamine moiety of formula

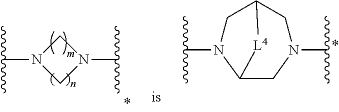

wherein * indicates the point of attachment to —Y-L²-L³-Z—SO₂—CH₂CH₂—NH₂; and wherein L⁴ is —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2.
20. The cyclic diamine moiety of formula

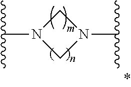

wherein * indicates the point of attachment to —Y-L²-L³-Z—SO₂—CH₂CH₂—NH₂, is selected from:

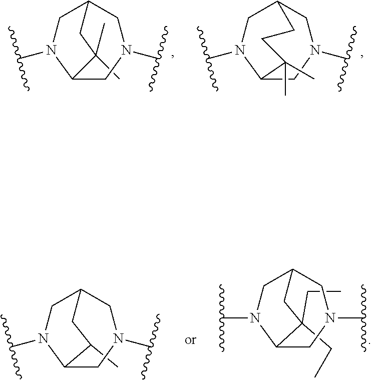 [SMILES: *N1CC2CN(*)C(C1)C(C)(C)C2 ; *N1CC2CCC(C)(C)C(C1)N(*)C2 ; *N1CC2CC(C)C(C1)N(*)C2 ; *N1CC2CN(*)C(C1)C(CC)(CC)C2]

21. The cyclic diamine moiety of formula

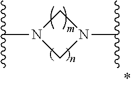

wherein * indicates the point of attachment to —Y-L²-L³-Z—SO₂—CH₂CH₂—NH₂, is

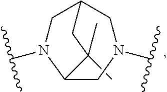 [SMILES: *N1CC2CN(*)C(C1)C(C)(C)C2]

22. The cyclic diamine moiety of formula

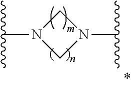

wherein * indicates the point of attachment to —Y-L²-L³-Z—SO₂—CH₂CH₂—NH₂, is selected from

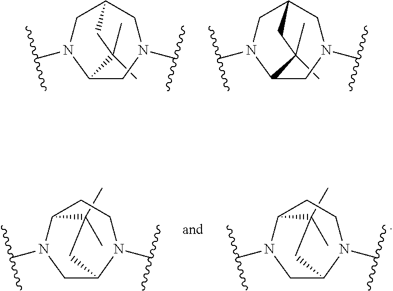

23 The cyclic diamine moiety of formula

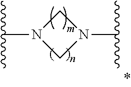

wherein * indicates the point of attachment to —Y-L²-L³-Z—SO₂—CH₂CH₂—NH₂, is linked through a spiro carbon atom.
24. The cyclic diamine moiety of formula

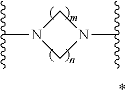

wherein * indicates the point of attachment to —Y-L²-L³-Z—SO₂—CH₂CH₂—NH₂, is linked through a spiro carbon atom and n and m are independently selected from 3 and 4.
25. The cyclic diamine moiety of formula

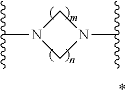

wherein * indicates the point of attachment to —Y-L²-L³-Z—SO₂—CH₂CH₂—NH₂, is linked through a spiro carbon atom and n and m are both 3, or n and m are both 4.
26. The cyclic diamine moiety of formula

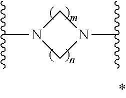

wherein * indicates the point of attachment to —Y-L²-L³-Z—SO₂—CH₂CH₂—NH₂, is selected from

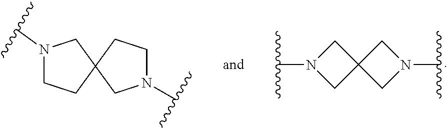 [SMILES: *N1CCC2(CCN(*)C2)C1 ; *N1CC2(C1)CN(*)C2]

27. m and n are each independently selected from 2 or 3, for example m is 2 and n is 2, m is 3 and n is 2, or m is 3 and n is 3.
28. R¹ is selected selected from C₁-C₆ alkyl, C₂-C₆ alkenyl, C₂-C₆ alkynyl or a 3- to 12-membered ring system (e.g. cycloalkyl, heterocyclyl, aryl or heteroaryl), including 0, 1, 2 or 3 heteroatoms selected from N, O or S in the ring system, where any alkyl, alkenyl or alkynyl in R¹ may be optionally substituted by one, two or three substituents independently selected from halo, cyano, amino, oxo, hydroxy, carboxy, R², —OR², —C(O)R², —C(O)OR², or —OC(O)R²; and any ring system in R¹ may be optionally substituted by one, two or three substituents independently selected from halo, cyano, oxo, hydroxy, carboxy, R², —OR², —C(O)R², —C(O)OR², —OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵,—NR³C(O)NR⁴R⁵, —NR³C(O)OR⁴ or —C(O)NR⁴R⁵.
29. R¹ is selected from C₁-C₆ alkyl, C₂-C₆ alkenyl, C₂-C₆ alkynyl, C₃-C₆ cycloalkyl, 3- to 7-membered heterocyclyl (e.g. 3- to 7-membered heterocycloalkyl), phenyl or 5- or 6-membered heteroaryl, where any heterocyclyl or heteroaryl in R¹ including 1, 2 or 3 heteroatoms selected from N, O or S in the ring; any alkyl, alkenyl or alkynyl in R¹ may be optionally substituted by one, two or three substituents independently selected from halo, cyano, amino, oxo, hydroxy, carboxy, R², —OR², —C(O)R², —C(O)OR², or —OC(O)R²; and any cycloalkyl, heterocyclyl, phenyl or heteroaryl in R¹ may be optionally substituted by one, two or three substituents independently selected from halo, cyano, oxo, hydroxy, carboxy, R², —OR², —C(O)R², —C(O)OR²,—OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵,—NR³C(O)NR⁴R⁵, —NR³C(O)OR⁴ or —C(O)NR⁴R⁵.
30. R¹ is selected from C₁-C₆ alkyl, C₂-C₆ alkenyl, C₂-C₆ alkynyl, C₃-C₆ cycloalkyl, 3- to 7-membered heterocyclyl (e.g. 3- to 7-membered heterocycloalkyl), phenyl or 5- or 6-membered heteroaryl, where any heterocyclyl or heteroaryl in R¹ including 1, 2 or 3 heteroatoms selected from N, O or S in the ring; any alkyl, alkenyl or alkynyl in R¹ may be optionally substituted by one, two or three substituents independently selected from halo, cyano, amino, oxo, hydroxy, carboxy, R², —OR², —C(O)R², —C(O)OR², or —OC(O)R²; and any cycloalkyl, heterocyclyl, phenyl or heteroaryl in R¹ may be optionally substituted by one, two or three substituents independently selected from halo, cyano, oxo, hydroxy, carboxy, R², —OR², —C(O)R², —C(O)OR² or—OC(O)R².
31. R¹ is selected from C₁-C₆ alkyl, C₃-C₆ cycloalkyl or 3- to 7-membered heterocycloalkyl, where any heterocycloalkyl in R¹ including 1 or 2 heteroatoms selected from N, O or S in the ring; any alkyl in R¹ may be optionally substituted by one or two substituents independently selected from halo, cyano, amino, oxo, hydroxy, carboxy, R², —OR², —C(O)R², —C(O)OR², or —OC(O)R²; any cycloalkyl or heterocycloalkyl in R¹ may be optionally substituted by one or two substituents independently selected from halo, cyano, oxo, hydroxy, carboxy, R², —OR², —C(O)R², —C(O)OR² or—OC(O)R²; and

R² is unsubstituted C₁-C₆ alkyl, in particular R² is unsubstituted C₁-C₄ alkyl.

32. R¹ is selected from C₁-C₆ alkyl, C₃-C₆ cycloalkyl or 3- to 7-membered heterocycloalkyl, where any heterocycloalkyl in R¹ including 1 or 2 heteroatoms selected from N, O or S in the ring; any alkyl in R¹ may be optionally substituted by one or two substituents independently selected from halo, cyano, oxo, hydroxy or carboxy; and any cycloalkyl or heterocycloalkyl in R¹ may be optionally substituted by one or two substituents independently selected from halo, cyano, oxo, hydroxy or carboxy.
33. R¹ is C₁-C₆ alkyl optionally substituted by one, two or three substituents independently selected from halo, cyano, amino, oxo, hydroxy or carboxy.
34. R¹ is C₁-C₆ alkyl optionally substituted by one or two substituents independently selected from halo, cyano, amino, oxo, hydroxy or carboxy.
35. R¹ is C₁-C₆ alkyl optionally substituted by one substituent selected from halo, cyano, amino, oxo, hydroxy or carboxy, in particular R¹ is C₁-C₆ alkyl optionally substituted by hydroxy or carboxy.
36. R¹ is unsubstituted C₁-C₆ alkyl or C₁-C₆ alkyl substituted by hydroxy.
37. R¹ is unsubstituted C₁-C₄ alkyl or C₁-C₄ alkyl substituted by hydroxy.
38. R¹ is unsubstituted C₁-C₄ alkyl.
39. R¹ is —NR^{1a}R^{1b} and R^{1a} and R^{1b} together with the nitrogen atom to which they are attached form:

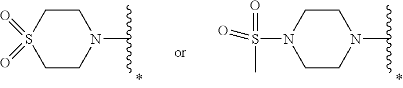 [SMILES: *N1CCS(=O)(=O)CC1 ; *N1CCN(S(C)(=O)=O)CC1]

wherein * indicates the point of attachment to the remainder of the compound.
40. R¹ is —NR^{1a}R^{1b} and R^{1a} and R^{1b} together with the nitrogen atom to which they are attached form:

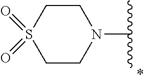 [SMILES: *N1CCS(=O)(=O)CC1]

wherein * indicates the point of attachment to the remainder of the compound.
41. R² is at each occurrence independently selected from C₁-C₆ alkyl, C₂-C₆ alkenyl, C₂-C₆ alkynyl or C₃-C₆ cycloalkyl, where any alkyl, alkenyl, alkynyl or cycloalkyl in R² may be optionally substituted by one, two or three substituents independently selected from halo, cyano, oxo, R^{2a}, —OR^{2a}, —C(O)R^{2a}, —C(O)OR^{2a} or —OC(O)R^{2a}.
42. R² is at each occurrence independently selected from unsubstituted C₁-C₆ alkyl, unsubstituted C₂-C₆ alkenyl or C₂—C alkynyl.
43. R² is at each occurrence independently selected from unsubstituted C₁-C₆ alkyl or unsubstituted C₂-C₆ alkenyl.
44. R² is at each occurrence independently selected from unsubstituted C₁-C₆ alkyl or unsubstituted C₃-C₆ cycloalkyl.
45. R² is at each occurrence independently selected from C₁-C₆ alkyl, where any alkyl in R² may be optionally substituted by one, two or three substituents independently selected from halo, cyano, oxo, R^{2a}, —OR^{2a}, —C(O)R^{2a}, —C(O)OR^{2a} or —OC(O)R^{2a}.
46. R² is at each occurrence independently selected from unsubstituted C₁-C₆ alkyl, in particular unsubstituted C₁-C₄ alkyl.
47. R³, R⁴ and R⁵ are at each occurrence independently selected from hydrogen, C₁-C₆ alkyl or C₃-C₆ cycloalkyl, where any alkyl or cycloalkyl in R³, R⁴ and R⁵ may be optionally substituted by one, two or three substituents independently selected from halo, cyano, oxo, hydroxy, carboxy, R², —OR², —C(O)R² or —C(O)OR².
48. R³, R⁴ and R⁵ are at each occurrence independently selected from hydrogen, C₁-C₆ alkyl or C₃-C₆ cycloalkyl, where any alkyl or cycloalkyl in R³, R⁴ and R⁵ may be optionally substituted by one, two or three substituents independently selected from halo, cyano, oxo, hydroxy, carboxy, R², —OR², —C(O)R² or —C(O)OR²; and

R² is selected from unsubstituted C₁-C₆ alkyl, in particular unsubstituted C₁-C₄ alkyl.

49. R³, R⁴ and R⁵ are at each occurrence independently selected from hydrogen or C₁-C₆ alkyl, where any alkyl in R³, R⁴ and R⁵ may be optionally substituted by one, two or three substituents independently selected from halo, cyano, oxo, hydroxy, carboxy.
50. R³, R⁴ and R⁵ are at each occurrence hydrogen.
51. R⁶, R⁷ and R⁸ are at each occurrence independently selected from hydrogen or C₁-C₆ alkyl, where any alkyl in R⁶, R⁷ and R⁸ may be optionally substituted by one, two or three substituents independently selected from halo, cyano, oxo, hydroxy, carboxy, R^{2a}, —OR^{2a}, —C(O)R^{2a}, —C(O)OR^{2a}, —C(O)NR^{2a}R^{2b}, —NR^{2a}C(O)R^{2b}, —NR^{2a}R^{2b}, —SO₂NR^{2a}R^{2b}, —NR^{2a}SO₂R^{2b}, —SR^{2a}, —SO₂R^{2a}, —SO₂OR^{2a}, —OSO₂R^{2a}, —NR₂SO₂NR^{2a}R^{2b}, .NR^{2a}C(O)NR^{2b}R^{2c}, —NR^{2a}C(O)OR^{2b} or —OC(O)NR^{2a}R^{2b}.
52. R⁶, R⁷ and R⁸ are at each occurrence independently selected from hydrogen or C₁-C₆ alkyl, where any alkyl in R⁶, R⁷ and R⁸ may be optionally substituted by one, two or three substituents independently selected from halo, cyano, oxo, hydroxy, carboxy, R^{2a}, —OR^{2a}, —C(O)R^{2a}, —C(O)OR^{2a}, —C(O)NR^{2a}R^{2b}, —NR^{2a}C(O)R^{2b}, —NR^{2a}R^{2b}, NR^{2a}C(O)NR^{2b}R^{2c}, —NR^{2a}C(O)OR^{2b} or —OC(O)NR^{2a}R^{2b}.
53. R⁶, R⁷ and R⁸ are at each occurrence independently selected from hydrogen or C₁-C₆ alkyl, where any alkyl in R⁶, R⁷ and R⁸ may be optionally substituted by one, two or three substituents independently selected from halo, cyano, oxo, hydroxy, carboxy, —OR^{2a}, —C(O)R^{2a}, —C(O)OR^{2a}, —C(O)NR^{2a}R^{2b}, —NR^{2a}C(O)R^{2b}, .NR^{2a}C(O)NR^{2b}R^{2c}, —NR^{2a}C(O)OR^{2b} or —OC(O)NR^{2a}R^{2b}.
54. R⁶, R⁷ and R⁸ are at each occurrence independently selected from hydrogen or C₁-C₆ alkyl, where any alkyl in R⁶, R⁷ and R⁸ may be optionally substituted by one, two or three substituents independently selected from halo, cyano, oxo, carboxy, —C(O)R^{2a}, —C(O)OR^{2a}, —C(O)NR^{2a}R^{2b}, —NR^{2a}C(O)R^{2b}, .NR^{2a}C(O)NR^{2b}R^{2c}, —NR^{2a}C(O)OR^{2b} or —OC(O)NR^{2a}R^{2b}.
55. R⁶, R⁷ and R⁸ are at each occurrence independently selected from hydrogen or C₁-C₆ alkyl, where any alkyl in R⁶, R⁷ and R⁸ may be optionally substituted by one, two or three substituents independently selected from halo, cyano, oxo, carboxy, —C(O)R^{2a}, —C(O)OR^{2a}, —NR^{2a}C(O)R^{2b} or —NR^{2a}C(O)OR^{2b}.
56. R⁶, R⁷ and R⁸ are at each occurrence independently selected from hydrogen or C₁-C₆ alkyl, where any alkyl in R⁶, R⁷ and R⁸ may be optionally substituted by one, two or three substituents independently selected from oxo, carboxy, —C(O)R^{2a}, —C(O)OR^{2a}, —NR^{2a}C(O)R^{2b} or —NR^{2a}C(O)OR^{2b}.
57. R⁶, R⁷ and R⁸ are at each occurrence independently selected from hydrogen or C₁-C₆ alkyl, where any alkyl in R⁶, R⁷ and R⁸ may be optionally substituted by one, two or three substituents independently selected from oxo, carboxy, —C(O)R^{2a} or —C(O)OR^{2a}.
58. R⁶, R⁷ and R⁸ are at each occurrence independently selected from hydrogen or unsubstituted C₁-C₆ alkyl, in particular hydrogen or unsubstituted C₁-C₄ alkyl.
59. R² is at each occurrence independently selected from unsubstituted C₁-C₆ alkyl or unsubstituted C₂-C₆ alkenyl;

R³, R⁴ and R⁵ are at each occurrence independently selected from hydrogen or C₁-C₆ alkyl, where any alkyl in R³, R⁴ and R⁵ may be optionally substituted by one, two or three substituents independently selected from halo, cyano, oxo, hydroxy, carboxy; and

R⁶, R⁷ and R⁸ are at each occurrence independently selected from hydrogen or C₁-C₆ alkyl, where any alkyl in R⁶, R⁷ and R⁸ may be optionally substituted by one, two or three substituents independently selected from halo, cyano, oxo, hydroxy, carboxy, —OR^{2a}, —C(O)R^{2a}, —C(O)OR^{2a}, —NR^{2a}C(O)R^{2b} or —NR^{2a}C(O)OR^{2b}.

60. X and Y are each independently selected from a bond, phenyl or 5- to 6-membered heteroaryl; any phenyl or 5- to 6-membered heteroaryl in X or Y may be optionally substituted by one, two or three substituents independently selected from halo, cyano, oxo, hydroxy, carboxy, R², —OR², —C(O)R², —C(O)OR²,—OC(O)R², —C(O)NR³R⁴, —NR³C(O)R⁴, —NR³R⁴, —SO₂NR³R⁴, —NR³SO₂R⁴, —SR³, —SO₂R³, —SO₂OR³, —OSO₂R³, —NR³SO₂NR⁴R⁵, —NR³C(O)NR⁴R⁵, —NR³C(O)OR⁴ or —OC(O)NR³R⁴.
61. X, Y and Z are each independently selected from a bond, phenyl or 5- to 6-membered heteroaryl; any phenyl or 5- to 6-membered heteroaryl in X, Y or Z may be optionally substituted by one, two or three substituents independently selected from halo, cyano, oxo, hydroxy, carboxy, R², —OR², —C(O)R², —C(O)OR²,—OC(O)R², —C(O)NR³R⁴, —NR³C(O)R⁴, —NR³R⁴, —SO₂NR³R⁴, —NR³SO₂R⁴, —SR³, —SO₂R³, —SO₂OR³, —OSO₂R³, —NR³SO₂NR⁴R⁵, —NR³C(O)NR⁴R⁵, —NR³C(O)OR⁴ or —OC(O)NR³R⁴.
62. X, Y and Z are each independently selected from a bond, phenyl or 5- to 6-membered heteroaryl; any phenyl or 5- to 6-membered heteroaryl in X, Y or Z may be optionally substituted by one or more substituents independently selected from halo, cyano, oxo, hydroxy, carboxy, R², —OR², —C(O)R², —C(O)OR²,—OC(O)R², —C(O)NR³R⁴, —NR³C(O)R⁴, —NR³R⁴, —SO₂NR³R⁴, —NR³SO₂R⁴, —SR³, —SO₂R³, —SO₂OR³, —OSO₂R³, —NR³SO₂NR⁴R⁵, —NR³C(O)NR⁴R⁵, —NR³C(O)OR⁴ or —OC(O)NR³R⁴.
63. At least one of X, Y and Z is selected from optionally substituted phenyl or optionally substituted 5- or 6-membered heteroaryl; and at least one of X, Y and Z is a bond.
64. At least one of X, Y and Z is selected from unsubstituted phenyl or unsubstituted 5- or 6-membered heteroaryl; and at least one of X, Y and Z is a bond.
65. At least one of X, Y and Z is unsubstituted phenyl; and at least one of X, Y and Z is a bond.
66. One of X and Y is unsubstituted phenyl or unsubstituted 5- to 6-membered heteroaryl; and one of X and Y is a bond.
67. One of X and Y is unsubstituted phenyl; and one of X and Y is a bond.
68. X is selected from a bond, unsubstituted phenyl or unsubstituted 5- to 6-membered heteroaryl.
69. X is an unsubstituted 5-membered heteroaryl, such as pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl or tetrazolyl.
70. X is an unsubstituted 6-membered heteroaryl, such as pyridyl, pyrazinyl, pyridazinyl, pyrimidinyl and triazinyl.
71. X is selected from a bond or unsubstituted phenyl.
72. X is unsubstituted phenyl.
73. X is a bond and L¹ is selected from a bond, —C(O)—, —OC(O)—, —NR³C(O)—, —NR³SO₂—, —SO₂— and —OSO₂—, particularly L¹ is selected from —C(O)— or —NHC(O)—.
74. When X is a bond and L¹ is a bond, then R¹ is not hydrogen.
75. X is unsubstituted phenyl; Y is a bond and L² is selected from a bond, —C(O)—, —C(O)O—, —C(O)NR³—, —SO₂NR³—, —SO₂— or —SO₂O, particularly L² is selected from —C(O)— or —C(O)NH—.
76. X is a bond; Y is unsubstituted phenyl and L¹ selected from a bond, —C(O)—, —OC(O)—,—NR³C(O)—, —NR³SO₂—, —SO₂— or —OSO₂, particularly L¹ is selected from —C(O)— or —NHC(O)—.
77. Y is selected from a bond, unsubstituted phenyl or unsubstituted 5- to 6-membered heteroaryl.
78. Y is an unsubstituted 5-membered heteroaryl, such as pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl or tetrazolyl.
79. Y is an unsubstituted 6-membered heteroaryl, such as pyridyl, pyrazinyl, pyridazinyl, pyrimidinyl and triazinyl.
80. Y is selected from a bond or unsubstituted phenyl.
81. Y is unsubstituted phenyl.
82. Y is a bond and L² is selected from a bond, —C(O)—, —C(O)O—, —C(O)NR³—, —SO₂NR³—, —SO₂— or —SO₂O, particularly L² is selected from —C(O)— or —C(O)NH—.
83. Z is selected from a bond, C₃-C₆ cycloalkyl, C₃-C₆ heterocycloalkyl, phenyl or 5- to 6-membered heteroaryl, where any cycloalkyl, heterocycloalkyl, phenyl or heteroaryl in Z may be optionally substituted by one, two or three substituents independently selected from halo, cyano, oxo, hydroxy or carboxy; and any heterocycloalkyl or heteroaryl in Z including 1 or 2 heteroatoms selected from N, O or S in the ring.
84. Z is selected from a bond, unsubstituted C₃-C₆ cycloalkyl, unsubstituted C₃-C₆ heterocycloalkyl, unsubstituted phenyl or unsubstituted 5- to 6-membered heteroaryl.
85. Z is unsubstituted C₃-C₆ heterocycloalkyl, such as cyclopropyl, cyclobutyl, cyclopentyl or cyclohexyl.
86. Z is unsubstituted C₃-C₆ heterocycloalkyl, such as azetidinyl, tetrahydrofuranyl, tetrahydropyranyl, pyrrolidinyl, morpholinyl, tetrahydrothienyl, thiomorpholinyl, piperidinyl or piperazinyl.
87. Z is an unsubstituted 5-membered heteroaryl, such as pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl or tetrazolyl.
88. Z is an unsubstituted 6-membered heteroaryl, such as pyridyl, pyrazinyl, pyridazinyl, pyrimidinyl and triazinyl.
89. Z is selected from a bond or unsubstituted phenyl.
90. Z is unsubstituted phenyl.
91. Z is a bond.
92. X is unsubstituted phenyl; Y is a bond; Z is a bond and L² is selected from a bond, —C(O)—, —C(O)O—, —C(O)NR³—, —SO₂NR³—, —SO₂— or —SO₂O, particularly L² is selected from —C(O)— or —C(O)NH—.
93. X is unsubstituted phenyl; Y is unsubstituted phenyl; and Z is a bond.
94. X is unsubstituted phenyl; Y is a bond; Z is unsubstituted phenyl and L² is selected from a bond, —C(O)—, —C(O)O—, —C(O)NR³—, —SO₂NR³—, —SO₂— or —SO₂O, particularly L² is selected from a bond, —C(O)— or —C(O)NH—.
95. X is a bond; Y is unsubstituted phenyl; Z is a bond and L¹ is selected from a bond, —C(O)—, —OC(O)—, —NR³C(O)—, —NR³SO₂—, —SO₂— or —OSO₂—, particularly L¹ is selected from —C(O)— or—NHC(O)—.
96. X is a bond; Y is a bond; Z is unsubstituted phenyl; L¹ is selected from a bond, —C(O)—, —OC(O)—, —NR³C(O)—, —NR³SO₂—, —SO₂— or —OSO₂—, particularly L¹ is selected from a bond, —C(O)— or —NHC(O)—; and L² is selected from a bond, —C(O)—, —C(O)O—, —C(O)NR³—, —SO₂NR³—, —SO₂— or —SO₂O, particularly L² is selected from a bond, —C(O)— or —C(O)NH—.
97. X is a bond; Y is unsubstituted phenyl; Z is unsubstituted phenyl and L¹ is selected from a bond, —C(O)—, —OC(O)—,—NR³C(O)—, —NR³SO₂—, —SO₂— or —OSO₂, particularly L¹ is selected from —C(O)— or —NHC(O)—.
98. L¹ is selected from a bond, —O—, —C(O)—, —C(O)O—, —OC(O)—, —C(O)NR³—, —NR³C(O)— or —SO₂—, in particular L¹ is selected from a bond, —O—, —C(O)—, —C(O)O—, —OC(O)—, —C(O)NH—, —NHC(O)— or —SO₂—.
99. L¹ is selected from a bond, —O—, —C(O)—, —C(O)NH— or —SO₂—.
100. L¹ is selected from a bond, —O—, —C(O)— or —C(O)NH—.
101. L¹ is selected from a bond or —O—, particularly L¹ is —O—.
102. L² is selected from a bond, —O—, —C(O)—, —C(O)O—, —OC(O)—, —C(O)NR³— or —NR³C(O)—, in particular L² is selected from a bond, —O—, —C(O)—, —C(O)O—, —OC(O)—, —C(O)NH— or —NHC(O)—.
103. L² is selected from a bond, —O—, —C(O)— or —C(O)NH—.
104. L² is selected from a bond, —C(O)— or —C(O)NH—.
105. L² is selected from a bond or —C(O)—, in particular L² is —C(O)—.
106. L³ is selected from a bond or C₁-C₄ alkylene, where any alkylene in L³ may be optionally substituted by one or two substituents independently selected from halo, cyano, oxo, hydroxy, carboxy, R², —OR²,—C(O)R², —C(O)OR²,—OC(O)R², —C(O)NR⁶R⁷, —NR⁶C(O)R⁷, —NR⁶R⁷, —SO₂NR⁶R⁷, —NR⁶SO₂R⁷, —SR⁶, —SO₂R⁶, —SO₂OR⁶, —OSO₂R⁶, —NR⁶SO₂NR⁷R⁸, —NR⁶C(O)NR⁷R⁸, —NR⁶C(O)OR⁷ or —OC(O)NR⁶R⁷, particularly any alkylene in L³ may be optionally substituted by one or two substituents independently selected from halo and unsubstituted C₁₋₄ alkyl, such as C₁-C₄ alkylene substituted by one or two halo (e.g. F) or C₁-C₄ alkylene substituted by one or two C₁₋₄ alkyl (e.g. methyl).
107. L³ is selected from a bond, unsubstituted C₁-C₄ alkylene, C₁-C₄ alkylene substituted by one or two C₁₋₄ alkyl or C₁-C₄ alkylene substituted by one or two halo (e.g. F).
108. L³ is selected from a bond or unsubstituted C₁-C₄ alkylene,
109. L³ is unsubstituted C₁-C₄ alkylene.
110. L³ is C₁-C₄ alkylene optionally substituted with one or more substituents selected from R², halo or cyano.
111. L³ is C₁-C₄ alkylene optionally substituted with one or more substituents selected from chloro, fluoro or methyl.
112. L³ is methylene optionally substituted with one or more substituents selected from R² or halo.
113. L³ is methylene optionally substituted with one or more substituents selected from chloro or fluoro.
114. Y is optionally substituted phenyl or optionally substituted 5- to 6-membered heteroaryl (optional substituents as defined above); and

L² is selected from a bond, —O—, —C(O)—, —C(O)O—, —OC(O)—, —C(O)NR³—, —NR³C(O)—, —NR³—, —SO₂NR³—, —NR³SO₂—, —S—, —SO₂—, —SO₂O—, —OSO₂—, —NR³SO₂NR⁴—, —NR³C(O)NR⁴—, —NR³C(O)O— or —OC(O)NR³.

115. Y is optionally substituted phenyl or optionally substituted 5- to 6-membered heteroaryl (optional substituents as defined above);

L² is selected from a bond, —O—, —C(O)—, —C(O)O—, —OC(O)—, —C(O)NR³—, —NR³C(O)—, —NR³—, —SO₂NR³—, —NR³SO₂—, —S—, —SO₂—, —SO₂O—, —OSO₂—, —NR³SO₂NR⁴—, —NR³C(O)NR⁴—, —NR³C(O)O— or —OC(O)NR³; and

Z is a bond.

116. Y is optionally substituted phenyl or optionally substituted 5- to 6-membered heteroaryl (optional substituents as defined above); and L² is —O—.
117. Y is optionally substituted phenyl or optionally substituted 5- to 6-membered heteroaryl (optional substituents as defined above); L² is —O—; and Z is a bond.
118. Y is unsubstituted phenyl; and L² is —O—.
119. Y is unsubstituted phenyl; L² is —O—; and Z is a bond.
120. Y is bond, L² is C(O), L³ is CH(F), Z is a bond and q is 0.
121. R⁹ and R¹⁰ are at each occurrence independently selected from hydrogen and methyl.

### Compounds of Formula (I)

In embodiments, there is provided a compound of Formula (I) or (Ia), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L¹ is selected from a bond, —O—, —C(O)— or —NHC(O)—;

L² is selected from a bond, —O—, —C(O)— or —C(O)NH—;

one of X and Y is unsubstituted phenyl or unsubstituted 5- to 6-membered heteroaryl and one of X and Y is a bond;

provided when X is a bond, then L¹ is selected from a bond, —C(O)— or —NHC(O)—;

provided when Y is a bond, then L² is selected from a bond, —C(O)— or —C(O)NH;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene;

Z is selected from a bond, C₃-C₆ cycloalkyl, C₃-C₆ heterocycloalkyl, phenyl or 5- to 6-membered heteroaryl, particularly Z is a bond, unsubstituted phenyl or unsubstituted 5- to 6-membered heteroaryl,
any cycloalkyl, heterocycloalkyl, phenyl or heteroaryl in Z may be optionally substituted by one, two or three substituents independently selected from halo, cyano, oxo, hydroxy or carboxy;
any heterocycloalkyl or heteroaryl in Z including 1 or 2 heteroatoms selected from N, O or S in the ring; and

R¹ is unsubstituted C₁-C₆ alkyl.

### Compounds of Formula (III)

In embodiments, there is provided a compound of Formula (III), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L¹ is selected from a bond, —O—, —C(O)— or —C(O)NH—, particularly L¹ is selected from a bond or —O—;

L² is selected from a bond, —O—, —C(O)— or —C(O)NH—, particularly L² is selected from a bond, —C(O)— or —C(O)NH—;

Y is a bond, unsubstituted phenyl or unsubstituted 5- to 6-membered heteroaryl;

provided when Y is a bond, then L² is selected from a bond, —C(O)— or —C(O)NH;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene;

Z is selected from a bond, C₃-C₆ cycloalkyl, C₃-C₆ heterocycloalkyl, phenyl or 5- to 6-membered heteroaryl, particularly Z is a bond, unsubstituted phenyl or unsubstituted 5- to 6-membered heteroaryl;
any cycloalkyl, heterocycloalkyl, phenyl or heteroaryl in Z may be optionally substituted by one, two or three substituents independently selected from halo, cyano, oxo, hydroxy or carboxy;
any heterocycloalkyl or heteroaryl in Z including 1 or 2 heteroatoms selected from N, O or S in the ring; and
   R¹ is unsubstituted C₁-C₆ alkyl.

In embodiments, there is provided a compound of Formula (III), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L¹ is selected from a bond, —O—, —C(O)— or —C(O)NH— particularly L¹ is selected from a bond or —O—;

L² is selected from a bond, —C(O)— or —C(O)NH;

Y is a bond;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene;

Z is a bond; and

R¹ is unsubstituted C₁-C₆ alkyl.

In embodiments, there is provided a compound of Formula (III), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L¹ is selected from a bond, —O—, —C(O)— or —C(O)NH—; particularly L¹ is selected from a bond or —O—;

L² is —C(O)—;

Y is a bond;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene;

Z is a bond; and

R¹ is unsubstituted C₁-C₆ alkyl.

In embodiments, there is provided a compound of Formula (III), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L¹ is selected from a bond, —O—, —C(O)— or —C(O)NH—, particularly L¹ is selected from a bond or —O—;

L² is —C(O)NH—;

Y is a bond;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene;

Z is a bond; and

R¹ is unsubstituted C₁-C₆ alkyl.

In embodiments, there is provided a compound of Formula (III), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L¹ is selected from a bond, —O—, —C(O)— or —C(O)NH—, particularly L¹ is selected from a bond or —O—;

L² is selected from a bond, —C(O)— or —C(O)NH;

Y is a bond;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene;

Z is unsubstituted phenyl; and

R¹ is unsubstituted C₁-C₆ alkyl.

In embodiments, there is provided a compound of Formula (III), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L¹ is selected from a bond, —O—, —C(O)— or —C(O)NH—, particularly L¹ is selected from a bond or —O—;

L² is —C(O)—;

Y is a bond;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene;

Z is unsubstituted phenyl; and

R¹ is unsubstituted C₁-C₆ alkyl.

In embodiments, there is provided a compound of Formula (III), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L¹ is selected from a bond, —O—, —C(O)— or —C(O)NH—, particularly L¹ is selected from a bond or —O—;

L² is —C(O)NH—;

Y is a bond;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene;

Z is unsubstituted phenyl; and

R¹ is unsubstituted C₁-C₆ alkyl.

In embodiments, there is provided a compound of Formula (III), or a pharmaceutically acceptable salt thereof, wherein wherein L¹ is a bond and R¹ is halogen.

### Compounds of Formula (VIII)

In embodiments, there is provided a compound of Formula (VIII), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L² is selected from a bond, —O—, —C(O)— or —C(O)NH—, particularly L² is selected from a bond, —C(O)— or —C(O)NH—;

Y is a bond, unsubstituted phenyl or unsubstituted 5- to 6-membered heteroaryl;

provided when Y is a bond, then L² is selected from a bond, —C(O)— or —C(O)NH;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene;

Z is selected from a bond, C₃-C₆ cycloalkyl, C₃-C₆ heterocycloalkyl, phenyl or 5- to 6-membered heteroaryl, particularly Z is a bond, unsubstituted phenyl or unsubstituted 5- to 6-membered heteroaryl;
any cycloalkyl, heterocycloalkyl, phenyl or heteroaryl in Z may be optionally substituted by one, two or three substituents independently selected from halo, cyano, oxo, hydroxy or carboxy;
any heterocycloalkyl or heteroaryl in Z including 1 or 2 heteroatoms selected from N, O or S in the ring; and
R^{1a} and R^{1b} together form a 3- to 7-membered heterocycloalkyl, optionally including one additional heteroatom selected from O, N or S in the ring, where said heterocyclalkyl formed by R^{1a} and R^{1b} may be optionally substituted by one or two substituents independently selected from oxo, —SO₂R³, —SO₂OR³ or —OSO₂R³, where R³ is unsubstituted C₁-C₆ alkyl, in particular R³ is unsubstituted C₁-C₄ alkyl.

In embodiments, there is provided a compound of Formula (VIII), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L² is selected from a bond, —C(O)— or —C(O)NH—;

Y is a bond;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene;

Z is a bond; and
R^{1a} and R^{1b} together form a 3- to 7-membered heterocycloalkyl, optionally including one additional heteroatom selected from O, N or S in the ring, where said heterocyclalkyl formed by R^{1a} and R^{1b} may be optionally substituted by one or two substituents independently selected from oxo, —SO₂R³, —SO₂OR³ or —OSO₂R³, where R³ is unsubstituted C₁-C₆ alkyl, in particular R³ is unsubstituted C₁-C₄ alkyl.

In embodiments, there is provided a compound of Formula (VIII), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L² is a bond or —C(O)—;

Y is a bond;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene;

Z is a bond; and
R^{1a} and R^{1b} together form a 3- to 7-membered heterocycloalkyl, optionally including one additional heteroatom selected from O, N or S in the ring, where said heterocyclalkyl formed by R^{1a} and R^{1b} may be optionally substituted by one or two substituents independently selected from oxo, —SO₂R³, —SO₂OR³ or —OSO₂R³, where R³ is unsubstituted C₁-C₆ alkyl, in particular R³ is unsubstituted C₁-C₄ alkyl.

### Compounds of Formula (IX)

In embodiments, there is provided a compound of Formula (IX), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L² is selected from a bond, —O—, —C(O)— or —C(O)NH—;

Y is a bond, unsubstituted phenyl or unsubstituted 5- to 6-membered heteroaryl;

provided when Y is a bond, then L² is selected from a bond, —C(O)— and —C(O)NH;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene; and

Z is selected from a bond, C₃-C₆ cycloalkyl, C₃-C₆ heterocycloalkyl, phenyl or 5- to 6-membered heteroaryl, particularly Z is a bond, unsubstituted phenyl or unsubstituted 5- to 6-membered heteroaryl,
any cycloalkyl, heterocycloalkyl, phenyl or heteroaryl in Z may be optionally substituted by one, two or three substituents independently selected from halo, cyano, oxo, hydroxy or carboxy;
any heterocycloalkyl or heteroaryl in Z including 1 or 2 heteroatoms selected from N, O or S in the ring.

In embodiments, there is provided a compound of Formula (IX), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L² is selected from a bond, —C(O)— or —C(O)NH—;

Y is a bond;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene; and

Z is a bond.

In embodiments, there is provided a compound of Formula (IX), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L² is a bond or —C(O)—;

Y is a bond;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene; and

Z is a bond.

### Compounds of Formula (X)

In embodiments, there is provided a compound of Formula (X), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L² is selected from a bond, —O—, —C(O)— or —C(O)NH—, particularly L² is selected from—a bond, —C(O)— or —C(O)NH;

Y is a bond, unsubstituted phenyl or unsubstituted 5- to 6-membered heteroaryl;

provided when Y is a bond, then L² is selected from a bond, —C(O)— or —C(O)NH;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene;

Z is selected from a bond, C₃-C₆ cycloalkyl, C₃-C₆ heterocycloalkyl, phenyl or 5- to 6-membered heteroaryl, particularly Z is a bond, unsubstituted phenyl or unsubstituted 5- to 6-membered heteroaryl;
any cycloalkyl, heterocycloalkyl, phenyl or heteroaryl in Z may be optionally substituted by one, two or three substituents independently selected from halo, cyano, oxo, hydroxy or carboxy;
any heterocycloalkyl or heteroaryl in Z including 1 or 2 heteroatoms selected from N, O or S in the ring; and

R¹ is unsubstituted C₁-C₆ alkyl.

In embodiments, there is provided a compound of Formula (X), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L² is —C(O)— or —C(O)NH—;

Y is a bond;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene;

Z is selected from a bond, C₃-C₆ cycloalkyl, C₃-C₆ heterocycloalkyl, phenyl or 5- to 6-membered heteroaryl, particularly Z is a bond, unsubstituted phenyl or unsubstituted 5- to 6-membered heteroaryl;
any cycloalkyl, heterocycloalkyl, phenyl or heteroaryl in Z may be optionally substituted by one, two or three substituents independently selected from halo, cyano, oxo, hydroxy or carboxy;
any heterocycloalkyl or heteroaryl in Z including 1 or 2 heteroatoms selected from N, O or S in the ring; and

R¹ is unsubstituted C₁-C₆ alkyl.

In embodiments, there is provided a compound of Formula (X), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L² is —C(O)— or —C(O)NH—;

Y is a bond;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene;

Z is a bond; and

R¹ is unsubstituted C₁-C₆ alkyl.

### Compounds of Formula (XIX)

In embodiments, there is provided a compound of Formula (XIX), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

X is unsubstituted phenyl;

L¹ is selected from a bond, —O—, —C(O)— or —C(O)NH—, particularly L¹ is selected from a bond or —O—;

L² is selected from a bond, —C(O)— or —C(O)NH—;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene; and

R¹ is unsubstituted C₁-C₆ alkyl.

### Compounds of Formula (XXII)

In embodiments, there is provided a compound of Formula (XXII), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L² is selected from a bond, —C(O)— or —C(O)NH—;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene; and

R¹ is unsubstituted C₁-C₆ alkyl.

In embodiments, there is provided a compound of Formula (XXII), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L² is —C(O)—;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene; and

R¹ is unsubstituted C₁-C₆ alkyl.

In embodiments, there is provided a compound of Formula (XXII), or a pharmaceutically acceptable salt thereof, wherein

m and n are each independently selected from 2 or 3;

the cyclic diamine moiety is bridged by —(CR⁹R¹⁰)ₒ—, where R⁹ and R¹⁰ are at each occurrence independently selected from H and unsubstituted C₁₋₄ alkyl and o is 1, 2, 3 or 4, particularly o is 1 or 2, and the ring carbon atoms of the cyclic diamine moiety is unsubstituted;

L² is a bond;

L³ is selected from a bond, unsubstituted C₁-C₄ alkylene, C₁-C₄ alkylene substituted by one or two halo (e.g. F), or C₁-C₄ alkylene substituted by one or two C₁₋₄ alkyl (e.g. methyl), particularly L³ is selected from a bond or unsubstituted C₁-C₄ alkylene; and

R¹ is unsubstituted C₁-C₆ alkyl.

In embodiments, there is provided a compound of the structure of Formula (XXVI): or a pharmaceutically acceptable salt thereof, wherein

X is selected from a bond, unsubstitued phenyl or phenyl substituted by one or more halo groups;

L¹ is selected from a bond, —O— and —SO₂—; and

R¹ is selected from hydrogen, halogen, C₁-C₆ alkyl, or a 3- to 12-membered ring system (e.g. cycloalkyl, heterocyclyl, aryl or heteroaryl), including 0, 1, 2 or 3 heteroatoms selected from N, O or S in the ring system, where
any ring system in R¹ may be optionally substituted by one or more substituents independently selected from halo, cyano, oxo, and SO₂R³.

In one embodiment there is provided a compound of Formula (XXVI) or a pharmaceutically acceptable salt thereof, wherein X is phenyl substituted by one or more halo groups, L¹ is a bond and R¹ is hydrogen.

In another embodiment there is provided a compound of Formula (XXVI) or a pharmaceutically acceptable salt thereof, wherein X is a bond, L¹ is a bond and R¹ is halogen.

In an embodiment, the compound of formula (I) is a compound selected from:

TABLE 1

| Chemical name | Structure |
|---|---|
| 2-((2-((1R,5S)-6-(4-ethoxyphenyl)-9,9-dimethyl- 3,6-diazabicyclo[3.2.2]nonan-3- yl)ethyl)sulfonyl)ethan-1-amine | 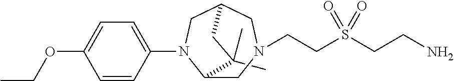 [SMILES: CCOc1ccc(N2C[C@H]3CN(CCS(=O)(=O)CCN)C[C@@H]2C(C)(C)C3)cc1] |
| 2-((4-((1R,5S)-6-(4-ethoxyphenyl)-9,9-dimethyl- 3,6-diazabicyclo[3.2.2]nonan-3- yl)phenyl)sulfonyl)ethan-1-amine | 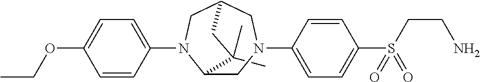 [SMILES: CCOc1ccc(N2C[C@H]3CN(c4ccc(S(=O)(=O)CCN)cc4)C[C@@H]2C(C)(C)C3)cc1] |
| (4-((2-aminoethyl)sulfonyl)phenyl)((1S,5S)-6-(4- ethoxyphenyl)-9,9-dimethyl-3,6- diazabicyclo[3.2.2]nonan-3-yl)methanone | 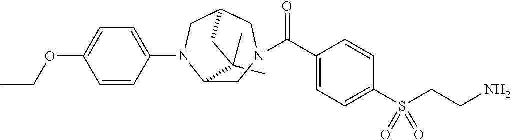 [SMILES: CCOc1ccc(N2C[C@H]3CN(C(=O)c4ccc(S(=O)(=O)CCN)cc4)C[C@@H]2C(C)(C)C3)cc1] |
| (3-((2-aminoethyl)sulfonyl)phenyl)((1S,5S)-6-(4- ethoxyphenyl)-9,9-dimethyl-3,6- diazabicyclo[3.2.2]nonan-3-yl)methanone | 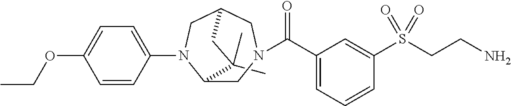 [SMILES: CCOc1ccc(N2C[C@H]3CN(C(=O)c4cccc(S(=O)(=O)CCN)c4)C[C@@H]2C(C)(C)C3)cc1] |
| 2-((2-aminoethyl)sulfonyl)-1-((1S,5S)-6-(4- ethoxyphenyl)-9,9-dimethyl-3,6- diazabicyclo[3.2.2] nonan-3-yl)ethan-1-one | 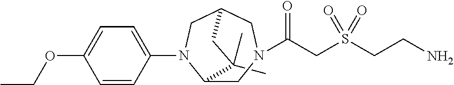 [SMILES: CCOc1ccc(N2C[C@H]3CN(C(=O)CS(=O)(=O)CCN)C[C@@H]2C(C)(C)C3)cc1] |
| (1S,5S)-N-(2-((2-aminoethyl)sulfonyl)ethyl)-6- (4-ethoxyphenyl)-9,9-dimethyl-3,6- diazabicyclo[3.2.2]nonane-3-carboxamide | 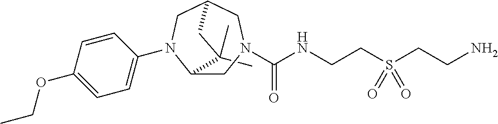 [SMILES: CCOc1ccc(N2C[C@H]3CN(C(=O)NCCS(=O)(=O)CCN)C[C@@H]2C(C)(C)C3)cc1] |
| 2-((2-aminoethyl)sulfonyl)-1-((1S,5S)-6-(4- ethoxyphenyl)-9,9-dimethyl-3,6- diazabicyclo[3.2.2]nonan-3-yl)-4-methylpentan- 1-one | 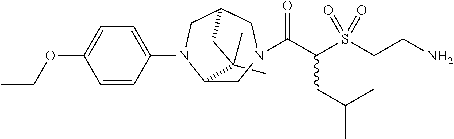 [SMILES: CCOc1ccc(N2C[C@H]3CN(C(=O)C(CC(C)C)S(=O)(=O)CCN)C[C@@H]2C(C)(C)C3)cc1] |
| 2-((3-((1R5S)-6-(4-ethoxyphenyl)-9,9-dimethyl- 3,6-diazabicyclo[3.2.2]nonan-3- yl)propyl)sulfonyl)ethan-1-amine | 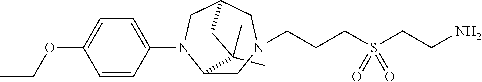 [SMILES: CCOc1ccc(N2C[C@H]3CN(CCCS(=O)(=O)CCN)C[C@@H]2C(C)(C)C3)cc1] |
| (1S,5S)-N-(4-((2-aminoethyl)sulfonyl)phenyl)-6- (4-ethoxyphenyl)-9,9-dimethyl-3,6- diazabicyclo[3.2.2]nonane-3-carboxamide | 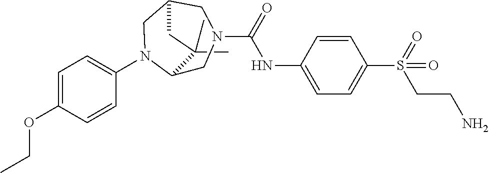 [SMILES: CCOc1ccc(N2C[C@H]3CN(C(=O)Nc4ccc(S(=O)(=O)CCN)cc4)C[C@@H]2C(C)(C)C3)cc1] |
| 2-((2-aminoethyl)sulfonyl)-1-((1S,5S)-6-(4- ethoxyphenyl)-3,6-diazabicyclo[3.2.2]nonan-3- yl)ethan-1-one | 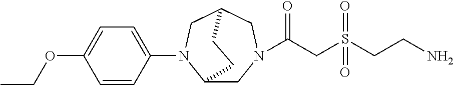 [SMILES: CCOc1ccc(N2C[C@@H]3CC[C@H]2CN(C(=O)CS(=O)(=O)CCN)C3)cc1] |
| 4-(4-(4-(2-((2-aminoethyl)sulfonyl)ethyl)-1,4- diazepan-1-yl)phenyl)thiomorpholine-1,1- dioxide | 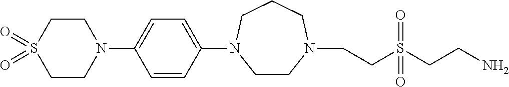 [SMILES: NCCS(=O)(=O)CCN1CCCN(c2ccc(N3CCS(=O)(=O)CC3)cc2)CC1] |
| 2-((2-((1S,4S)-5-(4-ethoxyphenyl)-2,5- diazabicyclo[2.2.1]heptan-2-yl)ethyl)sulfonyl)- ethan-1-amine | 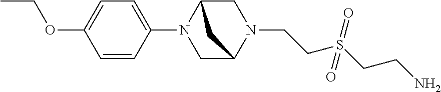 [SMILES: CCOc1ccc(N2C[C@@H]3C[C@H]2CN3CCS(=O)(=O)CCN)cc1] |
| 2-((2-aminoethyl)sulfonyl)-1-((1S,4S)-5-(4- ethoxyphenyl)-2,5-diazabicyclo[2.2.1]heptan-2- yl)ethan-1-one | 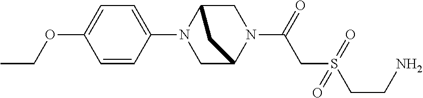 |
| 2-((2-aminoethyl)sulfonyl)-1-((1S,4S)-5-(4- ethoxyphenyl)-2,5-diazabicyclo[2.2.1]heptan-2- yl)-2-fluoroethan-1-one | 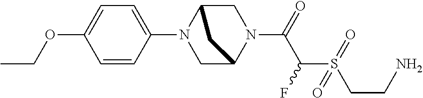 |
| ((1S,5S)-6-(4-(3-((2- aminoethyl)sulfonyl)propoxy)phenyl)-9,9- dimethyl-3,6-diazabicyclo[3.2.2]nonan-3-yl)(1,1- dioxidothiomorpholino)methanone | 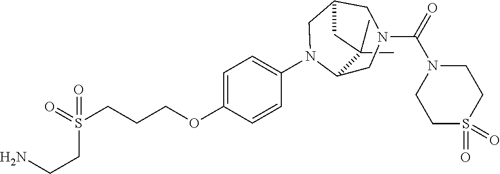 [SMILES: CC1(C)C[C@@H]2CN(C(=O)N3CCS(=O)(=O)CC3)C[C@H]1N(c1ccc(OCCCS(=O)(=O)CCN)cc1)C2] |
| (1S,5S)-6-(4-(3-((2- aminoethyl)sulfonyl)propoxy)phenyl)-N-ethyl- 9,9-dimethyl-3,6-diazabicyclo[3.2.2]nonane-3- carboxamide | 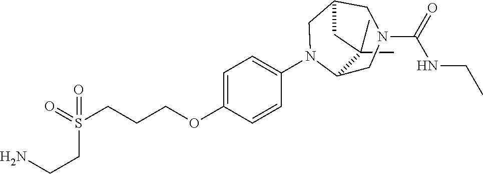 [SMILES: CCNC(=O)N1C[C@@H]2CN(c3ccc(OCCCS(=O)(=O)CCN)cc3)[C@H](C1)C(C)(C)C2] |
| (1S,5S)-6-(4-(3-((2- aminoethyl)sulfonyl)propoxy)phenyl)-N-(2- hydroxyethyl)-9,9-dimethyl-3,6- diazabicyclo[3.2.2]nonane-3-carboxamide | 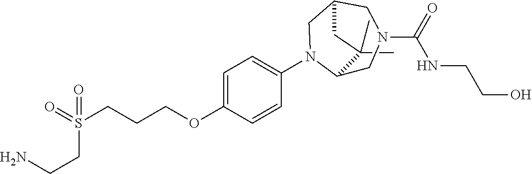 [SMILES: CC1(C)C[C@@H]2CN(C(=O)NCCO)C[C@H]1N(c1ccc(OCCCS(=O)(=O)CCN)cc1)C2] |
| 2-((2-aminoethyl)sulfonyl)-1-((1S,5S)-6-(4-(1,1- dioxidothiomorpholino)phenyl)-9,9-dimethyl- 3,6-diazabicyclo[3.2.2]nonan-3-yl)ethan-1-one | 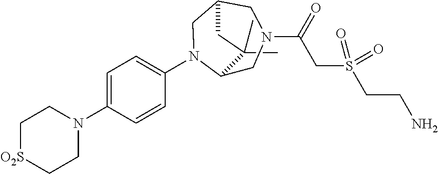 [SMILES: CC1(C)C[C@@H]2CN(C(=O)CS(=O)(=O)CCN)C[C@H]1N(c1ccc(N3CCS(=O)(=O)CC3)cc1)C2] |
| 4-(4-((1R,5S)-3-(2-((2- aminoethyl)sulfonyl)ethyl)-9,9-dimethyl-3,6- diazabicyclo[3.2.2]nonan-6- yl)phenyl)thiomorpholine 1,1-dioxide | 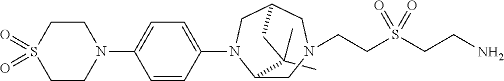 [SMILES: CC1(C)C[C@@H]2CN(CCS(=O)(=O)CCN)C[C@H]1N(c1ccc(N3CCS(=O)(=O)CC3)cc1)C2] |
| 2-((2-aminoethyl)sulfonyl)-1-((1S,5S)-6-(4-(1,1- dioxidothiomorpholino)phenyl)-9,9-dimethyl- 3,6-diazabicyclo[3.2.2]nonan-3-yl)-2- fluoroethan-1-one | 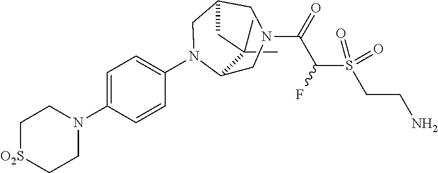 [SMILES: CC1(C)C[C@@H]2CN(C(=O)C(F)S(=O)(=O)CCN)C[C@H]1N(c1ccc(N3CCS(=O)(=O)CC3)cc1)C2] |
| 2-((2-aminoethyl)sulfonyl)-1-((1S,5S)-6-(4- ethoxyphenyl)-9,9-dimethyl-3,6- diazabicyclo[3.2.2]nonan-3-yl)-2-fluoroethan-1- one | 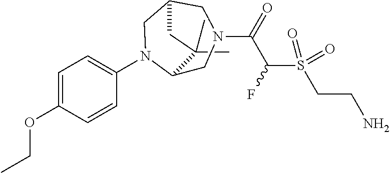 [SMILES: CCOc1ccc(N2C[C@H]3CN(C(=O)C(F)S(=O)(=O)CCN)C[C@@H]2C(C)(C)C3)cc1] |
| 2-((2-aminoethyl)sulfonyl)-1-((1S,5S)-6-(4- ethoxyphenyl)-9,9-dimethyl-3,6- diazabicyclo[3.2.2]nonan-3-yl)propan-1-one | 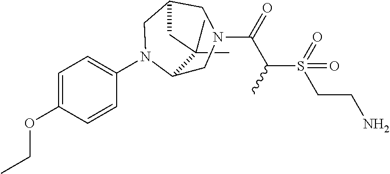 [SMILES: CCOc1ccc(N2C[C@H]3CN(C(=O)C(C)S(=O)(=O)CCN)C[C@@H]2C(C)(C)C3)cc1] |
| 2-((2-aminoethyl)sulfonyl)-1-((1S,5S)-6-(4- ethoxyphenyl)-9,9-dimethyl-3,6- diazabicyclo[3.2.2]nonan-3-yl)-2,2- difluoroethan-1-one | 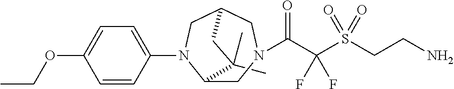 [SMILES: CCOc1ccc(N2C[C@H]3CN(C(=O)C(F)(F)S(=O)(=O)CCN)C[C@@H]2C(C)(C)C3)cc1] |
| 2-((2-(5-(4-ethoxyphenyl)hexahydropyrrolo[3,4- c]pyrrol-2(1H)-yl)ethyl)sulfonyl)ethan-1-amine | 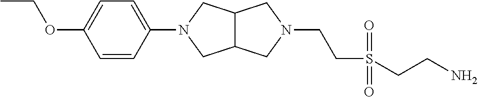 [SMILES: CCOc1ccc(N2CC3CN(CCS(=O)(=O)CCN)CC3C2)cc1] |

or a pharmaceutically acceptable salt of any of the foregoing compounds.

In an embodiment, the compound of formula (I) is a compound selected from:

TABLE 2

| Chemical name | Structure |
|---|---|
| Ethyl 4-((2-aminoethyl)sulfonyl)-5-((1S,4S)-5-(4- ethoxyphenyl)-2,5-diazabicyclo[2.2.1]heptan-2- yl)-5-oxopentanoate | 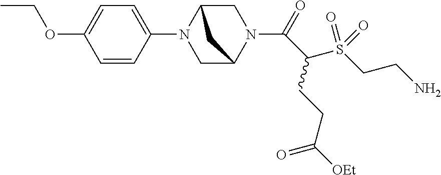 [SMILES: CCOC(=O)CCC(C(=O)N1C[C@@H]2C[C@H]1CN2c1ccc(OCC)cc1)S(=O)(=O)CCN] |
| (1-((2-Aminoethyl)sulfonyl)cyclopropyl)((1S,4S)- 5-(4-ethoxyphenyl)-2,5- diazabicyclo[2.2.1]heptan-2-yl)methanone | 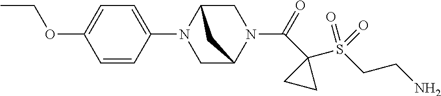 [SMILES: CCOc1ccc(N2C[C@@H]3C[C@H]2CN3C(=O)C2(S(=O)(=O)CCN)CC2)cc1] |
| 2-((2-Aminoethyl)sulfonyl)-1-(cis-5- tosylhexahydropyrrolo[3,4-c]pyrrol-2(1H)- yl)ethan-1-one | 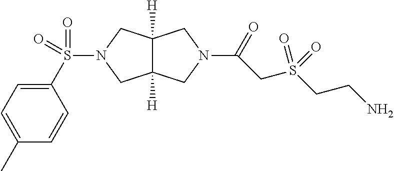 [SMILES: Cc1ccc(S(=O)(=O)N2C[C@H]3CN(C(=O)CS(=O)(=O)CCN)C[C@H]3C2)cc1] |
| (2-((2-Aminoethyl)sulfonyl)-2-fluoro-1-(cis-5-(4- fluorophenyl)hexahydropyrrolo[3,4-c]pyrrol- 2(1H)-yl)ethan-1-one | 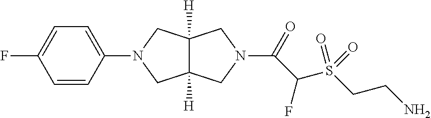 [SMILES: NCCS(=O)(=O)C(F)C(=O)N1C[C@@H]2CN(c3ccc(F)cc3)C[C@@H]2C1] |
| 2-((2-Aminoethyl)sulfonyl)-2-fluoro-1-(cis-5-(4- bromophenyl)hexahydropyrrolo[3,4-c]pyrrol- 2(1H)-yl)ethan-1-one | 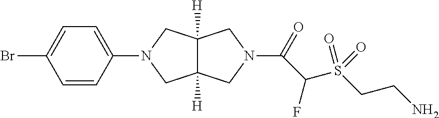 [SMILES: NCCS(=O)(=O)C(F)C(=O)N1C[C@@H]2CN(c3ccc(Br)cc3)C[C@@H]2C1] |
| 2-((2-Aminoethyl)sulfonyl)-2-fluoro-1-(cis-5- (perfluorophenyl)hexahydropyrrolo[3,4-c]pyrrol- 2(1H)-yl)ethan-1-one | 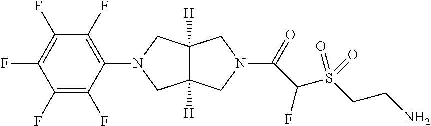 [SMILES: NCCS(=O)(=O)C(F)C(=O)N1C[C@@H]2CN(c3c(F)c(F)c(F)c(F)c3F)C[C@@H]2C1] |
| 2-((2-Aminoethyl)sulfonyl)-1-(4-(pyrimidin-2- yl)piperazin-1-yl)ethan-1-one | 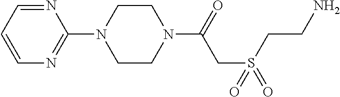 [SMILES: NCCS(=O)(=O)CC(=O)N1CCN(c2ncccn2)CC1] |
| 2-((2-Aminoethyl)sulfonyl)-1-(4-phenylpiperazin- 1-yl)ethan-1-one | 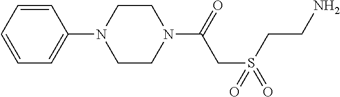 [SMILES: NCCS(=O)(=O)CC(=O)N1CCN(c2ccccc2)CC1] |
| 2-((2-Aminoethyl)sulfonyl)-1-(cis-5-(4- ethoxyphenyl)hexahydropyrrolo[3,4-c]pyrrol- 2(1H)-yl)ethan-1-one | 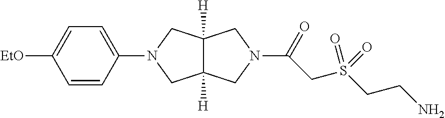 [SMILES: CCOc1ccc(N2C[C@H]3CN(C(=O)CS(=O)(=O)CCN)C[C@H]3C2)cc1] |
| 2-((2-Aminoethyl)sulfonyl)-2-fluoro-1-(4- phenylpiperazin-1-yl)ethan-1-one | 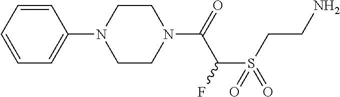 [SMILES: NCCS(=O)(=O)C(F)C(=O)N1CCN(c2ccccc2)CC1] |
| 2-((2-Aminoethyl)sulfonyl)-2-fluoro-1-(piperazin- 1-yl)ethan-1-one | 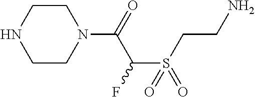 [SMILES: NCCS(=O)(=O)C(F)C(=O)N1CCNCC1] |
| 2-((2-Aminoethyl)sulfonyl)-2-fluoro-1-(4- (methylsulfonyl)piperazin-1-yl)ethan-1-one | 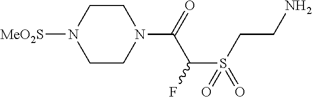 [SMILES: CS(=O)(=O)N1CCN(C(=O)C(F)S(=O)(=O)CCN)CC1] |
| 1-((1S,4S)-5-(4-((2-Aminoethyl)sulfonyl)phenyl)- 2,5-diazabicyclo[2.2.1]heptan-2-yl)-2- methylpropan-1-one | 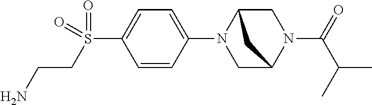 [SMILES: CC(C)C(=O)N1C[C@@H]2C[C@H]1CN2c1ccc(S(=O)(=O)CCN)cc1] |
| 4-((2-Aminoethyl)sulfonyl)phenyl(1S,5S)-6-(4- ethoxyphenyl)-9,9-dimethyl-3,6- diazabicyclo[3.2.2]nonane-3-carboxylate | 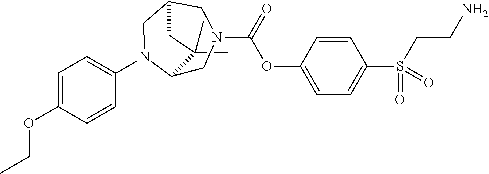 [SMILES: CCOc1ccc(N2C[C@H]3CN(C(=O)Oc4ccc(S(=O)(=O)CCN)cc4)C[C@@H]2C(C)(C)C3)cc1] |
| 1-((1S,4S)-5-(4-(((2- Aminoethyl)sulfonyl)methyl)phenyl)-2,5- diazabicyclo[2.2.1]heptan-2-yl)-2-methylpropan- 1-one | 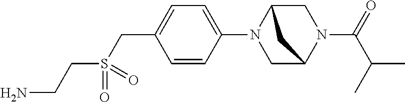 [SMILES: CC(C)C(=O)N1C[C@@H]2C[C@H]1CN2c1ccc(CS(=O)(=O)CCN)cc1] |
| (4-(((2- Aminoethyl)sulfonyl)methyl)phenyl)((1S,5S)-6- (4-ethoxyphenyl)-9,9-dimethyl-3,6- diazabicyclo[3.2.2]nonan-3-yl)methanone | 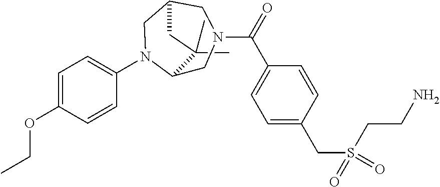 [SMILES: CCOc1ccc(N2C[C@H]3CN(C(=O)c4ccc(CS(=O)(=O)CCN)cc4)C[C@@H]2C(C)(C)C3)cc1] |
| 2-((2-Aminoethyl)sulfonyl)-1-((1S,5S)-9,9- dimethyl-6-(4-morpholinophenyl)-3,6- diazabicyclo[3.2.2]nonan-3-yl)-2-fluoroethan-1- one | 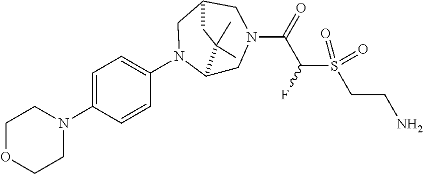 [SMILES: CC1(C)C[C@@H]2CN(C(=O)C(F)S(=O)(=O)CCN)C[C@H]1N(c1ccc(N3CCOCC3)cc1)C2] |
| 2-((2-Aminoethyl)sulfonyl)-1-(cis-3a,6a-dimethyl- 5-(4-(4-(methylsulfonyl)piperazin-1- yl)phenyl)hexahydropyrrolo[3,4-c]pyrrol-2(1H)- yl)-2-fluoroethan-1-one | 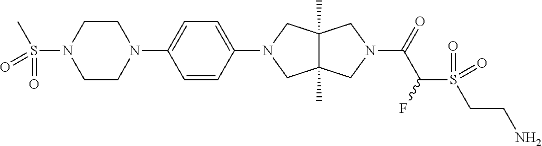 [SMILES: C[C@@]12CN(C(=O)C(F)S(=O)(=O)CCN)C[C@]1(C)CN(c1ccc(N3CCN(S(C)(=O)=O)CC3)cc1)C2] |
| 2-((2-Aminoethyl)sulfonyl)-1-(7-(4-(1,1- dioxidothiomorpholino)phenyl)-2,7- diazaspiro[4.4]nonan-2-yl)-2-fluoroethan-1-one | 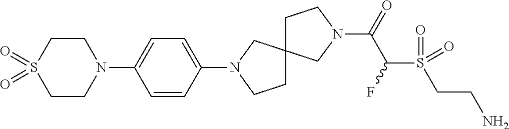 [SMILES: NCCS(=O)(=O)C(F)C(=O)N1CCC2(CCN(c3ccc(N4CCS(=O)(=O)CC4)cc3)C2)C1] |
| 2-((2-Aminoethyl)sulfonyl)-1-(cis-5-(4- chlorophenyl)hexahydropyrrolo[3,4-c]pyrrol- 2(1H)-yl)-2-fluoroethan-1-one | 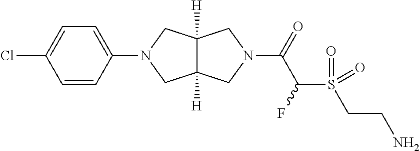 [SMILES: NCCS(=O)(=O)C(F)C(=O)N1C[C@@H]2CN(c3ccc(Cl)cc3)C[C@@H]2C1] |
| 2-((2-Aminoethyl)sulfonyl)-1-(cis-3a,6a-dimethyl- 5-(4-morpholinophenyl)hexahydropyrrolo[3,4- c]pyrrol-2(1H)-yl)-2-fluoroethan-1-one | 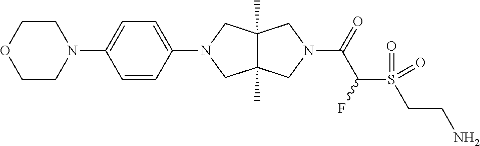 [SMILES: C[C@@]12CN(C(=O)C(F)S(=O)(=O)CCN)C[C@]1(C)CN(c1ccc(N3CCOCC3)cc1)C2] |
| 2-((2-Aminoethyl)sulfonyl)-1-(cis-5-(4-(1,1- dioxidothiomorpholino)phenyl)-3a,6a- dimethylhexahydropyrrolo[3,4-c]pyrrol-2(1H)-yl)- 2-fluoroethan-1-one | 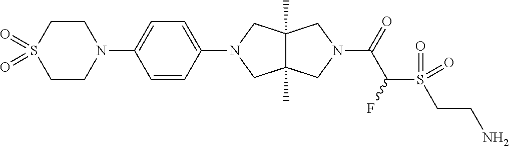 [SMILES: C[C@@]12CN(C(=O)C(F)S(=O)(=O)CCN)C[C@]1(C)CN(c1ccc(N3CCS(=O)(=O)CC3)cc1)C2] |

or a pharmaceutically acceptable salt of any of the foregoing compounds.

In one embodiment, the compound of formula (I) is selected from a compound of Table 1 or Table 2, and pharmaceutically acceptable salts thereof.

In an embodiment of the present invention the compound of formula (I) is a compound selected from:

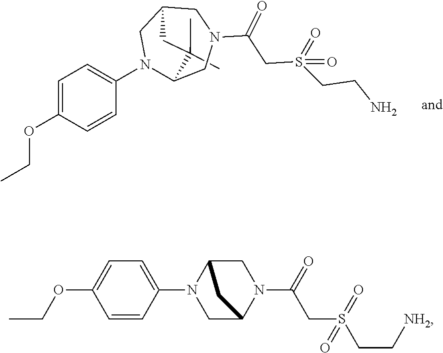

or a pharmaceutically acceptable salt thereof.

### Pharmaceutical Compositions

In accordance with another aspect, the present invention provides a pharmaceutical formulation comprising a compound of the invention, or a pharmaceutically acceptable salt thereof, and a pharmaceutically acceptable excipient.

Conventional procedures for the selection and preparation of suitable pharmaceutical formulations are described in, for example, “Pharmaceuticals—The Science of Dosage Form Designs”, M. E. Aulton, Churchill Livingstone, 1988.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy of a condition is an amount sufficient to symptomatically relieve in a warm-blooded animal, particularly a human the symptoms of the condition or to slow the progression of the condition.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the invention will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, a daily dose selected from 0.1 mg/kg to 100 mg/kg, 1 mg/kg to 75 mg/kg, 1 mg/kg to 50 mg/kg, 1 mg/kg to 20 mg/kg or 5 mg/kg to 10 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Suitably the compound of the invention is administered orally, for example in the form of a tablet, or capsule doasage form. The daily dose administered orally may be, for example a total daily dose selected from 1 mg to 2000 mg, 5 mg to 2000 mg, 5 mg to 1500 mg, 10 mg to 750 mg or 25 mg to 500 mg. Typically, unit dosage forms will contain about 0.5 mg to 0.5 g of a compound of this invention.

### Therapeutic Uses and Applications

In accordance with another aspect, the present invention provides a compound of the invention, or a pharmaceutically acceptable salt thereof, for use as a medicament.

A further aspect of the invention provides a compound of the invention, or a pharmaceutically acceptable salt thereof, for use in the treatment of a disease or medical condition mediated by LOX.

Also provided is the use of a compound of the invention, or a pharmaceutically acceptable salt thereof in the manufacture of a medicament for the treatment of a disease or medical condition mediated by LOX.

Also provided is a method of treating a disease or medical condition mediated by LOX in a subject in need thereof, the method comprising administering to the subject an effective amount of a compound of the invention, or a pharmaceutically acceptable salt thereof.

Unless stated otherwise reference to the treatment of a disease or medical condition mediated by LOX is intended to encompass diseases or medical conditions mediated by any one of LOX, LOXL1, LOXL2, LOXL3 or LOXL4.

In the following sections of the application reference is made to a compound of the invention, or a pharmaceutically acceptable salt for use in the treatment of certain diseases or conditions. It is to be understood that any reference herein to a compound for a particular use is also intended to be a reference to (i) the use of the compound of the invention, or pharmaceutically acceptable salt thereof, in the manufacture of a medicament for the treatment of that disease or condition; and (ii) a method of treating the disease or condition in a subject, the method comprising administering to the subject a therapeutically effective amount of the compound of the invention, or pharmaceutically acceptable salt thereof.

The disease of medical condition mediated by LOX may be any of the diseases or medical conditions listed in this application.

As discussed in the background to the invention the role of the LOX family of may have distinct roles in diseases such as cancer. Accordingly the selective inhibition of a LOX may be advantageous. In one embodiment there is provided a compound of the invention, or pharmaceutically acceptable salt thereof, for use in the selective inhibition of LOX, LOXL1, LOXL2, LOXL3 or LOXL4. In other embodiments it may be advantageous to inhibit two or more members of the LOX family. Accordingly in another embodiment there is provided a compound of the invention, or pharmaceutically acceptable salt thereof, for use in the inhibition of two or more members of the LOX family selected from LOX, LOXL1, LOXL2, LOXL3 or LOXL4.

### Proliferative Diseases—LOX and Cancer

A further aspect of the invention provides a compound of the invention, or pharmaceutically acceptable salt thereof, for use in the treatment of a proliferative disease. The proliferative disease may be malignant or non-malignant.

As mentioned in the Background to the invention, LOX plays a critical role in primary cancer and metastasis. Evidence supporting this role of LOX in primary tumour growth and metastasis is described below.

Studies have shown that LOX plays a fundamental role in the growth of primary tumours in colorectal and lung cancer (Gao, Xiao et al. 2010, Baker, Cox et al. 2011) and glioblastoma (Mammoto, Jiang et al. 2013). PDAC KRAS^{mut}/p53^{wt} cells (which endogenously express low levels of LOX) were engineered to express high levels of human LOX. In murine allograft models using these cells primary tumour growth is increased significantly (Miller, Morton et al. 2015). Lysyl oxidase activity participates in primary tumor growth in a transgenic mouse model of aggressive pancreatic ductal adenocarcinoma (PDAC) by directly impacting the senescence stability (Wiel, Augert et al. 2013).

Expression of LOX is elevated in more than 70% of breast cancer patients with Estrogen Receptor negative disease, in 80% of head and neck cancer patients, in 33% of primary colorectal carcinomas (CRC) and 48% of metastatic tissues from patients with CRC (Baker, Cox et al. 2011), and in cirrhotic HCC patients with a history of alcoholism (Huang, Ho et al. 2013). LOX is also overexpressed in lung adenocarcinoma (Wilgus, Borczuk et al. 2011), LKB1-mutant lung cancer(Gao, Xiao et al. 2010), aggressive prostate adenocarcinoma (Stewart, Gray et al. 2008), uveal melanoma (Abourbih, Di Cesare et al. 2010), oral and oropharyngeal squamous carcinoma (Albinger-Hegyi, Stoeckli et al. 2010), thyroid cancer (Boufraqech, Nilubol et al. 2015), clear cell renal cell carcinoma (Vitalba et al, 2016), myeloproliferative neoplasms, especially myelofibrosis (Papadantonakis, Matsuura et al. 2012, Tadmor, Bejar et al. 2013) and pancreatic cancer (Sansom 2012, Miller, Morton et al. 2015).

### Lysyl-Oxidase-Like Isoforms and Cancer

LOXL2 is another member of the LOX family that is involved in the cross-linking of extracellular collagens and elastin (Vadasz, Kessler et al. 2005) (Kim, Kim et al. 2010). In addition to conserved C-terminal region, the LOXL2 protein has scavenger receptor cysteine-rich regions that are commonly found in cell surface receptors and adhesion molecules, as well as a cytokine receptor-like domain.

LOXL2 expression has been found upregulated in breast, gastric, colon, esophageal, head and neck, lung and laryngeal carcinomas, as reviewed in Barker et al (Barker, Cox et al. 2012) and in renal cells carcinoma (Hase, Jingushi et al. 2014) (Nishikawa, Chiyomaru et al. 2015). High LOXL2 expression has been associated with poor prognosis in patients with squamous cell carcinoma, laryngeal, oesophagus and breast cancer, increased metastases in colon and breast cancer, as well as drug resistance in pancreatic cancer cells—reviewed in Barker et al (Barker, Cox et al. 2012). Additionally, it has been shown that LOXL2 up-regulation increases the invasiveness of otherwise non-invasive breast cancer cells (Akiri, Sabo et al. 2003). Furthermore, LOXL2 and LOXL4 are required for metastatic niche formation in a breast orthotopic mouse model (Wong et al, 2011). LOXL2 expression is associated with lymph node metastasis, histological grades and poor prognosis in cholangiocarcinoma, and knockdown of LOXL2 reduces invasion and metastasis (Xu, Li et al. 2014). HCC metastasis relies on LOXL2, which is overexpressed in tumor tissues and sera of HCC patients (Wong, Tse et al. 2014).

LOXL2 transcription is regulated by HIF-1 and upregulation of LOXL2 in hypoxia has been shown to downregulate E-cadherin leading to epithelial to mesenchymal transition (EMT) (Schietke, Warnecke et al. 2010) which is a key step in tumour progression, invasion and metastasis. This is in agreement with other reports where LOXL2 was shown to be involved in both EMT and tumour progression in murine squamous and spindle cell carcinomas (Fong, Dietzsch et al. 2007) (Moreno-Bueno, Salvador et al. 2011). LOXL2 expression is positively associated in CRC (Offenberg, Brunner et al. 2008). LOXL2 has also been linked to Src kinase/focal adhesion kinase (Src/FAK) pathway activation, and this appears to be the major pathway where secreted LOXL2 induces gastric tumour cell invasion and metastasis (Peng, Ran et al. 2009).

In certain cancers such as basal-like breast carcinoma and larynx squamous cell carcinoma perinuclear expression of LOXL2 is a marker of tumour aggressiveness and poor prognostic (Moreno-Bueno, Salvador et al. 2011) (Peinado, Moreno-Bueno et al. 2008).

Barry-Hamilton et al. reported that LOXL2 antibody treatment significantly reduces bone metastases from intracardiac injection of breast carcinoma cells (Barry-Hamilton, Spangler et al. 2010). In addition, Barker et al have provided preclinical evidence that LOXL2 inhibition is highly effective against spontaneous lung, liver and bone metastases of mammary carcinoma cells (Barker, Chang et al. 2011). Therefore, LOXL2 also represents a promising therapeutic target for the treatment of primary and metastatic cancer.

As mentioned in the Background to the Invention it is thought that although LOX and LOXL2 are involved in similar extra-cellular processes, it appears that they have distinct roles.

Other members of the LOX family, LOXL1, LOXL3 and LOXL3 are also implicated in proliferative conditions including cancer (see Background to the Invention).

Accordingly in one embodiment there is provided a compound of the invention, or pharmaceutically acceptable salt thereof for use in the treatment of a cancer. In one embodiment the cancer is non-metastatic. Accordingly, the compound of the invention, or a pharmaceutically acceptable salt thereof may be for use in the treatment of a primary tumour in a subject.

### The Role of LOX in Cancer Metastasis

Elevated LOX expression is associated with metastasis and decreased patient survival (Baker, Cox et al. 2011, Wilgus, Borczuk et al. 2011) Increased LOX expression is associated with disease grade, increased distant metastasis and lower overall survival in breast cancer patients with oestrogen receptor (ER)-negative tumours (Erler, Bennewith et al. 2006), in head and neck cancer patients (Albinger-Hegyi, Stoeckli et al. 2010, Toustrup, Sorensen et al. 2011), gastric cancer (Kasashima, Yashiro et al. 2015), hepatocellular carcinoma (Zhu, Huang et al. 2015), non-small cells lung cancer (Liu, Ping et al. 2014) and astrocytomas (da Silva, Uno et al. 2015), laryngeal cancer (Se, 2017). LOX expression is a determinant of poor survival in pancreatic cancer (Miller, Morton et al. 2015). Inhibition of LOX eliminates metastasis in mice with orthotopically grown human breast cancer (Erler, Bennewith et al. 2006) and inhibits tumour angiogenesis in a human colorectal cancer model (Baker, Bird et al. 2013).

A polyclonal antibody that was raised against LOX and shown to inhibit its enzymatic activity, was able to block the metastatic spread of tumour cells to the lungs and livers of recipient mice in an orthotopic model of metastatic human breast cancer (Erler et al, 2006). Suppression of LOX expression using shRNA blocks metastatic spread of the breast cancer cells and that BAPN, the non-selective small molecule inhibitor of LOX can block metastatic tumour growth of these cells in mice (Erler et al, 2006). Furthermore, inhibition of tumour-secreted LOX by genetic (shRNA), antibody (Ab) or the irreversible non-selective small molecule inhibitor BAPN, significantly reduced invasion and metastasis of orthotopic human breast tumours or circulating human breast cancer cells (Bondareva, Downey et al. 2009, Erler, Bennewith et al. 2009, Levental, Yu et al. 2009), CRC (Baker, Cox et al. 2011), HCC (Huang, Ho et al. 2013), LKB1-mutant lung adenocarcinoma (Gao, Xiao et al. 2010), anaplastic thyroid cancer (Boufraqech, Nilubol et al. 2015) and PDAC in mice (Sansom 2012; Miller, Morton et al. 2015). High expression of LOX in primary breast tumours leads to osteolytic lesion formation; silencing or inhibition of LOX activity abrogates tumour-driven bone metastases (Cox, Rumney et al. 2015). LOX inhibition with BAPN and new inhibitor CCT365623 significantly reduce metastatic lung tumour burden in a mouse model of spontaneous breast cancer that metastasizes to the lungs (Tang et al, 2017).

LOX family members (especially LOX and LOXL2) play a critical role in the metastatic spread of cancer cells (Erler, Bennewith et al. 2006, Bondareva, Downey et al. 2009, Erler, Bennewith et al. 2009, Levental, Yu et al. 2009, Gao, Xiao et al. 2010). In response to hypoxia (a condition that occurs due to inadequate blood supply when solid tumours exceed about 1 cm³ in size), cancer cells produce and secrete LOX into the circulation (Erler, Bennewith et al. 2009).

LOX regulates invasion of cancer cells in vitro. Thus, cancer cells expressing high levels of LOX show increased ability to invade 3D collagen I and Matrigel matrices (Kirschmann, Seftor et al. 2002) (Erler, Bennewith et al. 2006). Furthermore, experimental over-expression of LOX enhances invasion of cancer cells, whereas genetic knock-down of LOX using RNA interference (RNAi; with both short hairpin RNA [shRNA] or small interfering RNA [siRNA]) or antisense technology) inhibits the in vitro invasion activity of cancer cells (Kirschmann, Seftor et al. 2002) (Erler, Bennewith et al. 2006). Similarly, a non-selective small molecule inhibitor of LOX, beta-aminopropionitrile (BAPN) also blocks the in vitro invasion activity of several human cancer cell lines (Kirschmann, Seftor et al. 2002) (Erler, Bennewith et al. 2006). LOX enhances hypoxia-induced invasion and migration in cervical cancer cells mediated by the EMT which can be inhibited by BAPN (Yang, Li et al. 2013). These studies implicate LOX in the invasive behaviour of cancer cells.

One of the critical functions of LOX appears to be to act remotely to pre-condition the niche at future sites of metastasis. Tumour cell metastasis is facilitated by these “premetastatic niches” formed in destination organs using invading bone marrow-derived dendritic cells (BMDCs). This “nest-building” activity is initiated when LOX becomes deposited at discreet sites in the target organ (Erler, Bennewith et al. 2009). Studies have shown that bone marrow derived cell recruitment is an essential step in niche conditioning and metastatic spread of cancer (Kaplan et al, 2005). This mechanism underlines the importance of LOX for the invasive activity of cancer cells and for the earliest stages of metastasis, when the cancer cells first migrate out of the primary tumour. It has been shown that BMDCs and LOX co-localise in human metastatic tissue, and inhibition of LOX can prevent BMDC recruitment and metastasis in models of breast cancer metastasis (Erler, Bennewith et al. 2009).

In addition to its roles in the early phases of metastasis, there is evidence that LOX is necessary to maintain the growth of the cancer cells once they arrive at the new metastatic sites because inhibition of LOX causes regression of these lesions, even after the development of metastatic disease (Erler, Bennewith et al. 2006) (Erler, Bennewith et al. 2009) (Bondareva, Downey et al. 2009). It was shown that although depletion of LOX does not affect tumour cell proliferation on plastic, it suppresses their growth in recombinant basement membrane (Matrigel) matrices (Erler, Bennewith et al. 2006). Furthermore, cancer cells do not colonise the lungs efficiently when LOX is inhibited by shRNA (Erler et al, 2006) and it was found that metastatic lung tumours regress when mice are treated with LOX neutralising antibodies (Erler, Bennewith et al. 2006). Notably, the colonisation of the lung by human breast cancer cells was enhanced when the cells were co-injected with conditioned medium from cells expressing LOX, but this was blocked if the mice were treated with conditioned medium in the presence of BAPN or a LOX antibody (Erler, Bennewith et al. 2009). These findings demonstrate a requirement for tumour-secreted LOX to maintain metastatic growth.

LOX is essential for phosphorylation of the focal adhesion kinase (FAK) downstream of integrin signalling (Erler, Bennewith et al. 2006). FAK is a tyrosine kinase that interacts with several signalling molecules and is critical for cell survival (van Nimwegen and van de Water 2007). LOX-mediated collagen cross-linking results in increased tissue stiffness and activation of the FAK/SRC signalling in in vitro and in vivo models of CRC. Cells expressing high levels of enzymatically active LOX have an increased capacity to proliferate, invade and metastasise. Thus LOX have both cell-dependent and cell-autonomous roles in metastatic tumour growth at several levels: enhances the ability of cancer cells to invade locally, possibly by enhancing migration away from the primary site; conditions the future metastatic sites in preparation for the arrival of the BMDCs and then tumour cells; supports the survival/proliferation of the cancer cells once they colonise the niche.

Host response to tumour surgery can promote further lung metastases in a mechanism mediated by LOX. Blocking LOX activity reduces the risk of lung metastases following surgery (Chen, 2017).

Accordingly, the compound of the invention, or a pharmaceutically acceptable salt thereof may be for use in the treatment of metastatic cancer in a subject.

In another embodiment of the invention there is provided a compound of the invention, or a pharmaceutically acceptable salt thereof may be for use as an inhibitor of the motility of tumour cells. In another embodiment the compound of the invention, or a pharmaceutically acceptable salt thereof may be for use as an inhibitor of the dissemination and invasiveness of mammalian cancer cells leading to inhibition of metastatic tumour growth. In particular a compound of the invention, or a pharmaceutically acceptable salt thereof may be for use as an anti-invasive agent for use in the containment and/or treatment of solid tumour disease. In another embodiment a compound of the invention, or a pharmaceutically acceptable salt thereof may be for use in the prevention or inhibition of cancer metastasis.

### LOX Family, Fibroblasts and Stroma

Cancer associated fibroblasts are recruited by cancer cells recruit fibroblasts through various growth factors and cytokines and form a myofibroblastic microenvironment that promotes cancer growth, survival, local invasion and metastasis (Karagiannis, Poutahidis et al. 2012). Persistent presence of myofibroblasts in cancer contributes to desmoplasia, a cancer-specific type of fibrosis. Desmoplasia and increased fibrosis have been associated with progression of several cancers such as breast, pancreatic, colorectal, gastric and hepatocellular (Barker, Cox et al. 2012). Desmoplasia is also an intrinsic mechanism of resistance to immunotherapy in stromally-rich tumours (Zhao and Subramanian, 2017). LOX and LOX family members have an essential role in extracellular matrix remodelling and desmoplasia (Levental, 2009; Xiao, 2012). Lysyl oxidase family members expression, either secreted by cancer cells or by activated fibroblasts, has been found associated with tumour ECM, tumour stroma or tumour-associated vasculature of several cancers, such as colorectal, pancreatic, breast, laryngeal, endometrial, testicular, hepatocellular, renal (reviewed in Barker et al (Barker, Cox et al. 2012)), gastric cancer (Kasashima, Yashiro et al. 2014), and to be involved in their progression and metastasis (Akiri, Sabo et al. 2003, Barry-Hamilton, Spangler et al. 2010, Barker, Bird et al. 2013) (Pickup, Laklai et al. 2013). Expression of LOXL4 is enhanced in keratocystic odontogenic tumors (KCOT) stromal tissues and primary KCOT stromal fibroblasts (Jiang, Sima et al. 2014)

In one embodiment there is provided a compound of the invention, or a pharmaceutically acceptable salt thereof for use in the treatment of desmoplasia.

As discussed herein, the compound of the invention, or a pharmaceutically acceptable salt thereof may be for use in the treatment of a cancer, which may be non-metastatic or metastatic and which may be a solid tumour or a haematological (“liquid”) cancer selected from, for example:

(1) Carcinoma, including for example tumours derived from stratified squamous epithelia (squamous cell carcinomas) and tumours arising within organs or glands (adenocarcinomas). Examples include breast, colon, lung, prostate, ovary. esophageal carcinoma (including, but not limited to, esophageal adenocarcinoma and squamous cell carcinoma), basal-like breast carcinoma, basal cell carcinoma (a form of skin cancer), squamous cell carcinoma (various tissues), head and neck carcinoma (including, but not limited to, squamous cell carcinomas), stomach carcinoma (including, but not limited to, stomach adenocarcinoma, gastrointestinal stromal tumor), signet ring cell carcinoma, bladder carcinoma (including transitional cell carcinoma (a malignant neoplasm of the bladder)), bronchogenic carcinoma, colorectal carcinoma (including, but not limited to, colon carcinoma and rectal carcinoma), anal carcinoma, gastric carcinoma, lung carcinoma (including but not limited to small cell carcinoma (SCLC) and non-small cell carcinoma of the lung (NSCLC), lung adenocarcinoma, squamous cell carcinoma, large cell carcinoma, bronchioloalveolar carcinoma, and mesothelioma), neuroendocrine tumors (including but not limited to carcinoids of the gastrointestinal tract, breast, and other organs), adrenocortical carcinoma, thyroid carcinoma, pancreatic carcinoma (including, but not limited to, pancreatic ductal adenocarcinoma, pancreatic adenocarcinoma, acinar cell carcinoma, intraductal papillary mucinous neoplasm with invasive carcinoma, mucinous cystic neoplasm with invasive carcinoma, islet cell carcinoma and neuroendocrine tumors), breast carcinoma (including, but not limited to, ductal carcinoma, lobular carcinoma, inflammatory breast cancer, clear cell carcinoma, mucinous carcinoma), ovarian carcinoma (including, but not limited to, ovarian epithelial carcinoma or surface epithelial-stromal tumor including serous tumor, endometrioid tumor and mucinous cystadenocarcinoma, sex-cord-stromal tumor), liver and bile duct carcinoma (including, but not limited to, hepatocellular carcinoma, cholangiocarcinoma and hemangioma), prostate carcinoma, adenocarcinoma, brain tumours (including, but not limited to glioma, glioblastoma and medulloblastoma), germ cell tumors, sweat gland carcinoma, sebaceous gland carcinoma, papillary carcinoma, papillary adenocarcinoma, cystadenocarcinoma, kidney carcinoma (including, but not limited to, renal cell carcinoma, clear cell carcinoma and Wilm's tumor), medullary carcinoma, ductal carcinoma in situ or bile duct carcinoma, choriocarcinoma, seminoma, embryonal carcinoma, cervical carcinoma, uterine carcinoma (including, but not limited to, endometrial adenocarcinoma, uterine papillary serous carcinoma, uterine clear-cell carcinoma, uterine sarcomas and leiomyosarcomas, mixed mullerian tumors), testicular carcinoma, osteogenic carcinoma, epithelial carcinoma, sarcomatoid carcinoma, nasopharyngeal carcinoma, laryngeal carcinoma; oral and oropharyngeal squamous carcinoma;
(2) Sarcomas, including: osteosarcoma and osteogenic sarcoma (bone); chondrosarcoma (cartilage); leiomyosarcoma (smooth muscle); rhabdomyosarcoma (skeletal muscle); mesothelial sarcoma and mesothelioma (membranous lining of body cavities); fibrosarcoma (fibrous tissue); angiosarcoma and hemangioendothelioma (blood vessels); liposarcoma (adipose tissue); glioma and astrocytoma (neurogenic connective tissue found in the brain); myxosarcoma (primitive embryonic connective tissue); chordoma, endotheliosarcoma, lymphangiosarcoma, lymphangioendotheliosarcoma, synovioma, Ewing's sarcoma, mesenchymous and mixed mesodermal tumor (mixed connective tissue types) and other soft tissue sarcomas;
(3) Myeloma and multiple myeloma;
(4) Hematopoietic tumours, including: myelogenous and granulocytic leukemia (malignancy of the myeloid and granulocytic white blood cell series); lymphatic, lymphocytic, and lymphoblastic leukemia (malignancy of the lymphoid and lymphocytic blood cell series); polycythemia vera and erythremia (malignancy of various blood cell products, but with red cells predominating); myelofibrosis.
(5) Lymphomas, including: Hodgkin and Non-Hodgkin lymphomas;
(6) Solid tumors of the nervous system including medulloblastoma, craniopharyngioma, ependymoma, pinealoma, hemangioblastoma, acoustic neuroma, oligodendroglioma, meningioma, neuroblastoma and schwannoma;
(7) Melanoma, uveal melanoma and retinoblastoma; and
(8) Mixed Types, including, e.g., adenosquamous carcinoma, mixed mesodermal tumor, carcinosarcoma or teratocarcinoma.

In a particular embodiment, a compound of the invention, or a pharmaceutically acceptable salt thereof may be for use in the treatment of a cancer selected from pancreatic, colorectal, breast and lung cancer.

A compound of the invention, or a pharmaceutically acceptable salt thereof the invention may be for use in the treatment of a benign proliferative disease. The benign disease may be a benign tumour, for example hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas, pyogenic granulomas, moles, uterine fibroids, thyroid adenomas, adrenocortical adenomas or pituitary adenomas. The benign condition may be endometriosis or a keratocystic odontogenic tumor.

### Fibrotic Diseases

As discussed in the Background to the invention, LOX and LOXL are implicated in fibrotic diseases. Accordingly a compound of the invention or a pharmaceutically acceptable salt thereof may be for use in the treatment of a fibrotic disorder. The fibrotic disorder may be a disorder characterised by excess fibrosis, e.g., an excess of fibrous connective tissue in a tissue or organ, e.g., triggered by a reparative or reactive process, e.g., in response to injury (e.g., scarring, healing) or excess fibrotic tissue arising from a single cell line (e.g., fibroma).

LOX has been implicated in the pathogenesis of renal fibrosis and its inhibition with the alleviation of the symptoms (Di Donato, Ghiggeri et al. 1997, Haase 2009, Chen, Lin et al. 2015). Hyperuricemia results in hypertension, intrarenal vascular disease, and renal injury and is associated with increased expression of lysyl oxidase (LOX) and fibronectin in kidneys (Yang, Wang et al. 2010). Increased LOX activity has been linked to delayed graft failure after renal transplant, potentially due to increased local fibrosis (Zhi, 2017)

Similar involvement of LOX or LOXL2 in the pathology of disease and reduction in symptoms has been demonstrated for lung fibrosis (Barry-Hamilton, Spangler et al. 2010) (Haase 2009, Cox, Bird et al. 2013, Chien, Richards et al. 2014).

LOX and LOXL2 are involved in liver fibrosis (Kagan 1994, Marshall and Smith 2011) (Ricard-Blum, Bresson-Hadni et al. 1996) (Smith and Van Vlasselaer 2011) (Georges, Hui et al. 2007), liver cirrhosis (the last stage of liver fibrosis) (Kagan 1994) and related diseases such as Wilson's disease and primary biliary cirrhosis (Vadasz, Kessler et al. 2005). Therapeutic indications for LOX family inhibitors (such as simtuzumab, a humanized LOXL2 antibody) included a number of fibrotic conditons: myelofibrosis (Primary myelofibrosis, Post Polycythemia Vera or Post Essential Thrombocythemia Myelofibrosis), idiopathic pulmonary fibrosis (IPF), liver fibrosis due to non-alcoholic steatohepatitis (NASH), HIV and/or Hepatitis C-infection or primary sclerosing cholangitis (PSC) and compensated liver cirrhosis due to NASH. Levels of lysyl oxidase are increased in patients with scleroderma and systemic sclerosis (Chanoki, Ishii et al. 1995) (Rimar, Rosner et al. 2014).

LOX inhibitors assist in collagen remodeling and re-establishment of collagen architecture in human Dupuytren's, keloid and scar fibroblasts (Priyanka, 2016).

The fibrotic disorder may be any of those discussed in the above three paragraphs. In one embodiment the compound of the invention or a pharmaceutically acceptable salt thereof may be for use in the treatment of a fibrotic disorder selected from:

(i) a fibrotic condition affecting the lungs, for example pulmonary fibrosis secondary to cystic fibrosis; idiopathic pulmonary fibrosis; coal worker's progressive massive fibrosis; cryptogenic fibrosing alveolitis, chronic fibrosing interstitial pneumonia, interstitial lung disease (ILD), diffuse parenchymal lung disease (DPLD), emphysema and chronic obstructive pulmonary disease (COPD), or chronic asthma; or
(ii) a fibrotic condition affecting the liver, for example cirrhosis, and associated conditions such as chronic viral hepatitis B or C, Wilson's disease, non-alcoholic fatty liver disease (NAFLD), alcoholic steatohepatitis (ASH), non-alcoholic steatohepatitis (NASH), primary biliary cirrhosis (PBC), biliary cirrhosis or autoimmune hepatitis; or
(iii) a fibrotic condition affecting the kidneys, for example diabetic nephropathy, vesicoureteral reflux, tubulointerstitial renal fibrosis; glomerulonephritis or glomerular nephritis, including focal segmental glomerulosclerosis and membranous glomerulonephritis or mesangiocapillary glomerular nephritis;
(iv) a fibrotic condition affecting the heart or vascular system, for example endomyocardial fibrosis; old myocardial infarction; atrial fibrosis; congestive heart failure, cardiomyopathy, hypertensive heart disease (HHD), hypertension (for example pulmonary hypertension) and fibrosis associated with hypertension, atherosclerosis, restenosis (e.g. coronary, carotid, and cerebral lesions), and heart disease associated with cardiac ischemic events; or
(v) a fibrotic condition affecting the mediastinum, for example mediastinal fibrosis; or
(vi) a fibrotic condition affecting bone, for example myelofibrosis, including primary myelofibrosis, post polycythemia vera or post essential thrombocythemia myelofibrosis; or
(vii) a fibrotic condition affecting the retroperitoneum, for example retroperitoneal fibrosis skin; or
(viii) a fibrotic condition affecting the skin, for example nephrogenic systemic fibrosis, keloid formation and scarring, systemic sclerosis or scleroderma; or
(ix) a fibrotic condition affecting the GI tract, for example a fibrotic intestinal disorder, inflammatory bowel disease, ulcertative colitis or Crohn's disease; or
(x) a fibrotic condition affecting connective tissue, for example arthrofibrosis; or capsulitis; or
(xi) a fibrotic condition affecting the eye, for example ocular fibrosis following surgery or pseudoexfoliation syndrome glaucoma.

### LOX Family, Angiogenesis and Vasculature Permeability

Angiogenesis, the formation of new blood vessels, is essential for tumor growth and progression.

LOX and LOXL2 are key players in promoting angiogenesis in a number of tumour models, such as colorectal (Baker, Bird et al. 2013), ovarian, lung cancer (Zaffryar-Eilot, Marshall et al. 2013), melanoma (Osawa, Ohga et al. 2013), glioblastoma (Mammoto, Jiang et al. 2013). LOX is overexpressed in tumour endothelial cells (Osawa, Ohga et al. 2013). Increased LOX tumour expression is associated with increased VEGF expression (Mammoto, Jiang et al. 2013), (Baker, Bird et al. 2013).

Additionally, LOXL2 inhibition led to the normalisation of vasculature and increased tumour perfusion in ovarian xenograft and lung allograft mice models (Zaffryar-Eilot, Marshall et al. 2013).

Excessive angiogenesis is involved in a number of diseases in addition to cancer discussed above. LOX mediates vascular permeability by modulating the stiffness of the endothelial barrier. Abnormal vascular permeability, such as present in diseases such as pulmonary edema and acute respiratory distress syndrome (ARDS) or endotoxin-induced lung injury can be normalised by LOX inhibition (Mammoto, Mammoto et al. 2013) (Ingber and Mammoto 2014).

Accordingly a compound of the invention or a pharmaceutically acceptable salt thereof may be for use as an anti-angiogenic agent. A compound of the invention or a pharmaceutically acceptable salt thereof may be for use in vascular normalisation.

In one embodiment a compound of the invention or a pharmaceutically acceptable salt thereof may be for use in the treatment is treatment of pulmonary embolism, emphysema, pleural effusion, pulmonary oedema, brain swelling, plural effusion, pericardial effusion and ascites.

In one embodiment a compound of the invention or a pharmaceutically acceptable salt thereof may be for use in the treatment is treatment of ischemia; ischemic stroke, ischemic heart disease, cerebral infarct, peripheral vascular disease, elephantiasis, lymphatic obstruction.

In one embodiment, the treatment is treatment of age-related macular degeneration (AMD), diabetic retinopathy and retinopathy of prematurity.

### Inflammatory Disorders

Exacerbated inflammation and lung barrier dysfunction are hallmarks of acute respiratory distress syndrome (ARDS), a condition with dangerously high rates of morbidity and mortality. Increased LOX activity has been associated with bacterial lipopolysaccharide (LPS) induced inflammation. Inhibition of LPS-induced ECM crosslinking and stiffening by LOX suppression reduced EC inflammatory activation and lung dysfunction. Thus LOX inhibitors can be useful for the treatment of ARDS (Mambetsariev, Tian et al. 2014). LOX and LOXL1 reduction and collagen crosslinking reduction have been associated with decreased inflammation in an Angiotensin II induced model of hypertension (Gonzalez, Rhaleb et al. 2014).

In an embodiment there is provided a compound of the invention, or a pharmaceutically acceptable salt thereof may be useful in the treatment of an inflammatory condition. The inflammatory condition may be any of those described herein. For example, the compound of the invention, or a pharmaceutically acceptable salt thereof may be for use in the treatment of acute inflammation (e.g., mediated by an acute infection).

In an embodiment the compound of the invention, or a pharmaceutically acceptable salt thereof may be for use in the treatment of chronic inflammatory disease, for example a disease selected from inflammatory bowel diseases (e.g. Crohn's disease and ulcerative colitis), psoriasis, sarcoidosis, rheumatoid arthritis, osteoarthritis, psoriatic arthritis, Reiter's syndrome, traumatic arthritis, rubella arthritis, acute synovitis, gouty arthritis and spondylitis.

In an embodiment the compound of the invention, or a pharmaceutically acceptable salt thereof may be for use in the treatment of rheumatoid arthritis; osteoarthritis; psoriatic arthritis; Reiter's syndrome; traumatic arthritis; rubella arthritis; acute synovitis; gouty arthritis; or spondylitis; diabetes or gout.

In an embodiment the compound of the invention, or a pharmaceutically acceptable salt thereof may be for use in the treatment of psoriasis; eczema; sarcoidosis, allergic rhinitis; allergic conjunctivitis; asthma, acute respiratory distress syndrome, acute lung injury (ALI), acute respiratory distress syndrome (ARDS), endotoxin-induced lung injury, pulmonary inflammation, chronic obstructive pulmonary disease and systemic cachexia.

In an embodiment the compound of the invention, or a pharmaceutically acceptable salt thereof may be for use in the treatment of rheumatoid arthritis, osteoarthritis, psoriatic arthritis, Reiter's syndrome, traumatic arthritis, rubella arthritis, acute synovitis, gouty arthritis or spondylitis, diabetes or gout.

In an embodiment the compound of the invention, or a pharmaceutically acceptable salt thereof may be for use in the treatment of endotoxemia; toxic shock syndrome, inflammatory bowel disease, atherosclerosis, irritable bowel syndrome, Crohn's disease, ulcerative colitis, a bone resorption disease, osteoporosis, diabetes, reperfusion injury, graft versus host reaction, allograft rejection, sepsis, septic shock, endotoxic shock, Gram negative sepsis, glomerulonephritis, restenosis, vasculitis, or thrombosis.

In another embodiment the compound of the invention, or a pharmaceutically acceptable salt thereof may be for use in the treatment of polymyositis, systemic lupus or interstitial nephritis.

### Cardiovascular Disease

Interrupting collagen crosslinking by LOX with BAPN treatment reduces myocardial fibrosis in a mouse model, which is useful as potential therapeutic targeting of collagen regulation and thereby age-related myocardial fibrosis (Rosin, Sopel et al. 2015). Increased expression of LOX is associated with myocardial fibrosis and cardiac dysfunction (Zibadi, Vazquez et al. 2010) (Gao, Xiao et al. 2010) (Lopez, Gonzalez et al. 2010). Left atrial myocardium of patients with atrial fibrillation express higher levels of lysyl oxidase and fibronectin expression as well as collagen crosslinking. Fibronectin upregulation is mediated by LOX in cardiac fibroblasts (Adam, Theobald et al. 2011). LOX inhibitors can be useful for the prevention of fibrotic atrial remodelling. Inhibition of LOX by using a blocking antibody reduced cardiac fibrosis and infarct expansion in a mouse model (Gonzalez-Santamaria, 2016).

Lysyl oxidases play a causal role in experimental pulmonary hypertension and inhibition with BAPN reduces the symptoms (Nave, Mizikova et al. 2014). LOX facilitate the formation of crosslinked and therefore insoluble collagen and the subsequent left ventricle stiffness and systolic dysfunction in patients with hypertensive heart disease (HHD) and heart failure (HF) of hypertensive origin (Lopez, Gonzalez et al. 2013) (Lopez, Querejeta et al. 2012). A role for LOXL1 has been suggested in cardiac hypertrophy and BAPN administration inhibits angiotensin II-induced cardiac hypertrophy in vivo (Ohmura, Yasukawa et al. 2012). LOX knockdown attenuates cardiac and vascular fibrosis in high fat diet induced obesity (Martinez-Martinez, 2016).

Lysyl oxidase inhibition has been proposed as a therapeutic method for decreasing or preventing recurrent restenosis (Nuthakki, Fleser et al. 2004) (Brasselet, Durand et al. 2005). Increased LOX activity has been observed in atherosclerosis (Kagan, Raghavan et al. 1981). LOX is overexpressed in other pathologies associated with increased thrombosis, such as myeloproliferative neoplasms, chronic kidney disease and arterial stenosis and enhances platelets aggregation (Shinobu et al, 2016).

Accordingly in an embodiment compound of the invention, or a pharmaceutically acceptable salt thereof may be for use in the treatment of a cardiovascular disease, for example any one of the diseases mentioned in this section, e.g. the treatment of atherosclerosis, myocardial fibrosis, prevention of fibrotic atrial remodelling, old myocardial infarction; congestive heart failure, cardiomyopathy, hypertensive heart disease (HHD), hypertension (for example pulmonary hypertension) and fibrosis associated with hypertension, restenosis (e.g. coronary, carotid, and cerebral lesions), and heart disease associated with cardiac ischemic events.

### Neurological Conditions

As discussed in the Background to the Invention, LOX is associated with nurological conditions including Alzheimer's disease and other neurological conditions. Accordingly, in one embodiment there is provided a compound of the invention, or a pharmaceutically acceptable salt thereof may be for use in the treatment of a neurological condition mediated by LOX or LOXL. The neurological condition may be Alzheimer's disease (AD) and hereditary cerebral hemorrhage with amyloidosis of the Dutch type (HCHWA-D) or non-Alzheimer's dementia.

LOX is increased at the site of brain injury (Gilad, Kagan et al. 2001) and spinal cord injury and its inhibition lead to accelerated functional recovery in a unilateral spinal cord dissection model (Gilad and Gilad 2001). Accordingly, a compound of the invention, or a pharmaceutically acceptable salt thereof, may be for use in the treatment nerve damage, for example the promotion of nerve regrowth and/or recovery after spinal cord injury.

### Pulmonary Diseases

LOXL2 and LOXL3 are likely to have a role in Primary Alveolar Proteinosis (PAP) since both are expressed in PAP tissue, but not normal lung tissue (Neufeld and Brekhman 2009). Excessive lysyl oxidase activity was linked to the pathologic pulmonary features of bronchopulmonary dysplasia (Kumarasamy, Schmitt et al. 2009). A compound of the invention, or a pharmaceutically acceptable salt thereof may be for use in the treatment of primary alveolar proteinosis (PAP) or bronchopulmonary dysplasia.

### Eye Diseases

Increased LOXL2 levels have been associated with failure following glaucoma surgery and treatment with a LOXL2 antibody reduced pathological angiogenesis, inflammation, and ocular fibrosis (Park, Kim et al. 2014) (Van Bergen, Marshall et al. 2013)(Stalmans, Van Bergen et al. 2011). Expression of lysyl oxidase-type enzymes increases following laser-induced choroidal neovascularization (CNV) in a model of age-related macular degeneration (AMD), in parallel with fibrotic damage. Inhibition of LOX or LOXL2 prevents neovascularization and fibrosis following laser-induced CNV. Therefore LOX and LOXL inhbitors can be useful in the treatment of conditions characterized by neovascularization, such as age-related macular degeneration (AMD), diabetic retinopathy and retinopathy of prematurity (Stalmans, Marshall et al. 2010). LOXL1 expression is increased in the initial stages of abnormal fibrogenesis in pseudoexfoliation syndrome/glaucoma tissues (Zenkel, Krysta et al. 2011) (Schlotzer-Schrehardt, Pasutto et al. 2008).

A compound of the invention, or a pharmaceutically acceptable salt thereof, may be for use in the treatment of an ocular condition mediated by LOX or a LOXL, for example any of the ocular conditions listed in the paragraph above.

### Other Diseases

LOX is the main isoenzyme expressed in human adipose tissue and that its expression is strongly upregulated in samples from obese patients. β-aminopropionitrile reduces body weight gain and improves the metabolic profile in diet-induced obesity in rats (Miana, Galan et al. 2015) and reduces local adipose tissue inflammation (Halberg, Khan et al. 2009). In an embodiment a compound of the invention, or a pharmaceutically acceptable salt thereof may be for use in the treatment of obesity.

LOX has been suggested as a new therapeutic target in bacterial infections and subsequent fibrotic complications. LOX is upregulated in infections with Staphylococcus aureus and inhibition with BAPN influences resulting abscesses morphology and collagenisation (Beerlage, Greb et al. 2013). LOX is implicated also in some parasitic diseases: LOX and LOXLs are upregulated in the early stages of liver granuloma development in schistosomiasis (Decitre, Gleyzal et al. 1998), and BAPN inhibition reduces the size of the granulomas and reduces the egg load in combination with antiparasitic drug PZQ compared to PZQ alone (Giboda, Zenka et al. 1992),

In one embodiment, the compound is for use in the treatment of a bacterial infection, for example infection with Staphylococcus Aureus. The compound of the invention may be for use in the treatment or prevention of infection associated fibrosis, for example to prevent or inhibit abcess formation associated with the infection. The formation of abcesses can provide a favourable microenvironment for the bacteria to multiply. Inhibition of abcess formation may be beneficial in that it may provide enhanced exposure of the bacteria to antibiotics at the site of infection, because the shielding effect provided by the abcess would be reduced or eliminated. Thus, combination treatments comprising a compound of the invention together with an antibiotic agent may provide an enhanced antibacterial effect. The compound of the invention may also be for use in the prevention or inhibition of tissue fibrosis following eradication of the infection and healing of the infection sites.

In one embodiment, the compound is for use in the treatment of a parasitic infection, for example schistosomiasis.

### EGFR Mediated Conditions

Elevated levels of the epidermal growth factor receptor (EGFR), a growth-factor-receptor tyrosine kinase, and/or its ligands is observed in many cancer types and is involved in the promotion of tumour growth. EGFR inhibitors have been directed to a number of cancer types, including NSCLC, pancreatic cancer, squamous cells carcinoma, skin cancer, thyroid, colorectal, prostate, gastric, renal, breast, head and neck cancers, glioma, meningiomas, mesothelioma, cervical carcinomas epidermal carcinomas (reviewed in Bianco et al (Bianco, Gelardi et al. 2007)). Elevated EGFR was found to act as a strong indicator of poor prognosis in head and neck, ovarian, cervical, bladder and oesophageal cancers (Nicholson, Gee et al. 2001). EGFR inhibitors have also been proposed for the treatment of metastatic prostate cancer (Ree, Bratland et al. 2008), biliary cancer such as cholangiocarcinoma with a mutation in ERRF11 (Borad, Carpten et al. 2014).

Blockade of the kinase activity of EGFR does not reach maximum therapeutic efficacy. LOX inhibitors reduce the level of surface EGFR suggesting the possibility that these compounds will have an effect on reducing EGFR activation (Tang et al, 2017).

EGFR inhibition has been targeted as treatment for a number of other diseases, such as prevention and treatment of obesity (Threadgill and Barrick 2007), treatment of Alzheimer's disease (Ma 2013), treatment of Chlamydia infection and related diseases (Tsang and Furdui 2015), treatment of viral diseases (Jung 2010), promotion of axon regeneration (He and Koprivica 2007), treatment of genetic skin disorders characterized by hyperkeratosis, keratinocyte hyperplasia, and/or ichthyosis (Alexandrescu 2009).

Given the role of LOX inhibition in modulating the surface EGFR levels and EGFR signalling, LOX inhibitors could be useful in the treatment of diseases which can be targeted by EGFR inhibition.

In an embodiment, there is provided a compound of the invention, or a pharmaceutically acceptable salt thereof for use in the treatment of a disorder (e.g., a disease) that is ameliorated by the inhibition of EGFR. The EGFR mediated condition may be, for example, any of those listed in this section or elsewhere in the description. The compound of the invention, or a pharmaceutically acceptable salt thereof may be for use in the treatment of a cancer which over-expresses EGFR. The cancer over-expressing EGFR may be, for example NSCLC, pancreatic cancer, squamous cells carcinoma, skin cancer, thyroid, colorectal, prostate, renal, breast, head and neck cancers, glioma, mesothelioma, epidermal carcinomas ovarian, cervical, bladder and oesophageal cancers or a biliary cancer such as cholangiocarcinoma.

In an embodiment, there is provided a compound of the invention, or a pharmaceutically acceptable salt thereof, for use in the treatment of wherein the compound is for use in the treatment a fibrotic disease, such as liver fibrosis, lung fibrosis, kidney fibrosis, cardiac fibrosis, myelofibrosis or schleroderma.

In one embodiment, the compound is for use in the treatment of a viral infection, for example Rhinovirus, influenza virus, parainfluenza virus, coronavirus, adenovirus, respiratory syncytial virus, picornavirus, metapneumovirus, hantavirus, measles virus, Epstein-Barr virus, herpes simplex virus or cytomegalovirus.

In one embodiment, the compound is for use in the treatment of Chlamydia infection.

In one embodiment, the compound is for use in the treatment of a genetic skin disorder, for example a keratinization disorder is selected from among Darier's disease, Hailey-Hailey disease, erythrodermic autosomal recessive lamellar ichthyosis, nonerythrodermic autosomal recessive lamellar ichthyosis, autosomal dominant lamellar ichthyosis, bullous congenital ichthyosiform erythroderma, palmoplantar keratoderma, erythrokeratodermia variabilis, verrucous epidermal nevi, pityriasis rubra pilaris, Netherton syndrome, idiopathic vulgaris, ichthyosis vulgaris, monilethrix, keratosis piliaris, bullous ichthyosiform erythroderma, nonbullous congenital ichthyosis, Sjogren-Larsson syndrome, erythrokeratodermica variabilis, hyperkeratosis lenticularis perstans, eythrokeratodermia figurate variabilis, mutilating keratoderma of Vohwinkel, Harlequin ichthyosis and Tay's syndrome.

### LOX and EGFR

In one aspect, the present invention relates to a lysyl oxidase inhibitor for use in the treatment or prevention of a cancer associated with overexpression of EGFR.

In another aspect, the present invention relates to the use of a lysyl oxidase inhibitor in the manufacture of a medicament for the treatment or prevention of a cancer associated with overexpression of EGFR.

Suitably, in all aspects, the cancer may be selected from the group consisting of: small cell carcinoma (SCLC), non-small cell carcinoma of the lung (NSCLC), pancreatic cancer, squamous cells carcinoma, skin cancer, thyroid, colorectal, prostate, renal, breast, head and neck cancers, glioma, mesothelioma, epidermal carcinomas ovarian, cervical, bladder and oesophageal cancers and a biliary cancer such as cholangiocarcinoma.

Suitably, in all aspects, the lysyl oxidase inhibitor may be a compound of the present invention or a pharmaceutical composition of the present invention.

Suitably, in all aspects of the invention, the lysyl oxidase inhibitor of the invention may downregulate expression of MATN2 and/or activate SMAD2. Suitably, the lysyl oxidase inhibitor of the invention may downregulate expression of HTRA1. Optionally, in all aspects of the invention, the lysyl inhibitor of the invention may inhibit maturation of lysyl oxidase and/or inhibit the catalytic activity of lysyl oxidase. Suitably, the lysyl oxidase inhibitor of the invention may not inhibit MAO-A and/or MAO-B.

In a further aspect, the present invention relates to a method of treating or preventing cancer in a subject, said method comprising administering a therapeutically effective amount of a lysyl oxidase inhibitor of the invention to said subject, wherein said subject has a cancer associated with overexpression of EGFR.

Optionally, the method may comprise determining the level EGFR in a biological sample of said subject, and administering a lysyl oxidase inhibitor of the invention to said subject when the presence of EGFR is determined to be overexpressed in the biological sample.

Optionally, the method may further comprise the steps of determining the level of MATN2, pSMAD2 or HTRA1 or combinations thereof in a biological sample of said subject, and administering a lysyl oxidase inhibitor of the invention to said subject when:
a) the level of MATN2 is greater than a reference sample; and/or
b) the level of pSMAD2 is lower than a reference sample; and/or
c) the level of HTRA1 is greater than a reference sample.

Optionally, said subject may have a cancer selected from the group consisting of: small cell carcinoma (SCLC), non-small cell carcinoma of the lung (NSCLC), pancreatic cancer, squamous cells carcinoma, skin cancer, thyroid, colorectal, prostate, renal, breast, head and neck cancers, glioma, mesothelioma, epidermal carcinomas ovarian, cervical, bladder and oesophageal cancers and a biliary cancer such as cholangiocarcinoma.

Suitably, in all aspects of the invention, the lysyl oxidase inhibitor of the invention may downregulate expression of MATN2 or HTRA1 and/or activate SMAD2. Optionally, in all aspects of the invention, the lysyl inhibitor of the invention may inhibit maturation of lysyl oxidase and/or inhibit the catalytic activity of lysyl oxidase. Suitably, the lysyl oxidase inhibitor of the invention may not inhibit MAO-A and/or MAO-B.

Disclosed herein is a method of increasing the sensitivity rate (efficacy rate) of a lysyl oxidase inhibitor of the invention to treat cancer in a patient population said method comprising selecting a sub population which overexpresses an EGFR and/or MATN2 and/or HTRA1 biomarker. Optionally, said subgroup may underexpress pSMAD2.

Disclosed herein is a method of identifying a subject having increased likelihood of responsiveness or sensitivity to a lysyl oxidase inhibitor of the invention comprising:
a) determining the level of one or more of EGFR, MATN2, and HTRA1 in a biological sample of the subject;
   wherein increased levels EGFR, MATN2, HTRA1 or a combination thereof compared to a reference sample indicates an increased likelihood of responsiveness or sensitivity to a lysyl oxidase inhibitor in the subject.

Disclosed herein is a method of identifying a subject having responsiveness or sensitivity to a lysyl oxidase inhibitor of the invention comprising:
a) determining the level of one or more of EGFR, MATN2, and HTRA1 in a biological sample of the subject;
   wherein increased levels one or more of EGFR, MATN2, and HTRA1 compared to a reference sample identifies the subject as having responsiveness or sensitivity to a lysyl oxidase inhibitor.

Optionally, in all these methods, the methods may comprise a further step of administering a therapeutically effective amount of a lysyl oxidase inhibitor of the invention when the subject is identified as having increased likelihood of responsiveness of sensitivity to a lysyl oxidase inhibitor.

In a further aspect, the present invention relates to a method of determining a treatment regimen for a subject with cancer, comprising:
a) determining the level one or more of EGFR, MATN2, and HTRA1 in a biological sample; and
b) administering a treatment regimen comprising a therapeutically effective amount of a lysyl oxidase inhibitor of the invention, when levels one or more of EGFR, MATN2, and HTRA1 are elevated compared to a reference sample.

### Biomarkers

As disclosed herein, a clinical test is useful to predict response to LOX inhibition therapy, preferably prior to a subject commencing LOX inhibition therapy. Such a test will inform the clinician whether the patient is likely to respond to LOX inhibition therapy or not, and enable the clinician to commence alternative therapy if the patient is predicted to be unlikely to respond. This will benefit the patient by targeting their treatment with an appropriate therapy early, rather than relying on the current “trial and error” approach. Such a test will therefore enable better of targeting of LOX inhibition therapy to patients early in their disease, when maximum effect can be achieved, and may result in greater access to these drugs as they are used in a more cost-efficient manner.

This enables likely responders and non-responders to be identified, so that non-responders may be provided alternative treatment, and those who are not non-responders (and therefore may be a moderate or good responder) may be provided LOX inhibition therapy. As a result thereof, LOX inhibition therapies may therefore be used in a more targeted and cost-efficient manner.

For the purposes of the biomarker and stratification aspects disclosed herein a “LOX inhibitor” is an agent which is able to reduce the expression, reduce the catalytic activity or prevent maturation of LOX. Suitably the LOX inhibitor is a compound of the invention, or a pharmaceutically acceptable salt thereof.

Any suitable source of lysyl oxidase may be employed for the determination of LOX inhibition. The enzyme can be derived, isolated, or recombinantly produced from any source known in the art, including yeast, microbial, and mammalian, that will permit the generation of a suitable product that can generate a detectable reagent or will be biologically active in a suitable assay. In one embodiment, the lysyl oxidase is of human, bovine, or other mammalian origin. See, e.g., Williams, et al., Anal. Biochem. 113:336 (1985); Kirschmann et al., supra; Cancer Res. 62:4478-83 (2002); LOX may be obtained from Accession No. NP00238 (preprotein sequence); Accession No. NM02317 (DNA sequence). A functional fragment or a derivative of lysyl oxidase that still substantially retains its enzymatic activity catalyzing the oxidation of lysyl oxidase can also be used. The lysyl oxidase enzyme can sometimes be the pre-proprotein, proprotein, the protein, or a biologically active fragment thereof.

The enzymatic activity of lysyl oxidase can be assessed by any suitable method. Exemplary methods of assessing lysyl oxidase activity include that of Trackman et al., Anal. Biochem. 113:336-342 (1981); Kagan, et al., Methods Enzymol. 82A:637-49 (1982); Palamakumbura et al., Anal. Biochem. 300:245-51 (2002); Albini et al., Cancer Res. 47: 3239-45 (1987); Kamath et al, Cancer Res. 61:5933-40 (2001); for example.

The enzymatic activity of the lysyl oxidase may be assessed by detecting and/or quantitating “lysyl oxidase byproducts,” such as H₂O₂ production; collagen pyridinium residuesammonium production; aldehyde product production; lysyl oxidation, or deoxypyridinoline (Dpd). One may also detect and quantitate cellular invasive capacity in vitro; cellular adhesion and growth in vitro; and metastatic growth in vivo. In vivo models include, but are not limited to suitable syngeneic models, human tumor xenograft models, orthotopic models, metastatic models, transgenic models, and gene knockout models. See, e.g., Teicher, Tumors Models in Cancer Research (Humana Press 2001).

A compound is an inhibitor of lysyl oxidase expression or biological activity when the compound reduces the expression or activity or lysyl oxidase relative to that observed in the absence of the compound. In one embodiment, a compound is an inhibitor of lysyl oxidase when the compound reduces the incidence of metastasis relative to the observed in the absence of the compound and, in further testing, inhibits metastatic tumor growth.

The tumor inhibition can be quantified using any convenient method of measurement. For example, the incidence of metastasis can be assessed by examining relative dissemination (e.g., number of organ systems involved) and relative tumor burden in these sites. Metastatic growth can be ascertained by microscopic or macroscopic analysis, as appropriate. Tumor metastasis can be reduced by about 10%, 20%, 30%, 40%, 50%, 60%, 70%, 80%, 90%, 95% or greater.

Lysyl oxidase expression may be assessed using promoter analysis. Any convenient system for promoter activity analysis can be employed. Typically, the reporter gene system allows promoter activity to be detected using the lysyl oxidase promoter attached to a reporter molecule such that promoter activity results in the expression of the reporter molecule. See, e.g., Ausubel et al., Current Protocols in Molecular Biology (John Wiley & Sons, current edition) at chapter 9.6.

Also, LOX may be inhibited by degradation of its mRNA. An approach to this form of gene regulation is described in Wilson et al. “Modulation of LDL receptor mRNA stability by phorbol esters in human liver cell culture models,” Lipid Res. 38, 437-446 (1997).

The lysyl oxidase inhibitor compounds of the present invention may be used in the LOX inhibition therapy described herein.

### In Vitro Methods

The present invention also provides in vitro methods of internalising EGFR or reducing EGFR expression in a cell, said method comprising the step of contacting the cell with a LOX inhibitor of the invention.

In another aspect, the present invention further comprises an in vitro method of downregulating MATN2 expression in a cell, comprising the step of contacting the cell with a LOX inhibitor of the invention.

In a further aspect, the present invention also provides upregulating pSMAD2 in a cell comprising contacting a cell with a LOX inhibitor of the invention.

Suitably, in all aspects, the cell may be a cell-line, preferably a mammalian cell line.

Suitably, the cell may be a cancer cell, preferably a cancer cell associated with overexpression of EGFR.

### Combination Therapies e.g. for the Treatment of Cancer

LOX inhibition can be a useful method for improving the efficacy of other drugs or addressing resistance to drug treatment through a number of mechanisms. Specific inhibition of LOX with siRNA can induce apoptosis of laryngeal cancer Hep-2 cells and enhance the sensitivity of Hep-2 cells to chemotherapeutic drugs such as cisplatin (Dong, Lu et al. 2014) and to radiation (Dong, Xin et al. 2014). LOX-expression and secretion is increased in response to ionizing radiation (IR) and hypoxia, suggesting that LOX may contribute towards an IR-induced migratory phenotype in sublethally-irradiated tumor cells and tumor progression; therefore LOX inhibitors can be used in combination with radiotherapy to reduce side effects in surrounding tissues receiving a reduced radiation dose (Shen, Sharma et al. 2014). LOX and LOXL2 inhibition can alter vascular permeability or normalise vasculature in a tumour environment, which can enhance the delivery or effectiveness of drugs (Ingber and Mammoto 2014) (Marshall, Spangler et al. 2012), for example improved efficacy of treatment in ovarian xenograft and lung allograft mice models with chemotherapeutic agents such as taxol (Zaffryar-Eilot, Marshall et al. 2013). Pharmacological inhibition of lysyl oxidases improved drug delivery and reversed the negative effect of VEGF ablation on drug delivery and therapeutic response in anti-VEGF-resistant tumors (Roehrig et al, 2017). The extracellular matrix has been proposed to have an important role in the resistance to chemotherapeutics. It has been shown that inhibition of LOX for cells grown in collagen (as a surrogate of ECM) reverses their collagen-dependent increased resistance to chemotherapeutics such as erlotinib, cisplatin or methotrexate (Smith and Holzer 2010). Drug diffusion and efficacy is reduced by the enzymatic action of LOX and LOXLs on the ECM in a 3D cell culture (not in 2D) and sensitivity to doxorubicin and paclitaxel can be restored by inhibition with BAPN (Schuetze, Roehrig et al. 2015). LOX inhibition synergized with gemcitabine to kill tumors and significantly prolonged tumor-free survival in a pancreatic mouse model. This was associated with stromal alterations and increased infiltration of macrophages and neutrophils into tumors. Therefore, targeting LOX could improve outcome in surgically resectable disease (Miller, Morton et al. 2015).

The compounds of the invention may be used alone to provide a therapeutic effect. The compounds of the invention may also be used in combination with one or more additional anti-tumour agent and/or radiotherapy.

Such chemotherapy may include one or more of the following categories of anti-cancer agents:

(i) antiproliferative/antineoplastic drugs and combinations thereof, such as alkylating agents (for example cis-platin, oxaliplatin, carboplatin, cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophoporamine, carmustine, lomustine, stroptozocin and dacarbazine); antimetabolites (for example gemcitabine and antifolates such as fluoropyrimidines like 5-fluorouracil and tegafur, raltitrexed, methotrexate, pemetrexed, leucovorin, cytosine arabinoside, floxuridine, cytarabine, 6-mercaptopurine, 6-thioguanine, fludarabine phosphate, pentostatine, and gemcitabine and hydroxyurea, and trifluridine with trifluracil); antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors; eribulin); proteasome inhibitors, for example carfilzomib and bortezomib; interferon therapy; and topoisomerase inhibitors (for example epipodophyllotoxins like etoposide and teniposide, amsacrine, topotecan, irinotecan, mitoxantrone and camptothecin); bleomcin, dactinomycin, daunorubicin, doxorubicin, epirubicin, idarubicin, ara-C, paclitaxel (Taxol™), nabpaclitaxel, docetaxel, mithramycin, deoxyco-formycin, mitomycin-C, L-asparaginase, interferons (especially IFN-alpha), etoposide, teniposide, DNA-demethylating agents, (for example, azacitidine or decitabine); and histone de-acetylase (HDAC) inhibitors (for example vorinostat, MS-275, panobinostat, romidepsin, valproic acid, mocetinostat (MGCD0103) and pracinostat SB939; and belinostat, panobinostat); trabectedin;
(ii) cytostatic agents such as antiestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride; and navelbene, CPT-II, anastrazole, letrazole, capecitabine, cyclophosphamide, ifosamide, and droloxafine; and abiraterone, Enzalutamide; analogues of somatostatin such as lanreotide;
(iii) anti-invasion agents, for example dasatinib and bosutinib (SKI-606), and metalloproteinase inhibitors, inhibitors of urokinase plasminogen activator receptor function or antibodies to Heparanase;
(iv) inhibitors of growth factor function: for example such inhibitors include growth factor antibodies and growth factor receptor antibodies, for example the anti-erbB2 antibody trastuzumab [Herceptin™], the anti-HER2 antibody pertuzumab; the anti-EGFR antibody panitumumab, the anti-erbB1 antibody cetuximab, tyrosine kinase inhibitors, for example inhibitors of the epidermal growth factor family (for example EGFR family tyrosine kinase inhibitors such as gefitinib, erlotinib, 6-acrylamido-N-(3-chloro-4-fluorophenyl)-7-(3-morpholinopropoxy)-quinazolin-4-amine (CI 1033), afatinib, vandetanib, osimertinib and rociletinib) erbB2 tyrosine kinase inhibitors such as lapatinib) and antibodies to costimulatory molecules such as CTLA-4, 4-IBB and PD-I, or antibodies to cytokines (IL-10, TGF-beta); inhibitors of fibroblasts growth factor receptor family, such as ponatinib, nintedanib, lenvatinib, dovitinib, lucitanib, danusertinib, brivatinib, erdafitinib, PD173074, PD-166866, AZD4547, BGJ398, LY2874455, TAS-120, ARQ 087, BLU9931, DEBIO 1347, FGF401, BAY-1163877, FIIN-2, H3B-6527, PRN1371, BLU554, S49076, SU5416, SU6668, ENMD-2076, GP-369, IMCA1, PRO-001, R3mab; antibodies that block FGF ligand binding (ligand traps), such as FP-1039; antibodies that hinder FGFR dimerization such as MFGR1877S; antibody-drug conjugates targeting the FGFR family, such as BAY1187982; inhibitors of the hepatocyte growth factor family; inhibitors of the insulin growth factor family; modulators of protein regulators of cell apoptosis (for example Bcl-2 inhibitors); inhibitors of the platelet-derived growth factor family such as imatinib and/or nilotinib (AMN107); inhibitors of serine/threonine kinases (for example Ras/Raf signalling inhibitors such as farnesyl transferase inhibitors, sorafenib, tipifarnib and lonafarnib, vemurafenib, dabrafenib), inhibitors of cell signalling through MEK (such as trametinib, cobimetinib) and/or AKT kinases, c-kit inhibitors, abl kinase inhibitors such as ponatinib, PI3 kinase inhibitors, Plt3 kinase inhibitors, CSF-1R kinase inhibitors, IGF receptor, kinase inhibitors; aurora kinase inhibitors and cyclin dependent kinase inhibitors such as CDK2 and/or CDK4 inhibitors or CDK4/CDK6 inhibitors such as palbociclib, ribociclib, abemaciclib; CCR2, CCR4 or CCR6 antagonists; mTOR kinase inhibitors such as Everolimus; Janus kinase family inhibitors such as ruxolitinib; Brunton's tyrosine kinase inhibitors such as Ibrutinib; anaplastic lymphoma kinase—ALK—such as ceritinib, crizotinib, alectinib; c-Met kinase inhibitors such as cabozantinib; hedgehog signalling pathway inhibitors such as vismodegib, sonidegib; and RAF kinase inhibitors such as BAL3833 or other RAF inhibitors described in WO2006043090, WO2009077766, WO2011092469 or WO2015075483;
(v) antiangiogenic agents such as those which inhibit the effects of vascular endothelial growth factor, [for example the anti-vascular endothelial cell growth factor antibody bevacizumab (Avastin™), anti-VEGF2 antibody ramucirumab; recombinant fusion protein ziv-aflibercept]; thalidomide; pomalidomide; lenalidomide; and for example, a VEGF receptor tyrosine kinase inhibitor such as regorafenib, vandetanib, vatalanib, sunitinib, axitinib and pazopanib and lenvatinib;
(vi) gene therapy approaches, including for example approaches to replace aberrant genes such as aberrant p53 or aberrant BRCA1 or BRCA2; oncolytic viruses such as talimogene laherparepvec;
(vii) immunotherapy approaches, including for example antibody therapy such as denosumab, obinutuzumab, blinatomumab, dinutuximab, idarucizumab, daratumumab, durvalumab, necitumumab, elotuzumab, olaratumab, alemtuzumab, rituximab, ibritumomab tiuxetan (Zevalin®) and ofatumumab; interferons such as interferon α, peginterferon alpha-2b; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); gp100; dendritic cell-based vaccines (such as Ad.p53 DC); toll-like receptor modulators for example TLR-7 or TLR-9 agonists; PD-1, PD-L1, PD-L2 and CTL4-A modulators (for example Nivolumab, pembrolizumab, atezolizumab), antibodies and vaccines; other IDO inhibitors (such as indoximod); anti-PD-1 monoclonal antibodies (such as MK-3475 and nivolumab); anti-PDL1 monoclonal antibodies (such as MEDI-4736 and RG-7446); anti-PDL2 monoclonal antibodies; and anti-CTLA-4 antibodies (such as ipilumumab); antibody-drug conjugates such as Brentuximab vedotin, trastuzumab emtansine.
(viii) cytotoxic agents for example fludaribine (fludara), cladribine, pentostatin (Nipent™);
(ix) targeted therapies, for example PI3K inhibitors, for example idelalisib and perifosine; SMAC (second mitochondriaderived activator of caspases) mimetics, also known as Inhibitor of Apoptosis Proteins (IAP) antagonists (IAP antagonists). These agents act to supress IAPs, for example XIAP, cIAP1 and cIAP2, and thereby re-establish cellular apoptotic pathways. Particular SMAC mimetics include Birinapant (TL32711, TetraLogic Pharmaceuticals), LCL161 (Novartis), AEG40730 (Aegera Therapeutics), SM-164 (University of Michigan), LBW242 (Novartis), ML101 (Sanford-Burnham Medical Research Institute), AT-406 (Ascenta Therapeutics/University of Michigan), GDC-0917 (Genentech), AEG35156 (Aegera Therapeutic), and HGS1029 (Human Genome Sciences); and agents which target ubiquitin proteasome system (UPS), for example, bortezomib, ixazomib, carfilzomib, marizomib (NPI-0052), and MLN9708; and DNA repair inhibitors such as Olaparib, rucaparib; antiapoptotic BCL proteins family inhibitors such as venetoclax.
(xii) chimeric antigen receptors, anticancer vaccines and arginase inhibitors.

The additional anti-tumour agent may be a single agent or one or more of the additional agents listed herein.

Particular anti-cancer agents which may be used together with a compound of the invention include for example: Such combination treatment may be achieved by way of the simultaneous, sequential or separate dosing of the individual components of the treatment. Such combination products employ the compounds of this invention within a therapeutically effective dosage range described hereinbefore and the other pharmaceutically-active agent within its approved dosage range.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

In some embodiments in which a combination treatment is used, the amount of the compound of the invention and the amount of the other pharmaceutically active agent(s) are, when combined, therapeutically effective to treat a targeted disorder in the patient. In this context, the combined amounts are “therapeutically effective amount” if they are, when combined, sufficient to reduce or completely alleviate symptoms or other detrimental effects of the disorder; cure the disorder; reverse, completely stop, or slow the progress of the disorder; or reduce the risk of the disorder getting worse. Typically, such amounts may be determined by one skilled in the art by, for example, starting with the dosage range described in this specification for the compound of the invention and an approved or otherwise published dosage range(s) of the other pharmaceutically active compound(s).

According to a further aspect of the invention, there is provided a compound of the invention, or a pharmaceutically acceptable salt thereof, as defined hereinbefore and an additional anti-cancer agent as defined hereinbefore, for use in the conjoint treatment of cancer.

According to a further aspect of the invention, there is provided a pharmaceutical product comprising a compound of the invention, or a pharmaceutically acceptable salt thereof, as defined hereinbefore and an additional anti-cancer agent as defined hereinbefore for the conjoint treatment of cancer.

According to a further aspect of the invention, there is provided a method of treatment of a human or animal subject suffering from a cancer comprising administering to the subject a therapeutically effective amount of a compound of the invention, or a pharmaceutically acceptable salt thereof, simultaneously, sequentially or separately with an additional anti-cancer agent as defined hereinbefore.

According to a further aspect of the invention, there is provided a compound of the invention, or a pharmaceutically acceptable salt thereof, for use simultaneously, sequentially or separately with an additional anti-cancer agent as defined hereinbefore, in the treatment of a cancer.

The compound of the invention may also be used be used in combination with radiotherapy. Suitable radiotherapy treatments include, for example X-ray therapy, proton beam therapy or electron beam therapies. Radiotherapy may also encompass the use of radionuclide agents, for example ¹³¹I, ³²P, ⁹⁰Y, ⁸⁹Sr, ¹⁵³Sm or ²²³Ra. Such radionuclide therapies are well known and commercially available.

According to a further aspect of the invention, there is provided a compound of the invention, or a pharmaceutically acceptable salt thereof, as defined hereinbefore for use in the treatment of cancer conjointly with radiotherapy.

According to a further aspect of the invention, there is provided a method of treatment of a human or animal subject suffering from a cancer comprising administering to the subject a therapeutically effective amount of a compound of the invention, or a pharmaceutically acceptable salt thereof, simultaneously, sequentially or separately with radiotherapy.

### Synthesis

In the description of the synthetic methods described below and in the referenced synthetic methods that are used to prepare the staring materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively necessary starting materials are obtainable by analogous procedures to those illustrated which are within the ordinary skill of an organic chemist.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl or trifluoroacetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example BF₃.OEt₂. A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

### Abbreviations

The following abbreviations are used:
Ac acetyl
Bn benzyl
BOC tert-butyloxycarbonyl
BOC₂O di-tert-butyl dicarbonate
Bu butyl
^{t}Bu t-butyl
BnNH₂ benzylamine
CDCl₃ deuterated chloroform
cHex cyclohexane
DCM dichloromethane
DIPEA N,N-Diisopropylethylamine
DMF N,N-Dimethyl formamide
DMSO dimethyl sulfoxide
Eq Equivalent
Et ethyl
EtOH ethanol
EtOAc ethyl acetate
Et₂O diethyl ether
GP general procedure
hr(s) hour(s)
HATU (1-[Bis(dimethylamino)methylene]-1H-1,2,3-triazolo[4,5b]pyridinium 3-oxid hexafluorophosphate
HPLC high performance liquid chromatography
HRMS high resolution mass spectroscopy
m-CPBA meta-chloroperoxybenzoic acid
Me methyl
MeOH methanol
MgSO₄ magnesium sulfate
NMR nuclear magnetic resonance
Pd—C palladium-on-carbon
Pd₂(dba)₃ tris(dibenzylideneacetone)dipalladium(0)
min(s) minute(s)
rt room temperature
TEA triethylamine
THF tetrahydrofuran
XPhos 2-Dicyclohexylphosphino-2′,4′,6′-triisopropylbiphenyl

### Experimental Procedures

Unless otherwise stated, reactions were conducted in oven-dried glassware under an atmosphere of nitrogen or argon using anhydrous solvents. All commercially obtained reagents and solvents were used as received. Column chromatography was performed on a Biotage SP1 purification system using Biotage Flash silica cartridges (SNAP KP-Sil). Semipreparative HPLC was performed on an Agilent 6120 system, flow 20 mL/min, eluents 0.1% acetic acid in water and 0.1% acetic acid in methanol, gradient of 10-100% organic phase. ¹H NMR spectra were recorded on Bruker AMX500 (500 MHz) or AMX300 (300 MHz) spectrometers using an internal deuterium lock. Chemical shifts are quoted in parts per million (ppm) using the following internal references: CDCl₃ (bH 7.26), MeOD (δH 3.31), and DMSO-d₆ (bH 2.50). Signal multiplicities are recorded as singlet (s), doublet (d), triplet (t), quartet (q), multiplet (m), doublet of doublets (dd). Coupling constants, J, were measured to the nearest 0.1 Hz. High resolution mass spectra were recorded on an Agilent 1200 series HPLC and diode array detector coupled to a 6210 time-of-flight mass spectrometer with dual multimode APCI/ESI source or on a Waters Acquity UPLC and diode array detector coupled to a Waters G2 QToF mass spectrometer fitted with a multimode ESI/APCI source. Analytical separation was carried out according to the methods listed below. The mobile phase was a mixture of methanol (solvent A) and water (solvent B), both containing formic acid at 0.1%, UV detection was at 254 nm. Method I: Agilent 1200 series HPLC, Merck Purospher STAR (RP-18e, 30 mm×4 mm) column using a flow rate of 1.5 mL/min in a 4 min gradient elution. Gradient elution was as follows: 10:90 (A/B) to 90:10 (A/B) over 2.5 min, 90:10 (A/B) for 1 min, and then reversion back to 10:90 (A/B) over 0.3 min, finally 10:90 (A/B) for 0.2 min. Method II: Agilent 1200 series HPLC, Merck Chromolith flash column (RP-18e, 25 mm×2 mm) at 30° C. using a flow rate of 0.75 mL/min in a 4 min gradient elution. Gradient elution was as follows: 5:95 (A/B) to 100:0 (A/B) over 2.5 min, 100:0 (A/B) for 1 min, and then reversion back to 5:95 (A/B) over 0.1 min, finally 5:95 (A/B) for 0.4 min. Method III: Waters Acquity UPLC, Phenomenex Kinetex XB-C18 column (30 mm×2.1 mm, 1.7μ, 100 A) at 30° C. using flow rate of 0.3 mL/min in a 4 min gradient elution. Gradient elution was as follows: 10:90 (A/B) to 90:10 (A/B) over 3 min, 90:10 (A/B) for 0.5 min, and then reversion back to 10:90 (A/B) over 0.3 min, finally 10:90 (A/B) for 0.2 min; Method IV: Waters Acquity UPLC, Phenomenex Kinetex C18 column (30 mm×2.1 mm, 2.6μ, 100A), flow rate and gradient elution according to Method Ill. The following reference masses were used for HRMS analysis: Agilent 1200 series, caffeine [M+H]⁺ 195.087652, hexakis(1H,1H,3H-tetrafluoropentoxy)phosphazene [M+H]⁺ 922.009798, and hexakis-(2,2-difluoroethoxy)phosphazene [M+H]⁺ 622.02896 or reserpine [M+H]⁺ 609.280657. Waters Acquity UPLC: leucine enkephalin fragment ion [M+H]⁺ 397.1876. All compounds were >95% purity by LCMS analysis unless otherwise stated.

### General Procedures for the Synthesis of Cyclic Diamines

### General Procedure GP1

Intermediates according to the invention may be prepared by as illustrated in Scheme 1, wherein R and R′ correspond to the substituent of each specific compound described herein.

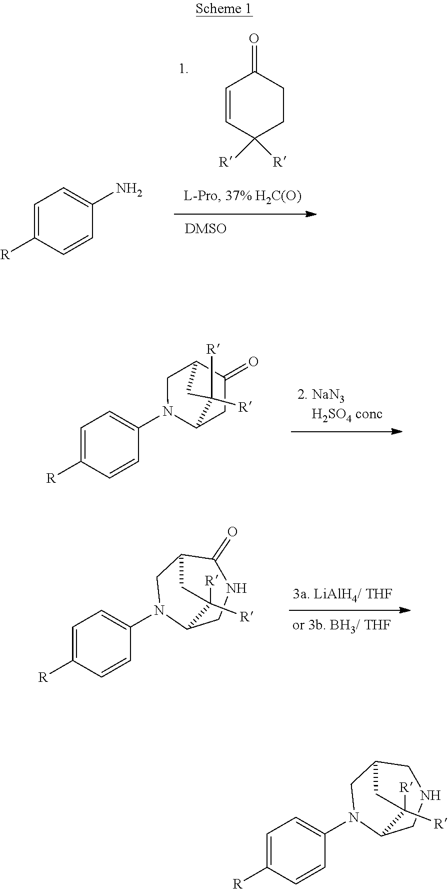

In a first step, a solution of the aniline (1 to 1.2 eq), 2-cyclohexen-1-one (1.6 to 2.5 eq), L-proline (0.3 eq) and 37% aqueous formaldehyde (1 eq.) in DMSO (1 to 2.5 M) was stirred at rt−50° C. for up to 24 hrs. After cooling down to rt, the mixture was diluted with EtOAc then washed with water and brine. The organic phase was separated, dried over MgSO₄, filtered and evaporated to dryness. The resulting residue was purified by flash chromatography (using a Biotage silica gel column and a gradient of EtOAc in cHex). This intermediate was used in the next step without further purification.

In a second step, to a solution of the bridged piperidin-4-one (1 eq) in concentrated H₂SO₄ (0.4 to 1M) was added at 0° C. and portionwise sodium azide (1.5 to 2.5 eq.). The reaction mixture was stirred at 0° C. for 30 mins then 30 mins-2 hrs at rt. The mixture was then cooled to 0° C., diluted with ice, made basic by the cautious addition of NaOH pellets (or a 3M aqueous solution of NaOH) and extracted with DCM (or EtOAc). The combined organic layers were dried over MgSO₄, filtered and evaporated under reduced pressure. The resulting residue was purified by flash chromatography (using a Biotage silica gel column and a gradient of MeOH in EtOAc (or a gradient of EtOAc in cHex) to give the two separate regioisomers.

In a third step (using conditions a), to a solution of the bridged homopiperazinone (1 eq) in anhydrous THF (0.3 to 1 M) was added dropwise at 0° C. a solution of LiAlH₄ in THF (1.0 M or 2.0 M, 2 to 6 eq). The mixture was stirred at reflux for 3-24 hrs then cooled to rt and water (0.05 mL per mmol of LiAlH₄), a 2.0 M aqueous solution of NaOH (0.1 mL per mmol of LiAlH₄), and again water (0.1 mL per mmol of LiAlH₄) were added. The mixture was stirred for 30 min at rt, diluted with EtOAc, dried over MgSO₄, filtered and concentrated under reduced pressure to give the bridged homopiperazine derivative. This material was used in the next step without further purification.

Alternatively (using conditions b), to a solution of the bridged homopiperazinone (1 eq) in THF (0.5 M) was added dropwise at 0° C. a BH₃. THFcomplex (1.0 M) (5 eq). The mixture was refluxed for 1-5 days then quenched with ethanol and stirred again at reflux for 1 day. After cooling down to rt, all the volatiles were removed under reduced pressure. The resulting residue was used in the next step without further purification.

### Intermediate 1: (1R,5S)-6-(4-ethoxyphenyl)-9,9-dimethyl-3,6-diazabicyclo[3.2.2]nonane

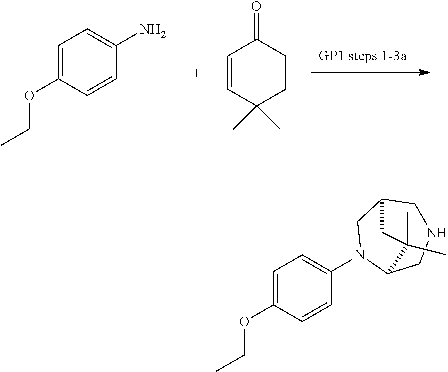

Using GP1 steps 1-3a with 4-ethoxyaniline (8.6 mL, 60 mmol) and 4,4-dimethylcyclohex-2-en-1-one (12.5 mL, 95 mmol), intermediate (1R,5S)-6-(4-ethoxyphenyl)-9,9-dimethyl-3,6-diazabicyclo[3.2.2]nonane was obtained and used in the next step without further purification (446 mg, 12%). ¹H NMR (500 MHz, CDCl₃) δ 6.85 (d, J=9.1 Hz, 2H), 6.56 (d, J=9.1 Hz, 2H), 3.98 (q, J=6.9 Hz, 2H), 3.32-3.31 (m, 1H), 3.29 (dd, J=10.1 and 2.1 Hz, 1H), 3.24 (dd, J=13.6 and 3.8 Hz, 1H), 3.14 (dt, J=10.1 and 2.8 Hz, 1H), 3.10-3.06 (m, 1H), 2.88 (dd, J=13.2 and 2.1 Hz, 1H), 2.84 (d, J=13.6, 1H), 2.32-2.20 (m, 1H), 1.77 (s, 1H) 1.71 (d, J=13.6 Hz, 1H), 1.49-1.45 (m, 1H), 1.38 (t, J=6.9 Hz, 3H), 1.15 (s, 3H), 0.89 (s, 3H); HRMS calcd for C₁₇H₂₇N₂O [M+H]⁺ 275.2123; found 275.2119.

### Intermediate 2: (1R,5S)-6-(4-ethoxyphenyl)-3,6-diazabicyclo[3.2.2]nonane

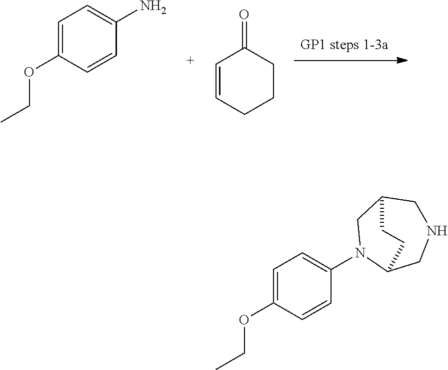

Using GP1 steps 1-3a with 4-ethoxyaniline (6.44 mL, 50 mmol) and 2-cyclohexenone (9.68 mL, 100 mmol), intermediate (1R,5S)-6-(4-ethoxyphenyl)-3,6-diazabicyclo[3.2.2]nonane was obtained (426.1 mg, 3% over 3 steps). HRMS calcd for C₁₅H₂₃N₂O [M+H]⁺ 247.1810; found 247.1858.

### Intermediate 3: 4-(4-((1R,5S)-9,9-dimethyl-3,6-diazabicyclo[3.2.2]nonan-6-yl)phenyl) thiomorpholine-1,1-dioxide

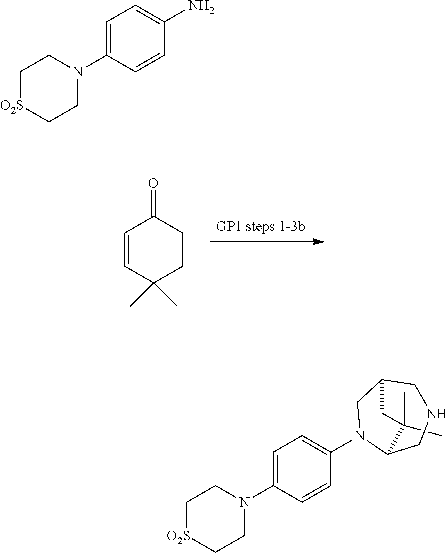

Using GP1 steps 1-3b with 4-(4-aminophenyl)thiomorpholine-1,1-dioxide (5 g, 22.1 mmol) and 4,4-dimethylcyclohex-2-en-1-one (4.65 mL, 35.4 mmol), 4-(4-((1R,5S)-9,9-dimethyl-3,6-diazabicyclo[3.2.2]nonan-6-yl)phenyl) thiomorpholine-1,1-dioxide was obtained and used in the next step without further purification. HRMS calculated for C₁₉H₃₀N₃O₂S [M+H]⁺ 364.2014; found 364.2112.

### Intermediate 4: (1S,4S)-2-(4-ethoxyphenyl)-2,5-diazabicyclo[2.2.1]heptane (HCl salt)

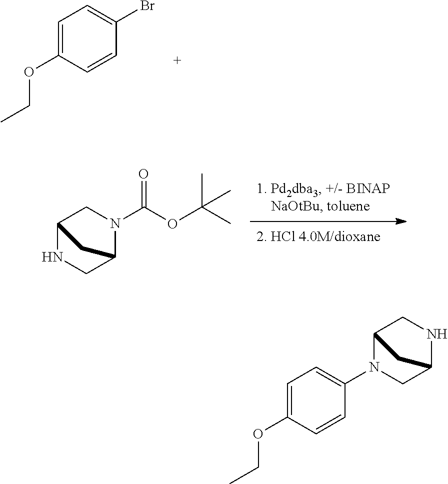

A solution of 4-bromophenetol (0.72 mL, 5.04 mmol) and (1S,4S)-2-BOC-2,5-diazabicyclo-[2,2,2]-heptane (1 g, 5.04 mmol) in toluene (21 mL) was reacted under reflux with Pd₂(dba)₃ (230.8 mg, 0.05 mmol), +/−BINAP (313.8 mg, 0.504 mmol) and NaOtBu (968.8 mg, 10.1 mmol) for 24 hrs. After cooling down to rt, the mixture was filtered through a celite pad then evaporated to dryness. The resulting residue was dissolved in DCM, washed with water, dried over MgSO₄, filtered and evaporated to dryness. The crude was purified by flash chromatography (using a Biotage silica gel column and a gradient of EtOAc in cHex, 0→50%) to give t-butyl-(1S,4S)-5-(4-ethoxyphenyl)-2,5-diazabicyclo[2.2.1]heptane-2-carboxylate (978.6 mg, 61%). ¹H NMR (500 MHz, DMSO-d₆) δ 6.81-6.75 (m, 2H), 6.55-6.49 (m, 2H), 4.44-4.32 (m, 2H), 3.94-3.84 (m, 2H), 3.56-3.47 (m, 1H), 3.30-3.16 (m, 2H), 2.87 (dd, 1H, J=12.4 and 5.8 Hz), 1.92-1.80 (m, 2H), 1.37, 1.32 (2s, 9H), 1.27 (t, 3H, J=6.9 Hz); HRMS calcd for C₁₈H₂₇N₂O₃ [M+H]⁺ 319.2022; found 319.1717.

A suspension of t-butyl (1S,4S)-5-(4-ethoxyphenyl)-2,5-diazabicyclo[2.2.1] heptane-2-carboxylate (965 mg, 3.03 mmol) in HCl/dioxane (4.0 M, 15 mL) was stirred at rt for 1 hr then evaporated to dryness to give (1 S,4S)-2-(4-ethoxyphenyl)-2,5-diazabicyclo[2.2.1]heptane (HCl salt). This intermediate was used in the next step without further purification. HRMS calcd for C₁₃H₁₉N₂O [M+H]⁺ 219.1497; found 219.1564.

### Intermediate 5: 2-(4-ethoxyphenyl)octahydropyrrolo[3,4-c]pyrrole (HCl salt)

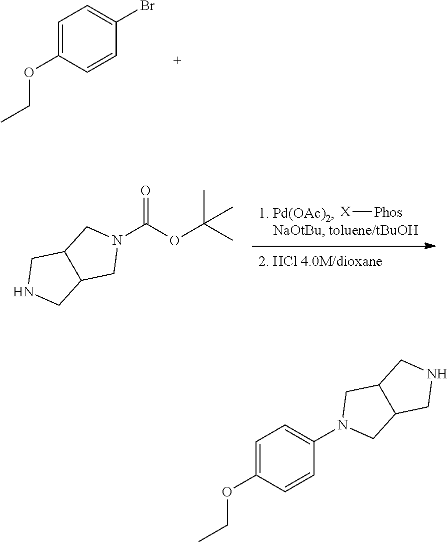

A solution of 4-bromophenetol (3.57 mL, 23.6 mmol) and t-butyl hexahydropyrrolo[3,4-c]pyrrole-2(1H)-carboxylate (5 g, 23.6 mmol) in toluene/t-BuOH (4:1, 118 mL) was reacted at 110° C. with Pd(OAc)₂ (265 mg, 5%), X-Phos (1.0 g, 10%) and NaOtBu (2.72 g, 28.3 mmol) for 16 hrs. After cooling down to rt, all the volatiles were removed under reduced pressure. The resulting residue was purified by flash chromatography (using a Biotage silica gel column and a gradient of EtOAc in cHex, 0→30%) to give t-butyl 5-(4-ethoxyphenyl)hexahydropyrrolo[3,4-c]pyrrole-2(1H)-carboxylate (7.0 g, 89%). ¹H NMR (500 MHz, CDCl₃) δ 6.89-6.81 (m, 2H), 6.56-6.48 (m, 2H), 3.98 (q, J=7.0 Hz, 2H), 3.68-3.59 (m, 2H), 3.50-3.41 (m, 2H), 3.38 (d, J=10.5 Hz, 1H), 3.25 (d, J=10.5 Hz, 1H), 3.17 (dd, J=9.3 Hz, 9.3 and 3.6 Hz, 2H), 2.98 (s, 2H), 1.46 (s, 9H), 1.38 (t, J=7.0 Hz, 3H); HRMS calcd for C₁₉H₂₉N₂O₃ [M+H]⁺ 333.2159; found 333.2173.

A suspension of t-butyl 5-(4-ethoxyphenyl)hexahydropyrrolo[3,4-c]pyrrole-2(1H)-carboxylate (7.0 g, 21 mmol) in HCl/dioxane (4.0 M, 105 mL) was stirred at rt for 1 hr then evaporated to dryness to give 2-(4-ethoxyphenyl)octahydropyrrolo[3,4-c]pyrrole (HCl salt). This residue was used in the next step without further purification.

### Intermediate 6: cis-2-(4-fluorophenyl)octahydropyrrolo[3,4-c]pyrrole (HCl salt)

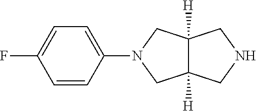 [SMILES: Fc1ccc(N2C[C@H]3CNC[C@H]3C2)cc1]

tert-Butyl cis-5-(4-fluorophenyl)hexahydropyrrolo[3,4-c]pyrrole-2(1H)-carboxylate was synthesised by methods described for intermediate 5 from Pd(OAc)₂ (96.1 mg, 5%), XPhos (409 mg, 10%), NaO^{t}Bu (988 mg, 10.3 mmol), 1-bromo-4-fluorobenzene (0.94 mL, 8.58 mmol), tert-butyl cis-hexahydropyrrolo[3,4-c]pyrrole-2(1H)-carboxylate (2.0 g, 9.43 mmol) and ^{t}BuOH/toluene (1:5, 43 mL); 90° C., 16 h. Chromatography (EtOAc/cyclohexane 0→30), white solid (1.91 g, 73%). ¹H NMR (500 MHz, Chloroform-d) δ 7.01-6.90 (m, 2H), 6.54-6.43 (m, 2H), 3.72-3.58 (m, 2H), 3.53-3.44 (m, 2H), 3.38 (br, 1H), 3.26 (br, 1H), 3.18 (dd, J=9.4, 3.7 Hz, 2H), 3.00 (br, 2H), 1.46 (s, 9H).

Intermediate 6 was obtained by treatment with 4 M HCl in dioxane as described for intermediate 5 and used in the next step without further purification.

### Intermediate 7: tert-butyl cis-5-(4-chlorophenyl)hexahydropyrrolo[3,4-c]pyrrole-2(1H)-carboxylate (HCl salt)

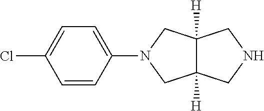 [SMILES: Clc1ccc(N2C[C@H]3CNC[C@H]3C2)cc1]

tert-Butyl cis-5-(4-chlorophenyl)hexahydropyrrolo[3,4-c]pyrrole-2(1H)-carboxylate was synthesised by methods described for intermediate 5 from Pd₂dba₃ (220 mg, 10%), XPhos (225 mg, 20%), NaO^{t}Bu (272 mg, 2.83 mmol), 1-chloro-4-iodobenzene (618 mg, 2.60 mmol), tert-butyl cis-hexahydropyrrolo[3,4-c]pyrrole-2(1H)-carboxylate (500 mg, 2.36 mmol) and ^{t}BuOH/toluene (1:5, 24 mL); 100° C., 16 h. Chromatography (basic alumina; EtOAc/pet ether 0→80%), pale yellow solid (590 mg, 78%). ¹H NMR (CDCl₃, 300 MHz) δ 7.16 (d, 2H, J=9.0 Hz), 6.45 (d, 2H, J=9.0 Hz), 3.67-3.61 (m, 2H), 3.53-3.46 (m, 2H), 3.38-3.21 (m, 2H), 3.17 (dd, 2H, J=9.6, 4.0 Hz), 3.04-2.94 (m, 2H), 1.45 (s, 9H) ppm; LCMS m/z 323.0 found (M+H)⁺, C₁₇H₂₄N₂O₂C₁.

Intermediate 7 was obtained by treatment with 4 M HCl in dioxane as described for intermediate 5 and used in the next step without further purification.

### Intermediate 8: 4-(4-(1,4-diazepan-1-yl)phenyl)thiomorpholine 1,1-dioxide (HCl salt)

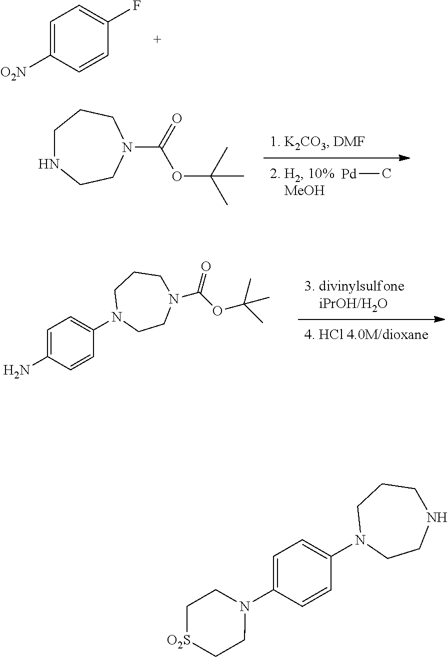

A solution of 1-fluoro-4-nitrobenzene (6.86 g, 48.6 mmol) and t-butyl 1,4-diazepane-1-carboxylate (9.74 g, 48.6 mmol) in DMF (73 mL) was reacted at 80° C. for 24 hrs. After cooling down to rt, water was added to the mixture and the resulting precipitate was filtered, washed with water and dried under high vacuum to give t-butyl-4-(4-nitrophenyl)-1,4-diazepane-1-carboxylate (14.8 g, 95%). HRMS calcd for C₁₂H₁₆N₃O₄ [M+H-tBu]⁺ 266.1141; found 266.1094.

A solution of t-butyl-4-(4-nitrophenyl)-1,4-diazepane-1-carboxylate (10.5 g, 32.7 mmol) in MeOH (654 mL) was reacted with H₂ generated by the H-Cube system using a 10% Pd—C cartridge. After evaporation of the volatiles, tert-butyl 4-(4-aminophenyl)-1,4-diazepane-1-carboxylate was recovered (7.2 g, 76%) and used in the next step without further purification.

A solution of t-butyl 4-(4-aminophenyl)-1,4-diazepane-1-carboxylate (7.2 g, 24.7 mmol) in a mixture of iPrOH/H₂O (3:1, v/v, 100 mL) was reacted with divinylsulfone (2.48 mL, 24.7 mmol) at 80° C. for 24 hrs. After cooling down to rt, the volatiles were removed under reduced pressure and the resulting residue purified by flash chromatography (using a Biotage silica gel column and a gradient of EtOAc in cHex, 0→80%). A fraction of this solid (1 g, 2.44 mmol) was then suspended in HCl/dioxane (4.0 M, 15 mL) and stirred at rt for 1 hr. After evaporation of the volatiles, 755 mg of 4-(4-(1,4-diazepan-1-yl)phenyl)thiomorpholine 1,1-dioxide was obtained and used in the next step without further purification. HRMS calcd for C₁₅H₂₃N₃O₂S [M+H]⁺ 309.1511, found 309.25.

### Intermediate 9: t-butyl-(2-((3-(4-((1R,5S)-9,9-dimethyl-3,6-diazabicyclo[3.2.2]nonan-6-yl)phenoxy) propyl)sulfonyl)ethyl)carbamate

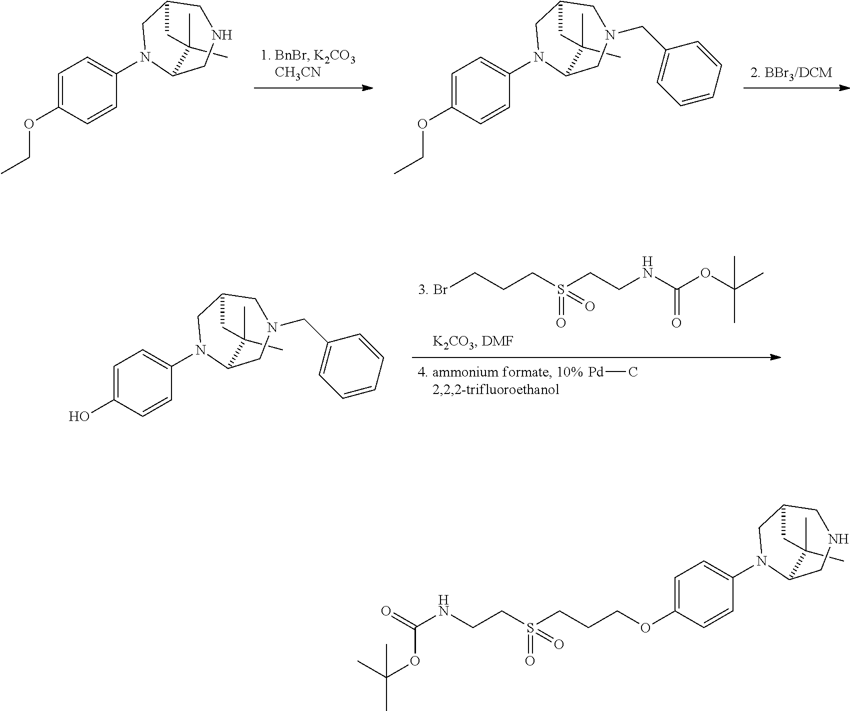

A solution of (1R,5S)-6-(4-ethoxyphenyl)-9,9-dimethyl-3,6-diazabicyclo[3.2.2]nonane (2 g, 7.29 mmol) and benzyl bromide (1.04 mL, 8.75 mmol) in CH₃CN (40 mL) was reacted with K₂CO₃ (2.01 g, 14.58 mmol) at 50° C. for 12 hrs. After cooling down to rt, the volatiles were removed and the residue dissolved in DCM and washed with water. The organic layer was separated, dried over MgSO₄, filtered and evaporated to dryness to give (1R,5S)-3-benzyl-6-(4-ethoxyphenyl)-9,9-dimethyl-3,6-diazabicyclo[3.2.2]nonane (3.01 g, quantitative). ¹H NMR (500 MHz, DMSO-d₆) δ 7.35-7.28 (m, 4H), 7.26-7.21 (m, 1H), 6.78-6.73 (m, 2H), 6.52-6.47 (m, 2H), 3.88 (q, J=6.9 Hz, 2H), 3.49 (s, 2H), 3.33 (d, J=4.9 Hz, 1H), 3.14 (dd, J=10.3, 2.5 Hz, 1H), 3.07-2.83 (m, 3H), 2.28 (t, J=7.4 Hz, 1H), 2.13 (dd, J=20.4, 11.6 Hz, 2H), 1.86 (d, J=12.7 Hz, 1H), 1.29-1.22 (m, 4H), 1.12 (s, 3H), 0.76 (s, 3H); HRMS calcd for C₂₄H₃₃N₂O [M+H]⁺ 365.2593; found 365.2601.

A solution of (1R,5S)-3-benzyl-6-(4-ethoxyphenyl)-9,9-dimethyl-3,6-diazabicyclo[3.2.2]nonane (2.88 g, 7.9 mmol) in DCM (35 mL) was reacted at 0° C. for 30 mins with a 1.0 M solution of boron tribromide in DCM (23.7 mL) then quenched at 0° C. with an aq. satd NaHCO₃ solution. The organic layer was recovered, dried over MgSO₄, filtered and evaporated to dryness. The resulting residue was purified by flash chromatography (using a Biotage column and a gradient of MeOH in EtOAc, 0→25%). The resulting residue was used in the next step without further purification. HRMS calcd for C₂₂H₂₉N₂O [M+H]+ 337.2280; found 337.2288.

A solution of 4-((1R,5S)-3-benzyl-9,9-dimethyl-3,6-diazabicyclo[3.2.2]nonan-6-yl)phenol (2.6 g, 7.73 mmol) and t-butyl-(2-((3-bromopropyl)sulfonyl)ethyl)carbamate (2.55 g, 7.73 mmol) in DMF (30 mL) was reacted with K₂CO₃ (2.16 g, 15.46 mmol) at 80° C. for 12 hrs. After cooling down to rt, the mixture was diluted DCM and washed with water. The organic layer was separated, dried over MgSO₄, filtered and evaporated to dryness. The residue was then purified by flash chromatography (using a Biotage silica gel column and a gradient of EtOAc in cHex, 0→50%) to give t-butyl-(2-((3-(4-((1R,5S)-3-benzyl-9,9-dimethyl-3,6-diazabicyclo[3.2.2]nonan-6-yl)phenoxy)propyl)sulfonyl)ethyl)carbamate (1.08 g, 24% over 2 steps). ¹H NMR (500 MHz, CDCl₃) δ 7.38-7.24 (m, 5H), 6.85-6.80 (m, 2H), 6.55-6.50 (m, 2H), 4.04 (t, J=5.7 Hz, 2H), 3.68 (q, J=5.8 Hz, 2H), 3.57-3.45 (m, 2H), 3.30-3.16 (m, 6H), 3.14-2.90 (m, 3H), 2.36-2.24 (m, 4H), 2.22-2.17 (m, 1H), 1.97 (dd, J=12.3, 2.1 Hz, 1H), 1.46 (s, 9H), 1.32 (dd, J=12.9, 6.6 Hz, 1H), 1.13 (s, 3H), 0.85 (s, 3H); HRMS calcd for C₃₂H₄₈N₃O₅S (M+H⁺) 586.3315; found 586.3530.

A solution of t-butyl (2-((3-(4-((1R,5S)-3-benzyl-9,9-dimethyl-3,6-diazabicyclo[3.2.2]nonan-6-yl)phenoxy)propyl)sulfonyl)ethyl)carbamate (1.06 g, 1.82 mmol) in 2,2,2-trifluoroethanol (20 mL) was reacted with ammonium formate (1.15 g, 18.2 mmol) and 10% Pd—C (212 mg, 20% wt) under reflux for 2 hrs. After cooling down to rt, the mixture was filtered through a celite pad and evaporated to dryness. The resulting residue was used in the next step without further purification. HRMS calcd for C₂₅H₄₂N₃O₅S (M+H⁺) 496.2845; found 496.2835.

### Intermediate 10: cis-2-(4-bromophenyl)octahydropyrrolo[3,4-c]pyrrole (HCl salt)

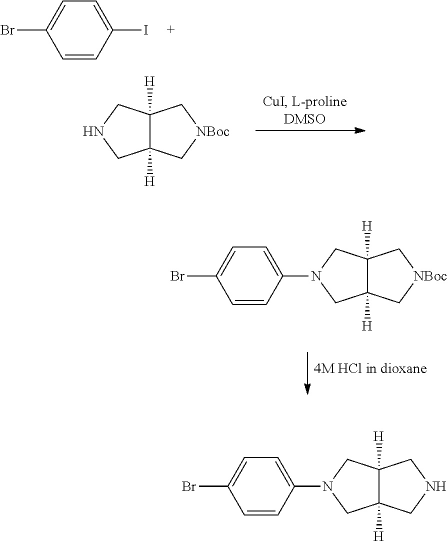

A mixture of 4-bromo-1-iodobenzene (1.39 g, 4.90 mmol), tert-butyl cis-hexahydropyrrolo[3,4-c]pyrrole-2(1H)-carboxylate (800 mg, 3.77 mmol), CuI (72.1 mg, 10%), L-proline (86.8 mg, 20%), K₂CO₃ (67.7 mg, 4.90 mmol) and DMSO (19 mL) was stirred at 90° C. for 16 h. After cooling to rt, the mixture was diluted with EtOAc. The organic phase was washed with H₂O (3×) and brine, dried over MgSO₄, filtered and the solvent was removed under reduced pressure. The crude was purified by chromatography (EtOAc/cyclohexane 0→25%) to afford tert-butyl cis-5-(4-bromophenyl)hexahydropyrrolo[3,4-c]pyrrole-2(1H)-carboxylate as a white solid (753 mg, 54%). ¹H NMR (500 MHz, Acetone-d₆) δ 7.31-7.25 (m, 2H), 6.55-6.50 (m, 2H), 3.67-3.55 (m, 2H), 3.55-3.44 (m, 2H), 3.26 (dd, J=11.4, 3.9 Hz, 2H), 3.23-3.14 (m, 2H), 3.11-3.01 (m, 2H), 1.42 (s, 9H). MS (ES+) m/z 367/369 (M+H)⁺.

Intermediate 10 was obtained by treatment with 4 M HCl in dioxane as described for intermediate 5 and used in the next step without further purification.

### Intermediate 11: cis-2-(perfluorophenyl)octahydropyrrolo[3,4-c]pyrrole (HCl salt)

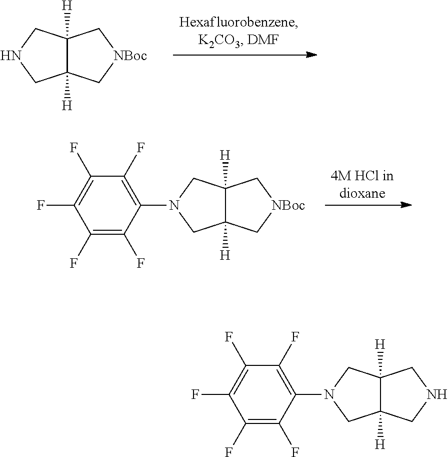

A mixture of tert-butyl cis-hexahydropyrrolo[3,4-c]pyrrole-2(1H)-carboxylate (0.50 g, 2.36 mmol), hexafluorobenzene (545 μL, 4.72 mmol), K₂CO₃ (391 mg, 2.83 mmol) and DMF (1.2 mL) was stirred at 70° C. for 5 h. After cooling to rt, the mixture was diluted with EtOAc (30 mL). The organic layer was washed with H₂O (3×30 mL), brine (30 mL), dried over MgSO₄, filtered and the solvent was removed under reduced pressure. The crude was purified by chromatography (EtOAc/pet ether 0→25%) to afford tert-butyl cis-5-(perfluorophenyl)hexahydropyrrolo[3,4-c]pyrrole-2(1H)-carboxylate as a colourless oil (439 mg, 49%). ¹⁹F NMR (282 MHz, Chloroform-d) δ −152.85-−153.25 (m), −164.08-−164.37 (m), −168.38-−168.67 (m). ¹H NMR (300 MHz, Chloroform-d) b 3.80-3.54 (m, 4H), 3.48-3.22 (m, 4H), 3.03-2.85 (m, 2H), 1.47 (s, 9H). MS (ESI) m/z 323 [M-^{t}Bu+2H]⁺.

Intermediate 11 was obtained by treatment with 4 M HCl in dioxane as decribed for intermediate 5 and used in the next step without further purification.

### Intermediate 12: tert-Butyl cis-5-(4-bromophenyl)-3a,6a-dimethylhexahydropyrrolo[3,4-c]pyrrole-2(1H)-carboxylate

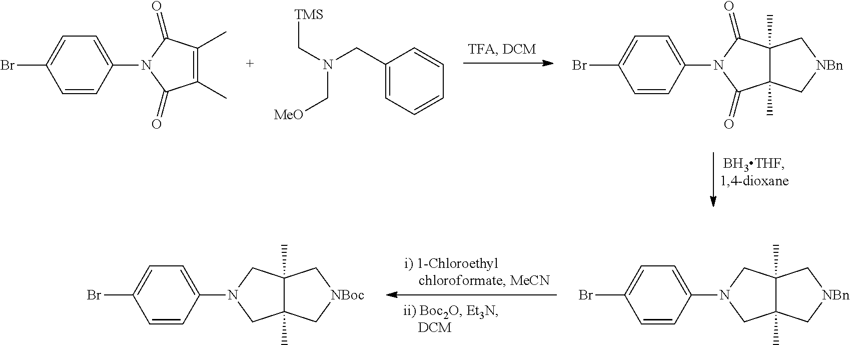

N-benzyl-1-methoxy-N-((trimethylsilyl)methyl)methanamine (165 mmol) was slowly added to a solution of trifluoroacetic acid (2.90 mL, 30%) and 1-(4-bromophenyl)-3,4-dimethyl-1H-pyrrole-2,5-dione (35.4 g, 127 mmol) in DCM (211 mL) by a dropping funnel. The mixture was stirred at rt for 20 h. 1M NaOH (200 mL) was added to the solids and the aqueous phase was extracted with DCM (2×150 mL), dried over MgSO₄, filtered and the solvent was removed under reduced pressure. The crude was recrystallized from EtOAc/cyclohexane to afford cis-5-benzyl-2-(4-bromophenyl)-3a,6a-dimethyltetrahydropyrrolo[3,4-c]pyrrole-1,3(2H,3aH)-dione as a white crystalline solid (38.5 g, 73%). ¹H NMR (500 MHz, Acetone-d₆) δ 7.74-7.67 (m, 2H), 7.35-7.21 (m, 7H), 3.59 (s, 2H), 3.42-3.34 (m, 2H), 2.28-2.23 (m, 2H), 1.33 (s, 6H).

BH₃. THF (1.0 M; 194 mL, 194 mmol) was added to a solution of cis-5-benzyl-2-(4-bromophenyl)-3a,6a-dimethyltetrahydropyrrolo[3,4-c]pyrrole-1,3(2H,3aH)-dione (20.0 g, 48.4 mmol) in 1,4-dioxane (194 mL) and the mixture was stirred at 90° C. for 16 h. After cooling to rt, it was carefully quenched with EtOH (˜200 mL) and stirred at 90° C. for 1 h. The solvent was removed under reduced pressure and the crude as purified by chromatography (THF/cyclohexane 0→25%) to afford cis-2-benzyl-5-(4-bromophenyl)-3a,6a-dimethyloctahydropyrrolo[3,4-c]pyrrole as a colourless syrup (13.8 g, 74%). ¹H NMR (500 MHz, Chloroform-d) δ 7.34-7.19 (m, 7H), 6.51-6.44 (m, 2H), 3.60 (s, 2H), 3.39 (d, J=9.3 Hz, 2H), 3.08 (d, J=9.3 Hz, 2H), 2.78 (d, J=9.2 Hz, 2H), 2.47-2.38 (m, 2H), 1.13 (s, 6H).

1-Chloroethyl chloroformate (4.62 mL, 42.8 mmol) was added to a solution of cis-2-benzyl-5-(4-bromophenyl)-3a,6a-dimethyloctahydropyrrolo[3,4-c]pyrrole (13.7 g, 35.7 mmol) in MeCN (119 mL) and the mixture was stirred at 70° C. for 1 h. After cooling to rt, the solvent was removed under reduced pressure. DCM (119 mL) was added, followed by Et₃N (14.9 mL, 107 mmol) and Boc₂O (9.82 mL, 42.8 mmol) and the mixture was stirred at rt for 16 h. The solvent was removed under reduced pressure. Cyclohexane (200 mL) was added and the suspension was stirred for 10 min. The mixture was filtered and the solids were washed with more cyclohexane portions. The filtrates were collected and the solvent was removed under reduced pressure. The crude was purified by chromatography (EtOAc/cyclohexane 0→20%) to afford tert-butyl cis-5-(4-bromophenyl)-3a,6a-dimethylhexahydropyrrolo[3,4-c]pyrrole-2(1H)-carboxylate as a white solid (14.1 g, quant.). ¹H NMR (500 MHz, Chloroform-d) δ 7.32-7.28 (m, 2H), 6.40-6.35 (m, 2H), 3.62-3.12 (m, 8H), 1.46 (s, 9H), 1.14 (s, 6H).

### General Procedure GP2

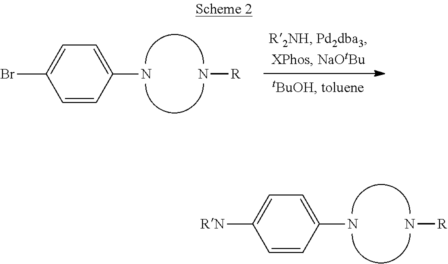

A mixture of aryl bromide (1.1 eqv.), amine R′NH₂ (1.0 eqv.), Pd₂dba₃ (10 mol %), XPhos (20 mol %) and sodium tert-butoxide (1.2 eqv) in toluene:^{t}BuOH (0.1-0.2 M) was degassed with argon for 5 minutes. and stirred at 100° C. overnight. After cooling to room temperature the reaction mixture was diluted with ethyl acetate and washed with water and brine, dried over anhydrous magnesium sulfate, filtered and concentrated to give the crude product, which was purified by chromatography.

### Intermediate 13: 4-(4-(cis-3a,6a-dimethylhexahydropyrrolo[3,4-c]pyrrol-2(1H)-yl)phenyl)morpholine (HCl salt)

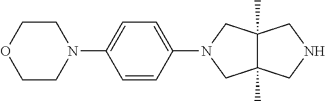 [SMILES: C[C@@]12CNC[C@]1(C)CN(c1ccc(N3CCOCC3)cc1)C2]

tert-Butyl cis-3a,6a-dimethyl-5-(4-morpholinophenyl)hexahydropyrrolo[3,4-c]pyrrole-2(1H)-carboxylate was synthesised by general procedure GP2 from morpholine (0.037 mL, 0.417 mmol), tert-butyl cis-5-(4-bromophenyl)-3a,6a-dimethylhexahydropyrrolo[3,4-c]pyrrole-2(1H)-carboxylate (150 mg, 0.379 mmol), Pd₂dba₃ (35 mg, 10 mol %), XPhos (36 mg, 20 mol %), sodium tert-butoxide (44 mg, 0.455 mmol) and ^{t}BuOH/toluene (1:4, 5.0 mL); 100° C., 16 h. Chromatography (basic alumina; EtOAc/pet ether 0→100%), off-white solid (90 mg, 59%). ¹H NMR (CDCl₃, 300 MHz) δ 6.90 (d, 2H, J=8.9 Hz), 6.46 (d, 2H, J=8.9 Hz), 3.87-3.84 (m, 4H), 3.55-3.19 (m, 8H), 3.02-2.99 (m, 4H), 1.44 (s, 9H), 1.12 (s, 6H) ppm; LCMS m/z 402.4 found (M+H)⁺, C₂₃H₃₆N₃O₃.

Intermediate 13 was obtained by treatment with 4 M HCl in dioxane as described for intermediate 5 and used in the next step without further purification.

### Intermediate 14: cis-3a,6a-dimethyl-2-(4-(4-(methylsulfonyl)piperazin-1-yl)phenyl)octahydropyrrolo[3,4-c]pyrrole (HCl salt)

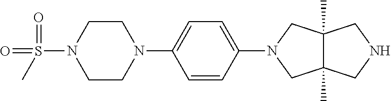 [SMILES: C[C@@]12CNC[C@]1(C)CN(c1ccc(N3CCN(S(C)(=O)=O)CC3)cc1)C2]

tert-Butyl cis-3a,6a-dimethyl-5-(4-(4-(methylsulfonyl)piperazin-1-yl)phenyl)hexahydropyrrolo[3,4-c]pyrrole-2(1H)-carboxylate was synthesised by general procedure GP2 from 1-(methylsulfonyl)piperazine (90 mg, 0.55 mmol), tert-butyl cis-5-(4-bromophenyl)-3a,6a-dimethylhexahydropyrrolo[3,4-c]pyrrole-2(1H)-carboxylate (200 mg, 0.50 mmol), Pd₂dba₃ (45 mg, 10 mol %), XPhos (48 mg, 20 mol %), sodium tert-butoxide (58 mg, 0.60 mmol) and toluene:^{t}BuOH (5:1, 6 mL); 100° C., 16 h. Chromatography (basic alumina; EtOAc/pet ether 0→100%), light brown solid (184 mg, 76%). ¹H NMR (CDCl₃, 300 MHz) δ 6.92 (d, 2H, J=8.9 Hz), 6.45 (d, 2H, J=8.9 Hz), 3.55-3.18 (m, 12H), 3.13-3.09 (m, 4H), 2.82 (s, 3H), 1.44 (s, 9H), 1.12 (s, 6H) ppm; LCMS m/z 479.4 found (M+H)⁺, C₂₄H₃₉N₄SO₄.

Intermediate 14 was obtained by treatment with 4 M HCl in dioxane as described for intermediate 5 and used in the next step without further purification.

### Intermediate 15: 4-(4-(cis-3a,6a-dimethylhexahydropyrrolo[3,4-c]pyrrol-2(1H)-yl)phenyl)thiomorpholine 1,1-dioxide (HCl salt)

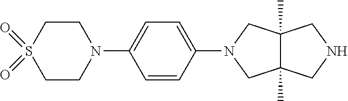 [SMILES: C[C@@]12CNC[C@]1(C)CN(c1ccc(N3CCS(=O)(=O)CC3)cc1)C2]

tert-Butyl cis-5-(4-(1,1-dioxidothiomorpholino)phenyl)-3a,6a-dimethylhexahydropyrrolo[3,4-c]pyrrole-2(1H)-carboxylate was synthesised by general procedure GP2 from thiomorpholine-1,1-dioxide (75 mg, 0.55 mmol) and tert-butyl cis-5-(4-bromophenyl)-3a,6a-dimethylhexahydropyrrolo[3,4-c]pyrrole-2(1H)-carboxylate (200 mg, 0.50 mmol), Pd₂dba₃ (45 mg, 10 mol %), XPhos (48 mg, 20 mol %), sodium tert-butoxide (58 mg, 0.60 mmol) and toluene:^{t}BuOH (5:1, 6 mL); 100° C., 16 h. Chromatography (basic alumina; EtOAc/pet ether 0→100%), off-white solid (184 mg, 81%). ¹H NMR (CDCl₃, 300 MHz) δ 6.92 (d, 2H, J=8.9 Hz), 6.44 (d, 2H, J=8.9 Hz), 3.61-3.58 (m, 4H), 3.54-3.19 (m, 8H), 3.16-3.12 (m, 4H), 1.44 (s, 9H), 1.13 (s, 6H) ppm; LCMS m/z 450.4 found (M+H)⁺, C₂₃H₃₆N₃SO₄.

Intermediate 15 was obtained by treatment with 4 M HCl in dioxane as described for intermediate 5 and used in the next step without further purification.

### Intermediate 16: 4-(4-(2,7-diazaspiro[4.4]nonan-2-yl)phenyl)thiomorpholine 1,1-dioxide (HCl salt)

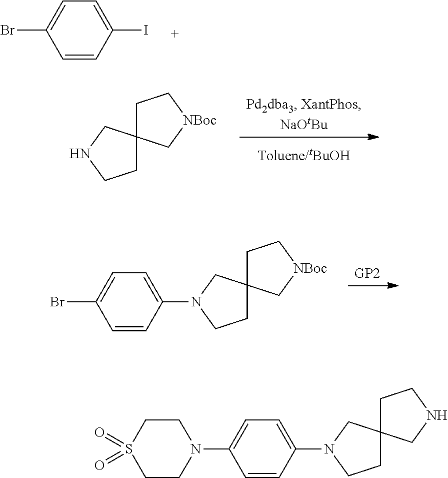 [SMILES: Brc1ccc(I)cc1 ; CC(C)(C)OC(=O)N1CCC2(CCNC2)C1 ; "Pd2dba3, XantPhos,NaOtBu" ; CC(C)(C)OC(=O)N1CCC2(CCN(c3ccc(Br)cc3)C2)C1 ; "GP2" ; O=S1(=O)CCN(c2ccc(N3CCC4(CCNC4)C3)cc2)CC1]

1-Bromo-4-iodobenzene (344 mg, 1.22 mmol) and tert-butyl 2,7-diazaspiro[4.4]nonane-2-carboxylate (250 mg, 1.10 mmol) were suspended in toluene:^{t}BuOH (5:1, 12 mL) at room temperature and the solution was degassed with argon for 5 minutes. Pd₂dba₃ (101 mg, 10 mol %), XantPhos (127 mg, 20 mol %) and sodium tert-butoxide (127 mg, 1.32 mmol) were then added with stirring, and the solution was bubbled with argon for a further 5 minutes before heating to 100° C., with stirring under nitrogen overnight. After cooling to room temperature the reaction mixture was diluted with ethyl acetate (20 mL) and washed with water and brine, dried over anhydrous magnesium sulfate, filtered and concentrated to give the crude product, which was purified using flash column chromatography over basic alumina, eluting with 0-100% ethyl acetate/petroleum ether, to give tert-butyl 7-(4-bromophenyl)-2,7-diazaspiro[4.4]nonane-2-carboxylate (300 mg, 71% yield) as a yellow oil. ¹H NMR (CDCl₃, 300 MHz) δ 7.28 (d, 2H, J=8.2 Hz), 6.39 (d, 2H, J=8.5 Hz), 3.51-3.42 (m, 2H), 3.37-3.27 (m, 4H), 3.23-3.16 (m, 2H), 2.04-1.81 (m, 4H), 1.46 (s, 9H) ppm; LCMS m/z 381.3 found (M+H)⁺, C₁₃H₂₆N₂O₂Br.

tert-Butyl 7-(4-(1,1-dioxidothiomorpholino)phenyl)-2,7-diazaspiro[4.4]nonane-2-carboxylate was synthesised by general procedure GP2 from thiomorpholine-1,1-dioxide (108 mg, 0.80 mmol), tert-butyl 7-(4-bromophenyl)-2,7-diazaspiro[4.4]nonane-2-carboxylate (278 mg, 0.73 mmol), Pd₂dba₃ (67 mg, 10 mol %), XPhos (70 mg, 20 mol %) and sodium tert-butoxide (84 mg, 0.88 mmol) and toluene:^{t}BuOH (4:1, 10 mL)); 100° C., 16 h. Chromatography (basic alumina; EtOAc/pet ether 30→100%), off-white solid (221 mg, 70%). ¹H NMR (CDCl₃, 300 MHz) δ 6.92 (d, 2H, J=8.3 Hz), 6.50 (d, 2H, J=8.2 Hz), 3.62-3.59 (m, 4H), 3.51-3.20 (m, 8H), 3.16-3.12 (m, 4H), 2.04-1.84 (m, 4H), 1.46 (s, 9H) ppm; LCMS m/z 436.2 found (M+H)⁺, C₂₂H₃₄N₃O₄S.

Intermediate 16 was obtained by treatment with 4 M HCl in dioxane as described for intermediate 5 and used in the next step without further purification.

### Intermediate 17: cis-2-tosyloctahydropyrrolo[3,4-c]pyrrole (HCl salt)

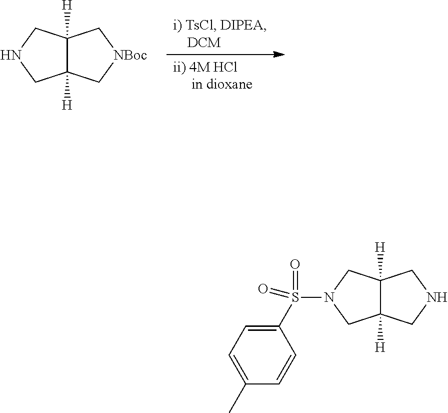

TsCl (1.89 g, 9.91 mmol) was added to a mixture of cis-tert-butyl-hexahydropyrrolo[3,4-c]pyrrole-2(1H)-carboxylate (2.0 g, 9.43 mmol) and DIPEA (2.50 mL, 14.2 mmol) in DCM (47 mL) and it was stirred at rt for 1 h before it was diluted with DCM (100 mL). The organic phase was washed with H₂O (150 mL), dried over MgSO₄, filtered and the solvent was removed under reduced pressure. The crude was dissolved in DCM (25 mL). 4 M HCl in dioxane (25 mL) was added and the mixture was stirred at rt for 3 h. EtOAc (50 mL) was added and the precipitated solid was filtered, washed with EtOAc and dried under vacuum to afford cis-2-tosyloctahydropyrrolo[3,4-c]pyrrole as a white solid (2.86 g, quant.). ¹H NMR (300 MHz, Methanol-d₄) δ 7.76-7.65 (m, 2H), 7.52-7.40 (m, 2H), 3.64-3.50 (m, 2H), 3.19-2.82 (m, 6H), 2.46 (s, 3H), 1.41-1.35 (m, 2H). MS (ESI) m/z 267 [M+H]⁺.

### Intermediate 18: tert-butyl (2-((4-bromobenzyl)sulfonyl)ethyl)carbamate

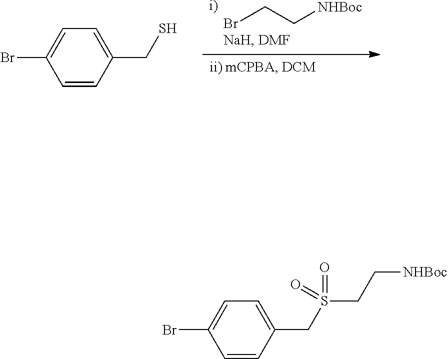

Sodium hydride (60%, 98 mg, 2.46 mmol) was added slowly to a solution of (4-bromophenyl)methanethiol (500 mg, 2.46 mmol) in anhydrous DMF (3 mL), with stirring at room temperature under nitrogen for 45 minutes. A solution of 2-(BOC-amino)ethyl bromide (368 mg, 1.64 mmol) in anhydrous DMF (2 mL) was then added drop-wise, with continued stirring overnight. The reaction mixture was then separated between diethyl ether and water, the organic phase was washed with water and brine, dried over anhydrous magnesium sulfate, filtered and concentrated to give the crude product, which was which was purified using flash column chromatography over silica, eluting with 0-100% ethyl acetate/petroleum ether, to give tert-butyl (2-((4-bromobenzyl)thio)ethyl)carbamate (482 mg, 57% yield) as a clear colourless oil. ¹H NMR (CDCl₃, 300 MHz) δ 7.42 (d, 2H, J=8.5 Hz), 7.19 (d, 2H, J=8.5 Hz), 4.83 (br s, 1H), 3.66 (s, 2H), 3.26 (q, 2H, J=6.2 Hz), 2.52 (t, 2H, J=6.6 Hz), 1.44 (s, 9H) ppm.

mCPBA (70%, 752 mg, 3.05 mmol) was added over 2 minutes to a solution of tert-butyl (2-((4-bromobenzyl)thio)ethyl)carbamate (480 mg, 1.39 mmol) in dichloromethane (15 mL), with stirring at room temperature under air for 18 hours. The reaction mixture was then quenched with aq. NaHCO₃ solution and extracted with dichloromethane. The combined organic extracts were washed with water and brine, dried over anhydrous sodium sulfate, filtered and concentrated to give the crude product, which was purified using flash column chromatography over silica, eluting with 10-100% ethyl acetate/cyclohexane, to give the title compound (379 mg, 72% yield) as a white solid. ¹H NMR (CDCl₃, 300 MHz) δ 7.55 (d, 2H, J=8.5 Hz), 7.29 (d, 2H, J=8.5 Hz), 5.11 (br s, 1H), 4.20 (s, 2H), 3.60 (q, 2H, J=6.1 Hz), 3.07 (t, 2H, J=6.0 Hz), 1.44 (s, 9H) ppm.

### Intermediate 19: tert-butyl (2-((4-hydroxyphenyl)sulfonyl)ethyl)carbamate

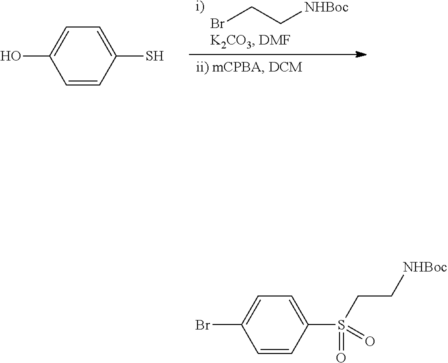

2-(BOC-Amino)ethyl bromide (888 mg, 3.96 mmol) was added to a suspension of 4-mercaptophenol (500 mg, 3.96 mmol) and potassium carbonate (602 mg, 4.36 mmol) in anhydrous DMF (5 mL), with stirring at room temperature under nitrogen overnight. The reaction mixture was then separated between diethyl ether and water, the organic phase was washed with water and brine, dried over anhydrous magnesium sulfate, filtered and concentrated to give the crude product, which was which was purified using flash column chromatography over silica, eluting with 5-100% ethyl acetate/petroleum ether, to give tert-butyl (2-((4-hydroxyphenyl)thio)ethyl)carbamate (1.04 g, 98% yield) as a clear colourless oil. ¹H NMR (CDCl₃, 300 MHz) δ 7.35 (br s, 1H), 7.28 (d, 2H, J=8.7 Hz), 6.77 (d, 2H, J=8.7 Hz), 5.06 (br s, 1H), 3.26 (q, 2H, J=5.8 Hz), 2.87 (t, 2H, J=6.4 Hz), 1.44 (s, 9H) ppm; LCMS m/z 214.1 found (M-^{t}Bu+2H)⁺.

2) mCPBA (70%, 2.09 g, 8.49 mmol) was added over 2 minutes to a solution of tert-butyl (2-((4-hydroxyphenyl)thio)ethyl)carbamate (1.04 g, 3.86 mmol) in dichloromethane (35 mL), with stirring at room temperature under air for 18 hours. The reaction mixture was then quenched with aq. NaHCO₃ solution and extracted with dichloromethane. The combined organic extracts were washed with water and brine, dried over anhydrous sodium sulfate, filtered and concentrated to give the crude product, which was purified using flash column chromatography over silica, eluting with 10-100% ethyl acetate/cyclohexane, to give the title compound (907 mg, 78% yield) as a white solid. ¹H NMR (DMSO-d₆, 300 MHz) δ 10.62 (s, 1H), 7.69 (d, 2H, J=8.8 Hz), 6.95 (d, 2H, J=8.8 Hz), 6.81 (br t, 1H, J=5.2 Hz), 3.30-3.26 (m, 2H), 3.19-3.12 (m, 2H), 1.32 (s, 9H) ppm; LCMS m/z 246.0 found (M-^{t}Bu+2H)⁺

### Intermediate 20: 4-(((2-((tert-butoxycarbonyl)amino)ethyl)sulfonyl) methyl)benzoic acid

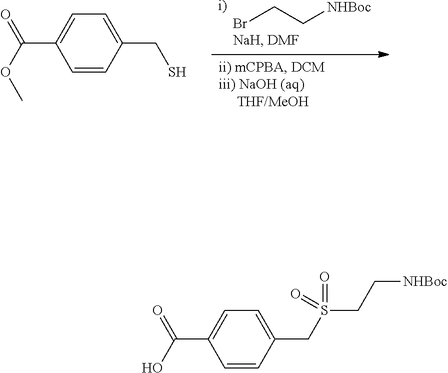

Sodium hydride (60%, 236 mg, 9.82 mmol) was added slowly to a solution of BOC-cysteamine (1.66 mL, 9.82 mmol) in anhydrous DMF (10 mL), with stirring at room temperature under nitrogen for 1 hour. A solution of methyl-4-bromomethyl benzoate (750 mg, 3.27 mmol) in anhydrous DMF (5 mL) was then added drop-wise, with continued stirring overnight. The reaction mixture was then separated between diethyl ether and water, the organic phase was washed with water and brine, dried over anhydrous magnesium sulfate, filtered and concentrated to give the crude product, which was which was purified using flash column chromatography over silica, eluting with 5-80% ethyl acetate/petroleum ether, to give methyl 4-(((2-((tert-butoxycarbonyl)amino)ethyl)thio)methyl) benzoate (664 mg, 62% yield) as a clear colourless oil. ¹H NMR (CDCl₃, 300 MHz) δ 7.97 (d, 2H, J=8.5 Hz), 7.38 (d, 2H, J=8.5 Hz), 4.84 (br s, 1H), 3.89 (s, 3H), 3.74 (s, 2H), 3.26 (q, 2H, J=6.2 Hz), 2.52 (t, 2H, J=6.6 Hz), 1.42 (s, 9H) ppm.

mCPBA (70%, 1.1 g, 4.46 mmol) was added over 2 minutes to a solution of methyl 4-(((2-((tert-butoxycarbonyl)amino)ethyl)thio)methyl) benzoate (660 mg, 2.03 mmol) in dichloromethane (25 mL), with stirring at room temperature under air for 18 hours. The reaction mixture was then quenched with aq. NaHCO₃ solution and extracted with dichloromethane. The combined organic extracts were washed with water and brine, dried over anhydrous sodium sulfate, filtered and concentrated to give the crude product, which was purified using flash column chromatography over silica, eluting with 20-100% ethyl acetate/cyclohexane, to give methyl 4-(((2-((tert-butoxycarbonyl)amino)ethyl)sulfonyl)methyl)benzoate (648 mg, 89% yield) as a white solid.

¹H NMR (CDCl₃, 300 MHz) δ 8.08 (d, 2H, J=8.5 Hz), 7.50 (d, 2H, J=8.5 Hz), 5.10 (br s, 1H), 4.31 (s, 2H), 3.93 (s, 3H), 3.60 (q, 2H, J=6.1 Hz), 3.08 (t, 2H, J=6.0 Hz), 1.44 (s, 9H) ppm.

1 M Aqueous NaOH (1.85 mL, 1.85 mmol) was added to a solution of methyl 4-(((2-((tert-butoxycarbonyl)amino)ethyl)sulfonyl)methyl)benzoate (300 mg, 0.839 mmol) in THF/methanol (2:1, 24 mL) with stirring at 25° C. overnight. The organic solvent was then removed and the reaction mixture was acidified to pH 4 with 1 M aqueous HCl, before separating between ethyl acetate and water. The organic phase was washed with brine, dried over anhydrous magnesium sulfate, filtered and concentrated to give the title compound (283 mg, 98% yield) as a white solid that did not require further purification. ¹H NMR (DMSO-d₆, 300 MHz) δ 13.04 (br s, 1H), 7.96 (d, 2H, J=8.4 Hz), 7.53 (d, 2H, J=8.4 Hz), 7.03 (br t, 1H, J=5.5 Hz), 4.62 (s, 2H), 3.38-3.29 (m, 2H), 3.18-3.14 (m, 2H), 1.38 (s, 9H) ppm; LCMS m/z 244.1 found (M-BOC+2H)⁺.

### Intermediate 21: tert-butyl (2-((4-fluorophenyl)sulfonyl)ethyl)carbamate

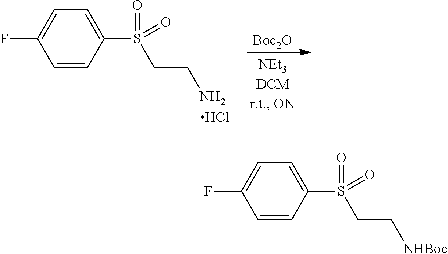 [SMILES: "Boc2O" ; NCCS(=O)(=O)c1ccc(F)cc1 ; Cl ; CC(C)(C)OC(=O)NCCS(=O)(=O)c1ccc(F)cc1]

Boc anhydride (273 mg, 1.25 mmol) was added to a solution of {2-[(4-fluorophenyl)sulfonyl]ethyl}amine hydrochloride (250 mg, 1.043 mmol) and triethylamine (0.36 mL, 2.608 mmol) in dichloromethane (10 mL), with stirring at room temperature under nitrogen overnight. The reaction mixture was then separated between dichloromethane and water, the organic phase was washed with brine, dried over anhydrous sodium sulfate, filtered and concentrated to give the crude product, which was which was purified using flash column chromatography over silica, eluting with 5-100% ethyl acetate/petroleum ether, to give the product (290 mg, 92% yield) as a pale yellow solid. ¹H NMR (CDCl₃, 300 MHz) δ 7.96-7.91 (m, 2H), 7.29-7.23 (m, 2H), 5.15 (br s, 1H), 3.58-3.52 (m, 2H), 3.33-3.29 (m, 2H), 1.41 (s, 9H) ppm; LCMS m/z 248.3 found (M-^{t}Bu+2H)⁺.

### General Procedures for the Synthesis of Aminoethyl Sulfone (AES) Compounds

### General Procedure GP3

Compounds according to the invention may be prepared by as illustrated in Scheme 3, wherein R correspond to the substituent of each specific compound described herein.

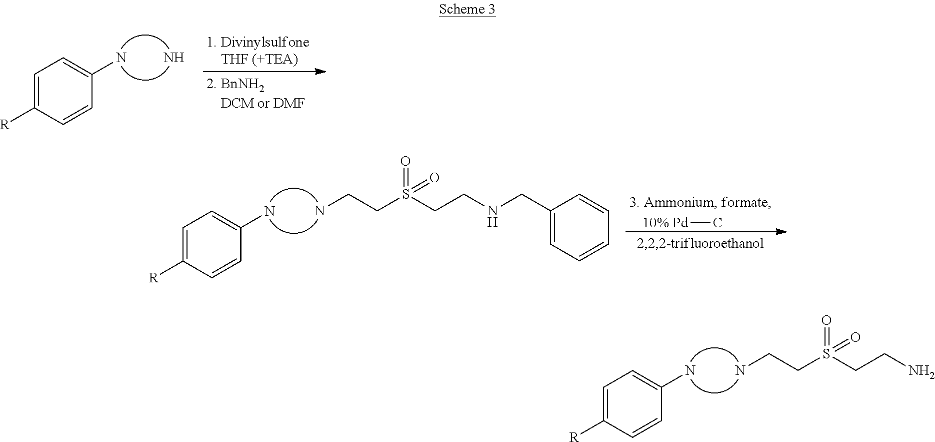

A suspension of the cyclic amine (1 eq) in THF (0.15 to 0.25 M) (and TEA (1 eq) if the amine is used as an HCl salt) is reacted with divinyl sulfone (1 eq) at 40° C. for 6-12 hrs. After completion of the reaction, all the volatiles are removed under reduced pressure.

The resulting residue is dissolved in either DCM or DMF (depending on its solubility) (0.15 M) and reacted with benzylamine (2 eq) at rt for 12 hrs. The mixture is then evaporated to dryness (if DCM is used as a solvent) or diluted with ethyl acetate, washed with water, dried with MgSO₄, filtered and evaporated to dryness (if DMF is used as a solvent). The crude mixture is purified by flash chromatography using a Biotage silica column and a gradient of EtOAc in cHex or a gradient of MeOH in EtOAc.

The residue obtained in the previous step is then dissolved in 2,2,2-trifluoroethanol (0.10 M) and reacted with ammonium formate (10 eq) and 10% Pd—C (20% wt) at 70° C. for 15-30 mins. After cooling down to rt, the mixture is then filtered through a celite pad and evaporated to dryness. The purification of the residue is facilitated by temporarily protecting the product with a carbamate group (the residue is reacted with an excess of BOC₂O in a mixture of DCM/TEA for 12 hrs at rt), purifying it by flash chromatography (using Biotage silica gel column and a gradient of EtOAc in cHex) then deprotecting it in acidic conditions (HCl 4.0 M in dioxane for 1 hr at rt).

### General Procedure GP4

Compounds according to the invention may be prepared by as illustrated in Scheme 4, wherein R correspond to the substituent of each specific compound described herein.

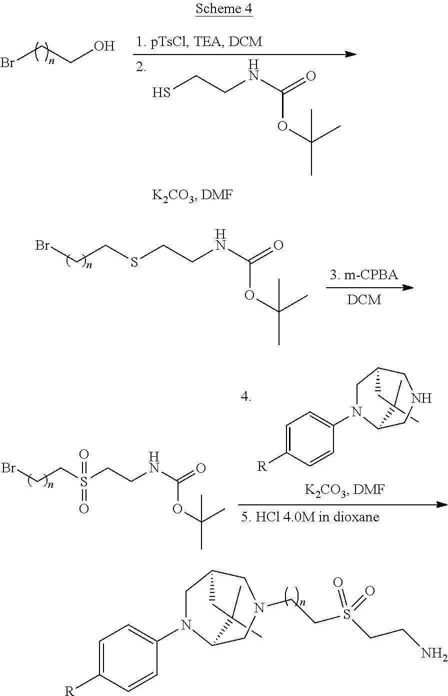

In a first step, to a solution of the bromo alcohol (1 eq) and p-toluenesulfonyl chloride (1.2 eq) in DCM (0.25 M) is added at 0° C. and dropwise TEA (2 eq). The mixture is stirred at rt for 12-48 hrs then washed with aq. NaOH 1M. The organic phase is separated, dried over MgSO₄, filtered and evaporated to dryness to give the bromoalkyl-4-methylbenzenesulfonate intermediate, used in the next step without further purification.

In a second step, the bromoalkyl-4-methylbenzenesulfonate intermediate (1 eq) and t-butyl-(2-mercaptoethyl)carbamate (1 eq) in DMF (0.5 M) are reacted with K₂CO₃ (1.2 eq) at 50° C. for 12 hrs. After cooling down to rt, the mixture is diluted with DCM and washed with water. The organic phase is separated, dried over MgSO₄, filtered and evaporated to dryness. The residue is then purified by flash chromatography (using a Biotage silica gel column and a gradient of EtOAc/cHex, 0→40%) to give the t-butyl(2-((bromoalkyl)thio)ethyl)carbamate.

In a third step, a solution of the t-butyl (2-((2-bromoalkyl)thio)ethyl)carbamate intermediate (1 eq) in DCM (0.1 M) is reacted with m-CPBA (6 eq) at rt for 12 hrs then quenched with aq. std NaHCO₃. The organic phase is separated, washed further with water, dried over MgSO₄, filtered and evaporated to dryness. The residue is then purified by flash chromatography (using a Biotage silica gel column and a gradient of EtOAc/cHex, 0→50%) to give the t-butyl(2-((2-bromoalkyll)sulfonyl)ethyl)carbamate intermediate.

In a fourth step, a solution of the appropriate homopiperazine compound (1 eq) and the t-butyl (2-((bromoalkyl)sulfonyl)ethyl)carbamate intermediate (1 eq) in DMF (0.1 M) is reacted with K₂CO₃ (4 eq) at 80° C. for 12 hrs. After cooling down to rt, the mixture is diluted with DCM and washed with water. The organic phase is separated, dried over MgSO₄, filtered and evaporated to dryness. The residue is then purified by flash chromatography (using a Biotage silica gel column and a gradient of EtOAc/cHex, 50→100%) to give the t-butyl(2-((bromoalkyl)thio)ethyl)carbamate-homopiperazine derivative

In a fifth step, the intermediate obtained previously is dissolved in a 4.0 M solution of HCl in dioxane (0.1 M), stirred at rt for 1 hr then evaporated to dryness to provide the desired AES-homopiperazine product.

### General Procedure GP5

Compounds according to the invention may be prepared by as illustrated in Scheme 5, wherein R, R′ and R″ correspond to the substituent of each specific compound described herein.

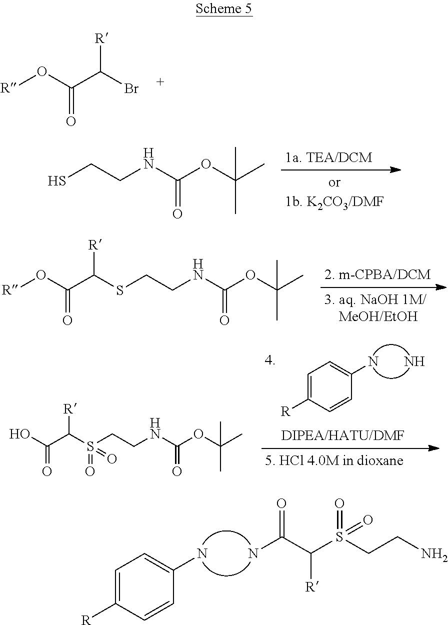

In a first step using conditions 1a, a solution of t-butyl-(2-mercaptoethyl)carbamate (1 eq) and the bromoacetate ester (1.2 eq) in DCM (0.3 M) is reacted with TEA (1.3 eq) at rt for 16 hrs then washed with 10% citric acid. The organic phase is separated, dried over MgSO₄, filtered and evaporated to dryness to give a residue that is used in the next step without further purification.

Alternatively, conditions 1b can be used where a solution of t-butyl-(2-mercaptoethyl)carbamate (1 eq) and the bromoacetate ester (1 eq) in DMF (0.1 to 0.5 M) or DMSO (1M) is reacted with K₂CO₃ (1-2.5 eq) at rt−80° C. for 2-24 hrs. After cooling down to rt, the mixture is diluted with DCM (or EtOAc) and washed with water. The organic phase is separated, dried over MgSO₄, filtered and evaporated to dryness.

In a second step, a solution of the intermediate from the previous step (1 eq) in DCM (0.1 M) is reacted with m-CPBA (5 eq) at rt for 3-12 hrs then quenched with aq. std NaHCO₃. The organic phase is separated, washed further with water, dried over MgSO₄, filtered and evaporated to dryness. The residue is then purified by flash chromatography (using a Biotage silica gel column and a gradient of EtOAc/DCM 0→50% or EtOAc/cHex, 0→50%) to give the sulfonylated intermediate.

In a third step, a solution of the sulfonylated intermediate (1 eq.) in EtOH (0.2 M) or a mixture of MeOH/DCM (3:1, v/v, 0.2 M) is reacted with a 1M aqueous solution of NaOH (or KOH) (0.4M) at rt−45° C. for 2-12 hrs then evaporated to dryness. This residue is used in the next step without further purification.

In a fourth step, to a solution of the residue from the previous step (1 eq) and the cyclic amine (1eq) in DMF (0.15 M) are added DIPEA (8.5 eq) and HATU (1.3 eq). The mixture is stirred at rt for 16 hrs, diluted with DCM and washed with satd. aq. NaHCO₃ and water. The organic layer is separated, dried over MgSO₄, filtered and evaporated to dryness. The residue is purified by flash chromatography using a Biotage silica gel column and a gradient of EtOAc/cHex, 50→100%.

In a fifth step, a suspension of the t-butyl carbamate (1 eq) in HCl 4.0 M in dioxane (0.1 M) is stirred at rt for 1-2 hrs. All the volatiles are then removed under reduced pressure to give the desired aminoethylsulfone as an HCl salt. Alternatively, the resulting residue can be triturated with Et₂O, filtered and dried under high vacuum or purified by preparative HPLC using a gradient of MeOH (+0.1% formic acid) in water (+0.1% formic acid), 10→100%.

### General Procedure GP6

Compounds according to the invention may be prepared by as illustrated in Scheme 6, wherein R and R′ correspond to the substituent of each specific compound described herein.

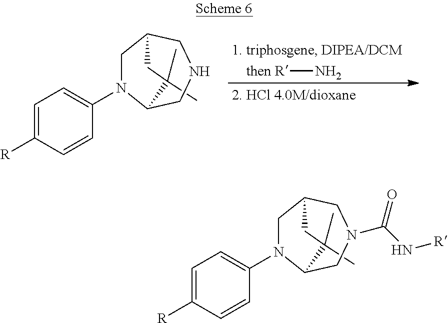

In a first step, to a solution of the appropriate homopiperazine compound (1 eq) in DCM (0.1 M) were added DIPEA (3 eq) (or TEA 6 eq) and triphosgene (0.5 to 0.8 eq). The reaction mixture was stirred at rt for 15 mins then the amine (1.1 to 3 eq) was added and stirring continued for 12-16 hrs. All the volatiles were removed under reduced pressure and the resulting residue purified by flash chromatography (using a Biotage silica gel column and a gradient of EtOAc in DCM, 0→20% or a gradient of MeOH in EtOAc, 0→10%).

In a second step, the intermediate from the previous step was suspended in a solution of HCl in dioxane (4.0 M, 0.1 M), stirred at rt for 1-1.5 hr then evaporated to dryness to give the desired compound as an HCl salt. Alternatively, the resulting residue can be triturated with Et₂O, filtered and dried under high vacuum or purified by preparative HPLC using a gradient of MeOH (+0.1% formic acid) in water (+0.1% formic acid), 10→100%.

### Example 1: 2-((2-((1R,5S)-6-(4-ethoxyphenyl)-9,9-dimethyl-3,6-diazabicyclo[3.2.2]nonan-3-yl)ethyl) sulfonyl)ethan-1-amine (HCl salt)

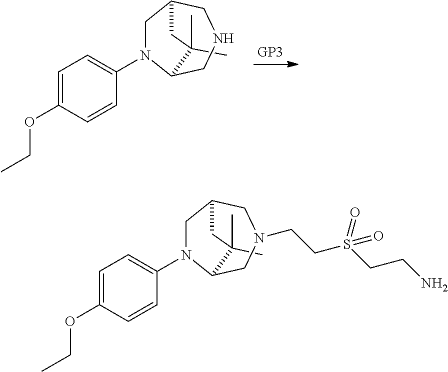 [SMILES: CCOc1ccc(N2C[C@H]3CNC[C@@H]2C(C)(C)C3)cc1 ; CCOc1ccc(N2C[C@H]3CN(CCS(=O)(=O)CCN)C[C@@H]2C(C)(C)C3)cc1]

Using GP3 with (1R,5S)-6-(4-ethoxyphenyl)-9,9-dimethyl-3,6-diazabicyclo[3.2.2]nonane (600 mg, 2.19 mmol), intermediate N-benzyl-2-((2-((1R,5S)-6-(4-ethoxyphenyl)-9,9-dimethyl-3,6-diazabicyclo[3.2.2]nonan-3-yl)ethyl)sulfonyl)ethan-1-amine was obtained as a colourless oil (569.2 mg, 52% over 2 steps). ¹H NMR (500 MHz, DMSO-d₆) δ 7.33-7.27 (m, 4H), 7.24-7.20 (m, 1H), 6.78-6.74 (m, 2H), 6.51-6.47 (m, 2H), 3.89 (q, J=7.0 Hz, 2H), 3.69 (s, 2H), 3.44-3.36 (m, 1H), 3.33-3.21 (m, 5H), 3.16-3.12 (m, 1H), 3.10-3.04 (m, 1H), 3.02-2.93 (m, 2H), 2.90 (t, J=6.5 Hz, 2H), 2.84-2.79 (m, 2H), 2.33-2.26 (m, 1H), 2.21 (dd, J=11.5, 6.0 Hz, 2H), 1.78 (d, J=12.8 Hz, 1H), 1.29-1.20 (m, 4H), 1.11 (s, 3H), 0.76 (s, 3H); HRMS calcd for C₂₈H₄₂N₃O₃S (M+H⁺) 500.2947; found 500.5716.

Debenzylation, purification via the carbamate protection, flash chromatography (EtOAc/cHex, 0→50%) followed by acidic deprotection route as per method GP3 gave 2-((2-((1R,5S)-6-(4-ethoxyphenyl)-9,9-dimethyl-3,6-diazabicyclo[3.2.2]nonan-3-yl)ethyl) sulfonyl)ethan-1-amine (HCl salt) as a white solid (68.3 mg, 18%). ¹H NMR (500 MHz, DMSO-d₆) δ 6.77-6.73 (m, 2H), 6.51-6.47 (m, 2H), 3.87 (q, J=7.0 Hz, 2H), 3.37-3.30 (m, 2H), 3.26-3.15 (m, 3H), 3.14-3.06 (m, 2H), 3.01-2.92 (m, 4H), 2.85-2.75 (m, 2H), 2.33-2.28 (m, 1H), 2.20 (t, J=10.3 Hz, 2H), 1.76 (d, J=13.0 Hz, 1H), 1.26-1.19 (m, 4H), 1.09 (s, 3H), 0.74 (s, 3H); HRMS calcd for O₂₁H₃₆N₃O₃S (M+H⁺) 410.2477; found 410.2481.

The following example compounds were prepared by the aforementioned methodology, using the appropriately substituted reagent.

| Example | Structure | Name | Analytical data |
|---|---|---|---|
| 2 | 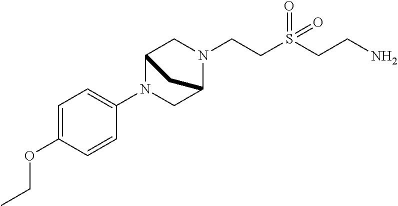 | 2-((2-((1S,4S)-5-(4- ethoxyphenyl)-2,5- diazabicyclo[2.2.1]heptan- 2-yl)ethyl)sulfonyl)- ethan-1-amine (HCl salt) | ¹H NMR (500 MHz, DMSO-d₆) δ 8.55-8.27 (br s, 3H), 6.84-6.78 (m, 2H), 6.61-6.56 (m, 2H), 4.57 (s, 2H), 3.92 (q, 2H, J = 7.0 Hz), 3.80-3.45 (m, 9H), 3.31-3.19 (m, 3H), 2.44 (d, 1H, J = 11.2 Hz), 2.11 (d, 1H, J = 11.2 Hz), 1.28 (t, J = 7.0 Hz, 3H); HRMS calcd for C₁₇H₂₈N₃O₃S [M + H]⁺ 354.1851; found |
| | | | 354.1411. |
| 3 | 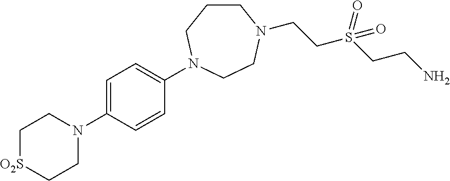 [SMILES: NCCS(=O)(=O)CCN1CCCN(c2ccc(N3CCS(=O)(=O)CC3)cc2)CC1] | 4-(4-(4-(2-((2- aminoethyl)sulfonyl) ethyl)-1,4-diazepan-1- yl)phenyl) thiomorpholine-1,1- dioxide (HCl salt) | ¹H NMR (500 MHz, DMSO-d₆) δ 6.90-6.85 (m, 2H), 6.63-6.58 (m, 2H), 3.51-3.46 (m, 4H), 3.44-3.39 (m, 2H), 3.36 (t, J = 6.2 Hz, 2H), 3.26- 3.17 (m, 4H), 3.13-3.08 (m, 4H), 2.95 (t, J = 6.8 Hz, 2H), 2.84 (t, J = 6.7 Hz, 2H), 2.70 (t, J = 4.9 Hz, 2H), 2.54-2.48 (m, 2H), 1.85-1.79 (m, 2H); |
| | | | HRMS calcd for |
| | | | C₁₉H₃₃N₄O₄S₂ (M + H⁺) |
| | | | 445.1943; found |
| | | | 445.1898. |
| 4 | 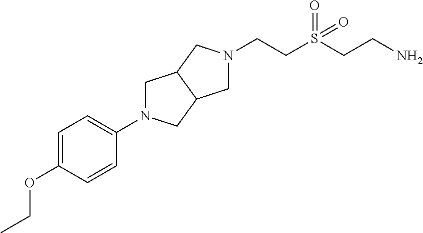 [SMILES: CCOc1ccc(N2CC3CN(CCS(=O)(=O)CCN)CC3C2)cc1] | 2-((2-(5-(4-ethoxy phenyl)hexahydropyrolo [3,4-c]pyrrol-2(1H)- yl)ethyl)sulfonyl)ethan- 1-amine (HCl salt) | ¹H NMR (300 MHz, DMSO-d₆) δ 6.97-6.81 (m, 4H), 3.93 (q, J = 6.9 Hz, 2H), 3.86-3.77 (m, 2H), 3.70-3.53 (m, 7H), 3.50-3.36 (m, 3H), 3.32- 3.08 (m, 6H), 1.26 (t, J = 7.0 Hz, 3H). HRMS calculated for C₁₈H₃₀N₃O₃S [M + H]⁺ 368.2008; found 368.1520. |

### Example 5: 4-(4-((1R,5S)-3-(2-((2-aminoethyl)sulfonyl)ethyl)-9,9-dimethyl-3,6-diazabicyclo[3.2.2]nonan-6-yl)phenyl)thiomorpholine 1,1-dioxide (HCl salt)

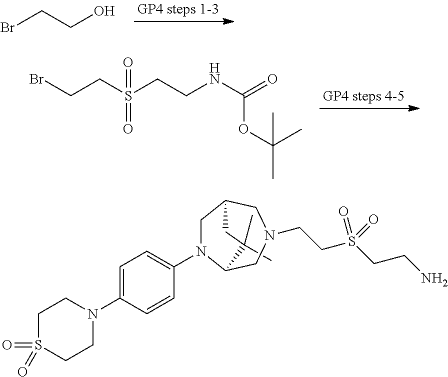

Using GP4 steps 1-3 with 2-bromoethanol (1.80 mL, 25.4 mmol), intermediate t-butyl (2-((2-bromoethyl)sulfonyl)ethyl)carbamate was obtained as a yellow solid (1.15 g, 38% over 3 steps). 1H NMR (500 MHz, CDCl3) δ 5.16 (br s, 1H), 3.72-3.64 (m, 4H), 3.55 (dd, J=8.4 and 6.9 Hz, 2H), 3.32 (dd, J=6.9 and 5.1 Hz, 2H), 1.46 (s, 9H).

Using GP4 steps 4-5 with 4-(4-((1R,5S)-9,9-dimethyl-3,6-diazabicyclo[3.2.2]nonan-6-yl)phenyl)thiomorpholine 1,1-dioxide (130 mg, 0.36 mmol), 4-(4-((1R,5S)-3-(2-((2-aminoethyl)sulfonyl)ethyl)-9,9-dimethyl-3,6-diazabicyclo[3.2.2]nonan-6-yl)phenyl) thiomorpholine-1,1-dioxide (HCl salt) was obtained (49.3 mg, 23% over 2 steps). 1H NMR (500 MHz, DMSO-d6) δ; HRMS calcd for C23H39N4O4S2 (M+H+) 499.2413; found 499.2418.

### Example 6: 2-((3-((1R,5S)-6-(4-ethoxyphenyl)-9,9-dimethyl-3,6-diazabicyclo[3.2.2]nonan-3-yl)propyl)sulfonyl)ethan-1-amine (HCl salt)

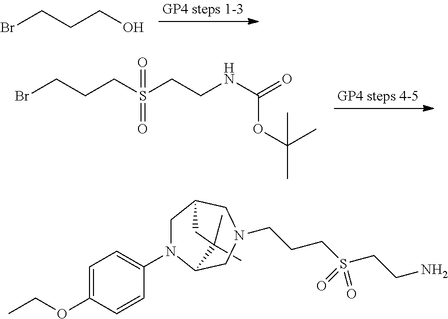

Using GP4 steps 1-3 with 3-bromopropanol (2.71 mL, 30 mmol), intermediate t-butyl (2-((3-bromopropyl)sulfonyl)ethyl)carbamate was obtained as a yellow solid (2.65 g, 27% over 3 steps). ¹H NMR (500 MHz, CDCl₃) δ 5.17 (br s, 1H), 3.67 (q, J=6.0 Hz, 2H), 3.56 (t, J=6.2 Hz, 2H), 3.25 (t, J=6.0 Hz, 2H), 3.23-3.19 (m, 2H), 2.46-2.40 (m, 2H), 1.46 (s, 9H).

Using GP4 steps 4-5 with (1R,5S)-6-(4-ethoxyphenyl)-9,9-dimethyl-3,6-diazabicyclo[3.2.2]nonane (83.1 mg, 0.30 mmol), 2-((3-((1R,5S)-6-(4-ethoxyphenyl)-9,9-dimethyl-3,6-diazabicyclo[3.2.2]nonan-3-yl)propyl)sulfonyl)ethan-1-amine (HCl salt) was obtained (90.1 mg, 98% over 2 steps). ¹H NMR (500 MHz, DMSO-d₆) δ 8.49, 8.39 (2 br s, 2H), 6.85-6.79 (m, 2H), 6.76-6.71 (m, 1H), 6.65-6.61 (m, 1H), 3.91 (2q, J=7.0 Hz, 2H), 3.80-2.98 (m, 15H), 2.60-2.47 (m, 1H), 2.38-2.04 (m, 2H), 1.76 (d, J=14.4 Hz, 1H), 1.64, 1.45 (2dd, J=14.4, 7.5 Hz, 1H), 1.27 (2t, J=7 Hz, 3H), 1.23, 1.09 (2s, 3H), 0.81, 0.74 (2s, 3H); HRMS calcd for C₂₂H₃₈N₃O₃S (M+H⁺) 424.2634; found 424.2882.

### Example 7: 2-((2-aminoethyl)sulfonyl)-1-((1S,5S)-6-(4-ethoxyphenyl)-9,9-dimethyl-3,6-diazabicyclo[3.2.2]nonan-3-yl)ethan-1-one (HCl salt)

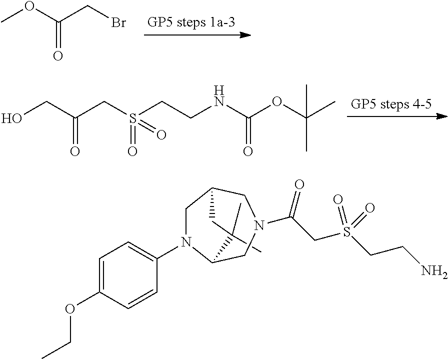

Using GP5 steps 1a-3 with methyl 2-bromoacetate (1.6 mL, 16.7 mmol), intermediate 2-((2-((tert-butoxycarbonyl)amino)ethyl)sulfonyl)acetic acid (sodium salt) was obtained and used in the next step without further purification.

Using GP5 steps 4-5 with (1R,5S)-6-(4-ethoxyphenyl)-9,9-dimethyl-3,6-diazabicyclo[3.2.2]nonane (877 mg, 3.2 mmol), 2-((2-aminoethyl)sulfonyl)-1-((1S,5S)-6-(4-ethoxyphenyl)-9,9-dimethyl-3,6-diazabicyclo[3.2.2] nonan-3-yl)ethan-1-one (HCl salt) was obtained (1.16 g, 97% over 2 steps). 1H NMR (500 MHz, DMSO-d6) δ 8.12 (s, 3H), 6.86-6.73 (m, 2H), 6.58 (dd, J=12.0, 9.1 Hz, 2H), 4.77 (s, 2H), 4.74-4.61 (m, 2H), 4.53-4.41 (m, 2H), 3.90 (qd, J=7.0, 2.8 Hz, 2H), 3.74-3.62 (m, 2H), 3.31-3.22 (m, 2H), 3.18 (d, J=8.2 Hz, 1H), 3.15-3.07 (m, 2H), 2.91, 2.75 (2d, J=13.8 Hz, 1H), 1.58 (d, J=13.7 Hz, 1H), 1.39 (m, 1H), 1.27 (t, J=7.0 Hz, 3H), 1.02, 0.91 (2s, 3H), 0.81, 0.78 (2s, 3H); LCMS m/z 424 (M+H)⁺.

The following example compounds were prepared by the aforementioned methodology, using the appropriately substituted reagent.

| Example | Structure | Name | Analytical data |
|---|---|---|---|
| 8 | 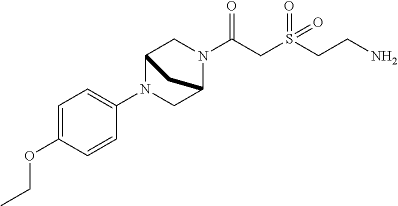 [SMILES: CCOc1ccc(N2C[C@@H]3C[C@H]2CN3C(=O)CS(=O)(=O)CCN)cc1] | 2-((2-aminoethyl)- sulfonyl)-1-((1S,4S)- 5-(4-ethoxyphenyl)- 2,5-diazabicyclo- [2.2.1]heptan-2-yl)- ethan-1-one (HCl salt) | ¹H NMR (500 MHz, DMSO-d₆) (mixture of rotamers) δ 8.57 (s, 3H), 6.86-6.76 (m, 2H), 6.70-6.60 (m, 2H), 4,99, 4.83 (2s, 1H), 4.75-4.65 (m, 1H), 4.62, 4.54 (2s, 1H), 4.42 (s, 1H), 3.93 (2q, J = 6.9 Hz, 2H), 3.72- 3.64 (m, 2H), 3.64- 3.54 (m, 2H), 3.40- |
| | | | 3.30 (m, 1H), 3.28- |
| | | | 3.14 (m, 2H), 2.68 |
| | | | (s, 1H), 2.20-1.85 |
| | | | (m, 2H), 1.25 (t, |
| | | | J = 7.0 Hz, 3H); |
| | | | HRMS calcd for |
| | | | C₁₇H₂₆N₃O₄S |
| | | | (M + H⁺) 368.1644; |
| | | | found 368.1776. |
| 9 | 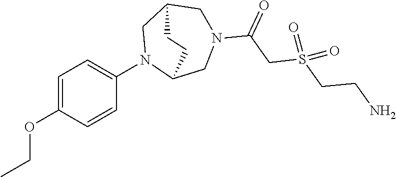 [SMILES: CCOc1ccc(N2C[C@@H]3CC[C@H]2CN(C(=O)CS(=O)(=O)CCN)C3)cc1] | 2-((2-aminoethyl)- sulfonyl)-1-((1S,5S)- 6-(4-ethoxyphenyl)- 3,6-diazabicyclo- [3.2.2]nonan-3-yl)- ethan-1-one (HCl salt) | ¹H NMR (500 MHz, DMSO-d₆) (mixture of rotamers) δ 6.79- 6.74 (m, 2H), 6.57- 6.52 (m, 2H), 4.30-4.19 (m, 1H), 4.12-4.03 (m, 1H), 3.93-3.85 (m, 4H), 3.24 (dd, J = 10.3 and 3.2 Hz, 1H), 3.08 (d, J = 10.2 Hz, |
| | | | 1H), 2.97-2.90 (m, |
| | | | 2H), 2.79-2.71 (m, |
| | | | 3H), 2.63 (d, J = |
| | | | 12.5 Hz, 1H), 2.31- |
| | | | 2.26 (m, 1H), 1.82- |
| | | | 1.66 (m, 3H). 1.49- |
| | | | 1.39 (m, 1H), 1.28- |
| | | | 1.23 (m, 3H); HRMS |
| | | | calcd for |
| | | | C₁₉H₃₀N₃O₄S |
| | | | (M + H⁺) 396.1957; |
| | | | found 396.1859. |
| 10 | 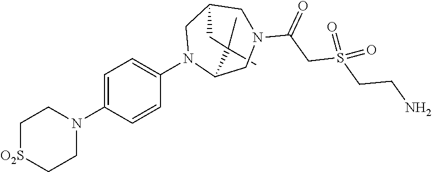 | 2-((2-aminoethyl)- sulfonyl)-1-((1S,5S)- 6-(4-(1,1-dioxido- thiomorpholino)- phenyl)-9,9- dimethyl-3,6-diaza- bicyclo[3.2.2]nonan- 3-yl)ethan-1-one (formic acid salt) | HRMS calcd for C₁₉H₃₀N₃O₄S (M + H⁺) 396.1957; found 396.1859. |
| 11 | 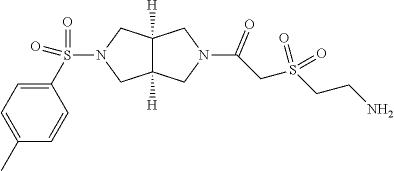 [SMILES: Cc1ccc(S(=O)(=O)N2C[C@H]3CN(C(=O)CS(=O)(=O)CCN)C[C@H]3C2)cc1] | 2-((2-aminoethyl)- sulfonyl)-1-(cis-5- tosylhexahydro- pyrrolo[3,4-c]pyrrol- 2(1H)-yl)ethan-1-one | ¹H NMR (300 MHz, Methanol-d₄) δ 7.78- 7.68 (m, 2H), 7.50- 7.39 (m, 2H), 3.81 (dd, J = 11.1, 7.2 Hz, 1H), 3.62 (dd, J = 13.0, 7.9 Hz, 1H), 3.52-3.35 (m, 5H), 3.25-3.11 (m, 5H), 3.03-2.80 (m, 2H), 2.47 (s, 3H). |
| | | | MS (ESI) m/z 416 |
| | | | [M + H]⁺. |
| 12 | 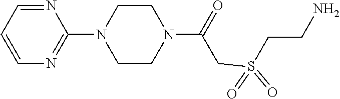 [SMILES: NCCS(=O)(=O)CC(=O)N1CCN(c2ncccn2)CC1] | 2-((2-Aminoethyl)- sulfonyl)-1-(4- (pyrimidin-2-yl)- piperazin-1-yl)ethan- 1-one (HCl salt) | 1H NMR (300 MHz, Methanol-d4) δ 8.36 (d, J = 4.8 Hz, 2H), 6.65 (t, J = 4.8 Hz, 1H), 3.96-3.89 (m, 2H), 3.88-3.82 (m, |
| | | | 2H), 3.79-3.64 (m, |
| | | | 13H); MS (ES+) |
| | | | m/z = 314 (M + H+, |
| | | | 100). |
| 13 | 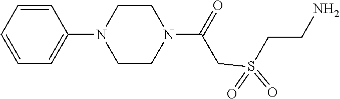 [SMILES: NCCS(=O)(=O)CC(=O)N1CCN(c2ccccc2)CC1] | 2-((2-Aminoethyl)- sulfonyl)-1-(4- phenylpiperazin-1- yl)ethan-1-one (di- HCl salt) | 1H NMR (300 MHz, DMSO-d6) δ 8.86 (br s, 1H), 8.29 (s, 3H), 7.31 (dd, J = 8.5, 7.1 Hz, 2H), 7.16 (d, J = 8.1 Hz, |
| | | | 2H), 6.96 (t, J = 7.2 |
| | | | Hz, 1H), 4.76 (s, |
| | | | 2H), 3.88-3.64 (m, |
| | | | 6H), 3.25 (dd, J = |
| | | | 13.8, 6.2 Hz, 6H); |
| | | | MS (ES+) m/z = |
| | | | 312 (M + H+, 100). |
| 14 | 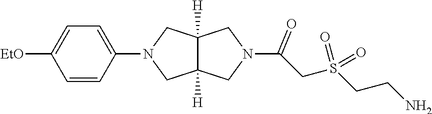 [SMILES: CCOc1ccc(N2C[C@H]3CN(C(=O)CS(=O)(=O)CCN)C[C@H]3C2)cc1] | 2-((2-Aminoethyl)- sulfonyl)-1-(cis-5- (4-ethoxyphenyl)- hexahydropyrrolo- [3,4-c]pyrrol-2(1H)- yl)ethan-1-one (HCl salt) | 1H NMR (300 MHz, DMSO-d6) δ 8.16 (s, 3H), 6.83 (d, J = 8.4 Hz, 2H), 6.68 (s, 2H), 4.55 (s, 2H), 3.92 (q, J = 6.9 Hz, 2H), 3.88-3.78 (m, |
| | | | 1H), 3.74-3.54 (m, |
| | | | 5H), 3.52-3.33 (m, |
| | | | 1H), 3.30-2.95 (m, |
| | | | 7H), 1.28 (t, J = 7.0 |
| | | | Hz, 3H); MS (ES+) |
| | | | m/z = 382 (M + H+, |
| | | | 100). |

### Example 15: 2-((2-aminoethyl)sulfonyl)-1-((1 S,5S)-6-(4-ethoxyphenyl)-9,9-dimethyl-3,6-diazabicyclo[3.2.2]nonan-3-yl)-2-fluoroethan-1-one (HCl salt)

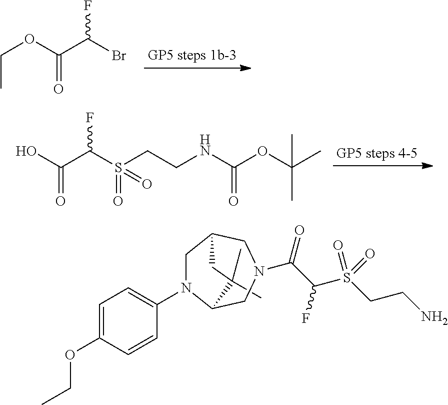

Using GP5 steps 1b-3 with ethyl 2-bromo-2-fluoroacetate (1.5 g, 8.11 mmol), intermediate 2-((2-((tert-butoxycarbonyl)amino)ethyl)sulfonyl)-2-fluoroacetic acid (sodium salt) was obtained and used in the next step without further purification.

Using GP5 steps 4-5 with (1R,5S)-6-(4-ethoxyphenyl)-9,9-dimethyl-3,6-diazabicyclo[3.2.2]nonane (274.4 mg, 1 mmol) and 2-((2-((tert-butoxycarbonyl)amino)-ethyl)sulfonyl)-2-fluoroacetic acid (285.3 mg, 1 mmol), 2-((2-aminoethyl)sulfonyl)-1-((1 S,5S)-6-(4-ethoxyphenyl)-9,9-dimethyl-3,6-diazabicyclo[3.2.2] nonan-3-yl)-2-fluoroethan-1-one (HCl salt) was obtained (353.7 mg, 46% over 2 steps). ¹H NMR (500 MHz, DMSO-d₆) (mixture of isomers) b 7.22, 7.17, 6.96 (3d, J=45 Hz, 1H), 6.82-6.73 (m, 2H), 6.64-6.53 (m, 2H), 4.73-4.65, 4.55-4.49 (2m, 1H), 4.36-4.30, 4.24-4.17, 4.16-4.09 (3m, 1H), 3.91-3.83 (m, 2H), 3.74-3.59 (m, 2H), 3.58-3.50 (m, 1H), 3.33-3.01 (m, 5H), 2.96, 2.84, 2.73 (3d, J=14 Hz, 1H), 2.58-2.43 (m, 1H), 1.73 (d, J=14 Hz, 1H), 1.56-1.27 (m, 1H), 1.26-1.21 (m, 3H), 1.05, 0.93, 0.88 (3s 3H), 0.82, 0.77, 0.72 (3s, 3H); HRMS calcd for C₂₁H₃₃FN₃O₄S (M+H⁺) 442.2176; found 442.2507.

The following example compounds were prepared by the aforementioned methodology, using the appropriately substituted reagent.

| Example | Structure | Name | Analytical data |
|---|---|---|---|
| 16 | 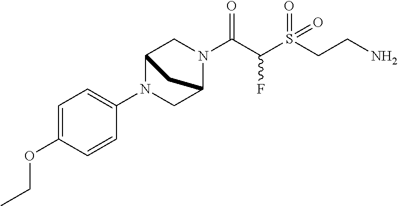 [SMILES: CCOc1ccc(N2C[C@@H]3C[C@H]2CN3C(=O)C(F)S(=O)(=O)CCN)cc1] | 2-((2-aminoethyl)- sulfonyl)-1-((1S,4S)- 5-(4-ethoxyphenyl)- 2,5-diazabicyclo- [2.2.1]heptan-2-yl)- 2-fluoroethan-1-one (HCl salt) | ¹H NMR (500 MHz, DMSO-d₆) (mixture of isomers) δ 6.90- 6.55 (m, 5H), 5.04, 4.92, 4.82 (3s, 1H), 4.56, 4.50 (2s, 1H), 2.96-2.85 (m, 10H), 2.16-1.78 (m, 2H), 1.31-1.20 (m, 3H); HRMS calcd for C₁₇H₂₅FN₃O₄S (M + H⁺) 386.1550; found 386.1879. |
| 17 | 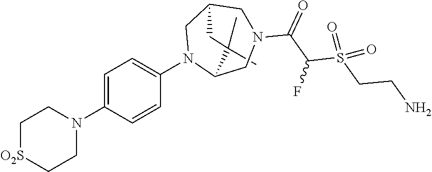 [SMILES: CC1(C)C[C@@H]2CN(C(=O)C(F)S(=O)(=O)CCN)C[C@H]1N(c1ccc(N3CCS(=O)(=O)CC3)cc1)C2] | 2-((2-aminoethyl)- sulfonyl)-1-((1S,5S)- 6-(4-(1,1-dioxido- thiomorpholino)- phenyl)-9,9- dimethyl-3,6-diaza- bicyclo[3.2.2]nonan- 3-yl)-2-fluoroethan- 1-one (HCl salt) | ¹H NMR (500 MHz, DMSO-d₆) (mixture of isomers) δ 7.13 (d, J = 45 Hz, 0.1 H), 6.96-6.88 (m, 2.9 H), 6.68-6.56 (m, 2H), 4.74-4.66 and 4.57- 4.50 (2m, 1H), 4.34- 4.28 and 4.23-4.10 (2m, 1H), 3.65-3.44 (m, 7H), 3.24-3.03 |
| | | | (m, 9H), 2.94, 2.83, |
| | | | 2.71 (3d, J = 14 Hz, |
| | | | 1H), 2.59-2.43 (m, |
| | | | 1H), 1.75 (d, J = 14 |
| | | | Hz, 0.35 H), 1.45- |
| | | | 1.37 (m, 1.05 H), |
| | | | 1.33 (dd, J = 14 and |
| | | | 6.4 Hz, 0.35 H), 1.24 |
| | | | (d, J = 14 Hz, |
| | | | 0.25H), 1.06, 0.94, |
| | | | 0.90, 0.82, 0.80, |
| | | | 0.78, 0.77 (7s, 6H); |
| | | | HRMS calcd for |
| | | | C₂₃H₃₆FN₄O₅S₂ |
| | | | (M + H⁺) 531.2111; |
| | | | found 531.2303. |
| 18 | 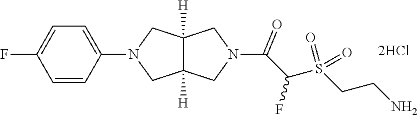 [SMILES: NCCS(=O)(=O)C(F)C(=O)N1C[C@@H]2CN(c3ccc(F)cc3)C[C@@H]2C1] | (2-((2-aminoethyl)- sulfonyl)-2-fluoro- 1-(cis-5-(4-fluoro- phenyl)hexahydro- pyrrolo[3,4-c]pyrrol- 2(1H)-yl)ethan-1- one (di-HCl salt) | ¹⁹F NMR (282 MHz, methanol-d₄) for a mixture of isomers/ rotamers δ −108.65, −108.74, −178.22, −179.78, −182.10, −182.22, −183.29, −183.59, −183.71. |
| | | | ¹H NMR (300 MHz, |
| | | | methanol-d₄) for a |
| | | | mixture of isomers/ |
| | | | rotamers δ 7.89- |
| | | | 7.74 (m, 2H), 7.42- |
| | | | 7.25 (m, 2H), 6.78- |
| | | | 6.61 (m, 1H), 4.29- |
| | | | 3.40 (m, 14H). MS |
| | | | (ESI) m/z 374 |
| | | | [M + H]⁺. |
| 19 | 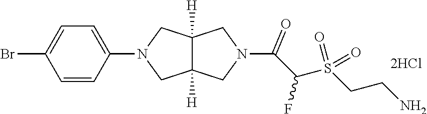 [SMILES: NCCS(=O)(=O)C(F)C(=O)N1C[C@@H]2CN(c3ccc(Br)cc3)C[C@@H]2C1] | 2-((2-Aminoethyl)- sulfonyl)-2-fluoro- 1-(cis-5-(4-bromo- phenyl)hexahydro- pyrrolo[3,4-c]pyrrol- 2(1H)-yl)ethan-1- one (di-HCl salt) | ¹H NMR (300 MHz, Methanol-d₄) δ 7.32- 7.22 (m, 2H), 6.57- 6.35 (m, 3H), 4.11- 3.43 (m, 10H), 3.29- 3.07 (m, 4H). ¹⁹F NMR (282 MHz, |
| | | | Methanol-d₄) for a |
| | | | mixture of diastereo- |
| | | | mers/rotamers δ |
| | | | −182.95, −183.50. |
| | | | MS (ESI) m/z 434, |
| | | | 436 [M + H]⁺. |
| 20 | 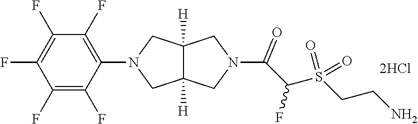 [SMILES: NCCS(=O)(=O)C(F)C(=O)N1C[C@@H]2CN(c3c(F)c(F)c(F)c(F)c3F)C[C@@H]2C1] | 2-((2-aminoethyl)- sulfonyl)-2-fluoro- 1-(cis-5-(perfluoro- phenyl)hexahydro- pyrrolo[3,4-c]pyrrol- 2(1H)-yl)ethan-1- one (di-HCl salt) | ¹⁹F NMR (282 MHz, Methanol-d₄) for a mixture of diastereo- mer/rotamer δ −154.36-−154.74 (m), −167.09-−167.45 (m), −171.40-−172.00 (m), −182.68, −183.00, |
| | | | −185.91-−186.00 |
| | | | (m), −186.19- |
| | | | −186.32 (m). ¹H |
| | | | NMR (300 MHz, |
| | | | Methanol-d₄) δ 6.74- |
| | | | 6.48 and 6.34-5.97 |
| | | | (each m, 1H), 4.19- |
| | | | 3.39 (m, 12H), 3.19- |
| | | | 3.03 (m, 2H). MS |
| | | | (ESI) m/z 446 |
| | | | [M + H]⁺. |
| 21 | 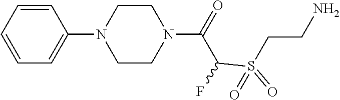 [SMILES: NCCS(=O)(=O)C(F)C(=O)N1CCN(c2ccccc2)CC1] | 2-((2-Aminoethyl)- sulfonyl)-2-fluoro-1- (4-phenylpiperazin- 1-yl)ethan-1-one (di- HCl salt) | ¹H NMR (300 MHz, DMSO d6) δ 8.34 (s, 3H), 7.99-7.49 (br s, 1H), 7.36-7.21 (m, 2.5H), 7.10 (m, 2.5H), 6.93 (t, J = 7.2 Hz, 1H), 3.89- |
| | | | 3.66 (m, 6H), 3.43- |
| | | | 3.11 (m, 6H); 19F |
| | | | NMR (282 MHz, |
| | | | DMSO-d6) δ 184.1; |
| | | | MS (ES+) m/z = |
| | | | 330 (M + H+, 100). |
| 22 | 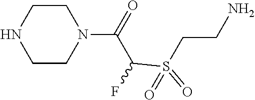 [SMILES: NCCS(=O)(=O)C(F)C(=O)N1CCNCC1] | 2-((2-Aminoethyl)- sulfonyl)-2-fluoro- 1-(piperazin-1-yl)- ethan-1-one (di- HCl salt) | ¹H NMR (300 MHz, Methanol-d4) (mix- ture of rotamers) δ 6.86 (d, J = 45.3 Hz, 0.4H), 6.01 (d, J = 47.4 Hz, 0.6H), 3.78-3.61 (m, 9H), |
| | | | 3.54-3.46 (m, 6H). |
| | | | 19F NMR (282 |
| | | | MHz, DMSO-d6) δ |
| | | | 184.5. |
| 23 | 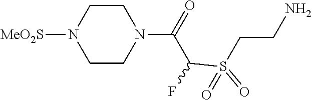 [SMILES: CS(=O)(=O)N1CCN(C(=O)C(F)S(=O)(=O)CCN)CC1] | 2-((2-Aminoethyl)- sulfonyl)-2-fluoro-1- (4-(methylsulfonyl)- piperazin-1-yl)ethan- 1-one (HCl salt) | ¹H NMR (300 MHz, Methanol-d₄) δ 6.78 (d, J = 45.4 Hz, 1H), 4.02-3.43 (m, 8H), 3.43-3.13 (m, 6H), 2.88 (d, J = 1.6 Hz, 3H); ¹⁹F NMR (282 |
| | | | MHz, DMSO-d₆) δ |
| | | | 184.0; MS (ES+) |
| | | | m/z = 332 (M + H⁺, |
| | | | 100). |
| 24 | 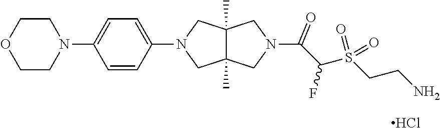 [SMILES: C[C@@]12CN(C(=O)C(F)S(=O)(=O)CCN)C[C@]1(C)CN(c1ccc(N3CCOCC3)cc1)C2 ; Cl] | 2-((2-Aminoethyl)- sulfonyl)-1-(cis- 3a,6a-dimethyl-5- (4-morpholino- phenyl)hexahydro- pyrrolo[3,4-c]pyrrol- 2(1H)-yl)-2-fluoro- ethan-1-one (HCl | ¹H NMR (DMSO-d₆, 300 MHz) δ 8.36 (br s, 3H), 7.71-7.52 (m, 2H), 6.80-6.61 (m, 1H), 6.57-6.52 (m, 2H), 4.10-3.88 (m, 4H), 3.80-3.66 (m, 4H), 3.53-3.35 (m, |
| | | salt) | 8H), 3.27-3.18 (m, |
| | | | 4H), 1.13-1.08 (m, |
| | | | 6H) ppm; ¹⁹F NMR |
| | | | (DMSO-d₆, 282 |
| | | | MHz) δ −182.2, |
| | | | −182.5 (2 isomers |
| | | | observed) ppm; |
| | | | LCMS (ES+) m/z |
| | | | 469.4 found |
| | | | (M + H)⁺, |
| | | | C₂₂H₃₄FN₄SO₄. |
| 25 | 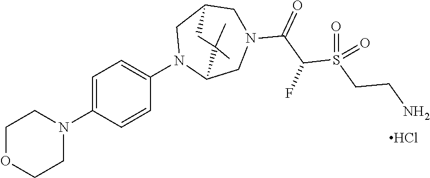 [SMILES: CC1(C)C[C@@H]2CN(C(=O)[C@@H](F)S(=O)(=O)CCN)C[C@H]1N(c1ccc(N3CCOCC3)cc1)C2 ; Cl] | 2-((2-Aminoethyl)- sulfonyl)-1-((1S,5S)- 9,9-dimethyl-6-(4- morpholinophenyl)- 3,6-diazabicyclo- [3.2.2]nonan-3-yl)- 2-fluoroethan-1-one (HCl salt) | ¹H NMR (DMSO-d₆, 300 MHz) δ 8.37 (br s, 3H), 7.71-7.56 (m, 2H), 7.44-7.03 (m, 1H), 6.84-6.67 (m, 2H), 4.81-4.18 (m, 2H), 4.14-3.94 (m, 4H), 3.81-3.61 (m, 5H), 3.51-3.39 (m, 3H), 3.30-3.20 (m, 4H), 3.13-2.73 |
| | | | (m, 2H), 1.83-1.28 |
| | | | (m, 2H), 1.02-0.92 |
| | | | (m, 3H), 0.85-0.79 |
| | | | (m, 3H) ppm; ¹⁹F |
| | | | NMR (DMSO-d₆, |
| | | | 282 MHz) δ −178.9, |
| | | | −180.7 (2 isomers |
| | | | observed) ppm; |
| | | | LCMS (ES+) m/z |
| | | | 483.4 found |
| | | | (M + H)⁺, |
| | | | C₂₃H₃₆FN₄SO₄. |
| 26 | 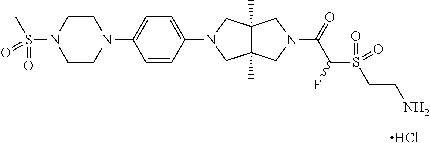 [SMILES: C[C@@]12CN(C(=O)C(F)S(=O)(=O)CCN)C[C@]1(C)CN(c1ccc(N3CCN(S(C)(=O)=O)CC3)cc1)C2 ; Cl] | 2-((2-Aminoethyl)- sulfonyl)-1-(cis- 3a,6a-dimethyl-5-(4- (4-(methylsulfonyl)- piperazin-1-yl)- phenyl)hexahydro- pyrrolo[3,4-c]pyrrol- 2(1H)-yl)-2-fluoro- ethan-1-one (HCl | ¹H NMR (DMSO-d₆, 300 MHz) δ 8.33 (br s, 3H), 7.65-7.43 (m, 2H), 6.79-6.61 (m, 1H), 6.58-6.51 (m, 2H), 3.91-3.68 (m, 8H), 3.52-3.35 (m, 8H), 3.27-3.15 (m, 4H), 3.03-2.98 (m, |
| | | salt) | 3H), 1.11-1.10 (m, |
| | | | 6H) ppm; ¹⁹F NMR |
| | | | (DMSO-d₆, 282 |
| | | | MHz) δ −182.3, |
| | | | −182.5 (2 isomers |
| | | | observed) ppm; |
| | | | LCMS (ES+) m/z |
| | | | 546.4 found |
| | | | (M + H)⁺, |
| | | | C₂₃H₃₇FN₅S₂O₅. |
| 27 | 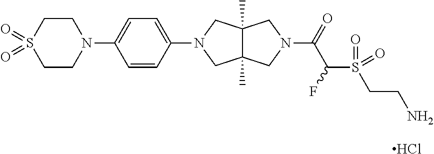 [SMILES: C[C@@]12CN(C(=O)C(F)S(=O)(=O)CCN)C[C@]1(C)CN(c1ccc(N3CCS(=O)(=O)CC3)cc1)C2 ; Cl] | 2-((2-Aminoethyl)- sulfonyl)-1-(cis-5- (4-(1,1-dioxidothio- morpholino)phenyl)- 3a,6a-dimethylhexa- hydropyrrolo[3,4-c]- pyrrol-2(1H)-yl)-2- fluoroethan-1-one (HCl salt) | ¹H NMR (DMSO-d₆, 300 MHz) δ 8.30 (br s, 3H), 7.16-7.06 (m, 2H), 6.79-6.60 (m, 1H), 6.52-6.42 (m, 2H), 3.92-3.59 (m, 8H), 3.52-3.09 (m, 12H), 1.14-1.05 (m, 6H) ppm; ¹⁹F NMR (DMSO-d₆, 282 |
| | | | MHz) δ −182.3, |
| | | | −182.5 (2 isomers |
| | | | observed) ppm; |
| | | | LCMS (ES+) m/z |
| | | | 517.0 found |
| | | | (M + H)⁺, |
| | | | C₂₂H₃₄FN₄S₂O₅. |
| 28 | 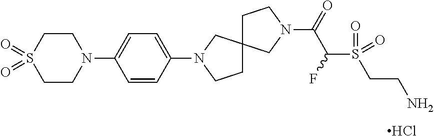 [SMILES: NCCS(=O)(=O)C(F)C(=O)N1CCC2(CCN(c3ccc(N4CCS(=O)(=O)CC4)cc3)C2)C1 ; Cl] | 2-((2-Aminoethyl)- sulfonyl)-1-(7-(4- (1,1-dioxidothio- morpholino)phenyl)- 2,7-diazaspiro[4.4]- nonan-2-yl)-2-fluoro- ethan-1-one (HCl salt) | LCMS (ES+) m/z 503.1 found (M + H)⁺, C₂₁H₃₂FN₄S₂O₅. |
| 29 | 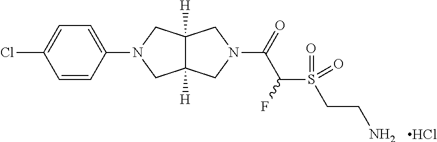 [SMILES: NCCS(=O)(=O)C(F)C(=O)N1C[C@@H]2CN(c3ccc(Cl)cc3)C[C@@H]2C1 ; Cl] | 2-((2-aminoethyl)- sulfonyl)-1-(cis-5- (4-chlorophenyl)- hexahydropyrrolo- [3,4-c]pyrrol-2(1H)- yl)-2-fluoroethan-1- one (HCl salt) | ¹H NMR (DMSO-d₆, 500 MHz) δ 8.36 (br s, 3H), 7.21-7.16 (m, 2H), 6.86-6.67 (m, 1H), 6.56-6.50 (m, 2H), 4.07-3.63 (m, 4H), 3.50-2.98 (m, 10H) ppm; ¹⁹F NMR (DMSO-d₆, 282 |
| | | | MHz) δ −181.8, |
| | | | −182.3 (2 isomers |
| | | | observed) ppm; |
| | | | LCMS m/z 389.9 |
| | | | found (M + H)⁺, |
| | | | C₁₆H₂₂FClN₃SO₃. |

### Example 30: 2-((2-aminoethyl)sulfonyl)-1-((1S,5S)-6-(4-ethoxyphenyl)-9,9-dimethyl-3,6-diazabicyclo[3.2.2]nonan-3-yl)-2,2-difluoroethan-1-one (formic acid salt)

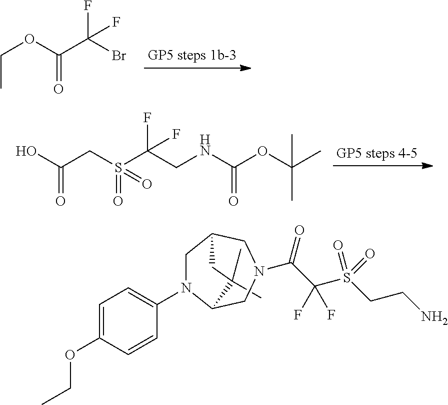

Using GP5 steps 1b-3 with ethyl 2-bromo-2,2-difluoroacetate (1.5 g, 7.39 mmol), intermediate 2-((2-((tert-butoxycarbonyl)amino)-1,1-difluoroethyl)sulfonyl)acetic acid (potassium salt) was obtained and used in the next step without further purification.

Using GP5 steps 4-5 with (1R,5S)-6-(4-ethoxyphenyl)-9,9-dimethyl-3,6-diazabicyclo[3.2.2]nonane (250 mg, 0.91 mmol) and 2-((2-((tert-butoxycarbonyl)amino)-1,1-difluoroethyl)sulfonyl)acetic acid (276.0 mg, 0.91 mmol), 2-((2-aminoethyl)sulfonyl)-1-((1 S,5S)-6-(4-ethoxyphenyl)-9,9-dimethyl-3,6-diazabicyclo[3.2.2]nonan-3-yl)-2,2-difluoroethan-1-one (formic acid salt) was obtained (78 mg, 17% over 2 steps). ¹H NMR (500 MHz, DMSO-d₆) (mixture of isomers) δ 8.38 (s, 1H), 6.82-6.76 (m, 2H), 6.65-6.59 (m, 2H), 4.56-4.53 (m, 1H), 4.35-4.28, 4.26-4.17 (2m, 1H), 3.90 (2q, J=7 Hz, 2H), 3.72-3.56 (m, 3H), 3.52-3.46, 3.30-3.22 (2m, 1H), 3.21-3.10 (m, 2H), 3.07-2.98 (m, 3H), 2.60-2.50 (m, 1H), 1.54 (d, J=14.2 Hz, 1H), 1.44-1.36 (m, 1H), 1.27 (2t, J=7 Hz, 3H), 1.04, 0.91, 0.79 (3s, 6H); HRMS calcd for C₂₁H₃₂F₂N₃O₄S (M+H⁺) 460.2082; found 460.2869.

### Example 31: (4-((2-aminoethyl)sulfonyl)phenyl)((1S,5S)-6-(4-ethoxyphenyl)-9,9-dimethyl-3,6-diazabicyclo[3.2.2]nonan-3-yl)methanone (HCl salt)

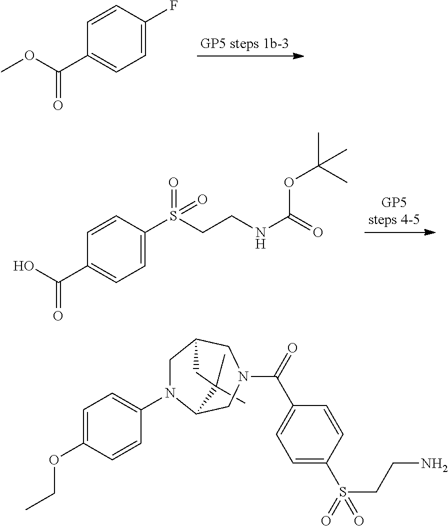

Using GP5 steps 1b-3 with methyl-4-fluorobenzoate (0.52 mL, 7 mmol), 4-((2-((tert-butoxycarbonyl)amino)ethyl)sulfonyl)benzoic acid (sodium salt) was obtained and used in the next step without further purification.

Using GP5 steps 4-5 with (1R,5S)-6-(4-ethoxyphenyl)-9,9-dimethyl-3,6-diazabicyclo[3.2.2]nonane (159.7 mg, 0.582 mmol) and 4-((2-((tert-butoxycarbonyl)amino)ethyl)sulfonyl)benzoic acid (191.7 mg, 0.582 mmol), (4-((2-aminoethyl)sulfonyl)phenyl)((1 S,5S)-6-(4-ethoxyphenyl)-9,9-dimethyl-3,6-diazabicyclo[3.2.2]nonan-3-yl)methanone (HCl salt) was obtained as a red solid (105.1 mg, 38% over 2 steps).

¹H NMR (500 MHz, DMSO-d₆) δ 8.00 (d, J=7.9 Hz, 1H), 7.97 (d, J=7.9 Hz, 1H), 7.65 (d, J=7.9 Hz, 1H), 7.61 (d, J=7.9 Hz, 1H), 6.79 (d, J=8.5 Hz, 1H), 6.75 (d, J=8.6 Hz, 1H), 6.61 (d, J=8.6 Hz, 1H), 6.53 (d, J=8.6 Hz, 1H), 4.84-4.78 (m, 0.5H), 4.60-4.51 (m, 0.5H), 3.94-3.84 (m, 2H), 3.76-3.58 (m, 3.5H), 3.56-3.53 (m, 0.5H), 3.48-3.38 (m, 1H), 3.23-3.02 (m, 4.5H), 2.97 (d, J=13.5 Hz, 0.5H), 2.59-2.53 (m, 0.5H), 2.36-2.29 (m, 0.5H), 1.51-1.35 (m, 1.5H), 1.31-1.19 (m, 3.5H), 1.06, 0.82, 0.80, 0.74 (4s, 6H); HRMS calcd for C₂₆H₃₆N₃O₄S (M+H⁺) 486.2427; found 486.2389.

### Example 32: (3-((2-aminoethyl)sulfonyl)phenyl)((1S,5S)-6-(4-ethoxyphenyl)-9,9-dimethyl-3,6-diazabicyclo[3.2.2]nonan-3-yl)methanone (HCl salt)

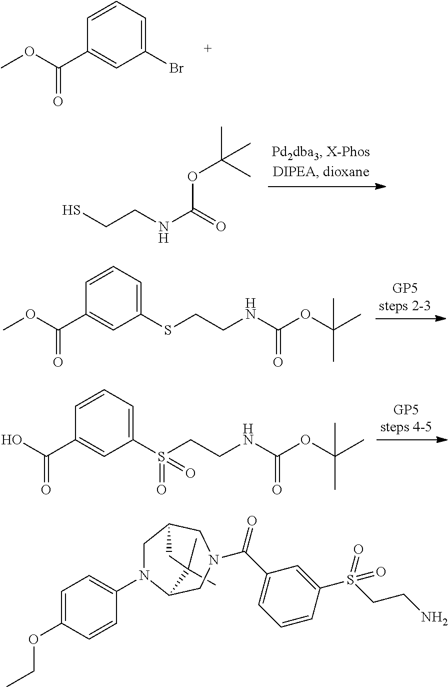

To a solution of methyl-3-bromobenzoate (500 mg, 2.33 mmol) and t-butyl-(2-mercaptoethyl)carbamate (0.39 mL, 2.33 mmol) in dioxane (6.5 mL) were successively added Pd₂dba₃ (53.3 mg, 0.058 mmol), X-Phos (67.4 mg, 0.117 mmol) and DIPEA (0.81 mL, 4.66 mmol). The mixture was stirred under reflux for 4 hrs, then quenched with satd. aq. NH₄Cl and extracted with EtOAc. The combined organic layers were dried over MgSO₄, filtered and evaporated to dryness. The resulting oil was used in the next step without further purification.

Using GP5 steps 2-3 with the residue from the previous step, 3-((2-((tert-butoxycarbonyl)amino)ethyl)sulfonyl)benzoic acid (sodium salt) was obtained and used in the next step without further purification.

Using GP5 steps 4-5 with (1R,5S)-6-(4-ethoxyphenyl)-9,9-dimethyl-3,6-diazabicyclo[3.2.2]nonane (159.7 mg, 0.582 mmol) and 3-((2-((tert-butoxycarbonyl)amino)ethyl)sulfonyl)benzoic acid (sodium salt) (191.7 mg, 0.582 mmol), (3-((2-aminoethyl)sulfonyl)phenyl)((1 S,5S)-6-(4-ethoxyphenyl)-9,9-dimethyl-3,6-diazabicyclo [3.2.2]nonan-3-yl)methanone (HCl salt) was obtained as a red solid (220.6 mg, 76% over 2 steps). ¹H NMR (500 MHz, DMSO-d₆) δ 8.38 (s, 3H), 8.05-7.98 (m, 1H), 7.95 and 7.84 (2s, 1H), 7.82-7.73 (m, 2H), 6.83-6.73 (m, 2H), 6.66-6.54 (m, 2H), 4.90-4.80 and 4.70-4.60 (2m, 1H), 3.94-3.86 (m, 2H), 3.81-3.62 (m, 3.5H), 3.50-3.43 (m, 1H), 3.28-2.96 (m, 5.5H), 2.60-2.54 and 2.42-2.34 (2m, 1H), 1.51-1.36 (m, 2H), 1.27 (t, J=7 Hz, 3H), 1.23, 1.08, 0.82, 0.76, 0.75 (5s, 6H); HRMS calcd for C₂₆H₃₆N₃O₄S (M+H⁺) 486.2427; found 486.2380.

### Example 33: 2-((4-((1R,5S)-6-(4-ethoxyphenyl)-9,9-dimethyl-3,6-diazabicyclo [3.2.2]nonan-3-yl)phenyl)sulfonyl)ethan-1-amine (HCl salt)

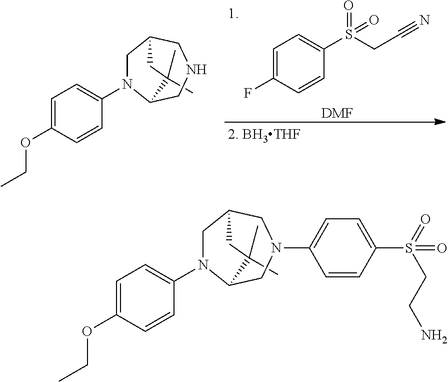

A solution of (1R,5S)-6-(4-ethoxyphenyl)-9,9-dimethyl-3,6-diazabicyclo[3.2.2]nonane (548.8 mg, 2 mmol) and 2-((4-fluorophenyl)sulfonyl)acetonitrile (200 mg, 1 mmol) in DMF (3 mL) was stirred at 100° C. for 3 days. After cooling down to rt, the mixture was diluted with DCM, washed with water, dried with MgSO₄, filtered and evaporated to dryness. The resulting residue was purified by flash chromatography (Biotage silica gel column, gradient of EtOAc in cHex, 60→100%) to give an orange oil that was used in the next step without further purification.

This material was subsequently dissolved in a solution of BH₃ in THF (1.0 M, 6 mL), stirred at rt for 12 hrs, quenched with EtOH (6 mL) at rt for 1 hr then evaporated to dryness. To facilitate the purification, the product was temporarily protected with a carbamate (as described previously, by reacting the material with an excess of BOC₂O in a mixture of DCM/TEA), purified by flash chromatography (Biotage silica gel column, gradient of EtOAc in cHex, 0→40%) then deprotected in acidic conditions (HCl 4.0 M in dioxane) to give 2-((4-((1R,5S)-6-(4-ethoxyphenyl)-9,9-dimethyl-3,6-diazabicyclo[3.2.2]nonan-3-yl)phenyl)sulfonyl) ethan-1-amine (HCl salt) (23.4 mg, 2% over 2 steps). ¹H NMR (500 MHz, DMSO-d₆) δ 7.68-7.62 (m, 2H), 7.12-7.07 (m, 2H), 6.83-6.78 (m, 2H), 6.66-6.60 (m, 2H), 4.17-4.05 (m, 1H), 3.90 (q, J=7.0 Hz, 2H), 3.73-3.68 (m, 1H), 3.65-3.61 (m, 1H), 3.55-3.50 (m, 1H), 3.28-3.24 (m, 1H), 3.19-3.14 (m, 1H), 3.12-2.93 (m, 5H), 2.63-2.57 (m, 1H), 1.53 (d, J=13.6 Hz, 1H), 1.44 (dd, J=13.7, 6.2 Hz, 1H), 1.27 (t, J=7.0 Hz, 3H), 0.93 (s, 3H), 0.84 (s, 3H); HRMS calcd for C₂₅H₃₆N₃O₃S (M+H⁺) 458.2447; found 458.2380.

### Example 34: (1S,5S)-6-(4-(3-((2-aminoethyl)sulfonyl)propoxy)phenyl)-N-(2-hydroxyethyl)-9,9-dimethyl-3,6-diazabicyclo[3.2.2]nonane-3-carboxamide (formic acid salt)

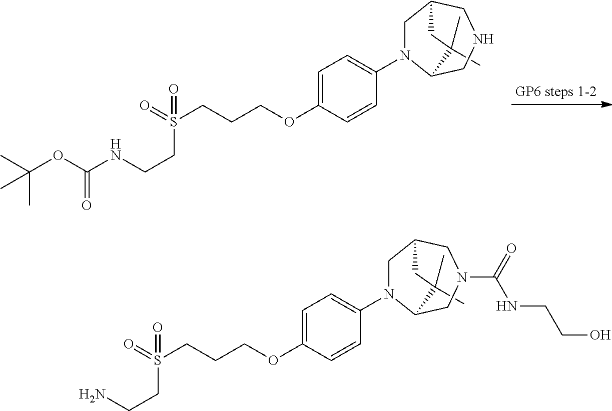 [SMILES: CC(C)(C)OC(=O)NCCS(=O)(=O)CCCOc1ccc(N2C[C@H]3CNC[C@@H]2C(C)(C)C3)cc1 ; CC1(C)C[C@@H]2CN(C(=O)NCCO)C[C@H]1N(c1ccc(OCCCS(=O)(=O)CCN)cc1)C2]

Using GP6 steps 1-2 with t-butyl (2-((3-(4-((1R,5S)-9,9-dimethyl-3,6-diazabicyclo[3.2.2]nonan-6-yl)phenoxy)propyl)sulfonyl)ethyl)carbamate (100.7 mg, 0.203 mmol) and ethanolamine (0.037 mL, 0.609 mmol), (1S,5S)-6-(4-(3-((2-aminoethyl)sulfonyl)propoxy)phenyl)-N-(2-hydroxyethyl)-9,9-dimethyl-3,6-diazabicyclo[3.2.2]nonane-3-carboxamide (formic acid salt) was obtained (13.8 mg, 13% over 2 steps). ¹H NMR (500 MHz, DMSO-d₆) δ 6.81 (d, J=9.1 Hz, 2H), 6.56 (d, J=9.2 Hz, 2H), 6.45 (t, J=5.5 Hz, 1H), 4.29-4.22 (m, 1H), 4.16 (dd, J=13.8 and 8.2 Hz, 1H), 3.97 (t, J=6.2 Hz, 2H), 3.44 (d, J=4.4 Hz, 1H), 3.37 (t, J=6.3 Hz, 2H), 3.33-3.22 (m, 4H), 3.15 (dd, J=10.4 and 2.5 Hz, 1H), 3.12-2.99 (m, 5H), 2.81 (d, J=13.3 Hz, 1H), 2.74 (d, J=13.7 Hz, 1H), 2.44-2.37 (m, 1H), 2.11-2.03 (m, 2H), 1.43 (d, J=13.6 Hz, 1H), 1.31 (dd, J=13.4 and 6.3 Hz, 1H), 0.95 (s, 3H), 0.77 (s, 3H); HRMS calcd for C₂₃H₃₉N₄O₅S (M+H⁺) 483.2641; found 483.2707.

The following example compound was prepared by the aforementioned methodology, using the appropriately substituted reagent.

| Example | Structure | Name | Analytical data |
|---|---|---|---|
| 35 | 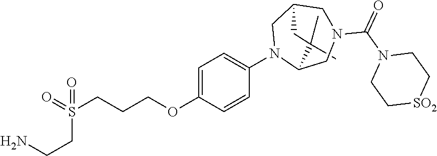 [SMILES: CC1(C)C[C@@H]2CN(C(=O)N3CCS(=O)(=O)CC3)C[C@H]1N(c1ccc(OCCCS(=O)(=O)CCN)cc1)C2] | ((1S,5S)-6-(4-(3-((2- aminoethyl)sulfonyl)- propoxy)phenyl)-9,9- dimethyl-3,6-diaza- bicyclo[3.2.2]nonan- 3-yl)(1,1-dioxido- thiomorpholino) methanone (HCl salt) | ¹H NMR (500 MHz, DMSO-d₆) δ 8.21 (s, 3H), 6.85-6.80 (m, 2H), 6.63-6.58 (m, 2H), 4.12-4.04 (m, 1H), 4.01-3.92 (m, 3H), 3.55-3.45 (m, 7H), 3.42-3.36 (m, 2H), 3.27-3.16 (m, 5H), 3.16-3.02 (m, |
| | | | 4H), 2.85 (d, J = |
| | | | 13.6 Hz, 1H), 2.49- |
| | | | 2.43 (m, 1H), 2.12- |
| | | | 2.05 (m, 2H), 1.56 |
| | | | (d, J = 13.7 Hz, 1H), |
| | | | 1.31 (dd, J = 13.5 |
| | | | and 6.5 Hz, 1H), 0.92 |
| | | | (s, 3H), 0.78 (s, 3H); |
| | | | HRMS calcd for |
| | | | C₂₅H₄₁N₄O₆S₂ |
| | | | (M + H⁺) 557.2468; |
| | | | found 557.2570. |

### Example 36: (1S,5S)-6-(4-(3-((2-aminoethyl)sulfonyl)propoxy)phenyl)-N-ethyl-9,9-dimethyl-3,6-diazabicyclo[3.2.2]nonane-3-carboxamide (HCl salt)

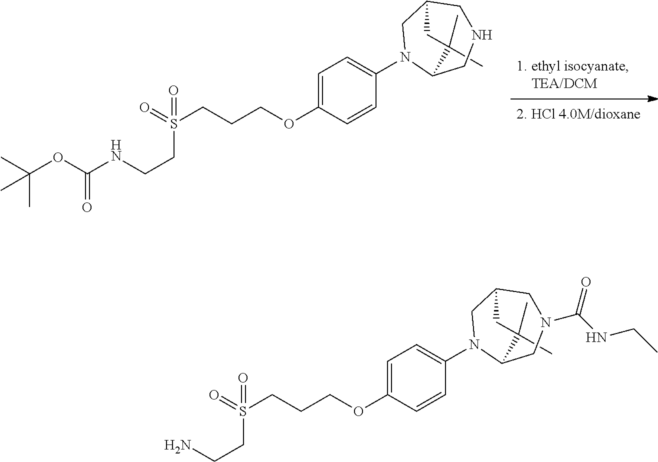 [SMILES: CC(C)(C)OC(=O)NCCS(=O)(=O)CCCOc1ccc(N2C[C@H]3CNC[C@@H]2C(C)(C)C3)cc1 ; CCNC(=O)N1C[C@@H]2CN(c3ccc(OCCCS(=O)(=O)CCN)cc3)[C@H](C1)C(C)(C)C2]

A solution of t-butyl-(2-((3-(4-((1R,5S)-9,9-dimethyl-3,6-diazabicyclo[3.2.2]nonan-6-yl)phenoxy)propyl)sulfonyl)ethyl)carbamate (100 mg, 0.202 mmol) in DCM (2 mL) was reacted with TEA (0.090 mL, 0.606 mmol) and ethyl isocyanate (0.016 mL, 0.202 mmol) at rt for 12 hrs. After evaporation of the volatiles and flash chromatography (using a Biotage silica gel column and a gradient of MeOH in EtOAc, 0→10%), the residue obtained was reacted with a 4.0 M solution of HCl in dioxane (2 mL) at rt for 1 hr. The mixture was evaporated to dryness to give (1 S,5S)-6-(4-(3-((2-aminoethyl)sulfonyl)propoxy)phenyl)-N-ethyl-9,9-dimethyl-3,6-diazabicyclo[3.2.2]nonane-3-carboxamide (HCl salt) (34.8 mg, 32% over 2 steps). ¹H NMR (500 MHz, DMSO-d₆) δ 8.27 (s, 3H), 6.82 (d, J=9.1 Hz, 2H), 6.56 (d, J=9.1 Hz, 2H), 4.29-4.22 (m, 1H), 4.20-4.13 (m, 1H), 3.98 (t, J=6.2 Hz, 2H), 3.57-3.49 (m, 2H), 3.44 (d, J=4.4 Hz, 1H), 3.42-3.36 (m, 2H), 3.26-3.18 (m, 2H), 3.15 (d, J=10.3 Hz, 1H), 3.10-3.00 (m, 3H), 2.80 (d, J=13.3 Hz, 1H), 2.72 (d, J=13.5 Hz, 1H), 2.44-2.37 (m, 1H), 2.13-2.04 (m, 2H), 1.43 (d, J=13.5 Hz, 1H), 1.31 (dd, J=13.5 and 6.4 Hz, 1H), 0.99 (t, J=7.1 Hz, 3H), 0.95 (s, 3H), 0.77 (s, 3H); HRMS calcd for C₂₅H₃₉N₄O₄S (M+H⁺) 467.2692; found 467.2807.

### Example 37: (1S,5S)—N-(4-((2-aminoethyl)sulfonyl)phenyl)-6-(4-ethoxyphenyl)-9,9-dimethyl-3,6-diazabicyclo[3.2.2]nonane-3-carboxamide (formic acid salt)

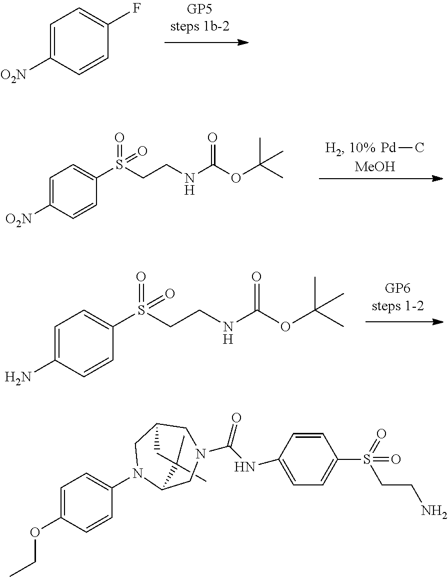

Using GP5 steps 1b-2 with 4-nitrofluorobenzene (705.5 mg, 5 mmol), tert-butyl (2-((4-nitrophenyl)sulfonyl)ethyl)carbamate (1 g, 60% over 2 steps) was obtained. ¹H NMR (500 MHz, CDCl₃) δ 8.47-8.42 (m, 2H), 8.17-8.13 (m, 2H), 5.11-5.65 (m, 1H), 3.60 (q, J=6.1 Hz, 2H), 3.41 (t, J=6.1 Hz, 2H), 1.41 (s, 9H); HRMS calcd for C₃H₁₁N₂O₄S (M-BOC+H⁺) 231.0440; found 231.0531.

A solution of t-butyl-(2-((4-nitrophenyl)sulfonyl)ethyl)carbamate (200 mg, 0.605 mmol) in MeOH (12 mL) was reacted with H₂ and 10% Pd—C using the H-Cube then evaporated to dryness to give t-butyl (2-((4-aminophenyl)sulfonyl)ethyl)carbamate (170 mg, 94%). ¹H NMR (500 MHz, DMSO-d₆) δ 7.48-7.43 (m, 2H), 6.81 (t, J=5.7 Hz, 1H), 6.67-6.62 (m, 2H), 3.21-3.10 (m, 4H), 1.33 (s, 9H); HRMS calcd for C₃H₁₂N₂O₂S (M-BOC+H⁺) 201.0698; found 201.0787.

Using GP6 steps 1-2 with t-butyl-(2-((4-aminophenyl)sulfonyl)ethyl)carbamate (158.8 mg, 0.529 mmol) and (1R,5S)-6-(4-ethoxyphenyl)-9,9-dimethyl-3,6-diazabicyclo[3.2.2]nonane (291 mg, 1.05 mmol), (1 S,5S)—N-(4-((2-aminoethyl)sulfonyl)phenyl)-6-(4-ethoxyphenyl)-9,9-dimethyl-3,6-diazabicyclo[3.2.2] nonane-3-carboxamide (formic acid salt) (37 mg, 12% over 2 steps). ¹H NMR (500 MHz, DMSO-d₆) δ 7.77-7.72 (m, 2H), 7.70-7.65 (m, 2H), 6.81-6.76 (m, 2H), 6.61-6.56 (m, 2H), 4.38-4.27 (m, 2H), 3.88 (q, J=7 Hz, 2H), 3.51 (d, J=4.1 Hz, 1H), 3.43 (t, J=7.3 Hz, 2H), 3.17 (d, J=10.1 Hz, 1H), 3.10 (d, J=10.2 Hz, 1H), 3.05 (d, J=13.5 Hz, 1H), 2.94-2.88 (m, 3H), 2.53-2.48 (m, 1H), 1.50 (d, J=13.7 Hz, 1H), 1.38 (dd, J=13.6 and 6.5 Hz, 1H), 1.25 (t, J=7.0 Hz, 3H), 0.94 (s, 3H), 0.78 (s, 3H); HRMS calcd for C₂₆H₃₇N₄O₄S (M+H⁺) 501.2536; found 501.2990.

### Example 38: (1S,5S)—N-(2-((2-aminoethyl)sulfonyl)ethyl)-6-(4-ethoxyphenyl)-9,9-dimethyl-3,6-diazabicyclo[3.2.2]nonane-3-carboxamide (HCl salt)

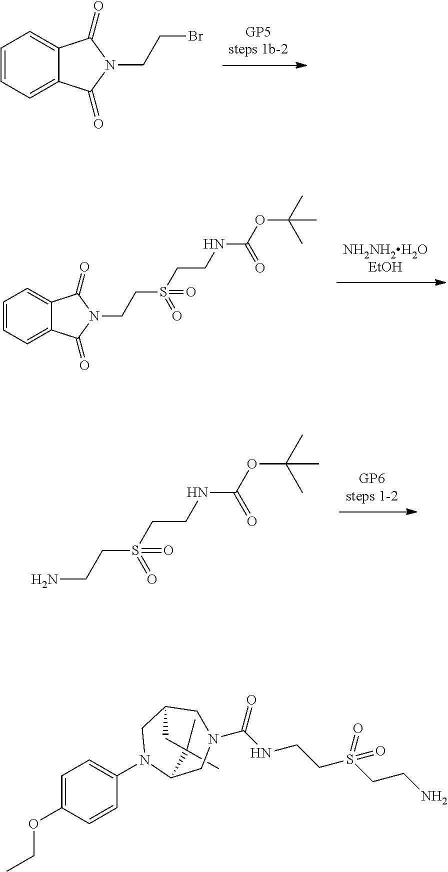

Using GP5 steps 1b-2 with bromoethyl phthalimide (889 mg, 3.5 mmol), t-butyl (2-((2-(1,3-dioxoisoindolin-2-yl)ethyl)sulfonyl)ethyl)carbamate was obtained (200 mg, 12% over 2 steps). ¹H NMR (500 MHz, DMSO-d₆) δ 7.98-7.78 (m, 4H), 7.03 (s, 1H), 4.01 (t, J=6.8 Hz, 2H), 3.51 (t, J=6.8 Hz, 2H), 3.32 (h, J=5.7 Hz, 4H), 1.37 (s, 9H); LCMS m/z 283 (M+H-Boc)⁺.

A suspension of t-butyl-(2-((2-(1,3-dioxoisoindolin-2-yl)ethyl)sulfonyl)ethyl)-carbamate (200 mg, 0.52 mmol) in EtOH (5 mL) was reacted at 80° C. with hydrazine hydrate (65 uL, 2 mmol) for 2 hrs then another 16 hrs at rt. The precipitate formed was filtered off and the filtrate evaporated to dryness to afford t-butyl-(2-((2-aminoethyl)sulfonyl)ethyl)-carbamate (140 mg, 100%). ¹H NMR (500 MHz, DMSO-d₆) δ 7.98 (ddd, J=95.7, 6.0, 3.3 Hz, 1H), 3.24 (t, J=7.0 Hz, 4H), 3.14 (t, J=6.6 Hz, 2H), 2.93 (t, J=6.6 Hz, 2H), 1.38 (s, 9H).

Using GP6 steps 1-2 with (1R,5S)-6-(4-ethoxyphenyl)-9,9-dimethyl-3,6-diazabicyclo[3.2.2]nonane (137 mg, 0.5 mmol) and t-butyl-(2-((2-aminoethyl)sulfonyl)ethyl)-carbamate (140 mg, 0.55 mmol), (1S,5S)—N-(2-((2-aminoethyl)sulfonyl)ethyl)-6-(4-ethoxyphenyl)-9,9-dimethyl-3,6-diazabicyclo[3.2.2]nonane-3-carboxamide (HCl salt) was obtained (40 mg, 33% over 2 steps). ¹H NMR (500 MHz, DMSO-d₆) δ 8.14 (s, 3H), 6.84 (d, J=6.4 Hz, 1H), 6.82-6.74 (m, 2H), 6.61-6.49 (m, 2H), 4.30-4.18 (m, 1H), 4.13 (dd, J=13.9, 8.0 Hz, 1H), 3.90 (q, J=6.9 Hz, 2H), 3.49 (t, J=7.3 Hz, 2H), 3.44 (dd, J=11.4, 4.9 Hz, 3H), 3.35 (t, J=6.8 Hz, 2H), 3.22 (p, J=6.2 Hz, 2H), 3.16 (dd, J=10.5, 2.5 Hz, 1H), 3.07 (d, J=10.3 Hz, 1H), 2.82 (dd, J=25.0, 13.5 Hz, 2H), 2.43 (d, J=7.4 Hz, 1H), 1.43 (d, J=13.6 Hz, 1H), 1.32 (dd, J=13.6, 6.4 Hz, 1H), 1.27 (t, J=7.0 Hz, 3H), 0.95 (s, 3H), 0.78 (s, 3H); LCMS m/z 453 (M+H)⁺.

### Example 39: 2-((2-aminoethyl)sulfonyl)-1-((1S,5S)-6-(4-ethoxyphenyl)-9,9-dimethyl-3,6-diazabicyclo[3.2.2]nonan-3-yl)propan-1-one (HCl salt)

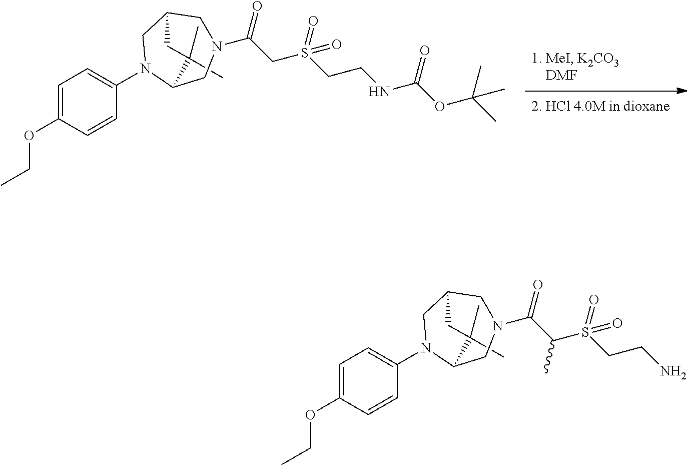

To a solution of t-butyl-(2-((2-((1 S,5S)-6-(4-ethoxyphenyl)-9,9-dimethyl-3,6-diazabicyclo[3.2.2]nonan-3-yl)-2-oxoethyl)sulfonyl)ethyl)carbamate (65 mg, 0.124 mmol) in DMF (2 mL) were added K₂CO₃ (48.6 mg, 0.273 mmol) and methyl iodide (0.023 mL, 0.372 mmol). The mixture was stirred at 60° C. for 12 hrs, then after cooling down to rt, diluted with DCM. This organic mixture was then washed with water, dried over MgSO₄, filtered and evaporated to dryness. The resulting residue was purified by flash chromatography (using a Biotage silica gel column and a gradient of EtOAc in cHex, 0→50%) to give t-butyl (2-((1-((1 S,5S)-6-(4-ethoxyphenyl)-9,9-dimethyl-3,6-diazabicyclo[3.2.2]nonan-3-yl)-1-oxopropan-2-yl)sulfonyl)ethyl) carbamate (56.3 mg, 84%). HRMS calcd for C₂₇H₄₄N₃O₆S (M+H⁺) 538.2951; found 538.3055.

This residue was suspended in a 4.0 M solution of HCl in dioxane (2 mL) and the mixture stirred at rt for 1 hr. After removal of the volatiles, the residue was dissolved in water, washed with Et₂O and evaporated to dryness to give 2-((2-aminoethyl)sulfonyl)-1-((1 S,5S)-6-(4-ethoxyphenyl)-9,9-dimethyl-3,6-diazabicyclo[3.2.2]nonan-3-yl)propan-1-one (HCl salt) (35.8 mg, 73%). ¹H NMR (500 MHz, DMSO-d₆) (mixture of isomers) δ 6.81-6.75 (m, 2H), 6.62-6.55 (m, 2H), 5.10, 4.98, 4.89 (3q, J=6.7 Hz, 1H), 4.78-4.58 (m, 1H), 4.46-4.40, 4.39-4.31, 4.23-4.15 (3m, 1H), 3.91-3.84 (m, 2H), 3.75-3.43 (m, 3H), 3.28-3.04 (m, 5H), 2.82-2.67 (m, 1H), 2.53-2.41 (m, 1H), 1.71-1.45 (m, 4H), 1.42-1.29 (m, 1H), 1.27-1.22 (m, 3H), 1.07, 0.93, 0.90, 0.85, 0.81, 0.79, 0.77, 0.76 (8s, 6H); HRMS calcd for C₂₂H₃₆N₃O₄S (M+H⁺) 438.2427; found 438.2531.

### Example 40: 2-((2-aminoethyl)sulfonyl)-1-((1S,5S)-6-(4-ethoxyphenyl)-9,9-dimethyl-3,6-diazabicyclo[3.2.2] nonan-3-yl)-4-methylpentan-1-one (HCl salt)

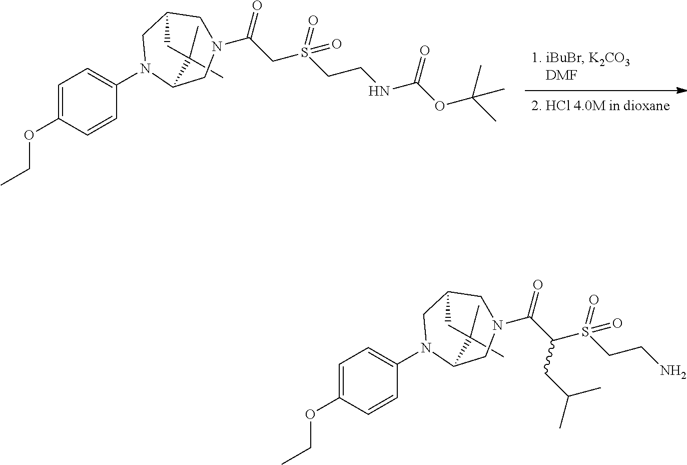

Using the same method with tert-butyl (2-((2-((1S,5S)-6-(4-Ethoxyphenyl)-9,9-dimethyl-3,6-diazabicyclo[3.2.2]nonan-3-yl)-2-oxoethyl)sulfonyl) ethyl)carbamate (165 mg, 0.31 mmol) and isobutyl bromide (40 uL, 0.37 mmol) 2-((2-aminoethyl)sulfonyl)-1-((1S,5S)-6-(4-ethoxyphenyl)-9,9-dimethyl-3,6-diazabicyclo[3.2.2] nonan-3-yl)-4-methyl pentan-1-one (10 mg, 40%) was obtained as a solid. LCMS m/z 480 (M+H)⁺.

### Example 41: ethyl 4-((2-aminoethyl)sulfonyl)-5-((1S,4S)-5-(4-ethoxyphenyl)-2,5-diazabicyclo[2.2.1]heptan-2-yl)-5-oxopentanoate

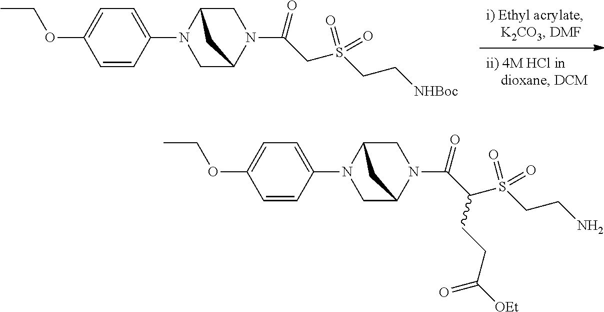 [SMILES: CCOc1ccc(N2C[C@@H]3C[C@H]2CN3C(=O)CS(=O)(=O)CCNC(=O)OC(C)(C)C)cc1 ; CCOC(=O)CCC(C(=O)N1C[C@@H]2C[C@H]1CN2c1ccc(OCC)cc1)S(=O)(=O)CCN]

A mixture of tert-butyl (2-((2-((1 S,4S)-5-(4-ethoxyphenyl)-2,5-diazabicyclo[2.2.1]heptan-2-yl)-2-oxoethyl)sulfonyl)ethyl)carbamate (54 mg, 0.116 mmol), ethyl acrylate (126 μL, 0.116 mmol) and K₂CO₃ (31.9 mg, 0.231 mmol) in DMF (1.0 mL) was stirred at rt for 1 h. Further portions of ethyl acrylate (126 μL) were added every hour for 4 hours and the mixture was stirred at rt for a further 16 h before it was diluted with EtOAc (20 mL). The organic layer was washed with H₂O (3×20 mL), dried over MgSO₄, filtered and the solvent was removed under reduced pressure. The crude was purified by chromatography (EtOAc/Pet ether 0→80%) to afford ethyl 4-((2-((tert-butoxycarbonyl)amino)ethyl)sulfonyl)-5-((1S,4S)-5-(4-ethoxyphenyl)-2,5-diazabicyclo[2.2.1]heptan-2-yl)-5-oxopentanoate as a colourless oil (59 mg, 90%). ¹H NMR (300 MHz, Chloroform-d) for a mixture of isomers δ 6.88-6.63 (m, 2H), 6.59-6.40 (m, 2H), 5.25-4.68 (m, 2H), 4.47-4.08 (m, 3H), 4.03-3.80 (m, 3H), 3.73-2.99 (m, 8H), 2.67-1.86 (m, 6H), 1.50-1.41 (m, 9H), 1.40-1.02 (m, 6H). MS (ESI) m/z 568 [M+H]⁺.

A mixture of ethyl 4-((2-((tert-butoxycarbonyl)amino)ethyl)sulfonyl)-5-((1 S,4S)-5-(4-ethoxyphenyl)-2,5-diazabicyclo[2.2.1]heptan-2-yl)-5-oxopentanoate (59 mg, 0.104 mmol), 4 M HCl in dioxane (1.0 mL) and DCM (1.0 mL) was stirred at rt for 3 h before it was diluted with DCM (20 mL). The organic layer was washed with sat. NaHCO₃ (2×20 nL), dried over MgSO₄, filtered and the solvent was removed under reduced pressure. The crude was purified by chromatography (MeOH/DCM 0→15%) to afford compound 41 as a colourless oil (22 mg, 45%). ¹H NMR (300 MHz, Chloroform-d) for a mixture of isomers δ 6.88-6.78 (m, 2H), 6.57-6.46 (m, 2H), 5.09-4.72 (m, 1H), 4.48-4.31 (m, 1H), 4.21-3.83 (m, 5H), 3.70-2.99 (m, 8H), 2.60-1.86 (m, 6H), 1.74 (br, 2H), 1.44-1.35, 1.31-1.23 and 1.12-1.02 (m, 6H). MS (ESI) m/z 468 [M+H]⁺.

### Example 42: (1-((2-Aminoethyl)sulfonyl)cyclopropyl)((1S,4S)-5-(4-ethoxyphenyl)-2,5-diazabicyclo[2.2.1]heptan-2-yl)methanone

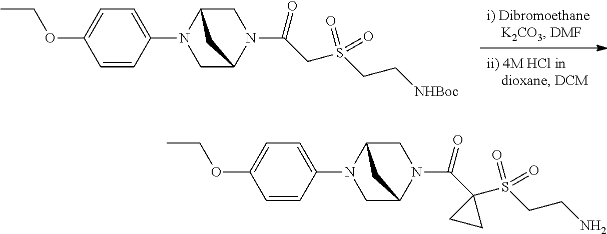 [SMILES: CCOc1ccc(N2C[C@@H]3C[C@H]2CN3C(=O)CS(=O)(=O)CCNC(=O)OC(C)(C)C)cc1 ; CCOc1ccc(N2C[C@@H]3C[C@H]2CN3C(=O)C2(S(=O)(=O)CCN)CC2)cc1]

A mixture of tert-butyl (2-((2-((1 S,4S)-5-(4-ethoxyphenyl)-2,5-diazabicyclo[2.2.1]heptan-2-yl)-2-oxoethyl)sulfonyl)ethyl)carbamate (74.3 mg, 0.159 mmol), 1,2-dibromoethane (68.6 μL, 0.800 mmol), K₂CO₃ (110 mg, 0.800 mmol) and DMF (1.6 mL) were stirred at 80° C. for 20 h. Further batches of dibromoethane were added after 1 h and 17 h. After cooling to rt, the mixture was diluted with EtOAc (20 mL), washed with H₂O (3×20 mL) and brine (20 mL), dried over MgSO₄ and filtered. The solvent was removed under reduced pressure and the crude was purified by chromatography to afford tert-butyl (2-((1-((1 S,4S)-5-(4-ethoxyphenyl)-2,5-diazabicyclo[2.2.1]heptane-2-carbonyl)cyclopropyl)sulfonyl)ethyl)carbamate as a colourless oil (16 mg, 33%). ¹H NMR (300 MHz, Chloroform-d) 5 for a mixture of isomers 6.90-6.77 (m, 2H), 6.58-6.43 (m, 2H), 5.48 (br, 1H), 5.08-4.93 (m, 1H), 4.39 (m, 1H), 3.96 (q, J=7.0 Hz, 2H), 3.90-3.49 (m, 5H), 3.46-3.34 (m, 2H), 3.08 (m, 1H), 2.20-1.73 (m, 4H), 1.61-1.33 (m, 14H). MS (ESI) m/z 494 [M+H]⁺.

A mixture of tert-butyl (2-((1-((1 S,4S)-5-(4-ethoxyphenyl)-2,5-diazabicyclo[2.2.1]heptane-2-carbonyl)cyclopropyl)sulfonyl)ethyl)carbamate (23.1 mg, 46.6 μmol), 4 M HCl in dioxane (1.0 mL) and DCM (1.0 mL) was stirred at rt for 3 h. The solvent was removed under reduced pressure and the crude was purified by HPLC (high pH) to afford compound 42 as a colourless oil (8.5 mg, 46%). ¹H NMR (300 MHz, Methanol-d₄) for a mixture of isomers δ 6.88-6.72 (m, 2H), 6.66-6.53 (m, 2H), 5.07 (br, 1H), 4.49 (br, 1H), 3.96 (q, J=7.0 Hz, 2H), 3.86-3.60 (m, 1H), 3.56-3.35 (m, 3H), 3.22-3.03 (m, 3H), 2.14-1.91 (m, 3H), 1.84-1.41 (m, 4H), 1.35 (t, J=7.0 Hz, 3H). MS (ESI) m/z 394 [M+H]⁺.

### Example 43: 1-((1S,4S)-5-(4-((2-aminoethyl)sulfonyl)phenyl)-2,5-diazabicyclo[2.2.1]heptan-2-yl)-2-methylpropan-1-one (HCl salt)

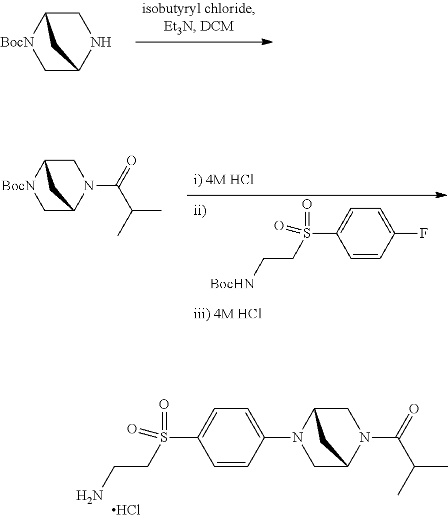 [SMILES: CC(C)(C)OC(=O)N1C[C@@H]2C[C@H]1CN2 ; CC(C)C(=O)N1C[C@@H]2C[C@H]1CN2C(=O)OC(C)(C)C ; CC(C)(C)OC(=O)NCCS(=O)(=O)c1ccc(F)cc1 ; CC(C)C(=O)N1C[C@@H]2C[C@H]1CN2c1ccc(S(=O)(=O)CCN)cc1 ; Cl]

Triethylamine (1.12 mL, 8.07 mmol) was added to a solution of tert-butyl (1S,4S)-2,5-diazabicyclo[2.2.1]heptane-2-carboxylate (800 mg, 4.0 mmol) and isobutyryl chloride (0.59 mL, 5.64 mmol) in anhydrous DCM (40 mL), with stirring at room temperature under nitrogen overnight. The reaction mixture was then separated between dichloromethane and aqueous NaHCO₃ and the organic phase was washed with water (×2) and brine, dried over anhydrous sodium sulfate, filtered and concentrated to give the crude product, which was which was purified using flash column chromatography over silica, eluting with 0-30% methanol/dichloromethane, to give tert-butyl (1 S,4S)-5-isobutyryl-2,5-diazabicyclo[2.2.1]heptane-2-carboxylate (1.1 g, 100% yield) as a white solid. ¹H NMR (CDCl₃, 300 MHz) δ 4.91-4.44 (m, 2H), 3.55-3.28 (m, 4H), 2.68-2.42 (m, 1H), 1.94-1.73 (m, 2H), 1.46-1.44 (m, 9H), 1.20-1.08 (m, 6H) ppm.

tert-Butyl (1S,4S)-5-isobutyryl-2,5-diazabicyclo[2.2.1]heptane-2-carboxylate (90 mg, 0.33 mmol) and 4 M HCl in dioxane (2.0 mL, 8 mmol) were stirred at room temperature under nitrogen for 2 hours before concentrating to dryness. The residue was combined with tert-butyl (2-((4-fluorophenyl)sulfonyl)ethyl)carbamate (85 mg, 0.28 mmol) and potassium carbonate (116 mg, 0.84 mmol) in anhydrous DMF (3 mL) and the reaction mixture was heated at 70° C. with stirring for 3 days. The reaction mixture was then cooled to room temperature diluted with diethyl ether (30 mL) and washed with water (3×30 mL) and brine (30 mL), dried over anhydrous magnesium sulfate, filtered and concentrated to give the crude product, which was purified using flash column chromatography over silica, eluting with 0-30% methanol/dichloromethane, to give tert-butyl (2-((4-((1 S,4S)-5-isobutyryl-2,5-diazabicyclo[2.2.1]heptan-2-yl)phenyl)sulfonyl)ethyl)carbamate (9 mg, 6% yield) as a yellow oil. ¹H NMR (CDCl₃, 300 MHz) δ 7.69 (d, 2H, J=9.0 Hz), 6.59 (d, 2H, J=8.9 Hz), 5.25-5.07 (m, 1H), 4.65-4.55 (m, 1H), 4.18-3.20 (m, 9H), 2.72-2.38 (m, 1H), 2.15-1.95 (m, 2H), 1.42 (s, 9H), 1.19-0.99 (m, 6H) ppm; LCMS m/z 452.4 found (M+H)⁺, C₂₂H₃₄N₃SO₅.

This intermediate was dissolved in 4 M HCl in dioxane (0.5 mL, 2.0 mmol) and the reaction mixture was stirred at room temperature under nitrogen for 3 hours before concentrating to dryness. Dichloromethane was added and the mixture was concentrated again, and the resulting residue was washed with diethyl ether and dried under vacuum to give the title compound (7 mg, 91% yield) isolated as an off-white solid. ¹H NMR (DMSO-d₆, 300 MHz) δ 8.11 (br s, 3H), 7.63 (d, 2H, J=8.4 Hz), 6.80 (d, 2H, J=8.6 Hz), 4.81-4.73 (m, 2H), 3.71-3.60 (m, 2H), 3.53-3.42 (m, 2H), 3.23-3.06 (m, 2H), 3.01-2.96 (m, 2H), 2.83-2.71 (m, 1H), 2.01-1.92 (m, 2H), 1.04-0.83 (m, 6H) ppm; LCMS m/z 352.3 found (M+H)⁺, C₁₇H₂₆N₃SO₃.

### Example 44: 4-((2-aminoethyl)sulfonyl)phenyl (1S,5S)-6-(4-ethoxyphenyl)-9,9-dimethyl-3,6-diazabicyclo[3.2.2]nonane-3-carboxylate hydrochloride

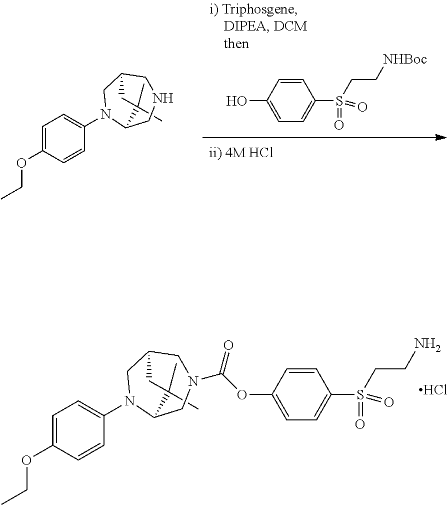 [SMILES: CCOc1ccc(N2C[C@H]3CNC[C@@H]2C(C)(C)C3)cc1 ; CC(C)(C)OC(=O)NCCS(=O)(=O)c1ccc(O)cc1 ; CCOc1ccc(N2C[C@H]3CN(C(=O)Oc4ccc(S(=O)(=O)CCN)cc4)C[C@@H]2C(C)(C)C3)cc1 ; Cl]

Triphosgene (41 mg, 0.137 mmol) was added to a solution of (1R,5S)-6-(4-ethoxyphenyl)-9,9-dimethyl-3,6-diazabicyclo[3.2.2]nonane (75 mg, 0.273 mmol) and DIPEA (0.29 mL, 1.64 mmol) in anhydrous DCM (5 mL), with stirring at room temperature under nitrogen for 1 hour. tert-Butyl (2-((4-hydroxyphenyl)sulfonyl)ethyl)carbamate (99 mg, 0.328 mmol) was then added, with continued stirring for 72 hours, before separating between dichloromethane and aqueous NaHCO₃ and the organic phase was washed with water (×2) and brine, dried over anhydrous sodium sulfate, filtered and concentrated to give the crude product, which was which was purified using flash column chromatography over basic alumina, eluting with 20-100% ethyl acetate/petroleum ether, to give 4-((2-((tert-butoxycarbonyl)amino)ethyl)sulfonyl)phenyl (1 S,5S)-6-(4-ethoxyphenyl)-9,9-dimethyl-3,6-diazabicyclo[3.2.2]nonane-3-carboxylate (71 mg, 43% yield) as a white solid. ¹H NMR (CDCl₃, 300 MHz) δ 7.93-7.88 (m, 2H), 7.34-7.24 (m, 2H), 6.88-6.84 (m, 2H), 6.60-6.57 (m, 2H), 5.25-5.19 (m, 1H), 4.53-4.32 (m, 2H), 3.98 (q, 2H, J=7.0 Hz), 3.58-3.15 (m, 9H), 2.58-2.53 (m, 1H), 1.66-1.51 (m, 2H), 1.43 (s, 9H), 1.38 (t, 3H, J=7.0 Hz), 1.16-1.11 (m, 3H), 0.94-0.93 (m, 3H) ppm; LCMS m/z 602.3 found (M+H)⁺, C₃₁H₄₄N₃O₇S.

This intermediate was dissolved in 4 M HCl in dioxane (1.0 mL, 4.0 mmol) and the reaction mixture was stirred at room temperature under nitrogen for 3 hours before concentrating to dryness. Dichloromethane was added and the mixture was concentrated again, and the resulting residue was washed with diethyl ether and dried under vacuum to give the title compound (56 mg, 88% yield) isolated as an off-white solid. ¹H NMR (DMSO-d₆, 300 MHz) δ 8.21 (br s, 3H), 7.99-7.94 (m, 2H), 7.47-7.33 (m, 2H), 6.80 (d, 2H, J=9.1 Hz), 6.62 (d, 2H, J=9.2 Hz), 4.45-4.13 (m, 2H), 3.91 (q, 2H, J=6.8 Hz), 3.72-3.60 (m 4H), 3.39-3.00 (m, 6H), 1.59-1.45 (m, 2H), 1.28 (t, 3H, J=7.0 Hz), 1.12-1.04 (m, 3H), 0.84-0.83 (m, 3H) ppm; LCMS m/z 502.3 found (M+H)⁺, C₂₆H₃₆N₃SO₅.

### Example 45: 1-((1S,4S)-5-(4-(((2-aminoethyl)sulfonyl)methyl)phenyl)-2,5-diazabicyclo[2.2.1]heptan-2-yl)-2-methylpropan-1-one hydrochloride

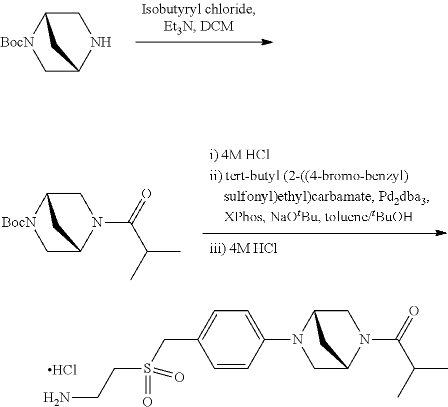 [SMILES: CC(C)(C)OC(=O)N1C[C@@H]2C[C@H]1CN2 ; CC(C)C(=O)N1C[C@@H]2C[C@H]1CN2C(=O)OC(C)(C)C ; Cl ; CC(C)C(=O)N1C[C@@H]2C[C@H]1CN2c1ccc(CS(=O)(=O)CCN)cc1]

Triethylamine (1.12 mL, 8.07 mmol) was added to a solution of tert-butyl (1S,2S)-2,5-diazabicyclo[2.2.1]heptane-2-carboxylate (800 mg, 4.0 mmol) and isobutyryl chloride (0.59 mL, 5.64 mmol) in anhydrous DCM (40 mL), with stirring at room temperature under nitrogen overnight. The reaction mixture was then separated between dichloromethane and aqueous NaHCO₃ and the organic phase was washed with water (×2) and brine, dried over anhydrous sodium sulfate, filtered and concentrated to give the crude product, which was which was purified using flash column chromatography over silica, eluting with 0-30% methanol/dichloromethane, to give tert-butyl (1 S,2S)-5-isobutyryl-2,5-diazabicyclo[2.2.1]heptane-2-carboxylate (1.1 g, 100% yield) as a white solid. ¹H NMR (CDCl₃, 300 MHz) δ 4.91-4.44 (m, 2H), 3.55-3.28 (m, 4H), 2.68-2.42 (m, 1H), 1.94-1.73 (m, 2H), 1.46-1.44 (m, 9H), 1.20-1.08 (m, 6H) ppm.

tert-Butyl (1 S,2S)-5-isobutyryl-2,5-diazabicyclo[2.2.1]heptane-2-carboxylate (150 mg, 0.556 mmol) and 4 M HCl in dioxane (3.5 mL, 14 mmol) were stirred at room temperature under nitrogen for 2 hours before concentrating to dryness. The residue was combined with tert-butyl (2-((4-bromo-benzyl)sulfonyl)ethyl)carbamate (231 mg, 0.61 mmol) in toluene:^{t}BuOH (5:1, 6 mL) and the solution was degassed with nitrogen for 5 minutes. Pd₂dba₃ (51 mg, 10 mol %), XPhos (53 mg, 20 mol %) and sodium tert-butoxide (134 mg, 1.39 mmol) were then added with stirring, and the solution was bubbled with nitrogen for a further 5 minutes before sealing the flask and stirring at room temperature for 1 hour and then heating to 100° C. for 18 hours. The reaction mixture was then diluted with ethyl acetate (30 mL) and washed with water (30 mL) and brine (30 mL), dried over anhydrous magnesium sulfate, filtered and concentrated to give the crude product, which was purified using flash column chromatography over silica, eluting with 80-100% ethyl acetate/cyclohexane then 0-30% methanol/ethyl acetate, to give tert-butyl (2-((4-((1 S,4S)-5-isobutyryl-2,5-diazabicyclo[2.2.1]heptan-2-yl)benzyl)sulfonyl)ethyl)carbamate (132 mg, 51% yield) as a yellow solid. ¹H NMR (CDCl₃, 300 MHz) δ 7.23 (d, 2H, J=8.7 Hz), 6.55 (d, 2H, J=8.7 Hz), 5.22-5.13 (m, 1H), 5.02-4.45 (m, 2H), 4.15 (s, 2H), 3.70-3.40 (m, 5H), 3.20-3.11 (m, 1H), 3.05-3.00 (m, 2H), 2.72-2.36 (m, 1H), 2.12-2.88 (m, 2H), 1.43 (s, 9H), 1.18-0.97 (m, 6H) ppm; LCMS m/z 466.3 found (M+H)⁺, C₂₃H₃₆N₃SO₅.

This intermediate was dissolved in 4 M HCl in dioxane (1.7 mL, 6.71 mmol) and the reaction mixture was stirred at room temperature under nitrogen for 3 hours before concentrating to dryness. Dichloromethane was added and the mixture was concentrated again, and the resulting residue was washed with diethyl ether and dried under vacuum to give the title compound (92 mg, 85% yield) isolated as an off-white solid. ¹H NMR (DMSO-d₆, 300 MHz) δ 8.23 (br s, 3H), 7.21 (d, 2H, J=7.4 Hz), 6.63 (d, 2H, J=8.0 Hz), 4.76-4.45 (m, 4H), 3.50-3.15 (m, 7H), 3.01-2.71 (m, 2H), 2.01-1.89 (m, 2H), 1.09-0.79 (m, 6H) ppm; LCMS m/z 366.2 found (M+H)⁺, C₁₈H₂₈N₃SO₃.

### Example 46: (4-(((2-aminoethyl)sulfonyl)methyl)phenyl)((1S,5S)-6-(4-ethoxyphenyl)-9,9-dimethyl-3,6-diazabicyclo[3.2.2]nonan-3-yl)methanone

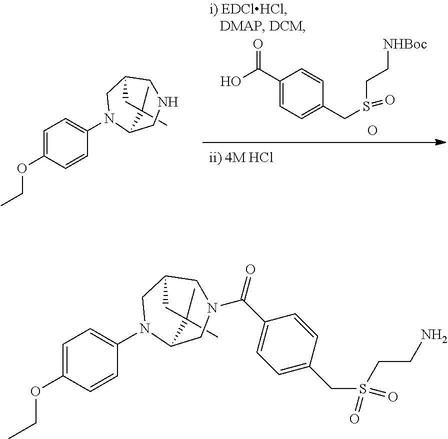

DMAP (44 mg, 0.364 mmol) and triethylamine (0.25 mL, 1.82 mmol) were added to a solution of (1R,5S)-6-(4-ethoxyphenyl)-9,9-dimethyl-3,6-diazabicyclo[3.2.2]nonane (105 mg, 0.383 mmol) and 4-(((2-((tert-butoxycarbonyl)amino)ethyl)sulfonyl)methyl)benzoic acid (125 mg, 0.364 mmol) in anhydrous dichloromethane (5 mL) with stirring under nitrogen. The reaction mixture was then cooled to 0° C. before adding EDCI.HCl (77 mg, 0.40 mmol), with continued stirring overnight warming to room temperature. The reaction mixture was then separated between dichloromethane and aqueous NaHCO₃ and the organic phase was washed with water (×2) and brine, dried over anhydrous sodium sulfate, filtered and concentrated to give the crude product, which was which was purified using flash column chromatography over basic alumina, eluting with 40-100% ethyl acetate/petroleum ether, to give the product (150 mg, 69% yield) as a white solid. ¹H NMR (CDCl₃, 300 MHz) δ 7.47-7.36 (m, 4H), 6.88-6.83 (m, 2H), 6.60-6.51 (m, 2H), 5.16-4.80 (m, 2H), 4.27 (s, 2H), 3.98 (q, 2H, J=7.0 Hz), 3.87-3.49 (m, 4H), 3.33-3.09 (m, 6H), 2.62-2.36 (m, 1H), 1.66-1.50 (m, 2H), 1.45 (s, 9H), 1.38 (t, 3H, J=7.0 Hz), 1.28-1.13 (m, 3H), 0.93-0.86 (m, 3H) ppm; LCMS m/z 600.3 found (M+H)⁺, C₃₂H₄₆N₃O₆S.

This intermediate was dissolved in 4 M HCl in dioxane (1.45 mL, 5.8 mmol) and the reaction mixture was stirred at room temperature under nitrogen for 3 hours before concentrating to dryness. Dichloromethane was added and the mixture was concentrated again, and the resulting residue was washed with diethyl ether and dried under vacuum to give the title compound (113 mg, 92% yield) isolated as an off-white solid. ¹H NMR (DMSO-d₆, 300 MHz) δ 8.33 (br s, 3H), 7.52-7.34 (m, 4H), 6.81-6.75 (m, 2H), 6.63-6.57 (m, 2H), 4.87-4.65 (m, 3H), 3.90 (q, 2H, J=6.9 Hz), 3.81-3.63 (m, 2H), 3.51-3.42 (m, 3H), 3.24-2.92 (m, 5H), 2.58-2.36 (m, 1H), 1.52-1.35 (m, 2H), 1.27 (t, 3H, J=7.0 Hz), 1.09-0.75 (m, 6H) ppm; LCMS m/z 500.3 found (M+H)⁺, C₂₇H₃₈N₃SO₄.

### Materials and Methods

### LOX Activity in Cysts Assay (Tang et al, 2017).

### Cell Culture and Transfection

All cell lines used in this study were purchased from American Type Culture Collection (ATCC). Mycoplasma contamination was routinely monitored by PCR. Cells used were not found to be Mycoplasma positive. MDCK cell lines were cultured in Dulbecco's Modified Eagle Medium (DMEM) supplemented with 10% fetal bovine serum (FBS) and 1% Penicillin Streptomycin solution (Pen Strep). For GFP constructs transfection in MDCK cells, lipofectamine 3000 was used according to manufactures protocols. Cells were either selected with G418 (Life Technologies) at 5 mg/ml. All cell culture reagents were purchased from Life Technologies.

To produce MDCK cysts, cells were cultured on Matrigel (Corning) with 2% Matrigel supplemented in DMEM with 10% FBS. Cysts were allowed to form for 10 days before subsequent studies.

### Cloning of LOX Expression Constructs

Mouse LOX cDNA was purchased from OriGene. Full length LOX cDNA was then PCR cloned into pEGFP-N1 (Clonetech), or biosensor vector proGFP2—N1 (Hanson, 2004) using the following primers, GAGAGAGCTAGCATGCGTTTCGCCTGGG (forward primer) and TCTCTCCTCGAGATACGGTGAAATTGTGCAGCC (reverse primer). For the insertion into pEGFP-N1 or proGFP2—N1, Nhel and Xhol restriction sites were added to forward and reverse primers accordingly. Mutant LOX constructs were made using QuickChange II site-directed mutagenesis kit (Agilent Technologies) following manufacture's protocol using LOX-GFP as template. To generate, roGFP2 versions of LOX mutant constructs, LOX mutant cDNA was transferred from pEGFP-N1 to proGFP2—N1 using Nhel and Xhol.

### Confocal Imaging and Imaging Analysis

All photomicrographs were taken with a Leica TCS SP8 X confocal system. For LOX biosensor imaging, live MDCK cysts were used. The oxidised biosensor was excited using a 405 nm laser, while the reduced biosensor was excited with a 488 nm laser. Emission of the biosensor was recorded at 500 nm-530 nm range using sequential scans. Ratio images were generated following a published protocol {Kardash, 2011 #376}. Note, while the published protocol generates YFP/CFP ratio images, we used it to generate Oxidised/Reduced (roGFP2 ratio) ratio images. The roGFP2 ratio at the basal surface of MDCK cysts was used to indicate LOX inhibition. LOX inhibitors were added 30 min prior to imaging.

The inhibition of LOX in cysts assay by LOX inhibitors as compared to control (DMSO vehicle treated) cysts is shown in Table 3a. For clarity, readout for DMSO treated cysts represents 0% inhibition (no inhibition) and readout for BAPN at 1 mM is used as 100% inhibition (full inhibition).

### LOX Isoform (LOX, LOXL2, LOXL3) Activity Assay

LOX was extracted from pig skin by previously described methods (Shackleton and Hulmes, 1990). LOXL2 and LOXL3 were obtained from R&D Systems/Bio-techne. The Promega ROS Glo assay kit (G8821) was used to assess enzyme activity. Overall, test compound was added to either 384 well assay plates (Greiner) using a Labcyte Echo™ Acoustic Handler or to 96 well plates for a final assay concentration of 100, 30, 10, 3, 1, 0.3, 0.1, 0.03, 0.01 and 0.003 μM final assay concentration. Enzymes were diluted according to specific pre-determined activity in assay buffer (100 mM CHES, pH 9, 0.05% (w/v) pluronic F-127, 0.5% (w/v) BSA, 1 mM MgCl2, 1 M urea, 100 mM NaCl) and added to the plate. For LOXL2, the mixture was incubated for 20 min, 1 h or 20 h; for LOX and LOXL3, incubation time was 1 h. Cadaverine and H₂O₂ substrate were then added to each well (final assay concentration 19.56 mM and 25 μM respectively) and the plates were incubated for 1.5 h (for LOX) or 1 h (for LOXL2 and LOXL3) at room temperature. Finally, luciferin detection reagent was added and plates were incubated for 20 min at room temperature, protected from light. Luminescence was measured using a BMG LABTECH PHERAstar FS reader and resulting IC50 values were calculated using GraphPad Prism software.

In another setting, LOXL2 enzyme was diluted according to specific pre-determined activity in assay buffer (100 mM CHES, pH 9, 0.05% (w/v) pluronic F-127, 0.5% (w/v) BSA, 1 mM MgCl2, 1 M urea, 100 mM NaCl). Overall, 0.5 μl test compound was added to 40 μl assay buffer+LOXL2 at 100, 30, 10, 3, 1, 0.3, 0.1, 0.03, 0.01 and 0.003 μM (final concentration) in a 96-well white polystyrene, flat bottom plates (Fisher) and incubated for the indicated time at room temperature. 10 μl of assay buffer containing 97.8 mM cadaverine and 125 μM H₂O₂ substrate (final assay concentration 19.56 mM and 25 μM respectively) were added to each well and plates were incubated for 1 h at room temperature. Finally, 50 μl per well luciferin detection reagent was added and plates were incubated for 20 min at room temperature, protected from light. Luminescence was measured with an integration time of 500 ms per well using a SpectaMax M5 plate reader and resulting IC50 values were calculated using GraphPad Prism software.

### Amine Oxidase Activity Assay (Tang et al, 2017).

The Promega MAO-Glo assay kit (V1402) was used to assess monoamine oxidase A (MAO-A) and monoamine oxidase B (MAO-B) activity. MAO substrate was prepared at 80 μM with the specific MAO-A/B reaction buffer. Overall, 0.5 μl test compound was added to 25 μl MAO substrate at 100, 30, 10, 3, 1, 0.3, 0.1, 0.03, 0.01 and 0.003 μM (final concentration) in a 96-well white, flat bottom plate. Overall, 25 μl of 1:520 dilution of MAO-A enzyme (Promega, V1452) or 25 μl of 1:52 dilution of MAO-B enzyme (Sigma, M7441) in specific MAO-A/B reaction buffer was added to each well (enzyme prepared at 2×final concentration) and plates were incubated for 1 h at room temperature. Finally, 50 μl per well luciferin detection reagent was added and plates were incubated for 20 min at room temperature, protected from light. Luminescence was measured with an integration time of 500 ms per well using a SpectaMax M5 plate reader and resulting IC50 values were calculated using GraphPad Prism software.

The diamine oxidase (DAO) catalytic activity was determined using the Promega ROS Glo assay kit (G8821); DAO was purchased from Sigma and cadaverine dihydrochloride was used as the substrate at a concentration of 97.8 mM. The concentrations of the chemical inhibitors were the same as in the MAO assays. Luminescence was measured with an integration time of 500 ms/well using a SpectaMax M5 plate reader and resulting IC50 values were calculated using GraphPad Prism software.

In another setting, the DAO and MAO assays were performed in 384-well plates (Greiner) and test compound was added using a Labcyte Echo™ Acoustic Handler for the same final concentrations as in the 96-well setting. Luminescence was measured with BMG LABTECH PHERAstar FS plate reader.

TABLE 3a
in vitro data

| | | | | | LOXL2 | | | | |
|---|---|---|---|---|---|---|---|---|---|
| | | | | | IC₅₀ | | | | |
| | | | Cyst | | (μM) | | | | |
| | | Cyst LOX | LOX | LOX | a) 20 min | LOXL3 | MAO A | MAO B | DAO |
| Example | | @ 10 uM | IC₅₀ | IC₅₀ | b) 1 hr | IC₅₀ | IC₅₀ | IC₅₀ | IC₅₀ |
| No. | Example, Structure | (% inhibition) | (μM) | (μM) | c) 20 hr | (μM) | (μM) | (μM) | (μM) |
| 1 | 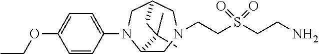 [SMILES: CCOc1ccc(N2C[C@H]3CN(CCS(=O)(=O)CCN)C[C@@H]2C(C)(C)C3)cc1] | \ | \ | \ | c) 1.6 | \ | >100 | >100 | >100 |
| 2 | 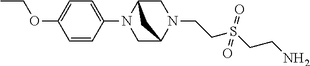 [SMILES: CCOc1ccc(N2C[C@@H]3C[C@H]2CN3CCS(=O)(=O)CCN)cc1] | \ | \ | \ | c) 0.4 | \ | >100 | >100 | >100 |
| 3 | 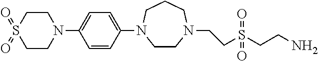 [SMILES: NCCS(=O)(=O)CCN1CCCN(c2ccc(N3CCS(=O)(=O)CC3)cc2)CC1] | \ | \ | \ | a) 8.1 | \ | >100 | >100 | \ |
| 4 | 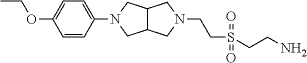 [SMILES: CCOc1ccc(N2CC3CN(CCS(=O)(=O)CCN)CC3C2)cc1] | 100 | \ | \ | a) 7.3 | \ | >100 | >100 | \ |
| 5 | 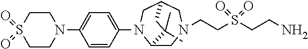 [SMILES: CC1(C)C[C@@H]2CN(CCS(=O)(=O)CCN)C[C@H]1N(c1ccc(N3CCS(=O)(=O)CC3)cc1)C2] | \ | \ | \ | c) 0.48 | \ | \ | \ | \ |
| 6 | 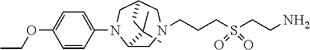 [SMILES: CCOc1ccc(N2C[C@H]3CN(CCCS(=O)(=O)CCN)C[C@@H]2C(C)(C)C3)cc1] | \ | \ | 58 | c) 2.5 | \ | \ | \ | \ |
| 7 | 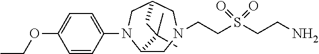 [SMILES: CCOc1ccc(N2C[C@H]3CN(CCS(=O)(=O)CCN)C[C@@H]2C(C)(C)C3)cc1] | 79 | \ | \ | c) 0.27 | \ | >100 | >100 | >100 |
| 8 | 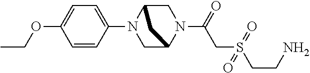 | 46% (@ 2.5 uM) | 0.37 | \ | c) 0.15 | 0.55 | >100 | >100 | >100 |
| 9 | 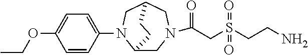 [SMILES: CCOc1ccc(N2C[C@@H]3CC[C@H]2CN(C(=O)CS(=O)(=O)CCN)C3)cc1] | \ | \ | >100 | c) 0.34 | \ | \ | \ | \ |
| 10 | 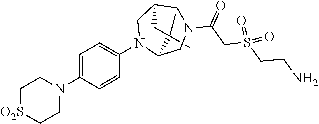 [SMILES: CC1(C)C[C@@H]2CN(C(=O)CS(=O)(=O)CCN)C[C@H]1N(c1ccc(N3CCS(=O)(=O)CC3)cc1)C2] | \ | \ | \ | c) 0.94 | \ | \ | \ | \ |
| 15 | 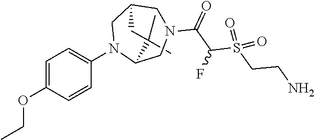 [SMILES: CCOc1ccc(N2C[C@H]3CN(C(=O)C(F)S(=O)(=O)CCN)C[C@@H]2C(C)(C)C3)cc1] | 78% (2.5 uM) | \ | 2.2 | c) 0.12 | \ | \ | \ | \ |
| 16 | 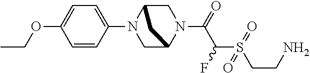 | 73% (2.5 uM) | \ | \ | c) 0.14 | \ | \ | \ | \ |
| 17 | 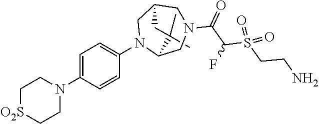 [SMILES: CC1(C)C[C@@H]2CN(C(=O)C(F)S(=O)(=O)CCN)C[C@H]1N(c1ccc(N3CCS(=O)(=O)CC3)cc1)C2] | \ | \ | \ | b) 0.11 | \ | \ | \ | \ |
| 30 | 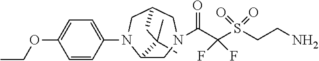 [SMILES: CCOc1ccc(N2C[C@H]3CN(C(=O)C(F)(F)S(=O)(=O)CCN)C[C@@H]2C(C)(C)C3)cc1] | \ | \ | 10 | b) 2.5 | \ | \ | \ | \ |
| 31 | 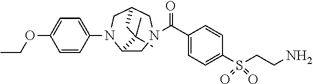 [SMILES: CCOc1ccc(N2C[C@H]3CN(C(=O)c4ccc(S(=O)(=O)CCN)cc4)C[C@@H]2C(C)(C)C3)cc1] | \ | \ | 96 | a) 16 | \ | \ | \ | \ |
| 32 | 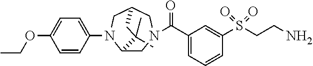 [SMILES: CCOc1ccc(N2C[C@H]3CN(C(=O)c4cccc(S(=O)(=O)CCN)c4)C[C@@H]2C(C)(C)C3)cc1] | \ | \ | 23 | c) 3.0 | \ | >100 | >100 | \ |
| 33 | 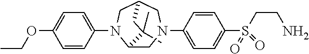 [SMILES: CCOc1ccc(N2C[C@H]3CN(c4ccc(S(=O)(=O)CCN)cc4)C[C@@H]2C(C)(C)C3)cc1] | 100 | \ | \ | c) 16 | \ | >100 | >100 | \ |
| 34 | 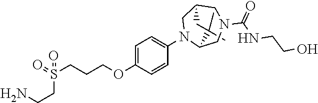 [SMILES: CC1(C)C[C@@H]2CN(C(=O)NCCO)C[C@H]1N(c1ccc(OCCCS(=O)(=O)CCN)cc1)C2] | \ | \ | \ | c) 9.3 | \ | \ | \ | \ |
| 35 | 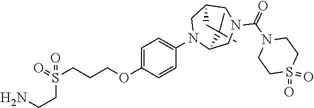 [SMILES: CC1(C)C[C@@H]2CN(C(=O)N3CCS(=O)(=O)CC3)C[C@H]1N(c1ccc(OCCCS(=O)(=O)CCN)cc1)C2] | \ | \ | \ | c) 6.7 | \ | \ | \ | \ |
| 36 | 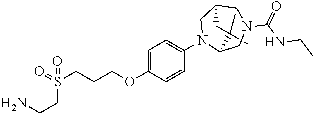 [SMILES: CCNC(=O)N1C[C@@H]2CN(c3ccc(OCCCS(=O)(=O)CCN)cc3)[C@H](C1)C(C)(C)C2] | \ | \ | 4.5 | c) 5.1 | \ | \ | \ | \ |
| 37 | 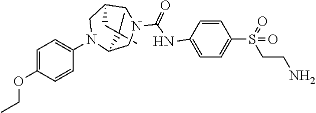 [SMILES: CCOc1ccc(N2C[C@H]3CN(C(=O)Nc4ccc(S(=O)(=O)CCN)cc4)C[C@@H]2C(C)(C)C3)cc1] | 78 | \ | 26 | b) 20 | \ | \ | \ | \ |
| 38 | 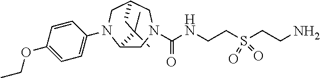 [SMILES: CCOc1ccc(N2C[C@H]3CN(C(=O)NCCS(=O)(=O)CCN)C[C@@H]2C(C)(C)C3)cc1] | 70 | \ | 8.8 | b) 0.58 | \ | \ | \ | >100 |
| 39 | 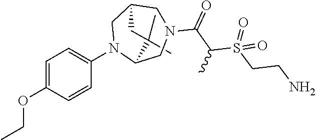 [SMILES: CCOc1ccc(N2C[C@H]3CN(C(=O)C(C)S(=O)(=O)CCN)C[C@@H]2C(C)(C)C3)cc1] | \ | \ | 10 | b) 0.41 | \ | \ | \ | \ |
| 40 | 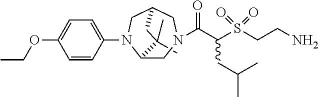 [SMILES: CCOc1ccc(N2C[C@H]3CN(C(=O)C(CC(C)C)S(=O)(=O)CCN)C[C@@H]2C(C)(C)C3)cc1] | 79 | \ | \ | b) 1.1 | \ | \ | \ | \ |

TABLE 3b
in vitro data

| | | | LOXL2 | | | | |
|---|---|---|---|---|---|---|---|
| | | | IC₅₀ (μM) | | MAO | MAO | |
| | | LOX | a) 20 min | LOXL3 | A | B | DAO |
| | | IC₅₀ | b) 1 hr | IC₅₀ | IC₅₀ | IC₅₀ | IC₅₀ |
| Ex. | Structure | (μM) | c) 20 hr | (μM) | (μM) | (μM) | (μM) |
| 41 | 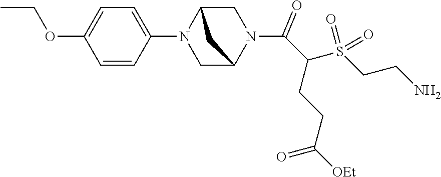 [SMILES: CCOC(=O)CCC(C(=O)N1C[C@@H]2C[C@H]1CN2c1ccc(OCC)cc1)S(=O)(=O)CCN] | 41 | b) 3.1 | \ | >100 | >100 | >100 |
| 42 | 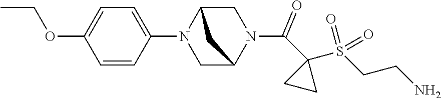 [SMILES: CCOc1ccc(N2C[C@@H]3C[C@H]2CN3C(=O)C2(S(=O)(=O)CCN)CC2)cc1] | >100 | b) 11 | \ | >100 | >100 | >100 |
| 11 | 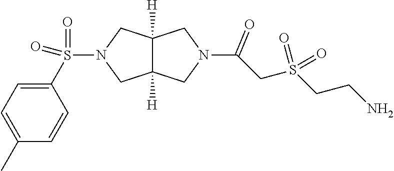 [SMILES: Cc1ccc(S(=O)(=O)N2C[C@H]3CN(C(=O)CS(=O)(=O)CCN)C[C@H]3C2)cc1] | \ | b) 0.17 | \ | >100 | >100 | >100 |
| 18 | 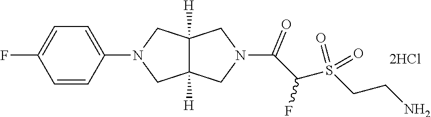 [SMILES: NCCS(=O)(=O)C(F)C(=O)N1C[C@@H]2CN(c3ccc(F)cc3)C[C@@H]2C1] | 2.3 | b) 0.23 | 0.44 | \ | \ | \ |
| 19 | 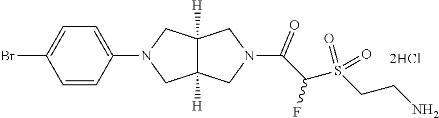 [SMILES: NCCS(=O)(=O)C(F)C(=O)N1C[C@@H]2CN(c3ccc(Br)cc3)C[C@@H]2C1] | 0.66 | b) 0.15 | 0.22 | \ | \ | \ |
| 20 | 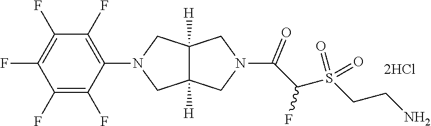 [SMILES: NCCS(=O)(=O)C(F)C(=O)N1C[C@@H]2CN(c3c(F)c(F)c(F)c(F)c3F)C[C@@H]2C1] | 2.4 | b) 0.24 | 0.34 | \ | \ | \ |
| 12 | 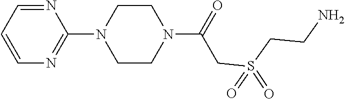 [SMILES: NCCS(=O)(=O)CC(=O)N1CCN(c2ncccn2)CC1] | \ | b) 0.43 | \ | >100 | >100 | >100 |
| 13 | 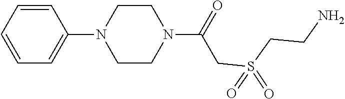 [SMILES: NCCS(=O)(=O)CC(=O)N1CCN(c2ccccc2)CC1] | 25 | b) 0.40 | 0.28 | >100 | >100 | >100 |

### In Vivo Assessment of LOX Inhibitors

### Animal Procedures

All procedures involving animals were approved by the Animal Welfare and Ethical Review Body of the Institute of Cancer Research and Cancer Research UK Manchester Institute in accordance with National Home Office regulations under the Animals (Scientific Procedures) Act 1986 and according to the guidelines of the Committee of the National Cancer Research Institute Tumour size was determined by caliper measurements of tumour length, width and depth and volume was calculated as volume=0.5236×length×width×depth (mm). In accordance with our licence to perform animal experiments, animals were excluded from the experiments if they displayed signs of distress, excessive bodyweight loss (>20%) or illness.

### Oral Tolerability of LOX Inhibitors

Two CD1, NCR or Balb/c female mice at 6 weeks of age were dosed po by metal gavage once a day for 4 consecutive days with suspension of the test compound at the dose planned for therapy (200 mg/kg/day) in 5.25% Tween20/saline (v:v) or 5% DMSO in water at 0.2 ml per 20 g bodyweight.

The mice were observed for up to 15 days after last dose and their bodyweight measured every 4 days. A compound is considered tolerated if the bodyweight does not fall by >20% for over 72 hrs.

Compounds of this invention tested in vivo show good tolerability at the dose tested and exhibit <5% bodyweight loss or show bodyweight gain in the tolerability study and in further longer therapy studies.

### In Vivo Tumour Models Studies

MDA-MB-231 xenografts. Ncr nude female mice at 6 weeks old from Charles River were injected into the third upper nipple mammary fat pad with MDA-MB-231 Luc 4×10\hat{ }6 in 100 ul PB (50:50 Matrigel). When tumours reach a mean of 80 mm³ around 10 days post cell inoculation the animals are allocated in 4 groups of 8. LOX inhibitor treatment is then administrated by oral gavage dosing, at 0.2 ml/20 g bodyweight once daily for up to 28 consecutive days. Tumours and weights are measured twice weekly using calipers and the animals can be imaged using non invasive method by bioluminescence using IVIS 200 imaging machine, weekly using 150 mg/kg luciferin administrate intraperitoneal or subcutaneous. At the end of the study the animals are culled, and samples taken, fixed or snap frozen in liquid nitrogen. Frozen samples kept at −80 degree centigrade until being analyzed and the fixed samples stained according to desired marker.

### LOX Inhibitor Treatment of a Transgenic Mouse Breast Cancer Model

MMTV-PyMT (Guy et al, 1992) (FVB) female mice were randomized by non-statistical methods to LOX inhibitor treatment groups from day 70 post-birth, when animals had no detectable tumour. Mice were treated daily by oral gavage with LOX inhibitor in vehicle, or daily vehicle (5% DMSO/2.5% Tween20 in water) by oral gavage. Tumour size was determined unblinded by caliper measurements of tumour length, width and depth and volume was calculated as 0.5236×length×width×depth (mm). In all experiments, mice were humanely killed and mammary tumours and lungs were collected when the primary tumours reached ethical limits or signs of ill health.

For therapeutic efficacy assessment, the ratio of average tumour volume between compound treated and vehicle control treated (T/C) is calculated. Reduction in tumour volume in the compound treated group compared to vehicle-treated control group results in T/C<1. The efficacy of LOX inhibitors described in this invention, as measured by T/C in breast cancer models is shown in Table 4 and is significant (p<0.05) for all the data presented.

For lung metastases quantification, all mouse tissue samples were fixed in 10% formalin (Sigma) and embedded in paraffin. Samples were sectioned and hematoxylin and eosin (H&E) stained. Samples were imaged with a Leica SCN400 slide scanner. Lung metastases were manually selected using Pen tool in ImageScope. Lung metastases number was counted and area was measured using ImageScope. The investigator was blinded to the experimental groups. The ratio of average metastases surface between compound treated and vehicle control treated (T/C) is calculated. The ratio of average metastases numbers between compound treated and vehicle control treated (T/C) is also calculated. Reduction in metastases area and/or in metastatses number in the compound treated group compared to vehicle-treated control group results in T/C<1. The antimetastatic efficacy of LOX inhibitors described in this invention, as measured by T/C in a model of breast cancer metastasising to lungs is shown in Table 4 and is significant (p<0.05) for all the data presented.

TABLE 4
in vivo data

| | MDA-MB-231 | MMT-PyMT | MMT-PyMT |
|---|---|---|---|
| | human | breast | breast trans- |
| | breast adeno- | transgenic | genic model- |
| | carcinoma- | model- | metastases |
| | primary | primary | a) Count |
| Example and structure | tumour | tumour | b) Area |
| 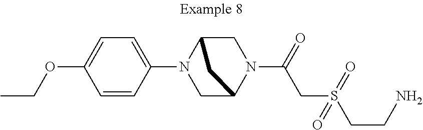 [SMILES: CCOc1ccc(N2C[C@@H]3C[C@H]2CN3C(=O)CS(=O)(=O)CCN)cc1] | 0.54 | 0.6 | a) 0.14 |

Significant, p<0.05
All values are T/C, all doses are 200 mg/kg po qd unless otherwise stated.

The present disclosure relates to embodiments as disclosed in clauses 1 to 64 below:
1. A compound having the structure of Formula (I):

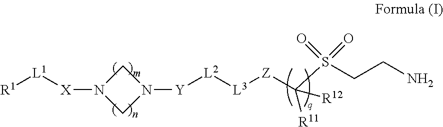

or a pharmaceutically acceptable salt thereof, wherein
m and n are each independently selected from 1, 2, 3 or 4, and where two ring carbon atoms of the cyclic diamine moiety of formula

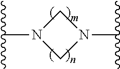

may be

(i) optionally linked by a bond,

(ii) optionally bridged by —(CR⁹R¹⁰)ₒ—, where R⁹ and R¹⁰ are at each occurrence independently selected from H and unsubstituted C₁₋₄ alkyl and o is 1, 2, 3 or 4; or

(iii) optionally linked by a spiro carbon atom; and
each ring carbon atom of said cyclic diamine moiety may be optionally substituted by one or two substituents independently selected from C₁-C₆ alkyl, C₁-C₆ alkyl substituted with —OR^{2a}, or C₁-C₆ alkyl substituted with —NR^{2a}R^{2b};
   L¹ and L² are each independently selected from a bond, —O—, —C(O)—, —C(O)O—, —OC(O)—, —C(O)NR³—, —NR³C(O)—, —NR³—, —SO₂NR³—, —NR³SO₂—, —S—, —SO₂—, —SO₂O—, —OSO₂—, —NR³SO₂NR⁴—, —NR³C(O)NR⁴—, —NR³C(O)O— or —OC(O)NR³—;
   L³ is selected from a bond, C₁-C₄ alkylene, C₂-C₄ alkenylene or C₂-C₄ alkynylene, where
any alkylene, alkenylene or alkynylene in L³ may be optionally substituted by one or two substituents independently selected from halo, cyano, oxo, hydroxy, carboxy, R², —OR², —C(O)R², —C(O)OR², —OC(O)R², —C(O)NR⁶R⁷, —NR⁶C(O)R⁷, —NR⁶R⁷, —SO₂NR⁶R⁷, —NR⁶SO₂R⁷, —SR⁶, —SO₂R⁶, —SO₂OR⁶, —OSO₂R⁶, —NR⁶SO₂NR⁷R⁸, —NR⁶C(O)NR⁷R⁸, —NR⁶C(O)OR⁷ or —OC(O)NR⁶R⁷;
   X, Y and Z are each independently selected from a bond or a 3- to 12-membered ring system, including 0, 1, 2 or 3 heteroatoms selected from N, O or S in the ring system, where
any ring system in X, Y and Z may be optionally substituted by one or more substituents independently selected from halo, cyano, oxo, hydroxy, carboxy, R², —OR², —C(O)R², —C(O)OR²,—OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵, —R³C(O)NR⁴R⁵, —NR³C(O)OR⁴ or —OC(O)NR⁴R⁵;
   R¹ is selected from hydrogen, halogen, C₁-C₆ alkyl, C₂-C₆ alkenyl, C₂-C₆ alkynyl or a 3- to 12-membered ring system (e.g. cycloalkyl, heterocyclyl, aryl or heteroaryl), including 0, 1, 2 or 3 heteroatoms selected from N, O or S in the ring system, where
any alkyl, alkenyl or alkynyl in R¹ may be optionally substituted by one, two or three substituents independently selected from halo, cyano, amino, oxo, hydroxy, carboxy, R², —OR², —C(O)R², —C(O)OR², —OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵, —NR³C(O)NR⁴R⁵, —NR³C(O)OR⁴ or —OC(O)NR⁴R⁵; and
any ring system in R¹ may be optionally substituted by one or more substituents independently selected from halo, cyano, oxo, hydroxy, carboxy, R², —OR², —C(O)R², —C(O)OR²,—OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵,—NR³C(O)NR⁴R⁵, —NR³C(O)OR⁴ or —OC(O)NR⁴R⁵;
   R² is at each occurrence independently selected from C₁-C₆ alkyl, C₂-C₆ alkenyl, C₂-C₆ alkynyl or C₃-C₆ cycloalkyl, where
any alkyl, alkenyl, alkynyl or cycloalkyl in R² may be optionally substituted by one, two or three substituents independently selected from halo, cyano, oxo, R^{2a}, —OR^{2a}, —C(O)R^{2a}, —C(O)OR^{2a}, —OC(O)R^{2a}, —C(O)NR^{2a}R^{2b}, —NR^{2a}C(O)R^{2b}, —NR^{2a}R^{2b}, —SO₂NR^{2a}R^{2b}, —NR^{2a}SO₂R^{2b}, —SR^{2a}, —SO₂R^{2a}, —SO₂OR^{2a}, —OSO₂R^{2a}, —NR^{2a}SO₂NR^{2b}R², —NR^{2a}C(O)NR^{2b}R^{2c}, —NR^{2a}C(O)OR^{2b} or —OC(O)NR^{2a}R^{2b};
   R^{2a}, R^{2b} and R^{2c} are at each occurrence independently selected from hydrogen or unsubstituted C₁-C₄ alkyl;
   R³, R⁴ and R⁵ are at each occurrence independently selected from hydrogen, C₁-C₆ alkyl or C₃-C₆ cycloalkyl, where
any alkyl or cycloalkyl in R³, R⁴ and R⁵ may be optionally substituted by one, two or three substituents independently selected from halo, cyano, oxo, hydroxy, carboxy, R², —OR², —C(O)R² or —C(O)OR², and
when R⁴ is optionally substituted C₁-C₆ alkyl and R⁵ is optionally substituted C₁-C₆ alkyl, then R⁴ and R⁵ together with the nitrogen atom to which they are attached in —C(O)NR⁴R⁵, —NR⁴R⁵, —SO₂NR⁴R⁵, —NR³SO₂NR⁴R⁵, —R³C(O)NR⁴R⁵ or —OC(O)NR⁴R⁵ may form a 3- to 6-membered heterocycloalkyl;
   R⁶, R⁷ and R⁸ are at each occurrence independently selected from hydrogen, C₁-C₆ alkyl or C₃-C₆ cycloalkyl, where
any alkyl or cycloalkyl in R⁶, R⁷ and R⁸ may be optionally substituted by one, two or three substituents independently selected from halo, cyano, oxo, hydroxy, carboxy, R^{2a}, —OR^{2a}, —C(O)R^{2a}, —C(O)OR^{2a}, —C(O)NR^{2a}R^{2b}, —NR^{2a}C(O)R^{2b}, —NR^{2a}R^{2b}, —SO₂NR^{2a}R^{2b}, —NR^{2a}SO₂R^{2b}, —SR^{2a}, —SO₂R^{2a}, —SO₂OR^{2a}, —OSO₂R^{2a}, —NR₂SO₂NR^{2a}R^{2b}, —NR^{2a}C(O)NR^{2b}R^{2c}, —NR^{2a}C(O)OR^{2b} or —OC(O)NR^{2a}R^{2b};
   R¹¹ and R¹² are independently selected from hydrogen and C₁-C₆ alkyl; and
   q is 0, 1 or 2;
   provided at least one of L², Y, L³ and Z is not a bond;
   provided —SO₂—(CH₂)₂—NH₂ in Formula I is linked to the remainder of the compound of Formula I via a carbon atom;
   provided when X is a bond, then L¹ is selected from a bond, —C(O)—, —OC(O)—, —NR³C(O)—, —NR³SO₂—, —SO₂— and —OSO₂—;
   provided when Y is a bond, then L² is selected from a bond, —C(O)—, —C(O)O—, —C(O)NR³—, —SO₂NR³—, —SO₂— and —SO₂O; and
   provided the compound is not

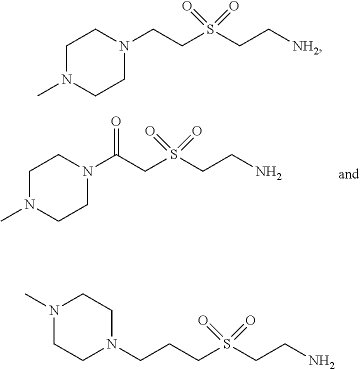

2. A compound having the structure of Formula Ia:

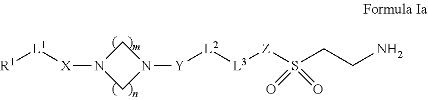

or a pharmaceutically acceptable salt thereof, wherein

m and n are each independently selected from 1, 2, 3 or 4, and where two ring carbon atoms of the cyclic diamine moiety of formula

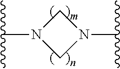

may be optionally linked by a bond or optionally bridged by —(CR⁹R¹⁰)ₒ—, where R⁹ and R¹⁰ are at each occurrence independently selected from H and unsubstituted C₁₋₄ alkyl and o is 1, 2, 3 or 4;
each ring carbon atom of said cyclic diamine moiety may be optionally substituted by one or two substituents independently selected from C₁-C₆ alkyl, C₁-C₆ alkyl substituted with —OR^{2a}, or C₁-C₆ alkyl substituted with —NR^{2a}R^{2b};

L¹ and L² are each independently selected from a bond, —O—, —C(O)—, —C(O)O—, —OC(O)—, —C(O)NR³—, —NR³C(O)—, —NR³—, —SO₂NR³—, —NR³SO₂—, —S—, —SO₂—, —SO₂O—, —OSO₂—, —NR³SO₂NR⁴—, —NR³C(O)NR⁴—, —NR³C(O)O— or —OC(O)NR³—;

L³ is selected from a bond, C₁-C₄ alkylene, C₂-C₄ alkenylene or C₂-C₄ alkynylene, where
any alkylene, alkenylene or alkynylene in L³ may be optionally substituted by one or two substituents independently selected from halo, cyano, oxo, hydroxy, carboxy, R², —OR², —C(O)R², —C(O)OR², —OC(O)R², —C(O)NR⁶R⁷, —NR⁶C(O)R⁷, —NR⁶R⁷, —SO₂NR⁶R⁷, —NR⁶SO₂R⁷, —SR⁶, —SO₂R⁶, —SO₂OR⁶, —OSO₂R⁶, —NR⁶SO₂NR⁷R⁸, —NR⁶C(O)NR⁷R⁸, —NR⁶C(O)OR⁷ or —OC(O)NR⁶R⁷.

X, Y and Z are each independently selected from a bond or a 3- to 12-membered ring system, including 0, 1, 2 or 3 heteroatoms selected from N, O or S in the ring system, where
any ring system in X, Y and Z may be optionally substituted by one, two or three substituents independently selected from halo, cyano, oxo, hydroxy, carboxy, R², —OR², —C(O)R², —C(O)OR², —OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵, —R³C(O)NR⁴R⁵, —NR³C(O)OR⁴ or —OC(O)NR⁴R⁵;

R¹ is selected from hydrogen, C₁-C₆ alkyl, C₂-C₆ alkenyl, C₂-C₆ alkynyl or a 3- to 12-membered ring system, including 0, 1, 2 or 3 heteroatoms selected from N, O or S in the ring system, where
any alkyl, alkenyl or alkynyl in R¹ may be optionally substituted by one, two or three substituents independently selected from halo, cyano, amino, oxo, hydroxy, carboxy, R², —OR², —C(O)R², —C(O)OR², —OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵, —NR³C(O)NR⁴R⁵, —NR³C(O)OR⁴ or —OC(O)NR⁴R⁵, and
any ring system in R¹ may be optionally substituted by one, two or three substituents independently selected from halo, cyano, oxo, hydroxy, carboxy, R², —OR², —C(O)R², —C(O)OR²,—OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵,—NR³C(O)NR⁴R⁵, —NR³C(O)OR⁴ or —OC(O)NR⁴R⁵;

R² is at each occurrence independently selected from C₁-C₆ alkyl, C₂-C₆ alkenyl, C₂-C₆ alkynyl or C₃-C₆ cycloalkyl, where
any alkyl, alkenyl, alkynyl or cycloalkyl in R² may be optionally substituted by one, two or three substituents independently selected from halo, cyano, oxo, R^{2a}, —OR^{2a}, —C(O)R^{2a}, —C(O)OR^{2a}, —OC(O)R^{2a}, —C(O)NR^{2a}R^{2b}, —NR^{2a}C(O)R^{2b}, —NR^{2a}R^{2b}, —SO₂NR^{2a}R^{2b}, —NR^{2a}SO₂R^{2b}, —SR^{2a}, —SO₂R^{2a}, —SO₂OR^{2a}, —OSO₂R^{2a}, —NR^{2a}SO₂NR^{2b}R^{2c}, —NR^{2a}C(O)NR^{2b}R^{2c}, —NR^{2a}C(O)OR^{2b} or —OC(O)NR^{2a}R^{2b};

R^{2a}, R^{2b} and R^{2c} are at each occurrence independently selected from hydrogen or unsubstituted C₁-C₄ alkyl;

R³, R⁴ and R⁵ are at each occurrence independently selected from hydrogen, C₁-C₆ alkyl or C₃-C₆ cycloalkyl, where
any alkyl or cycloalkyl in R³, R⁴ and R⁵ may be optionally substituted by one, two or three substituents independently selected from halo, cyano, oxo, hydroxy, carboxy, R², —OR², —C(O)R² or —C(O)OR², and
when R⁴ is optionally substituted C₁-C₆ alkyl and R⁵ is optionally substituted C₁-C₆ alkyl, then R⁴ and R⁵ together with the nitrogen atom to which they are attached in —C(O)NR⁴R⁵, —NR⁴R⁵, —SO₂NR⁴R⁵, —NR³SO₂NR⁴R⁵, —R³C(O)NR⁴R⁵ or —OC(O)NR⁴R⁵ may form a 3- to 6-membered heterocycloalkyl;

R⁶, R⁷ and R⁸ are at each occurrence independently selected from hydrogen, C₁-C₆ alkyl or C₃-C₆ cycloalkyl, where
any alkyl or cycloalkyl in R⁶, R⁷ and R⁸ may be optionally substituted by one, two or three substituents independently selected from halo, cyano, oxo, hydroxy, carboxy, R^{2a}, —OR^{2a}, —C(O)R^{2a}, —C(O)OR^{2a}, —C(O)NR^{2a}R^{2b}, —NR^{2a}C(O)R^{2b}, —NR^{2a}R^{2b}, —SO₂NR^{2a}R^{2b}, —NR^{2a}SO₂R^{2b}, —SR^{2a}, —SO₂R^{2a}, —SO₂OR^{2a}, —OSO₂R^{2a}, —NR₂SO₂NR^{2a}R^{2b},—NR^{2a}C(O)NR^{2b}R^{2c}, —NR^{2a}C(O)OR^{2b} or —OC(O)NR^{2a}R^{2b};

provided at least one of Y, L², L³ and Z is not a bond;

provided —SO₂—(CH₂)₂—NH₂ in Formula Ia is linked to the remainder of the compound of Formula Ia via a carbon atom;

provided when X is a bond, then L¹ is selected from a bond, —C(O)—, —OC(O)—, —NR³C(O)—, —NR³SO₂—, —SO₂— and —OSO₂—;

provided when Y is a bond, then L² is selected from a bond, —C(O)—, —C(O)O—, —C(O)NR³—, —SO₂NR³—, —SO₂— and —SO₂O; and

provided the compound is not

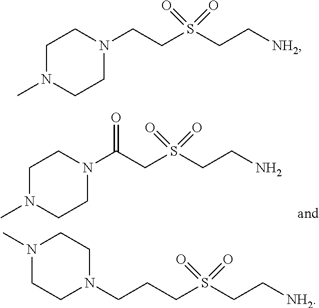

3. A compound of clause 1 or clause 2, or a pharmaceutically acceptable salt thereof, wherein the ring carbon atoms of the cyclic diamine moiety are unsubstituted.
4. A compound of any one of the preceding clauses, or a pharmaceutically acceptable salt thereof, wherein the cyclic diamine moiety is bridged by —(CR⁹R¹⁰)ₒ—, where R⁹ and R¹⁰ are at each occurrence independently selected from H and unsubstituted C₁-2 alkyl.
5. A compound of any one of the preceding clauses, or a pharmaceutically acceptable salt thereof, wherein the cyclic diamine moiety is bridged by —(CR⁹R¹⁰)ₒ—, where o is 1 or 2.
6. A compound of any one of clauses 1, 3 and 4 wherein the cyclic diamine moiety is linked through a spiro carbon atom and the numbers n and m are independently selected from 3 and 4.
7. A compound of any one of the preceding clauses, or a pharmaceutically acceptable salt thereof, wherein X is selected from a bond, unsubstituted phenyl or unsubstituted 5- to 6-membered heteroaryl.
8. A compound of any one of the preceding clauses, or a pharmaceutically acceptable salt thereof, wherein X is selected from a bond or unsubstituted phenyl.
9. A compound of any one of the preceding clauses, wherein the compound of the structure of Formula (I) is a compound of the structure of Formula (III):

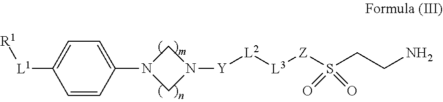

or a pharmaceutically acceptable salt thereof.
10. A compound of any one of the preceding clauses, or a pharmaceutically acceptable salt thereof, wherein R¹ is C₁-C₆ alkyl optionally substituted by one, two or three substituents independently selected from halo, cyano, amino, oxo, hydroxy or carboxy.
11. A compound of any one of the preceding clauses, or a pharmaceutically acceptable salt thereof, wherein R¹ is unsubstituted C₁-C₆ alkyl or C₁-C₆ alkyl substituted by hydroxy.
12. A compound of any one of the preceding clauses, or a pharmaceutically acceptable salt thereof, wherein R¹ is unsubstituted C₁-C₄ alkyl.
13. A compound of any one of the preceding clauses, or a pharmaceutically acceptable salt thereof, wherein L¹ is selected from a bond, —O—, —C(O)—, —C(O)O—, —OC(O)—, —C(O)NR³—, —NR³C(O)— or —SO₂—, in particular L¹ is selected from a bond, —O—, —C(O)—, —C(O)O—, —OC(O)—, —C(O)NH—, —NHC(O)— or —SO₂—.
14. A compound of any one of the preceding clauses, or a pharmaceutically acceptable salt thereof, wherein L¹ is selected from a bond, —O—, —C(O)— or —C(O)NH—, particularly L¹ is selected from a bond or —O—.
15. A compound according to any one of the preceding clauses wherein L¹ is selected from a bond, —O—, —C(O)—, —C(O)NH— or —SO₂—
16. A compound of any one of clauses 1-5, wherein the compound of the structure of Formula (I) is a compound of the structure of Formula (VIII):

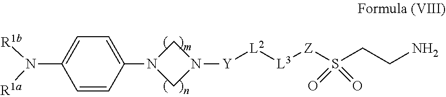

or a pharmaceutically acceptable salt thereof, wherein

R^{1a} and R^{1b} together form a 3- to 7-membered heterocycloalkyl, optionally including one additional heteroatom selected from O, N or S in the ring,
said heterocyclalkyl formed by R^{1a} and R^{1b} may be optionally substituted by one, two or three substituents independently selected from halo, cyano, oxo, hydroxy, carboxy, R², —OR², —C(O)R², —C(O)OR²,—OC(O)R², —C(O)NR⁴R⁵, —NR³C(O)R⁴, —NR³R⁴, —SO₂NR³R⁴, —NR³SO₂R⁴, —SR³, —SO₂R³, —SO₂OR³, —OSO₂R³, —NR₂SO₂NR⁴R⁵, —NR³C(O)NR⁴R⁵, —NR³C(O)OR⁴ or —OC(O)NR⁴R⁵.
17. A compound of any one of clauses 1-5, wherein the compound of the structure of Formula (I) is a compound of the structure of Formula (X):

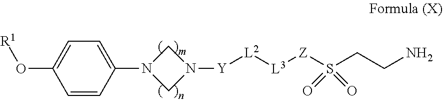

or a pharmaceutically acceptable salt thereof, wherein
R¹ is C₁-C₆ alkyl optionally substituted by one, two or three substituents independently selected from halo, cyano, amino, oxo, hydroxy or carboxy, in particular R¹ is unsubstituted C₁-C₆ alkyl or C₁-C₆ alkyl substituted by hydroxy, more particularly R¹ is unsubstituted C₁-C₄ alkyl.
18. A compound of any one of the preceding clauses, or a pharmaceutically acceptable salt thereof, wherein L² is selected from a bond, —O—, —C(O)—, —C(O)O—, —OC(O)—, —C(O)NR³— or—NR³C(O)—, in particular L² is selected from a bond, —O—, —C(O)—, —C(O)O—, —OC(O)—, —C(O)NH— or —NHC(O)—, more particularly L² is selected from a bond, —C(O)— or —C(O)NH—.
19. A compound of any one of the preceding clauses, or a pharmaceutically acceptable salt thereof, wherein L² is selected from a bond, —O—, —C(O)— or —C(O)NH—.
20. A compound of any one of the preceding clauses, or a pharmaceutically acceptable salt thereof, wherein L² is selected from a bond or —C(O)—, in particular —C(O)—.
21. A compound of any one of the preceding clauses, or a pharmaceutically acceptable salt thereof, wherein L³ is selected from a bond or C₁-C₄ alkylene, where
   any alkylene L₃ may be optionally substituted by one or two substituents independently selected from halo, cyano, oxo, hydroxy, carboxy, R², —OR², —C(O)R², —C(O)OR²,—OC(O)R², —C(O)NR⁶R⁷, —NR⁶C(O)R⁷, —NR⁶R⁷, —SO₂NR⁶R⁷, —NR⁶SO₂R⁷, —SR⁶, —SO₂R⁶, —SO₂OR⁶, —OSO₂R⁶, —NR⁶SO₂NR⁷R⁸, —NR⁶C(O)NR⁷R⁸, —NR⁶C(O)OR⁷ or —OC(O)NR⁶R⁷.
22. A compound of any one of the preceding clauses, or a pharmaceutically acceptable salt thereof, wherein L³ is selected from a bond, unsubstituted C₁-C₄ alkylene, C₁-C₄ alkylene substituted by one or two C₁₋₄ alkyl or C₁-C₄ alkylene substituted by one or two halo (e.g. F), particularly L³ is selected from a bond or unsubstituted C₁-C₄ alkylene.
23. A compound of any one of the preceding clauses, or a pharmaceutically acceptable salt thereof, wherein L³ is unsubstituted C₁-C₄ alkylene.
24. A compound of any one of the preceding clauses, or a pharmaceutically acceptable salt thereof, wherein Y is selected from a bond, unsubstituted phenyl or unsubstituted 5- to 6-membered heteroaryl.
25. A compound of any one of the preceding clauses, or a pharmaceutically acceptable salt thereof, wherein Y is selected from a bond or unsubstituted phenyl.
26. A compound of any one of the preceding clauses, or a pharmaceutically acceptable salt thereof, wherein Y is a bond.
27. A compound of any one of the preceding clauses, or a pharmaceutically acceptable salt thereof, wherein Z is selected from a bond, C₃-C₆ cycloalkyl, C₃-C₆ heterocycloalkyl, phenyl or 5- to 6-membered heteroaryl,
   any cycloalkyl, heterocycloalkyl, phenyl or heteroaryl in Z may be optionally substituted by one, two or three substituents independently selected from halo, cyano, oxo, hydroxy or carboxy; and
   any heterocycloalkyl or heteroaryl in Z including 1 or 2 heteroatoms selected from N, O or S in the ring.
28. A compound of any one of the preceding clauses, or a pharmaceutically acceptable salt thereof, wherein Z is selected from a bond or unsubstituted phenyl.
29. A compound of any one of the preceding clauses, or a pharmaceutically acceptable salt thereof, wherein Z is a bond.
30. A compound of any of the preceding clauses, or a pharmaceutically acceptable salt thereof, wherein m and n are each independently selected from 2 or 3.
31. A compound according to any one of the preceding clauses wherein L³ is unsubstituted C₁-C₄ alkylene.
32. A compound according to any one of the preceding clauses wherein L³ is C₁-C₄ alkylene optionally substituted with one or more substituents selected from R², halo or cyano.
33. A compound according to any one of the preceding clauses wherein L³ is methylene optionally substituted with one or more substituents selected from chloro, fluoro or methyl.
34. A compound according to any one of the preceding clauses wherein Y is bond, L² is C(O), L³ is CH(F), Z is a bond and q is 0.
35. A compound of any of the preceding clauses, or a pharmaceutically acceptable salt thereof, wherein the cyclic diamine moiety of Formula I or Ia is selected from:

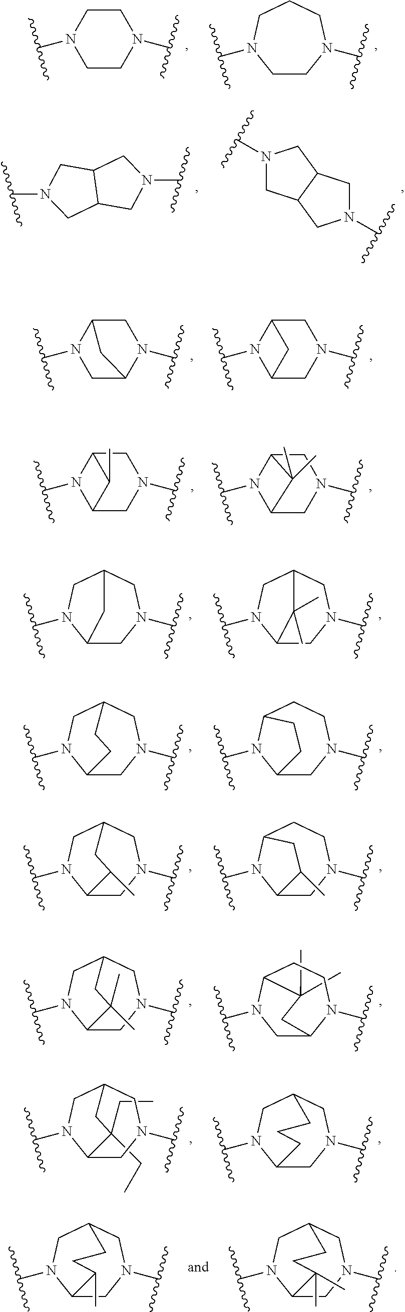

36. A compound of any one of the preceding clauses, or a pharmaceutically acceptable salt thereof, wherein the cyclic diamine moiety of Formula I or Ia is selected from:

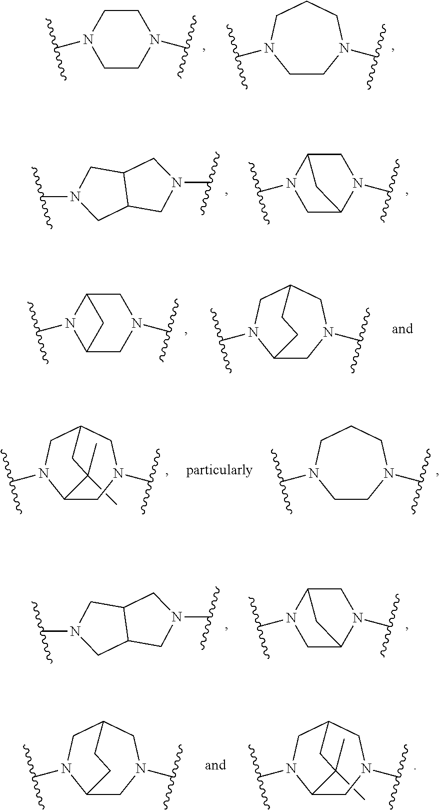

37. A compound of any one of the preceding clauses, or a pharmaceutically acceptable salt thereof, wherein the cyclic diamine moiety of Formula I or 1a is selected from:

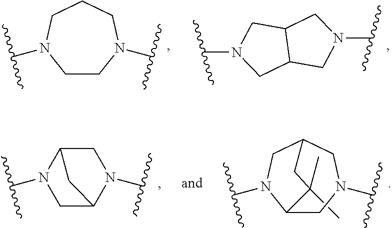 [SMILES: *N1CCCN(*)CC1 ; *N1CC2CN(*)CC2C1 ; *N1CC2CC1CN2* ; *N1CC2CN(*)C(C1)C(C)(C)C2]

38. A compound of any one of the preceding clauses, or a pharmaceutically acceptable salt thereof, wherein the cyclic diamine moiety of Formula I or 1a is

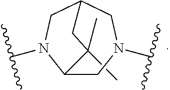 [SMILES: *N1CC2CN(*)C(C1)C(C)(C)C2]

39. A compound in accordance with clause 1, wherein the compound is selected from:

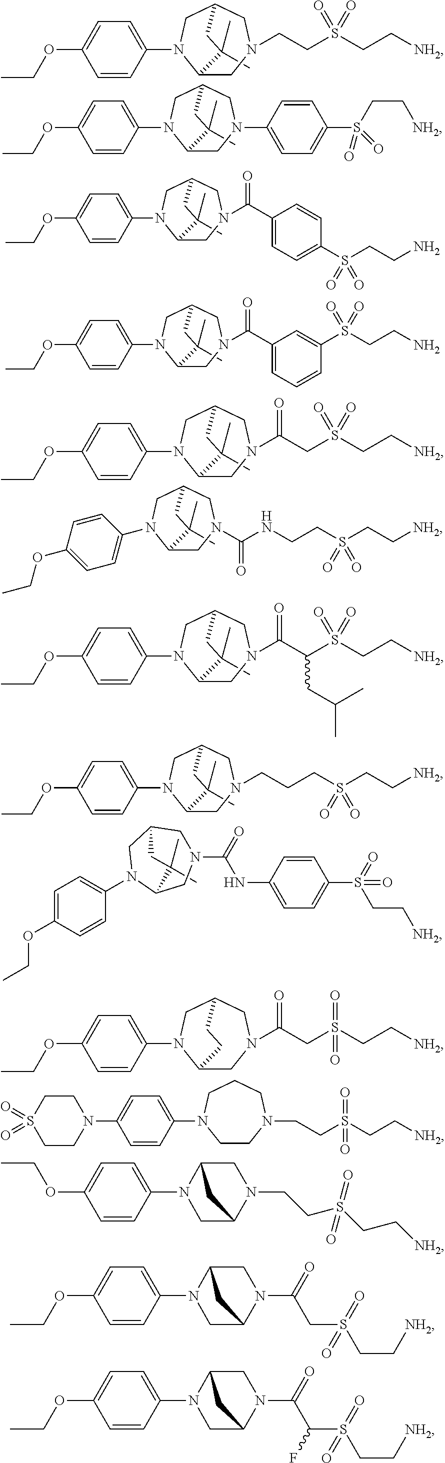

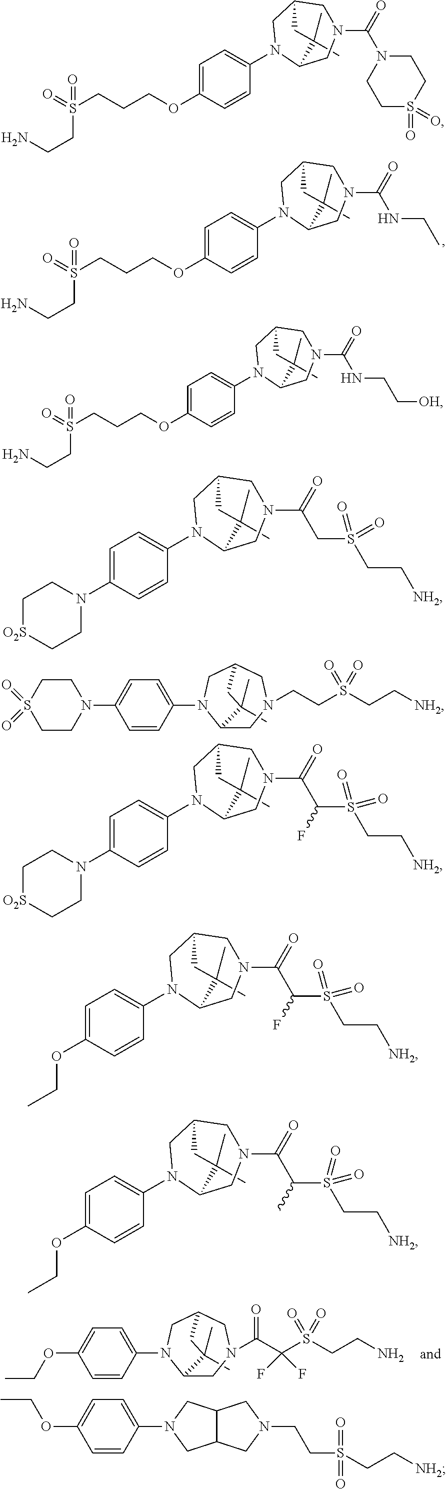

or a compound selected from Table 2;
or a pharmaceutically acceptable salt of any of the foregoing compounds.
40. A compound in accordance with of any one of clauses 1 to 39 for use as a medicament.
41. A compound in accordance with of any one of clauses 1 to 39, wherein the compound is for use in the treatment of a disease or medical condition mediated by LOX.
42. A compound in accordance with of any one of clauses 1 to 39, wherein the compound is for use in the manufacture of a medicament for the treatment of a disease or medical condition mediated by LOX.
43. A method of treating a disease or medical condition mediated by LOX in a subject in need thereof, the method comprising administering to the subject an effective amount of a compound in accordance with any one of claims 1 to 39, or a pharmaceutically acceptable salt thereof.
44. A compound of any one of clauses 1 to 39, wherein the compound is for use in the treatment of a proliferative disease.
45. A compound of clause 44, wherein the proliferative disease is cancer.
46. A compound of any one of clauses 1 to 39 for use in the treatment or prevention of cancer associated with overexpression of EGFR.
47. A compound for use in accordance with clause 46, wherein the cancer is selected from the group consisting of: small cell carcinoma (SCLC), non-small cell carcinoma of the lung (NSCLC), pancreatic cancer, squamous cells carcinoma, skin cancer, thyroid cancer, colorectal cancer, prostate cancer, renal cancer, breast cancer, head and neck cancer, glioma, mesothelioma, epidermal carcinomas, ovarian cancer, cervical cancer, bladder cancer, oesophageal cancer and a biliary cancer, such as cholangiocarcinoma.
48. A compound for use in accordance with clause 46 or 47, wherein the compound is a lysyl oxidase inhibitor and downregulates expression of MATN2 and/or activation of SMAD2 as measured by upregulation of pSMAD2.
49. A compound for use in accordance with clause 46 or 47, wherein the compound is a lysyl inhibitor and inhibits maturation of lysyl oxidase.
50. A compound for use in accordance with clause 46 or 47, wherein the compound is a lysyl inhibitor and inhibits the catalytic activity of lysyl oxidase.
51. A compound for use in accordance with clause 46 or 47, wherein the compound is a lysyl oxidase inhibitor that does not inhibit MAO-A and/or MAO-B.
52. A compound for use in accordance with clause 51, wherein the compound is a lysyl oxidase inhibitor that does not inhibit DAO.
53. A compound of any one of clauses 1 to 39, wherein the compound is for use in the treatment a fibrotic disease, such as liver fibrosis, lung fibrosis, kidney fibrosis, cardiac fibrosis, myelofibrosis or schleroderma.
54. A method of treating or preventing cancer in a subject, said method comprising administering a therapeutically effective amount of a lysyl oxidase inhibitor to said subject, wherein said subject has a cancer associated with overexpression of EGFR and the lysyl oxidase inhibitor is a compound in accordance with any one of clauses 1 to 39.
55. A method in accordance with clause 54, wherein said method comprises determining the level EGFR in a biological sample of said subject, and administering a lysyl oxidase inhibitor to said subject when the presence of EGFR is determined to be overexpressed in the biological sample.
56. A method in accordance with clause 54 or claim 55, wherein the method further comprises the steps of determining the level of one or more of MATN2, pSMAD2 or HTRA1 in a biological sample of said subject, and administering a lysyl oxidase inhibitor to said subject in response to one or more of the following:
   a) the level of MATN2 is greater than a reference sample;
   b) the level of pSMAD2 is lower than a reference sample; or
   c) the level of HTRA1 is greater than a reference sample and the level of pSMAD2 is lower than a reference sample.
57. A method in accordance with any of clauses 54 to 56, wherein said subject has a cancer selected from the group consisting of: small cell carcinoma (SCLC), non-small cell carcinoma of the lung (NSCLC), pancreatic cancer, squamous cells carcinoma, skin cancer, thyroid cancer, colorectal cancer, prostate cancer, renal cancer, breast cancer, head and neck cancer, glioma, mesothelioma, epidermal carcinomas, ovarian cancer, cervical cancer, bladder cancer and oesophageal cancer and a biliary cancer, such as cholangiocarcinoma.
58. A method in accordance with any of clauses 54 to 57, wherein the lysyl oxidase inhibitor downregulates expression of MATN2 and/or upregulates pSMAD2.
59. A method in accordance with any of clauses 54 to 58, wherein the lysyl inhibitor inhibits: maturation of lysyl oxidase, catalytic activity of lysyl oxidase or both maturation and catalytic activity.
60. A method in accordance with any of clauses 54 to 59, wherein the lysyl oxidase inhibitor does not inhibit MAO-A and/or MAO-B.
61. A method of determining a treatment regimen for a subject with cancer, comprising:
   a) determining the level of one or more of EGFR, MATN2 and HTRA1 in a biological sample; and
   b) administering a treatment regimen comprising a therapeutically effective amount of a compound of any one of clauses 1 to 39, when levels of one or more of EGFR, MATN2 and HTRA1 are elevated compared to a reference sample. 62. The method in accordance with clause 61, wherein the HTRA1 is homotrimeric HTRA1.
63. A pharmaceutical composition comprising a compound according to any one of clauses 1 to 39 and a pharmaceutically acceptable carrier.
64. A pharmaceutical composition according to clause 63, further comprising an additional therapeutically active ingredient.

### REFERENCES

Guy, C. T., Cardiff, R. D. & Muller, W. J. Induction of mammary tumors by expression of polyomavirus middle T oncogene: a transgenic mouse model for metastatic disease. Mol Cell Biol 12, 954-961 (1992)
Hanson, G. T., Aggeler, R., Oglesbee, D., Cannon, M., Capaldi, R. A., Tsien, R. Y., Remington J. S. (2004). Investigating mitochondrial redox potential with redox-sensitive green fluorescent protein indicators. J. Biol. Chem. 279, 13044-13053.
Abourbih, D. A., et al. (2010). “Lysyl oxidase expression and inhibition in uveal melanoma.” Melanoma research 20(2): 97-106.
Adam, O., et al. (2011). “Increased lysyl oxidase expression and collagen cross-linking during atrial fibrillation.” J. Mol. Cell. Cardiol. 50(4): 678-685.
Akiri, G., et al. (2003). “Lysyl oxidase-related protein-1 promotes tumor fibrosis and tumor progression in vivo.” Cancer research 63(7): 1657-1666.
Albinger-Hegyi, A., et al. (2010). “Lysyl oxidase expression is an independent marker of prognosis and a predictor of lymph node metastasis in oral and oropharyngeal squamous cell carcinoma (OSCC).” International journal of cancer Journal international du cancer 126(11): 2653-2662.
Alexandrescu, D. T. (2009). “Treatment of skin disorders with EGFR inhibitors”. WO2009091889A1
Anderson, C., et al. (2007). “Chemical genetics suggests a critical role for lysyl oxidase in zebrafish notochord morphogenesis.” Mol Biosyst 3(1): 51-59.
Aslam, T., et al. (2015). “Optical molecular imaging of lysyl oxidase activity—detection of active fibrogenesis in human lung tissue.” Chemical Science 6(8): 4946-4953.
Baker, A.-M., et al. (2013). “Lysyl oxidase plays a critical role in endothelial cell stimulation to drive tumor angiogenesis.” Cancer research 73(2): 583-594.
Baker, A.-M., et al. (2011). “The role of lysyl oxidase in SRC-dependent proliferation and metastasis of colorectal cancer.” Journal of the National Cancer Institute 103(5): 407-424.
Barker, H. E., et al. (2013). “Tumor-Secreted LOXL2 Activates Fibroblasts through FAK Signaling.” Molecular Cancer Research 11(11): 1425-1436.
Barker, H. E., et al. (2011). “LOXL2-mediated matrix remodeling in metastasis and mammary gland involution.” Cancer research 71(5): 1561-1572.
Barker, H. E., et al. (2012). “The rationale for targeting the LOX family in cancer.” Nature reviews Cancer 12(8): 540-552.
Barry-Hamilton, V., et al. (2010). “Allosteric inhibition of lysyl oxidase-like-2 impedes the development of a pathologic microenvironment.” Nat Med 16(9): 1009-1017.
Beerlage, C., et al. (2013). “Hypoxia-inducible factor 1-regulated lysyl oxidase is involved in Staphylococcus aureus abscess formation.” Infect. Immun. 81(7): 2562-2573.
Bianco, R., et al. (2007). “Rational bases for the development of EGFR inhibitors for cancer treatment.” The International Journal of Biochemistry & Cell Biology 39(7-8): 1416-1431.
Bondareva, A., et al. (2009). “The lysyl oxidase inhibitor, beta-aminopropionitrile, diminishes the metastatic colonization potential of circulating breast cancer cells.” PLoS One 4(5): e5620.
Borad, M. J., et al. (2014). “Targeted chemotherapy of cancer using EGFR inhibitors”. WO2014145751A2
Boufraqech, M., et al. (2015). “miR30a Inhibits LOX Expression and Anaplastic Thyroid Cancer Progression.” Cancer research 75(2): 367-377.
Brasselet, C., et al. (2005). “Collagen and elastin cross-linking: A mechanism of constrictive remodeling after arterial injury.” Am. J. Physiol. 289(5, Pt. 2): H2228-H2233.
Burchardt, E. R. (2006). “Preparation of 2-phenyl-3-pyridazinones as lysyl oxidase inhibitors”. DE102004056226A1
Burke A. A., et al (2017) Comparing hydrazine-derived reactive groups as inhibitors of quinone-dependent amine oxidases, Journal of Enzyme Inhibition and Medicinal Chemistry, 32:1, 496-503,
Carrington, M. J., et al. (1984). “The inhibition of lysyl oxidase in vivo by isoniazid and its reversal by pyridoxal. Effect on collagen cross-linking in the chick embryo.” Biochem J 221(3): 837-843.
Chang, J. et al (2017) Pre-clinical evaluation of small molecule LOXL2 inhibitors in breast cancer. Oncotarget, 8(16):26066-26078
Chanoki, M., et al. (1995). “Increased expression of lysyl oxidase in skin with scleroderma.” Br J Dermatol 133(5): 710-715.
Chen, W.-C., et al. (2015). “Matrix-Stiffness-Regulated Inverse Expression of Kruppel-Like Factor 5 and Kruppel-Like Factor 4 in the Pathogenesis of Renal Fibrosis.” The American Journal of Pathology 185(9): 2468-2481.
Chen, R T et al. (2017) “Blocking Surgically Induced Lysyl Oxidase Activity Reduces the Risk of Lung Metastases”. Cell Reports 19(4), 774-784
Chien, J. W., et al. (2014). “Serum lysyl oxidase-like 2 levels and idiopathic pulmonary fibrosis disease progression.” European Respiratory Journal 43(5): 1430-1438.
Cox, T. R., et al. (2013). “LOX-Mediated Collagen Crosslinking Is Responsible for Fibrosis-Enhanced Metastasis.” Cancer research 73(6): 1721-1732.
Cox, T. R., et al. (2015). “The hypoxic cancer secretome induces pre-metastatic bone lesions through lysyl oxidase.” Nature 522(7554): 106-110.
Crowley, V. et al. (2016). “Development of Glucose Regulated Protein 94-Selective Inhibitors Based on the Bnlm and Radamide Scaffold.” J Med. Chem. 59, 3471-3488.
Curtis, M. et al. (2013). “Phenicol antibacterials.” US2013/0237502A1.
da Silva, R., et al. (2015). “LOX Expression and Functional Analysis in Astrocytomas and Impact of IDH1 Mutation.” PLoS One 10(3): e0119781.
Decitre, M., et al. (1998). “Lysyl oxidase-like protein localizes to sites of de novo fibrinogenesis in fibrosis and in the early stromal reaction of ductal breast carcinomas.” Lab. Invest. 78(2): 143-151.
Dentillo, D. B., et al. (2010). “Deregulation of LOXL1 and HTRA1 Gene Expression in Endometriosis.” Reproductive Sciences 17(11): 1016-1023.
Di Donato, A., et al. (1997). “Lysyl oxidase expression and collagen cross-linking during chronic adriamycin nephropathy.” Nephron 76(2): 192-200.
Dong, K.-f., et al. (2014). “Effects on apoptosis and chemosensitivity in human laryngeal cancer Hep-2 cells by silencing the lysyl oxidase gene expression.” Zhongguo Xiandai Yixue Zazhi 24(29): 13-17.
Dong, K., et al. (2014). “Effects of lox gene expression on proliferation, invasion and radiosensitivity of laryngeal cancer hep-2 cells.” Tianjin Yiyao 42(5): 417-420.
Erler, J. T., et al. (2009). “Hypoxia-induced lysyl oxidase is a critical mediator of bone marrow cell recruitment to form the premetastatic niche.” Cancer Cell 15(1): 35-44.
Erler, J. T., et al. (2006). “Lysyl oxidase is essential for hypoxia-induced metastasis.” Nature 440(7088): 1222-1226.
Fong, S. F., et al. (2007). “Lysyl oxidase-like 2 expression is increased in colon and esophageal tumors and associated with less differentiated colon tumors.” Genes Chromosomes Cancer 46(7): 644-655.
Gao, Y., et al. (2010). “LKB1 inhibits lung cancer progression through lysyl oxidase and extracellular matrix remodeling.” Proceedings of the National Academy of Sciences 107(44): 18892-18897.
Georges, P. C., et al. (2007). “Increased stiffness of the rat liver precedes matrix deposition: implications for fibrosis.” Am. J. Physiol. 293(6, Pt. 1): G1147-G1154.
Giboda, M., et al. (1992). “Experimental schistosomiasis mansoni: modulation of granulomas by inhibition of collagen cross-link formation. Preliminary report.” Ann Trop Med Parasitol 86(6): 631-636.
Gilad, G. M. and V. H. Gilad (2001). “β-Aminopropionitrile treatment can accelerate recovery of mice after spinal cord injury.” Eur. J. Pharmacol. 430(1): 69-72.
Gilad, G. M., et al. (2001). “Lysyl oxidase, the extracellular matrix-forming enzyme, in rat brain injury sites.” Neurosci. Lett. 310(1): 45-48.
Gilad, G. M., et al. (2005). “Evidence for increased lysyl oxidase, the extracellular matrix-forming enzyme, in Alzheimer's disease brain.” Neurosci Lett 376(3): 210-214.
Goeroegh, T., et al. (2007). “Selective upregulation and amplification of the lysyl oxidase like-4 (LOXL4) gene in head and neck squamous cell carcinoma.” J Pathol 212(1): 74-82.
Gonzalez-Santamaria, J et al, (2016) “Matrix cross-linking lysyl oxidases are induced in response to myocardial infarction and promote cardiac dysfunction”. Cardiovascular Research 109, (1), 67-78.
Gonzalez, G. E., et al. (2014). “N-acetyl-seryl-aspartyl-lysyl-proline reduces cardiac collagen cross-linking and inflammation in angiotensin II-induced hypertensive rats.” Clin. Sci. 126(1): 85-94.
Gopalan Balasubramanian. (1990) “Biphenyl-substituted guanidine derivatives useful as hypoglycaemic agents.” U.S. Pat. No. 5,302,720.
Haase, V. H. (2009). “Pathophysiological Consequences of HIF Activation.” Annals of the New York Academy of Sciences 1177(1): 57-65.
Halberg, N., et al. (2009). “Hypoxia-inducible factor 1a induces fibrosis and insulin resistance in white adipose tissue.” Mol. Cell. Biol. 29(16): 4467-4483.
Hase, H., et al. (2014). “LOXL2 Status Correlates with Tumor Stage and Regulates Integrin Levels to Promote Tumor Progression in ccRCC.” Molecular Cancer Research 12(12): 1807-1817.
He, Z. and V. Koprivica (2007). “EGFR inhibitors promote axon regeneration”. WO2007008338A1
Herranz, N., et al. (2012). “Lysyl Oxidase-like 2 Deaminates Lysine 4 in Histone H3.” Molecular Cell 46(3): 369-376.
Hornstra, I. K., et al. (2003). “Lysyl oxidase is required for vascular and diaphragmatic development in mice.” J Biol Chem 278(16): 14387-14393.
Huang, C.-S., et al. (2013). “Long-term ethanol exposure-induced hepatocellular carcinoma cell migration and invasion through lysyl oxidase activation are attenuated by combined treatment with pterostilbene and curcumin analogues.” Journal of Agricultural and Food Chemistry 61(18): 4326-4335.
Hynes, J. et al. (2009). “Imidazopyridine and imidazopyrazine compounds useful as kinase inhibitors.” WO2009/155388A1.
Ingber, D. E. and A. Mammoto (2014). “Methods of altering vascular permeability by changing the mechanical properties of extracellular matrixes using agents such as lysyl oxidase (LOX)-modulating agents and uses for treatment of diseases”. WO2014152122A2
Jiang, W.-P., et al. (2014). “Identification of the involvement of LOXL4 in generation of keratocystic odontogenic tumors by RNA-Seq analysis.” In J Oral Sci 6(1): 31-38.
Jourdan-Le Saux, C., et al. (1994). “Lysyl oxidase cDNA of myofibroblast from mouse fibrotic liver.” Biochem Biophys Res Commun 199(2): 587-592.
Jung, B. (2010). “Use of quinazoline derivatives for the treatment of viral diseases”. WO2010026029A1
Kagan, H. M. (1994). “Lysyl oxidase: mechanism, regulation and relationship to liver fibrosis.” Pathol Res Pract 190(9-10): 910-919.
Kagan, H. M. and W. Li (2003). “Lysyl oxidase: properties, specificity, and biological roles inside and outside of the cell.” Journal of cellular biochemistry 88(4): 660-672.
Kagan, H. M., et al. (1981). “Changes in aortic lysyl oxidase activity in diet-induced atherosclerosis in the rabbit.” Arteriosclerosis 1(4): 287-291.
Karagiannis, G. S., et al. (2012). “Cancer-Associated Fibroblasts Drive the Progression of Metastasis through both Paracrine and Mechanical Pressure on Cancer Tissue.” Molecular Cancer Research 10(11): 1403-1418.
Kasashima, H., et al. (2014). “Lysyl oxidase-like 2 (LOXL2) from stromal fibroblasts stimulates the progression of gastric cancer.” Cancer Letters 354(2): 438-446.
Kasashima, H., et al. (2015). “Lysyl oxidase is associated with the epithelial-mesenchymal transition of gastric cancer cells in hypoxia.” Gastric Cancer: 1-12.
Kim, Y.-M., et al. (2010). “The human lysyl oxidase-like 2 protein functions as an amine oxidase toward collagen and elastin.” Mol. Biol. Rep. 38(1): 145-149.
Kim, Y., et al. (1999). “Coexpression of the lysyl oxidase-like gene (LOXL) and the gene encoding type Ill procollagen in induced liver fibrosis.” Journal of cellular biochemistry 72(2): 181-188.
Kirschmann, D. A., et al. (2002). “A molecular role for lysyl oxidase in breast cancer invasion.” Cancer research 62(15): 4478-4483.
Kumarasamy, A., et al. (2009). “Lysyl oxidase activity is dysregulated during impaired alveolarization of mouse and human lungs.” Am. J. Respir. Crit. Care Med. 180(12): 1239-1252.
Lee, G.-H., et al. (2011). “Lysyl oxidase-like-1 enhances lung metastasis when lactate accumulation and monocarboxylate transporter expression are involved.” Oncology Letters 2(5): 831-838.
Levene, C. I., et al. (1992). “Inhibition of chick embryo lysyl oxidase by various lathyrogens and the antagonistic effect of pyridoxal.” Int J Exp Pathol 73(5): 613-624.
Levental, K. R., et al. (2009). “Matrix crosslinking forces tumor progression by enhancing integrin signaling.” Cell 139(5): 891-906.
Li, R.-k., et al. (2015). “Lysyl oxidase-like 4 (LOXL4) promotes proliferation and metastasis of gastric cancer via FAK/Src pathway.” Journal of Cancer Research and Clinical Oncology 141(2): 269-281.
Li, W., et al. (2003). “Lysyl oxidase oxidizes basic fibroblast growth factor and inactivates its mitogenic potential.” Journal of cellular biochemistry 88(1): 152-164.
Liu, G., et al. (1997). “Irreversible inhibition of lysyl oxidase by homocysteine thiolactone and its selenium and oxygen analogues. Implications for homocystinuria.” J Biol Chem 272(51): 32370-32377.
Liu, J., et al. (2014). “Correlations of lysyl oxidase with MMP2/MMP9 expression and its prognostic value in non-small cell lung cancer.” Int J Clin Exp Pathol 7(9): 6040-6047.
Lopez, B., et al. (2010). “Role of lysyl oxidase in myocardial fibrosis: from basic science to clinical aspects.” Am. J. Physiol. 299(1, Pt. 2): H1-H9.
Lopez, B., et al. (2013). “Osteopontin-mediated myocardial fibrosis in heart failure: a role for lysyl oxidase?” Cardiovasc. Res. 99(1): 111-120.
Lopez, B., et al. (2012). “Collagen Cross-Linking But Not Collagen Amount Associates With Elevated Filling Pressures in Hypertensive Patients With Stage C Heart Failure: Potential Role of Lysyl Oxidase.” Hypertension 60(3): 677-683.
Lucero, H. A. and H. M. Kagan (2006). “Lysyl oxidase: an oxidative enzyme and effector of cell function.” Cellular and molecular life sciences: CMLS 63(19-20): 2304-2316.
Lucero, H. A., et al. (2008). “Lysyl oxidase oxidizes cell membrane proteins and enhances the chemotactic response of vascular smooth muscle cells.” J Biol Chem 283(35): 24103-24117.
Ma, W. (2013). “Methods for treating alzheimer's disease by administering certain synthetic compounds”. WO2013111013A2 Maki, J. M., et al. (2002). “Inactivation of the lysyl oxidase gene Lox leads to aortic aneurysms, cardiovascular dysfunction, and perinatal death in mice.” Circulation 106(19): 2503-2509.
Mambetsariev, I., et al. (2014). “Stiffness-activated GEF-H1 expression exacerbates LPS-induced lung inflammation.” PLoS One 9(4): e92670/92671-e92670/92612, 92612 pp.
Mammoto, A., et al. (2013). “Control of lung vascular permeability and endotoxin-induced pulmonary oedema by changes in extracellular matrix mechanics.” Nature communications 4: 1759.
Mammoto, T., et al. (2013). “Role of Collagen Matrix in Tumor Angiogenesis and Glioblastoma Multiforme Progression.” The American Journal of Pathology 183(4): 1293-1305.
Marshall, D. and V. Smith (2011). “In vivo screening assays for identifying inhibitors of LOXL2 activity”. WO 2011022670
Marshall, D., et al. (2012). “Anti-LOXL2 antibody, siRNA, shRNA, ribozyme and triplex oligonucleotide to increase efficacy of antitumor agent and treat cancer”. WO2012139045A1
Martinez-Martinez, E et al. (2016). The lysyl oxidase inhibitor (β-aminopropionitrile) reduces leptin profibrotic effects and ameliorates cardiovascular remodeling in diet-induced obesity in rats. Journal of Molecular and Cellular Cardiology, 92, 96-104
Miana, M., et al. (2015). “The lysyl oxidase inhibitor β-aminopropionitrile reduces body weight gain and improves the metabolic profile in diet-induced obesity in rats.” Dis. Models Mech. 8(6): 543-551.
Millanes-Romero, A., et al. (2013). “Regulation of Heterochromatin Transcription by Snail1/LOXL2 during Epithelial-to-Mesenchymal Transition.” Molecular Cell 52(5): 746-757.
Miller, B. W., et al. (2015). “Targeting the LOX/hypoxia axis reverses many of the features that make pancreatic cancer deadly: inhibition of LOX abrogates metastasis and enhances drug efficacy.” EMBO Mol Med 7(8): 1063-1076.
Moreno-Bueno, G., et al. (2011). “Lysyl oxidase-like 2 (LOXL2), a new regulator of cell polarity required for metastatic dissemination of basal-like breast carcinomas.” EMBO Mol Med 3(9): 528-544.
Murawaki, Y., et al. (1991). “Serum lysyl oxidase activity in chronic liver disease in comparison with serum levels of prolyl hydroxylase and laminin.” Hepatology 14(6): 1167-1173.
Nave, A. H., et al. (2014). “Lysyl Oxidases Play a Causal Role in Vascular Remodeling in Clinical and Experimental Pulmonary Arterial Hypertension.” Arterioscler., Thromb., Vasc. Biol. 34(7): 1446-1458.
Neufeld, G. and V. Brekhman (2009). “Use of shRNA targeting LOXL2 gene in modulating angiogenesis and treating tumors, metastasis, fibrosis, and pulmonary alveolar proteinosis”. WO2009010974A2
Nicholson, R. I., et al. (2001). “EGFR and cancer prognosis.” European Journal of Cancer 37, Supplement 4: 9-15.
Nishikawa, R., et al. (2015). “Tumour-suppressive microRNA-29s directly regulate LOXL2 expression and inhibit cancer cell migration and invasion in renal cell carcinoma.” FEBS letters 589(16): 2136-2145.
Nuthakki, V. K., et al. (2004). “Lysyl oxidase expression in a rat model of arterial balloon injury.” J Vasc Surg 40(1): 123-129.
Offenberg, H., et al. (2008). “TIMP-1 expression in human colorectal cancer is associated with TGF-B1, LOXL2, INHBA1, TNF-AIP6 and TIMP-2 transcript profiles.” Mol Oncol 2(3): 233-240.
Ohmura, H., et al. (2012). “Cardiomyocyte-specific transgenic expression of lysyl oxidase-like protein-1 induces cardiac hypertrophy in mice.” Hypertens. Res. 35(11): 1063-1068.
Osawa, T., et al. (2013). “Lysyl oxidase secreted by tumour endothelial cells promotes angiogenesis and metastasis.” Br J Cancer 109(8): 2237-2247.
Palfreyman, M. G., et al. (1989). “Preparation of allylamines, inhibitors of lysyl oxidase”. EP330218A2
Papadantonakis, N., et al. (2012). “Megakaryocyte pathology and bone marrow fibrosis: the lysyl oxidase connection.” Blood 120(9): 1774-1781.
Park, H.-Y. L., et al. (2014). “Lysyl oxidase-like 2 level and glaucoma surgical outcomes.” Invest. Ophthalmol. Visual Sci. 55(5): 3337-3343.
Peinado, H., et al. (2005). “A molecular role for lysyl oxidase-like 2 enzyme in snail regulation and tumor progression.” EMBO J 24(19): 3446-3458.
Peinado, H., et al. (2008). “Lysyl Oxidase-Like 2 as a New Poor Prognosis Marker of Squamous Cell Carcinomas.” Cancer research 68(12): 4541-4550.
Peng, L., et al. (2009). “Secreted LOXL2 is a novel therapeutic target that promotes gastric cancer metastasis via the Src/FAK pathway.” Carcinogenesis 30(10): 1660-1669.
Pickup, M. W., et al. (2013). “Stromally Derived Lysyl Oxidase Promotes Metastasis of Transforming Growth Factor-β-Deficient Mouse Mammary Carcinomas.” Cancer Res. 73(17): 5336-5346.
Pinnell, S. R. and G. R. Martin (1968). “The cross-linking of collagen and elastin: enzymatic conversion of lysine in peptide linkage to alpha-aminoadipic-delta-semialdehyde (allysine) by an extract from bone.” Proceedings of the National Academy of Sciences 61(2): 708-716.
Priyanka, T. et al. (2016) “Lysyl oxidase (LOX) inhibitors as anti-scarring agents” Abstracts of Papers, 252nd ACS National Meeting & Exposition, Philadelphia, Pa., United States, Aug. 21-25, 2016, BIOL-98
Ree, A. H., et al. (2008). “Treatment and diagnosis of metastatic prostate cancer with inhibitors of epidermal growth factor receptor (EGFR)”. WO2008125633A2 Reynault, O. et al. (1997). “A convenient synthesis of new halothienyl β-aminoacids as versatile building block.” Org. Prep. Proc. Int. 29(4): 488-494.
Ricard-Blum, S., et al. (1996). “Mechanism of collagen network stabilization in human irreversible granulomatous liver fibrosis.” Gastroenterology 111(1): 172-182.
Rimar, D., et al. (2014). “Brief report: lysyl oxidase is a potential biomarker of fibrosis in systemic sclerosis.” Arthritis Rheumatol 66(3): 726-730.
Roehrig, F et al. (2017) “VEGF-ablation therapy reduces drug delivery and therapeutic response in ECM-dense tumors” Oncogene 36(1), 1-12.
Romero, F. et al. (2016). “4,5,6,7-Tetrahydro-1-H-pyrazolo[4,3-C]pyrimidine-3-amine compounds as CBP and/or EP300 inhibitors.” WO2016/086200A1
Rosin, N. L., et al. (2015). “Disruption of Collagen Homeostasis Can Reverse Established Age-Related Myocardial Fibrosis.” Am. J. Pathol. 185(3): 631-642.
Rowbottom, M. W. et al. (2016a). “Preparation of substituted pyridinylmethylamine compounds as lysyl oxidase-like 2 inhibitors”. WO2016144702
Rowbottom, M. W. et al. (2016b). “Preparation of fluorinated pyridine derivatives as lysyl oxidase-like 2 inhibitors and uses thereof.” WO2016144703
Rowbottom, M. W.; Hutchinson, J. H. (2017a). “Preparation of pyrimidine derivatives as Lysyl oxidase-like 2 inhibitors useful for the treatment of fibrosis.” WO2017003862
Rowbottom, M. W.; Hutchinson, J. H. (2017b). “Lysyl oxidase-like 2 inhibitors and uses thereof.” WO2017015221
Ruiz, L. A., et al. (2011). “Single-nucleotide polymorphisms in the lysyl oxidase-like protein 4 and complement component 3 genes are associated with increased risk for endometriosis and endometriosis-associated infertility.” Fertil Steril 96(2): 512-515.
Sansom, O. (2012). Targeting the tumour microenvironment in pancreatic cancer. EACR-IACR Joint Conference: Tumour Microenvironment, Dublin, Ireland.
Sayre, L. M. (2007). “Amine compounds for amine oxidase inhibitors, and therapeutic use”. WO2007005737A2
Schietke, R., et al. (2010). “The lysyl oxidases LOX and LOXL2 are necessary and sufficient to repress E-cadherin in hypoxia: insights into cellular transformation processes mediated by HIF-1.” J Biol Chem 285(9): 6658-6669.
Schlotzer-Schrehardt, U., et al. (2008). “Genotype-correlated expression of lysyl oxidase-like 1 in ocular tissues of patients with pseudoexfoliation syndrome/glaucoma and normal patients.” American Journal of Pathology 173(6): 1724-1735.
Schohe-Loop, R., et al. (2003). “Preparation of 2-phenyl-3(2H)-pyridazinones as lysyl oxidase inhibitors for preventing and treating fibrosis”. DE10216144A1
Schuetze, F., et al. (2015). “Inhibition of Lysyl Oxidases Improves Drug Diffusion and Increases Efficacy of Cytotoxic Treatment in 3D Tumor Models.” Sci. Rep. 5: 17576.
Schweighauser, L., et al. (2015). “Attraction or Repulsion? London Dispersion Forces Control Azobenzene Switches.” Angew. Chem. Int ed 54(45): 13436-13439.
Scola, N. and T. Gorogh (2010). “LOXL4 as a selective molecular marker in primary and metastatic head/neck carcinoma.” Anticancer Res 30(11): 4567-4571.
Se, L Y et al. (2017) “Expression of Lysyl Oxidase Predictive of Distant Metastasis of Laryngeal Cancer”. Otolaryngology—head and neck surgery; 156(3), 489-497
Shen, C. J., et al. (2014). “Ionizing radiation induces tumor cell lysyl oxidase secretion.” BMC Cancer 14: 532/531-532/510.
Shinobu, M et al. (2016) “Lysyl oxidase is associated with increased thrombosis and platelet reactivity”. Blood; 127(11), 1493-1501
Siegel, R. C., et al. (1978). “Biochemical and immunochemical study of lysyl oxidase in experimental hepatic fibrosis in the rat.” Proceedings of the National Academy of Sciences 75(6): 2945-2949.
Smith, V. and A. K. Holzer (2010). “Chemotherapeutic methods and compositions for treating cancer by inhibiting activity of lysyl oxidase-type enzyme”. WO2010080769A2
Smith, V. and P. Van Vlasselaer (2011). “Tumor therapy by inhibiting the activity or expression of lysyl oxidase-like 2 with antibodies or inhibitory nucleic acids”. WO2011022710A1
Stalmans, I., et al. (2010). “Use of lysyl oxidase related protein inhibitors for treatment of ocular neovascularization and fibrotic damage”. WO2010091279A1
Stalmans, I., et al. (2011). “Methods of treatmenting ocular fibrosis by modulating the activity of lysyl oxidase-type enzymes”. US20110076285A1
Stewart, G. D., et al. (2008). “Analysis of hypoxia-associated gene expression in prostate cancer: lysyl oxidase and glucose transporter-1 expression correlate with Gleason score.” Oncol Rep 20(6): 1561-1567.
Tadmor, T., et al. (2013). “The expression of lysyl-oxidase gene family members in myeloproliferative neoplasms.” American Journal of Hematology 88(5): 355-358.
Tang, H et al. “Lysyl oxidase drives tumour progression by trapping EGF receptors at the cell surface.” Nat Commun. 8:14909
Tang, S. S., et al. (1984). “Beta-substituted ethylamine derivatives as suicide inhibitors of lysyl oxidase.” J Biol Chem 259(2): 975-979.
Threadgill, D. and C. J. Barrick (2007). “Use of EGFR inhibitors to prevent or treat obesity”. WO2007011702A2
Toustrup, K., et al. (2011). “Development of a hypoxia gene expression classifier with predictive impact for hypoxic modification of radiotherapy in head and neck cancer.” Cancer research 71(17): 5923-5931.
Tsang, A. W. and C. M. Furdui (2015). “Methods and compositions comprising epidermal growth factor receptor (EGFR) inhibitors for the treatment of Chlamydia infection and related diseases and disorders”. WO2015038755A1
Uzel, M. I., et al. (2001). “Multiple bone morphogenetic protein 1-related mammalian metalloproteinases process pro-lysyl oxidase at the correct physiological site and control lysyl oxidase activation in mouse embryo fibroblast cultures.” J Biol Chem 276(25): 22537-22543.
Vadasz, Z., et al. (2005). “Abnormal deposition of collagen around hepatocytes in Wilson's disease is associated with hepatocyte specific expression of lysyl oxidase and lysyl oxidase like protein-2.” J Hepatol 43(3): 499-507.
Van Bergen, T., et al. (2013). “The role of LOX and LOXL2 in scar formation after glaucoma surgery.” Invest. Ophthalmol. Visual Sci. 54(8): 5788-5796.
Van Bierbeek, A and Gingras, M. (1998) “Polysulfurated branched molecules containing functionalized m-phenylene sulfides.” Tetrahedron Lett, 39(35): 6283-6286.
van Nimwegen, M. J. and B. van de Water (2007). “Focal adhesion kinase: a potential target in cancer therapy.” Biochem Pharmacol 73(5): 597-609.
Vitalba, D S et al. (2016). “Major Action of Endogenous Lysyl Oxidase in Clear Cell Renal Cell Carcinoma Progression and Collagen Stiffness Revealed by Primary Cell Cultures” Am.
J. Pathol.; 186(9), 2473-2485 Weihua, Z., et al. (2008). “Survival of Cancer Cells Is Maintained by EGFR Independent of Its Kinase Activity.” Cancer Cell 13(5): 385-393.
Wiel, C., et al. (2013). “Lysyl oxidase activity regulates oncogenic stress response and tumorigenesis.” Cell Death Dis 4: e855.
Wilgus, M.-L., et al. (2011). “Lysyl oxidase: A lung adenocarcinoma biomarker of invasion and survival.” Cancer 117(10): 2186-2191.
Wilhelmus, M. M. M., et al. (2013). “Extracellular matrix modulator lysyl oxidase colocalizes with amyloid-beta pathology in Alzheimer's disease and hereditary cerebral hemorrhage with amyloidosis-Dutch type.” Exp. Gerontol. 48(2): 109-114.
Williamson, P. R. and H. M. Kagan (1987). “Electronegativity of aromatic amines as a basis for the development of ground state inhibitors of lysyl oxidase.” J Biol Chem 262(30): 14520-14524.
Wong, C. C.-L., et al. (2011). “Hypoxia-inducible factor 1 is a master regulator of breast cancer metastatic niche formation.” Proceedings of the National Academy of Sciences 108(39): 16369-16374.
Wong, C. C.-L., et al. (2014). “Lysyl oxidase-like 2 is critical to tumor microenvironment and metastatic niche formation in hepatocellular carcinoma.” Hepatology (Hoboken, N.J., U. S.) 60(5): 1645-1658.
Xiao Q and Ge G. (2012) “Lysyl Oxidase, Extracellular Matrix Remodeling and Cancer Metastasis” Cancer Microenviron. 5(3): 261-273
Xu, J., et al. (2014). “67 laminin receptor promotes the malignant potential of tumour cells up-regulating lysyl oxidase-like 2 expression in cholangiocarcinoma.” Digestive and Liver Disease 46(8): 750-757.
Yang, X., et al. (2013). “Inactivation of lysyl oxidase by β-aminopropionitrile inhibits hypoxia-induced invasion and migration of cervical cancer cells.” Oncol Rep 29(2): 542-548.
Yang, Z., et al. (2010). “Uric acid increases fibronectin synthesis through upregulation of lysyl oxidase expression in rat renal tubular epithelial cells.” Am. J. Physiol. 299(2, Pt. 2): F336-F346.
Zaffryar-Eilot, S., et al. (2013). “Lysyl oxidase-like-2 promotes tumour angiogenesis and is a potential therapeutic target in angiogenic tumours.” Carcinogenesis 34(10): 2370-2379.
Zenkel, M., et al. (2011). “Regulation of lysyl oxidase-like 1 (LOXL1) and elastin-related genes by pathogenic factors associated with pseudoexfoliation syndrome.” Invest Ophthalmol Vis Sci 52(11): 8488-8495.
Zhao X and Subramaian S. (2017). “Intrinsic Resistance of Solid Tumors to Immune Checkpoint Blockade Therapy” Cancer Res; 77(4), 817-822
Zhi, C et al. (2017) “Elevated ischaemia-associated lysyl oxidase activity in delayed graft failure 6-12 months after renal transplantation” Experimental Physiology 102(2), 282-287
Zhu, J., et al. (2015). “Lysyl Oxidase Is Predictive of Unfavorable Outcomes and Essential for Regulation of Vascular Endothelial Growth Factor in Hepatocellular Carcinoma.” Digestive Diseases and Sciences: 1-13.
Zibadi, S., et al. (2010). “T lymphocyte regulation of lysyl oxidase in diet-induced cardiac fibrosis.” Cardiovasc Toxicol 10(3): 190-198.

1. A compound having the structure of Formula (I): 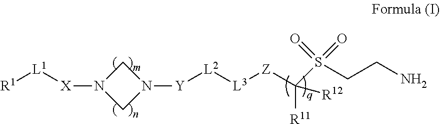
or a pharmaceutically acceptable salt thereof, wherein
m and n are each independently selected from 1, 2, 3 or 4, and where two ring carbon atoms of the cyclic diamine moiety of formula 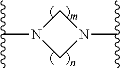
may be
(i) optionally linked by a bond,
(ii) optionally bridged by —(CR⁹R¹⁰)ₒ—, where R⁹ and R¹⁰ are at each occurrence independently selected from H and unsubstituted C₁₋₄ alkyl and o is 1, 2, 3 or 4; or
(iii) optionally linked by a spiro carbon atom; and
each ring carbon atom of said cyclic diamine moiety may be optionally substituted by one or two substituents independently selected from C₁-C₆ alkyl, C₁-C₆ alkyl substituted with —OR^{2a}, or C₁-C₆ alkyl substituted with —NR^{2a}R^{2b};
L¹ and L² are each independently selected from a bond, —O—, —C(O)—, —C(O)O—, —OC(O)—, —C(O)NR³—, —NR³C(O)—, —NR³—, —SO₂NR³—, —NR³SO₂—, —S—, —SO₂—, —SO₂O—, —OSO₂—, —NR³SO₂NR⁴—, —NR³C(O)NR⁴—, —NR³C(O)O— or —OC(O)NR³—;
L³ is selected from a bond, C₁-C₄ alkylene, C₂-C₄ alkenylene or C₂-C₄ alkynylene, where
any alkylene, alkenylene or alkynylene in L³ may be optionally substituted by one or two substituents independently selected from halo, cyano, oxo, hydroxy, carboxy, R², —OR², —C(O)R², —C(O)OR², —OC(O)R², —C(O)NR⁶R⁷, —NR⁶C(O)R⁷, —NR⁶R⁷, —SO₂NR⁶R⁷, —NR⁶SO₂R⁷, —SR⁶, —SO₂R⁶, —SO₂OR⁶, —OSO₂R⁶, —NR⁶SO₂NR⁷R⁸, —NR⁶C(O)NR⁷R⁸, —NR⁶C(O)OR⁷ or —OC(O)NR⁶R⁷;
X, Y and Z are each independently selected from a bond or a 3- to 12-membered ring system, including 0, 1, 2 or 3 heteroatoms selected from N, O or S in the ring system, where
any ring system in X, Y and Z may be optionally substituted by one or more substituents independently selected from halo, cyano, oxo, hydroxy, carboxy, R², —OR², —C(O)R², —C(O)OR²,—OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵, —R³C(O)NR⁴R⁵, —NR³C(O)OR⁴ or —OC(O)NR⁴R⁵;
R¹ is selected from hydrogen, halogen, C₁-C₆ alkyl, C₂-C₆ alkenyl, C₂-C₆ alkynyl or a 3- to 12-membered ring system (e.g. cycloalkyl, heterocyclyl, aryl or heteroaryl), including 0, 1, 2 or 3 heteroatoms selected from N, O or S in the ring system, where
any alkyl, alkenyl or alkynyl in R¹ may be optionally substituted by one, two or three substituents independently selected from halo, cyano, amino, oxo, hydroxy, carboxy, R², —OR², —C(O)R², —C(O)OR², —OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵, —NR³C(O)NR⁴R⁵, —NR³C(O)OR⁴ or —OC(O)NR⁴R⁵; and
any ring system in R¹ may be optionally substituted by one or more substituents independently selected from halo, cyano, oxo, hydroxy, carboxy, R², —OR², —C(O)R², —C(O)OR²,—OC(O)R², —C(O)NR⁴R⁵, —NR³C(O)R⁴, —NR⁴R⁵, —SO₂NR⁴R⁵, —NR³SO₂R⁴, —SR³, —SO₂R³, —SO₂OR³, —OSO₂R³, —NR³SO₂NR⁴R⁵,—NR³C(O)NR⁴R⁵, —NR³C(O)OR⁴ or —OC(O)NR⁴R⁵;
R² is at each occurrence independently selected from C₁-C₆ alkyl, C₂-C₆ alkenyl, C₂-C₆ alkynyl or C₃-C₆ cycloalkyl, where
any alkyl, alkenyl, alkynyl or cycloalkyl in R² may be optionally substituted by one, two or three substituents independently selected from halo, cyano, oxo, R^{2a}, —OR^{2a}, —C(O)R^{2a}, —C(O)OR^{2a}, —OC(O)R^{2a}, —C(O)NR^{2a}R^{2b}, —NR^{2a}C(O)R^{2b}, —NR^{2a}R^{2b}, —SO₂NR^{2a}R^{2b}, —NR^{2a}SO₂R^{2b}, —SR^{2a}, —SO₂R^{2a}, —SO₂OR^{2a}, —OSO₂R^{2a}, —NR^{2a}SO₂NR^{2b}R², —NR^{2a}C(O)NR^{2b}R^{2c}, —NR^{2a}C(O)OR^{2b} or —OC(O)NR^{2a}R^{2b};
R^{2a}, R^{2b} and R^{2c} are at each occurrence independently selected from hydrogen or unsubstituted C₁-C₄ alkyl;
R³, R⁴ and R⁵ are at each occurrence independently selected from hydrogen, C₁-C₆ alkyl or C₃-C₆ cycloalkyl, where
any alkyl or cycloalkyl in R³, R⁴ and R⁵ may be optionally substituted by one, two or three substituents independently selected from halo, cyano, oxo, hydroxy, carboxy, R², —OR², —C(O)R² or —C(O)OR², and
when R⁴ is optionally substituted C₁-C₆ alkyl and R⁵ is optionally substituted C₁-C₆ alkyl, then R⁴ and R⁵ together with the nitrogen atom to which they are attached in —C(O)NR⁴R⁵, —NR⁴R⁵, —SO₂NR⁴R⁵, —NR³SO₂NR⁴R⁵, —R³C(O)NR⁴R⁵ or —OC(O)NR⁴R⁵ may form a 3- to 6-membered heterocycloalkyl;
R⁶, R⁷ and R⁸ are at each occurrence independently selected from hydrogen, C₁-C₆ alkyl or C₃-C₆ cycloalkyl, where
any alkyl or cycloalkyl in R⁶, R⁷ and R⁸ may be optionally substituted by one, two or three substituents independently selected from halo, cyano, oxo, hydroxy, carboxy, R^{2a}, —OR^{2a}, —C(O)R^{2a}, —C(O)OR^{2a}, —C(O)NR^{2a}R^{2b}, —NR^{2a}C(O)R^{2b}, —NR^{2a}R^{2b}, —SO₂NR^{2a}R^{2b}, —NR^{2a}SO₂R^{2b}, —SR^{2a}, —SO₂R^{2a}, —SO₂OR^{2a}, —OSO₂R^{2a}, —NR₂SO₂NR^{2a}R^{2b}, —NR^{2a}C(O)NR^{2b}R^{2c}, —NR^{2a}C(O)OR^{2b} or —OC(O)NR^{2a}R^{2b};
R¹¹ and R¹² are independently selected from hydrogen and C₁-C₆ alkyl; and
q is 0, 1 or 2;
provided at least one of L², Y, L³ and Z is not a bond;
provided —SO₂—(CH₂)₂—NH₂ in Formula I is linked to the remainder of the compound of Formula I via a carbon atom;
provided when X is a bond, then L¹ is selected from a bond, —C(O)—, —OC(O)—, —NR³C(O)—, —NR³SO₂—, —SO₂— and —OSO₂—;
provided when Y is a bond, then L² is selected from a bond, —C(O)—, —C(O)O—, —C(O)NR³—, —SO₂NR³—, —SO₂— and —SO₂O; and
provided the compound is not

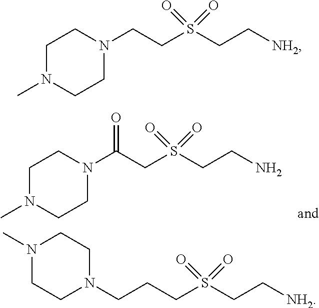

2. A compound of claim 1, or a pharmaceutically acceptable salt thereof, wherein the cyclic diamine moiety is (i) optionally linked by a bond or (ii) optionally bridged by —(CR⁹R¹⁰)ₒ—, where R⁹ and R¹⁰ are at each occurrence independently selected from H and unsubstituted C₁₋₄ alkyl and o is 1, 2, 3 or 4; and q is 0.

3. A compound of any one of claims 1 and 2, or a pharmaceutically acceptable salt thereof, wherein the ring carbon atoms of the cyclic diamine moiety are unsubstituted.

4. A compound of any one of the preceding claims, or a pharmaceutically acceptable salt thereof, wherein the cyclic diamine moiety is bridged by —(CR⁹R¹⁰)ₒ—, where R⁹ and R¹⁰ are at each occurrence independently selected from H and unsubstituted C₁₋₂ alkyl.

5. A compound of any one of the preceding claims, or a pharmaceutically acceptable salt thereof, wherein the cyclic diamine moiety is bridged by —(CR⁹R¹⁰)ₒ—, where o is 1 or 2.

6. A compound of any one of the preceding claims, or a pharmaceutically acceptable salt thereof, wherein X is selected from a bond, unsubstituted phenyl or unsubstituted 5- to 6-membered heteroaryl, particularly X is selected from a bond or unsubstituted phenyl.

7. A compound of any one of the preceding claims, wherein the compound of the structure of Formula (I) is a compound of the structure of Formula (III): 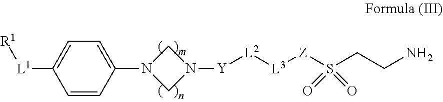
or a pharmaceutically acceptable salt thereof.

8. A compound of any one of the preceding claims, or a pharmaceutically acceptable salt thereof, wherein R¹ is C₁-C₆ alkyl optionally substituted by one, two or three substituents independently selected from halo, cyano, amino, oxo, hydroxy or carboxy, particularly R¹ is unsubstituted C₁-C₆ alkyl or C₁-C₆ alkyl substituted by hydroxy, more particularly R¹ is unsubstituted C₁-C₄ alkyl.

9. A compound of any one of the preceding claims, or a pharmaceutically acceptable salt thereof, wherein
L¹ is selected from a bond, —O—, —C(O)—, —C(O)O—, —OC(O)—, —C(O)NR³—, —NR³C(O)— or —SO₂—; or
L¹ is selected from a bond, —O—, —C(O)—, —C(O)O—, —OC(O)—, —C(O)NH—, —NHC(O)— or —SO₂—; or
L¹ is selected from a bond, —O—, —C(O)— or —C(O)NH—; or
L¹ is selected from a bond or —O—; or
L¹ is —O—.

10. A compound according to any one of the preceding claims wherein L¹ is a bond and R¹ is halogen.

11. A compound of any one of claims 1-5, wherein the compound of the structure of Formula (I) is a compound of the structure of Formula (VIII): 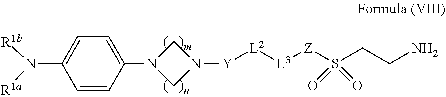
or a pharmaceutically acceptable salt thereof, wherein
R^{1a} and R^{1b} together form a 3- to 7-membered heterocycloalkyl, optionally including one additional heteroatom selected from O, N or S in the ring,
said heterocyclalkyl formed by R^{1a} and R^{1b} may be optionally substituted by one, two or three substituents independently selected from halo, cyano, oxo, hydroxy, carboxy, R², —OR², —C(O)R², —C(O)OR²,—OC(O)R², —C(O)NR⁴R⁵, —NR³C(O)R⁴, —NR³R⁴, —SO₂NR³R⁴, —NR³SO₂R⁴, —SR³, —SO₂R³, —SO₂OR³, —OSO₂R³, —NR₂SO₂NR⁴R⁵, —NR³C(O)NR⁴R⁵, —NR³C(O)OR⁴ or —OC(O)NR⁴R⁵.

12. A compound of any one of claims 1-5, wherein the compound of the structure of Formula (I) is a compound of the structure of Formula (X): 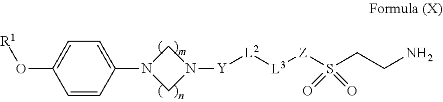
or a pharmaceutically acceptable salt thereof, wherein
R¹ is C₁-C₆ alkyl optionally substituted by one, two or three substituents independently selected from halo, cyano, amino, oxo, hydroxy or carboxy, in particular R¹ is unsubstituted C₁-C₆ alkyl or C₁-C₆ alkyl substituted by hydroxy, more particularly R¹ is unsubstituted C₁-C₄ alkyl.

13. A compound of any one of the preceding claims, or a pharmaceutically acceptable salt thereof, wherein
L² is selected from a bond, —O—, —C(O)—, —C(O)O—, —OC(O)—, —C(O)NR³— or —NR³C(O)—; or
L² is selected from a bond, —O—, —C(O)—, —C(O)O—, —OC(O)—, —C(O)NH— or —NHC(O)—; or
L² is selected from a bond, —O—, —C(O)— or —C(O)NH—; or
L² is selected from a bond, —C(O)— or —C(O)NH—;
L² is selected from a bond or —C(O)—; or
L² is —C(O)—.

14. A compound of any one of the preceding claims, or a pharmaceutically acceptable salt thereof, wherein L³ is selected from a bond or C₁-C₄ alkylene, where
any alkylene L³ may be optionally substituted by one or two substituents independently selected from halo, cyano, oxo, hydroxy, carboxy, R², —OR²,—C(O)R², —C(O)OR²,—OC(O)R², —C(O)NR⁶R⁷, —NR⁶C(O)R⁷, —NR⁶R⁷, —SO₂NR⁶R⁷, —NR⁶SO₂R⁷, —SR⁶, —SO₂R⁶, —SO₂OR⁶, —OSO₂R⁶, —NR⁶SO₂NR⁷R⁸, —NR⁶C(O)NR⁷R⁸, —NR⁶C(O)OR⁷ or —OC(O)NR⁶R⁷;
particularly L³ is selected from a bond or unsubstituted C₁-C₄ alkylene,
more particularly L³ is unsubstituted C₁-C₄ alkylene.

15. A compound of any one of the preceding claims, or a pharmaceutically acceptable salt thereof, wherein Y is selected from a bond, unsubstituted phenyl or unsubstituted 5- to 6-membered heteroaryl, particularly Y is selected from a bond or unsubstituted phenyl, more particularly Y is a bond.

16. A compound of any one of the preceding claims, or a pharmaceutically acceptable salt thereof, wherein Z is selected from a bond, C₃-C₆ cycloalkyl, C₃-C₆ heterocycloalkyl, phenyl or 5- to 6-membered heteroaryl,
any cycloalkyl, heterocycloalkyl, phenyl or heteroaryl in Z may be optionally substituted by one, two or three substituents independently selected from halo, cyano, oxo, hydroxy or carboxy; and
any heterocycloalkyl or heteroaryl in Z including 1 or 2 heteroatoms selected from N, O or S in the ring.
Particularly Z is selected from a bond or unsubstituted phenyl, more particularly Z is a bond.

17. A compound of any of the preceding claims, or a pharmaceutically acceptable salt thereof, wherein m and n are each independently selected from 2 or 3.

18. A compound of any of the preceding claims, or a pharmaceutically acceptable salt thereof, wherein the cyclic diamine moiety of Formula I is selected from:

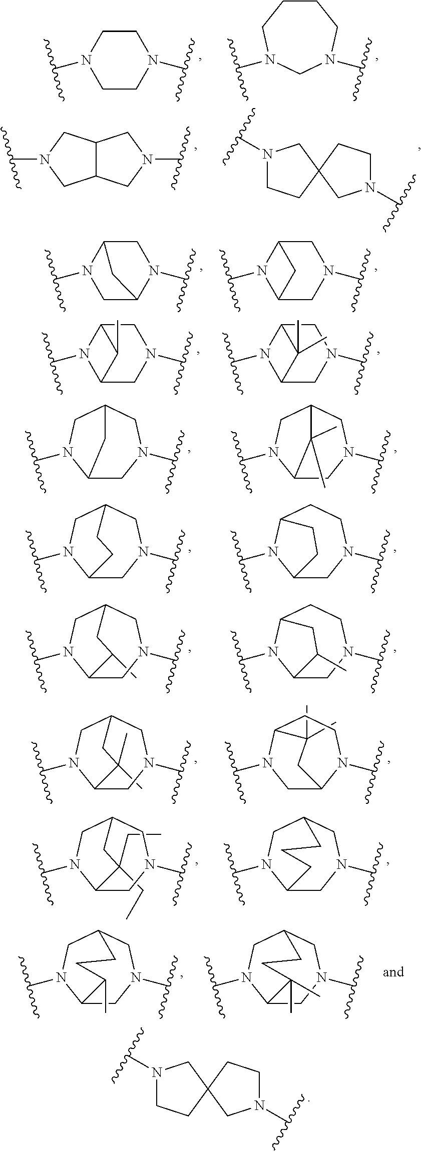

19. A compound of any one of the preceding claims, or a pharmaceutically acceptable salt thereof, wherein the cyclic diamine moiety of Formula I is selected from:

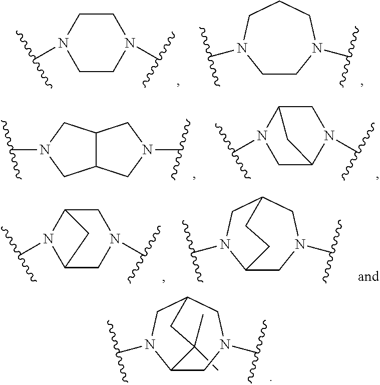

20. A compound of any one of the preceding claims, or a pharmaceutically acceptable salt thereof, wherein the cyclic diamine moiety of Formula I is selected from:

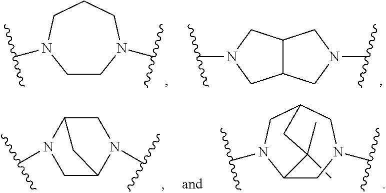

21. A compound of any one of the preceding claims, or a pharmaceutically acceptable salt thereof, wherein the cyclic diamine moiety of Formula I is

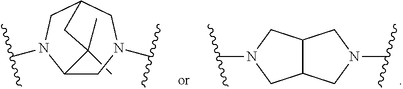 [SMILES: *N1CC2CN(*)C(C1)C(C)(C)C2 ; *N1CC2CN(*)CC2C1]

22. A compound in accordance with claim 1, wherein the compound is selected from: 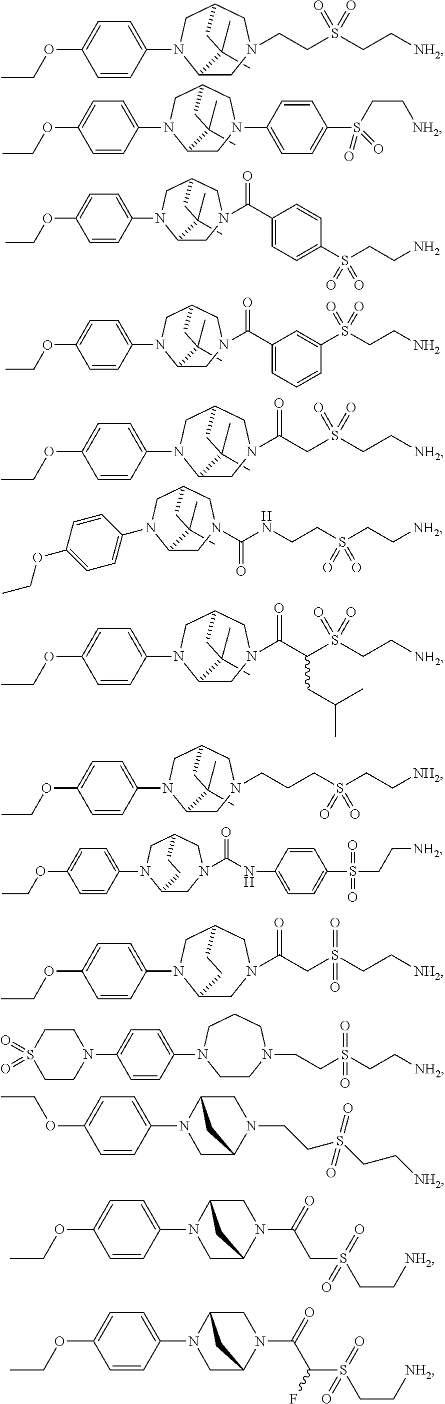 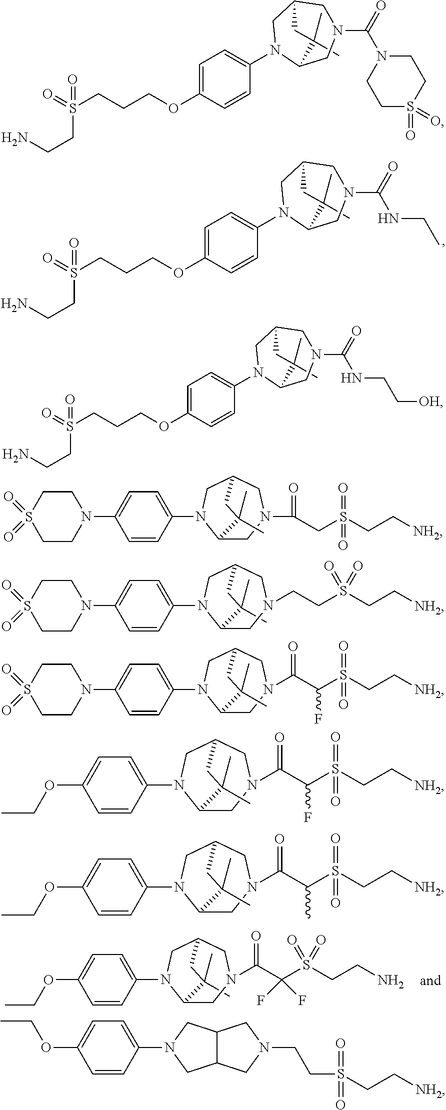
or a pharmaceutically acceptable salt of any of the foregoing compounds.

23. A compound according to claim 1, or a pharmaceutically acceptable salt thereof, selected from:

| Chemical name | Structure |
|---|---|
| Ethyl 4-((2-aminoethyl)sulfonyl)- 5-((1S,4S)-5-(4-ethoxyphenyl)- 2,5-diazabicyclo[2.2.1]heptan-2- yl)-5-oxopentanoate | 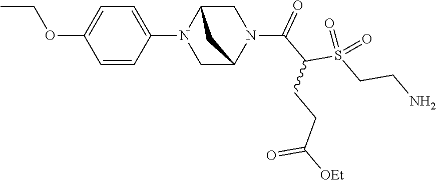 [SMILES: CCOC(=O)CCC(C(=O)N1C[C@@H]2C[C@H]1CN2c1ccc(OCC)cc1)S(=O)(=O)CCN] |
| (1-((2-Aminoethyl)sulfonyl)- cyclopropyl)((1S,4S)-5-(4- ethoxyphenyl)-2,5-diazabicyclo- [2.2.1]heptan-2-yl)methanone | 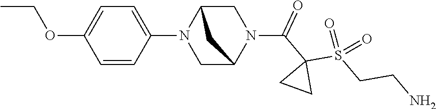 [SMILES: CCOc1ccc(N2C[C@@H]3C[C@H]2CN3C(=O)C2(S(=O)(=O)CCN)CC2)cc1] |
| 2-((2-Aminoethyl)sulfonyl)-1- (cis-5-tosylhexahydropyrrolo- [3,4-c]pyrrol-2(1H)-yl)ethan- 1-one | 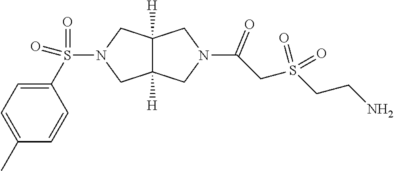 [SMILES: Cc1ccc(S(=O)(=O)N2C[C@H]3CN(C(=O)CS(=O)(=O)CCN)C[C@H]3C2)cc1] |
| (2-((2-Aminoethyl)sulfonyl)-2- fluoro-1-(cis-5-(4-fluorophenyl)- hexahydropyrrolo[3,4-c]pyrrol- 2(1H)-yl)ethan-1-one | 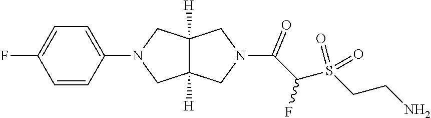 [SMILES: NCCS(=O)(=O)C(F)C(=O)N1C[C@@H]2CN(c3ccc(F)cc3)C[C@@H]2C1] |
| 2-((2-Aminoethyl)sulfonyl)-2- fluoro-1-(cis-5-(4-bromophenyl)- hexahydropyrrolo[3,4-c]pyrrol- 2(1H)-yl)ethan-1-one | 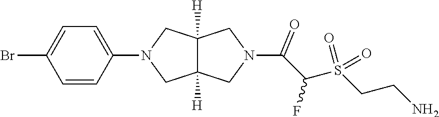 [SMILES: NCCS(=O)(=O)C(F)C(=O)N1C[C@@H]2CN(c3ccc(Br)cc3)C[C@@H]2C1] |
| 2-((2-Aminoethyl)sulfonyl)-2- fluoro-1-(cis-5-(perfluorophenyl)- hexahydropyrrolo[3,4-c]pyrrol- 2(1H)-yl)ethan-1-one | 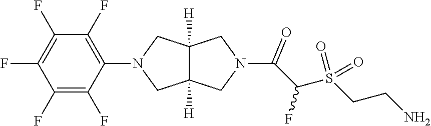 [SMILES: NCCS(=O)(=O)C(F)C(=O)N1C[C@@H]2CN(c3c(F)c(F)c(F)c(F)c3F)C[C@@H]2C1] |
| 2-((2-Aminoethyl)sulfonyl)-1- (4-(pyrimidin-2-yl)piperazin-1- yl)ethan-1-one | 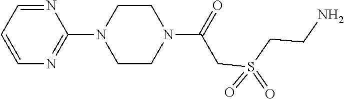 [SMILES: NCCS(=O)(=O)CC(=O)N1CCN(c2ncccn2)CC1] |
| 2-((2-Aminoethyl)sulfonyl)-1- (4-phenylpiperazin-1-yl)ethan- 1-one | 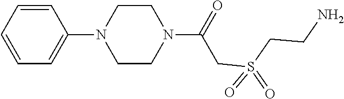 [SMILES: NCCS(=O)(=O)CC(=O)N1CCN(c2ccccc2)CC1] |
| 2-((2-Aminoethyl)sulfonyl)- 1-(cis-5-(4-ethoxyphenyl)hexa- hydropyrrolo[3,4-c]pyrrol- 2(1H)-yl)ethan-1-one | 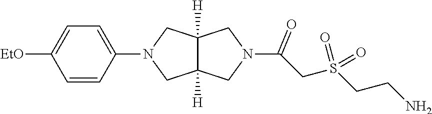 [SMILES: CCOc1ccc(N2C[C@H]3CN(C(=O)CS(=O)(=O)CCN)C[C@H]3C2)cc1] |
| 2-((2-Aminoethyl)sulfonyl)-2- fluoro-1-(4-phenylpiperazin-1- yl)ethan-1-one | 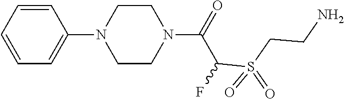 [SMILES: NCCS(=O)(=O)C(F)C(=O)N1CCN(c2ccccc2)CC1] |
| 2-((2-Aminoethyl)sulfonyl)-2- fluoro-1-(piperazin-1-yl)ethan- 1-one | 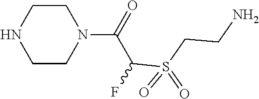 [SMILES: NCCS(=O)(=O)C(F)C(=O)N1CCNCC1] |
| 2-((2-Aminoethyl)sulfonyl)-2- fluoro-1-(4-(methylsulfonyl)- piperazin-1-yl)ethan-1-one | 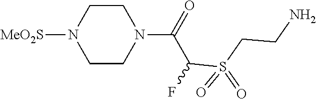 [SMILES: CS(=O)(=O)N1CCN(C(=O)C(F)S(=O)(=O)CCN)CC1] |
| 1-((1S,4S)-5-(4-((2-Aminoethyl)- sulfonyl)phenyl)-2,5-diaza- bicyclo[2.2.1]heptan-2-yl)-2- methyl propan-1-one | 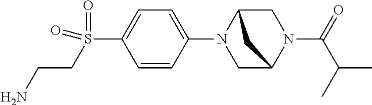 [SMILES: CC(C)C(=O)N1C[C@@H]2C[C@H]1CN2c1ccc(S(=O)(=O)CCN)cc1] |
| 4-((2-Aminoethyl)sulfonyl)- phenyl (1S,5S)-6-(4-ethoxy- phenyl)-9,9-dimethyl-3,6- diazabicyclo[3.2.2]nonane-3- carboxylate | 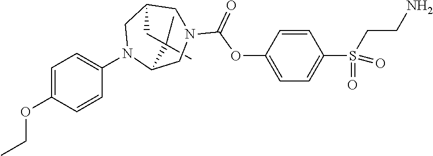 [SMILES: CCOc1ccc(N2C[C@H]3CN(C(=O)Oc4ccc(S(=O)(=O)CCN)cc4)C[C@@H]2C(C)(C)C3)cc1] |
| 1-((1S,4S)-5-(4-(((2-Aminoethyl)- sulfonyl)methyl)phenyl)-2,5- diazabicyclo[2.2.1]heptan-2- yl)-2-methylpropan-1-one | 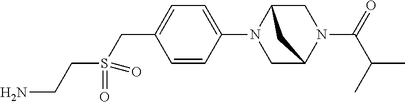 [SMILES: CC(C)C(=O)N1C[C@@H]2C[C@H]1CN2c1ccc(CS(=O)(=O)CCN)cc1] |
| (4-(((2-Aminoethyl)sulfonyl)- methyl)phenyl)((1S,5S)-6-(4- ethoxyphenyl)-9,9-dimethyl- 3,6-diazabicyclo[3.2.2]nonan- 3-yl)methanone | 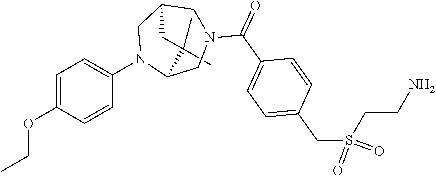 [SMILES: CCOc1ccc(N2C[C@H]3CN(C(=O)c4ccc(CS(=O)(=O)CCN)cc4)C[C@@H]2C(C)(C)C3)cc1] |
| 2-((2-Aminoethyl)sulfonyl)-1- ((1S,5S)-9,9-dimethyl-6-(4- morpholinophenyl)-3,6-diaza- bicyclo[3.2.2]nonan-3-yl)-2- fluoroethan-1-one | 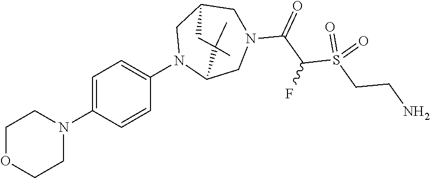 [SMILES: CC1(C)C[C@@H]2CN(C(=O)C(F)S(=O)(=O)CCN)C[C@H]1N(c1ccc(N3CCOCC3)cc1)C2] |
| 2-((2-Aminoethyl)sulfonyl)-1- (cis-3a,6a-dimethyl-5-(4-(4- (methylsulfonyl)piperazin-1- yl)phenyl)hexahydropyrrolo- [3,4-c]pyrrol-2(1H)-yl)-2- fluoroethan-1-one | 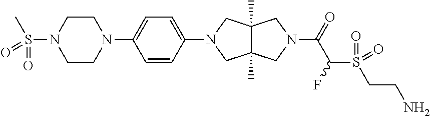 [SMILES: C[C@@]12CN(C(=O)C(F)S(=O)(=O)CCN)C[C@]1(C)CN(c1ccc(N3CCN(S(C)(=O)=O)CC3)cc1)C2] |
| 2-((2-Aminoethyl)sulfonyl)-1- (7-(4-(1,1-dioxidothiomor- pholino)phenyl)-2,7-diazaspiro- [4.4]nonan-2-yl)-2-fluoroethan- 1-one | 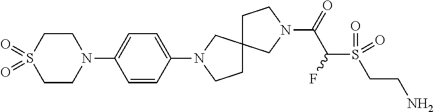 [SMILES: NCCS(=O)(=O)C(F)C(=O)N1CCC2(CCN(c3ccc(N4CCS(=O)(=O)CC4)cc3)C2)C1] |
| 2-((2-aminoethyl)sulfonyl)-1- (cis-5-(4-chlorophenyl)hexa- hydropyrrolo[3,4-c]pyrrol- 2(1H)-yl)-2-fluoroethan-1-one | 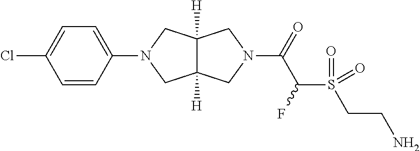 [SMILES: NCCS(=O)(=O)C(F)C(=O)N1C[C@@H]2CN(c3ccc(Cl)cc3)C[C@@H]2C1] |
| 2-((2-Aminoethyl)sulfonyl)-1- (cis-3a,6a-dimethyl-5-(4- morpholinophenyl)hexahydro- pyrrolo[3,4-c]pyrrol-2(1H)-yl)- 2-fluoroethan-1-one | 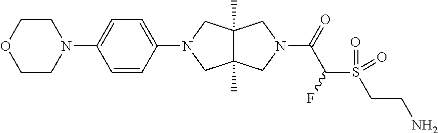 [SMILES: C[C@@]12CN(C(=O)C(F)S(=O)(=O)CCN)C[C@]1(C)CN(c1ccc(N3CCOCC3)cc1)C2] |
| 2-((2-Aminoethyl)sulfonyl)-1- (cis-5-(4-(1,1-dioxidothio- morpholino)phenyl)-3a,6a- dimethylhexahydropyrrolo- [3,4-c]pyrrol-2(1H)-yl)-2- fluoroethan-1-one | 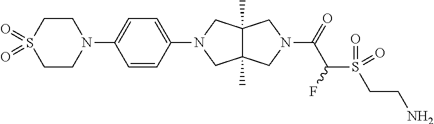 [SMILES: C[C@@]12CN(C(=O)C(F)S(=O)(=O)CCN)C[C@]1(C)CN(c1ccc(N3CCS(=O)(=O)CC3)cc1)C2] |

24. A compound in accordance with of any one of claims 1 to 23 for use as a medicament, such as for use in the treatment of a disease or medical condition mediated by LOX.

25. A compound of any one of claims 1 to 23, wherein the compound is for use in the treatment of a proliferative disease, such as cancer.

26. A compound of any one of claims 1 to 23, wherein the compound is for use in the treatment a fibrotic disease, such as liver fibrosis, lung fibrosis, kidney fibrosis, cardiac fibrosis, myelofibrosis or schleroderma.


SEQUENCE LISTING (2 sequences)

SEQ ID NO: 1 - DNA, 28 bp, Artificial Sequence
  Forward primer used for cloning of LOX cDNA into pEGFP-N1/proGFP2-N1
gagagagctagcatgcgtttcgcctggg

SEQ ID NO: 2 - DNA, 33 bp, Artificial Sequence
  Reverse primer used for cloning of LOX cDNA into pEGFP-N1/proGFP2-N1
tctctcctcgagatacggtgaaattgtgcagcc